(19) United States
(12) Patent Application Publication
(10) Pub. No.: US 2021/0280791 A1
(43) Pub. Date: Sep. 9, 2021

(54) ORGANIC SEMICONDUCTING POLYMERS

(71) Applicant: RAYNERGY TEK INCORPORATION, HSINCHU (TW)

(72) Inventors: NICOLAS BLOUIN, DARMSTADT (DE); ANGNIESZKA PRON, EASTLEIGH (GB); WILLIAM MITCHELL, CHANDLER'S FORD (GB); JONATHAN SNOW, SALISBURY (GB); IGNASI BURGUES, SOUTHAMPTON (GB); QUENTIN HUAULME, Southampton (GB)

(21) Appl. No.: 17/050,754

(22) PCT Filed: Apr. 24, 2019
(86) PCT No.: PCT/EP2019/060409

§ 371 Date: Oct. 26, 2020

(30) Foreign Application Priority Data
Apr. 27, 2018 (EP) 18169779.8

(51) Int. Cl. H01L 51/00; C08G 61/12; C07D 495/22

(52) U.S. Cl. CPC H01L 51/0036; H01L 51/0566; H01L 51/0043; H01L 51/0046; H01L 51/0047; C07D 495/22; C08G 2261/124; C08G 2261/1412; C08G 2261/148; C08G 2261/18; C08G 2261/228; C08G 2261/95; C08G 2261/94; C08G 2261/91; C08G 2261/3223; C08G 2261/3243; C08G 2261/3325; C08G 61/126

(57) ABSTRACT

The invention relates to novel organic semiconducting (OSC) polymers containing a polycyclic acceptor-donor-acceptor (A-D-A) type repeating unit, to methods for their preparation and educts or intermediates used therein, to compositions and formulations containing them, to the use of the polymers and compositions as organic semiconductors in, or for the preparation of, organic electronic (OE) devices, especially organic photovoltaic (OPV) devices, perovskite-based solar cell (PSC) devices, organic photo-detectors (OPD), organic field effect transistors (OFET) and organic light emitting diodes (OLED), and to OE, OPV, PSC, OPD, OFET and OLED devices comprising these polymers or compositions.

### TECHNICAL FIELD

The invention relates to novel organic semiconducting (OSC) polymers containing a polycyclic acceptor-donor-acceptor (A-D-A) type repeating unit, to methods for their preparation and educts or intermediates used therein, to compositions and formulations containing them, to the use of the polymers and compositions as organic semiconductors in, or for the preparation of, organic electronic (OE) devices, especially organic photovoltaic (OPV) devices, perovskite-based solar cell (PSC) devices, organic photo-detectors (OPD), organic field effect transistors (OFET) and organic light emitting diodes (OLED), and to OE, OPV, PSC, OPD, OFET and OLED devices comprising these polymers or compositions.

### BACKGROUND

In recent years, there has been development of organic semiconducting (OSC) materials in order to produce more versatile, lower cost electronic devices. Such materials find application in a wide range of devices or apparatus, including organic field effect transistors (OFETs), organic light emitting diodes (OLEDs), organic photodetectors (OPDs), organic photovoltaic (OPV) cells, perovskite-based solar cell (PSC) devices, sensors, memory elements and logic circuits to name just a few. The organic semiconducting materials are typically present in the electronic device in the form of a thin layer, for example of between 50 and 300 nm thickness.

One particular area of importance is organic photovoltaics (OPV). OSC polymers have found use in OPVs as they allow devices to be manufactured by solution-processing techniques such as spin casting, dip coating or ink jet printing. Solution processing can be carried out cheaper and on a larger scale compared to the evaporative techniques used to make inorganic thin film devices. Currently, polymer based photovoltaic devices are achieving efficiencies above 10%.

Another particular area of importance is OFETs. The performance of OFET devices is principally based upon the charge carrier mobility of the semiconducting material and the current on/off ratio, so the ideal semiconductor should have a low conductivity in the off state, combined with high charge carrier mobility (>1×10⁻³ cm²V⁻¹ s⁻¹). In addition, it is important that the semiconducting material is stable to oxidation i.e. it has a high ionisation potential, as oxidation leads to reduced device performance. Further requirements for the semiconducting material are good processibility, especially for large-scale production of thin layers and desired patterns, and high stability, film uniformity and integrity of the organic semiconductor layer.

Organic photodetectors (OPDs) are a further particular area of importance, for which OSC light-absorbing polymers offer the hope of allowing efficient devices to be produced by solution-processing technologies, such as spin casting, dip coating or ink jet printing, to name a few only.

The photosensitive layer in an OPV or OPD device is usually composed of at least two materials, a p-type semiconductor, which is typically a conjugated OSC polymer, an oligomer or a defined molecular unit, and an n-type semiconductor, which is typically a fullerene or substituted fullerene, graphene, a metal oxide, or quantum dots.

However, the OSC materials disclosed in prior art for use in OE devices have several drawbacks. They are often difficult to synthesize or purify (fullerenes), and/or do not absorb light strongly in the near IR spectrum >700 nm. In addition, other OSC materials do not often form a favourable morphology and/or donor phase miscibility for use in organic photovoltaics or organic photodetectors.

Therefore there is still a need for OSC materials for use in OE devices like OPVs, PSCs, OPDs and OFETs, which have advantageous properties, in particular good processability, a high solubility in organic solvents, good structural organization and film-forming properties. In addition, the OSC materials should be easy to synthesize, especially by methods suitable for mass production. For use in OPV cells, the OSC materials should especially have a low bandgap, which enables improved light harvesting by the photoactive layer and can lead to higher cell efficiencies, high stability and long lifetime. For use in OFETs the OSC materials should especially have high charge-carrier mobility, high on/off ratio in transistor devices, high oxidative stability and long lifetime.

In particular there is a need for n-type OSC polymers. These can be used for example in all-polymer photodiodes or solar cells, where they enable better control of the morphology thus leading to higher power conversion efficiency (PCE) and better thermal stability of the device. However, despite the recent progress of organic photovoltaics there is still a lack of n-type polymers showing satisfactory performance, like for example sufficiently high PCE in OPV devices and sufficiently high external quantum efficiency (EQE) in OPD devices.

It was an aim of the present invention to provide new OSC compounds, especially n-type OSC polymers, which can overcome the drawbacks of the OSCs from prior art, and which provide one or more of the above-mentioned advantageous properties, especially easy synthesis by methods suitable for mass production, good processability, high stability, long lifetime in OE devices, good solubility in organic solvents, high charge carrier mobility, and a low bandgap. Another aim of the invention was to extend the pool of n-type OSC polymers available to the expert. Other aims of the present invention are immediately evident to the expert from the following detailed description.

The inventors of the present invention have found that one or more of the above aims can be achieved by providing OSC polymers as disclosed and claimed hereinafter. These polymers comprise a repeating unit that consists of an electron donating polycyclic core that is flanked by two electron withdrawing moieties, and optionally further comprises one or more aromatic or heteroaromatic spacer groups, which are located between the polycyclic core and the electron withdrawing moieties and which can be electron withdrawing or electron donating relative to the polycyclic core. As a result the repeating unit has an acceptor-donor-acceptor (A-D-A) structure.

It was surprisingly found that such OSC polymers can be used as organic semiconductors in OE devices, especially as electron acceptor or n-type OSC component, for example in a donor-acceptor blend in the photoactive layer of an organic photodiode or solar cell, where they show superior performance compared to OSC polymers of prior art.

Zhang et al., Angew. Chem. Int. Ed. 2017, 56, 13503 discloses the n-type polymer PZ1, which has an A-D-A type repeating unit, and its use as polymer acceptor in an all-polymer solar cell:

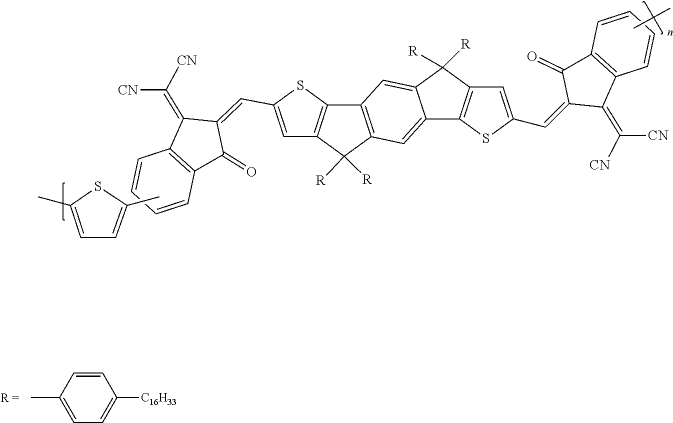

However, the polymers as disclosed and claimed hereinafter have hitherto not been disclosed in prior art for use as n-type OSCs.

### SUMMARY

The invention relates to a polymer comprising one or more repeating units of formula I

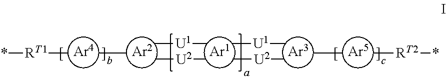

wherein the individual radicals, independently of each other and on each occurrence identically or differently, have the following meanings each pair of U¹ and U² is attached to two directly adjacent C atoms in a ring Ar¹, Ar² or Ar³ respectively, and in each of said pairs of U¹ and U² one of U¹ and U² is a single bond and the other is CR¹R², SiR¹R², GeR¹R², C═CR¹R², C═O or NR¹, so that each pair of U¹ and U² is forming a five-membered unsaturated ring together with the C atoms to which they are attached,
R^{T1}, R^{T2} a group of formula T

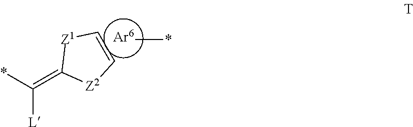

Z¹, Z² C═O, C═CX¹X², SO₂,
X¹, X² CN, C(═O)R^{a},
Ar¹, Ar², Ar³ arylene or heteroarylene which has from 5 to 20 ring atoms, is mono- or polycyclic, optionally contains fused rings, and is unsubstituted or substituted by one or more identical or different groups L,
Ar⁴, Ar⁵ —CY¹═CY²—, —C≡C—, or arylene or heteroarylene which has from 5 to 20 ring atoms, is mono- or polycyclic, optionally contains fused rings, and is unsubstituted or substituted by one or more identical or different groups L,
Ar⁶ arylene or heteroarylene which has from 5 to 20 ring atoms, is mono- or polycyclic, optionally contains fused rings, and is unsubstituted or substituted by one or more identical or different groups L,
Y¹, Y²H, F, Cl or CN,
R¹, R²H, F, Cl, CN, or straight-chain, branched or cyclic alkyl with 1 to 30, preferably 1 to 20, C atoms, in which one or more CH₂ groups are each optionally replaced by —O—, —S—, —C(═O)—, —C(═S)—, —C(═O)—O—, —O—C(═O)—, —NR⁰—, —SiR⁰R⁰⁰—, —CF₂—, —CR⁰═CR⁰⁰—, —CY¹═CY²— or —C≡C— in such a manner that O and/or S atoms are not linked directly to one another, and in which one or more H atoms are each optionally replaced by F, Cl, Br, I or CN, and in which one or more CH₂ or CH₃ groups are each optionally replaced by a cationic or anionic group, or aryl, heteroaryl, arylalkyl, heteroarylalkyl, aryloxy or heteroaryloxy, wherein each of the aforementioned cyclic groups has 5 to 20 ring atoms, is mono- or polycyclic, optionally contains fused rings, and is unsubstituted or substituted by one or more identical or different groups L,
   and the pair of R¹ and R², together with the C, Si or Ge atom to which they are attached, may also form a spiro group with 5 to 20 ring atoms which is mono- or polycyclic, optionally contains fused rings, and is unsubstituted or substituted by one or more identical or different groups L,
R^{a} R⁰ or aryl or heteroaryl, each having from 4 to 30 ring atoms, optionally containing fused rings and being unsubstituted or substituted with one or more groups L,
L F, Cl, —NO₂, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —CF₃, —SF₅, or optionally substituted silyl, or carbyl or hydrocarbyl with 1 to 30, preferably 1 to 20 C atoms that is optionally substituted and optionally comprises one or more hetero atoms, preferably F, —CN, R⁰, —OR⁰, —SR⁰, —C(═O)—R⁰, —C(═O)—OR⁰, —O—C(═O)—R⁰, —O—C(═O)—OR⁰, —C(═O)—NHR⁰, or —C(═O)—NR⁰R⁰⁰,
L′ H or one of the meanings given for L,
R⁰, R⁰⁰H or straight-chain or branched alkyl with 1 to 20, preferably 1 to 12, C atoms that is optionally fluorinated,
X⁰ halogen, preferably F or Cl,
a 0, 1, 2 or 3,
c 0, 1, 2 or 3,

characterized in that, if a>0, Ar¹ contains at least one thiophene ring.

The invention further relates to novel synthesis methods for preparing repeating units of formula I and polymers comprising them, and novel intermediates used therein.

The invention further relates to a polymer according to the present invention which comprises two or more repeating units, at least one of which is selected from formula I or its subformulae.

The invention further relates to a polymer compound according to the present invention comprising one or more, preferably two or more, units of formula I or its subformulae, and one or more co-units selected from the group consisting of C═C double bonds that are optionally substituted by F, Cl or CN, C≡C triple bonds, and arylene or heteroarylene units that are different from formula I and its subformulae, have from 5 to 20 ring atoms, are mono- or polycyclic, do optionally contain fused rings, and are unsubstituted or substituted by one or more identical or different groups L as defined in formula I.

The invention further relates to a polymer as described above wherein one or more of these additional arylene or heteroarylene units have electron donor property. The invention further relates to a polymer as described above wherein one or more of these additional arylene or heteroarylene units have electron acceptor property.

The invention further relates to a polymer compound according to the present invention comprising, alternatively or addition to the co-units selected from optionally substituted C═C double bonds, C≡C triple bonds, and arylene or heteroarylene units, one or more co-units selected from alkylene, (hetero)arylene-alkylene and (hetero)arylene-alkylene-(hetero)arylene units.

The invention further relates to a monomer comprising a divalent unit of formula I or its subformulae, optionally further comprising one or more additional arylene or heteroarylene units, and further comprising one or more reactive groups which can be reacted to form a polymer according to the present invention as described above and below.

The invention further relates to the use of a polymer according to the present invention as electron acceptor or n-type semiconductor.

The invention further relates to the use of a polymer according to the present invention as electron acceptor component in a semiconducting material, formulation, polymer blend, device or component of a device.

The invention further relates to a semiconducting material, formulation, polymer blend, device or component of a device comprising a polymer according to the present invention as electron acceptor component, and preferably further comprising one or more compounds having electron donor properties.

The invention further relates to a composition, which may also be a polymer blend, comprising one or more polymers according to the present invention, and further comprising one or more additional compounds selected from compounds having one or more of semiconducting, charge transport, hole or electron transport, hole or electron blocking, electrically conducting, photoconducting or light emitting properties.

The invention further relates to a composition comprising one or more polymers according to the present invention, and further comprising one or more p-type organic semiconductors, preferably selected from conjugated polymers.

The invention further relates to a composition comprising a first n-type semiconductor which is a polymer according to the present invention, a second n-type semiconductor, which is preferably a fullerene or fullerene derivative, a non-fullerene acceptor small molecule, or an n-type conjugated polymer, and a p-type semiconductor, which is a conjugated polymer.

The invention further relates to a bulk heterojunction (BHJ) formed from a composition comprising a polymer according to the present invention as electron acceptor or n-type semiconductor, and one or more compounds which are electron donor or p-type semiconductors and are preferably selected from conjugated polymers.

The invention further relates to a formulation comprising one or more polymers or a composition according to the present invention, and further comprising one or more solvents, preferably selected from organic solvents.

The invention further relates to an organic semiconducting formulation comprising one or more polymers according to the present invention, and further comprising one or more organic binders or precursors thereof, preferably having a permittivity ε at 1,000 Hz and 20° C. of 3.3 or less, and optionally one or more solvents preferably selected from organic solvents.

The invention further relates to an optical, electronic, optoelectronic, electroluminescent or photoluminescent device, or a component thereof, or an assembly comprising it, which is prepared using a formulation according to the present invention.

The invention further relates to the use of a polymer or composition according to the present invention as semiconducting, charge transport, electrically conducting, photoconducting or light emitting material, or in an optical, electronic, optoelectronic, electroluminescent or photoluminescent device, or in a component of such a device or in an assembly comprising such a device or component

The invention further relates to a semiconducting, charge transport, electrically conducting, photoconducting or light emitting material comprising a polymer or composition according to the present invention.

The invention further relates to an optical, electronic, optoelectronic, electroluminescent or photoluminescent device, or a component thereof, or an assembly comprising it, which comprises a polymer or composition according to the present invention, or comprises a semiconducting, charge transport, electrically conducting, photoconducting or light emitting material according to the present invention.

The optical, electronic, optoelectronic, electroluminescent and photoluminescent device include, without limitation, organic field effect transistors (OFET), organic thin film transistors (OTFT), organic light emitting diodes (OLED), organic light emitting transistors (OLET), organic photovoltaic devices (OPV), organic photodetectors (OPD), organic solar cells, dye-sensitized solar cells (DSSC), perovskite-based solar cells (PSC), laser diodes, Schottky diodes, photoconductors and photodetectors.

Preferred devices are OFETs, OTFTs, OPVs, PSCs, OPDs and OLEDs, in particular OTFTs, PSCs, OPDs and bulk heterojunction (BHJ) OPVs or inverted BHJ OPVs.

Further preferred is the use of a polymer or composition according to the present invention as dye in a DSSC or a PSC. Further preferred is a DSSC or PSC comprising a polymer or composition according to the present invention.

The component of the above devices includes, without limitation, charge injection layers, charge transport layers, interlayers, planarising layers, antistatic films, polymer electrolyte membranes (PEM), conducting substrates and conducting patterns.

The assembly comprising such a device or component includes, without limitation, integrated circuits (IC), radio frequency identification (RFID) tags or security markings or security devices containing them, flat panel displays or backlights thereof, electrophotographic devices, electrophotographic recording devices, organic memory devices, sensor devices, biosensors and biochips.

In addition the polymers, compositions and formulations of the present invention can be used as electrode materials in batteries and in components or devices for detecting and discriminating DNA sequences.

The invention further relates to a bulk heterojunction which comprises, or is being formed from, a composition comprising one or more polymers according to the present invention and one or more p-type organic semiconductors that are preferably selected from conjugated polymers. The invention further relates to a bulk heterojunction (BHJ) OPV or OPD device or inverted BHJ OPV or OPD device, comprising such a bulk heterojunction.

### Terms and Definitions

Unless stated otherwise, in the units, polymers and compounds according to the present invention the electron withdrawing groups R^{T1} and R^{T2} are understood to be electron withdrawing relative to the polycyclic core.

As used herein, the terms “indaceno-type group” and “indaceno group” mean a group comprising two cyclopentadiene rings, or heterocyclic or vinylidene derivatives thereof, that are fused to a central aromatic or heteroaromatic aromatic ring Ar, and which can have cis- or trans-configuration, as exemplarily shown below

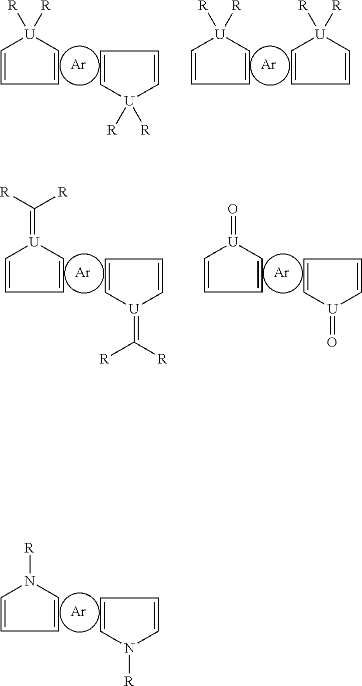

wherein U is e.g. C, Si or Ge and R is a carbyl or hydrocarbyl group.

Unless stated otherwise, in a polymer according to the present invention the units of formula I have electron acceptor property (“acceptor units”).

As used herein, the terms “donor” or “donating” and “acceptor” or “accepting” will be understood to mean an electron donor or electron acceptor, respectively. “Electron donor” will be understood to mean a chemical entity that donates electrons to another compound or another group of atoms of a compound. “Electron acceptor” will be understood to mean a chemical entity that accepts electrons transferred to it from another compound or another group of atoms of a compound. See also International Union of Pure and Applied Chemistry, Compendium of Chemical Technology, Gold Book, Version 2.3.2, 19. August 2012, pages 477 and 480.

As used herein, the term “donor unit” will be understood to mean a unit, preferably a conjugated arylene or heteroarylene unit, which has an electron donating or electron pushing property towards a neighboured conjugated unit. The term “acceptor unit” will be understood to mean a unit, preferably a conjugated arylene or heteroarylene unit, which has an electron accepting or electron withdrawing property towards a neighboured conjugated unit. The term “spacer unit” will be understood to mean a unit which can be conjugated or non-conjugated and is located between a donor and an acceptor unit, and is preferably selected such that it does not have electron accepting property towards a neighboured donor unit.

As used herein, the term “n-type” or “n-type semiconductor” will be understood to mean an extrinsic semiconductor in which the conduction electron density is in excess of the mobile hole density, and the term “p-type” or “p-type semiconductor” will be understood to mean an extrinsic semiconductor in which mobile hole density is in excess of the conduction electron density (see also, J. Thewlis, Concise Dictionary of Physics, Pergamon Press, Oxford, 1973).

As used herein, the term “conjugated” will be understood to mean a compound (for example a polymer) that contains mainly C atoms with sp²-hybridization (or optionally also sp-hybridization), and wherein these C atoms may also be replaced by hetero atoms. In the simplest case this is for example a compound with alternating C—C single and double (or triple) bonds, but is also inclusive of compounds with aromatic units like for example 1,4-phenylene. The term “mainly” in this connection will be understood to mean that a compound with naturally (spontaneously) occurring defects, or with defects included by design, which may lead to interruption of the conjugation, is still regarded as a conjugated compound.

Based on this definition of “conjugated” the repeating units of formula I, and the polymers comprising them, are not fully conjugated due to the presence of the groups of formula T which interrupt conjugation, since the electrons cannot move (as shown in the structure below by the arrows) from the electron withdrawing group of formula T to the polycyclic core.

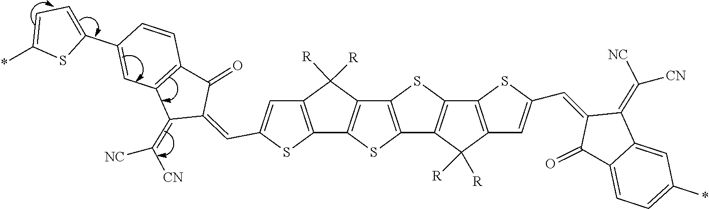

As used herein, the term “polymer” will be understood to mean a molecule of high relative molecular mass, the structure of which essentially comprises multiple repetitions of units derived, actually or conceptually, from molecules of low relative molecular mass (Pure Appl. Chem., 1996, 68, 2291). The term “oligomer” will be understood to mean a molecule of intermediate relative molecular mass, the structure of which essentially comprises a small plurality of units derived, actually or conceptually, from molecules of lower relative molecular mass (Pure Appl. Chem., 1996, 68, 2291). In a preferred meaning as used herein present invention a polymer will be understood to mean a compound having >1, i.e. at least 2 repeat units, preferably ≥5, very preferably ≥10, repeat units, and an oligomer will be understood to mean a compound with >1 and <10, preferably <5, repeat units.

Further, as used herein, the term “polymer” will be understood to mean a molecule that encompasses a backbone (also referred to as “main chain”) of one or more distinct types of repeat units (the smallest constitutional unit of the molecule) and is inclusive of the commonly known terms “oligomer”, “copolymer”, “homopolymer”, “random polymer” and the like. Further, it will be understood that the term polymer is inclusive of, in addition to the polymer itself, residues from initiators, catalysts and other elements attendant to the synthesis of such a polymer, where such residues are understood as not being covalently incorporated thereto. Further, such residues and other elements, while normally removed during post polymerization purification processes, are typically mixed or co-mingled with the polymer such that they generally remain with the polymer when it is transferred between vessels or between solvents or dispersion media.

As used herein, in a formula showing a polymer or a repeat unit an asterisk (*) will be understood to mean a chemical linkage, usually a single bond, to an adjacent unit or to a terminal group in the polymer backbone. In a ring, like for example a benzene or thiophene ring, an asterisk (*) will be understood to mean a C atom that is fused to an adjacent ring.

As used herein, in a formula showing a ring, a polymer or a repeat unit a dashed line (-----) will be understood to mean a single bond.

As used herein, the terms “repeat unit”, “repeating unit” and “monomeric unit” are used interchangeably and will be understood to mean the constitutional repeating unit (CRU), which is the smallest constitutional unit the repetition of which constitutes a regular macromolecule, a regular oligomer molecule, a regular block or a regular chain (Pure Appl. Chem., 1996, 68, 2291). As further used herein, the term “unit” will be understood to mean a structural unit which can be a repeating unit on its own, or can together with other units form a constitutional repeating unit.

As used herein, a “terminal group” will be understood to mean a group that terminates a polymer backbone. The expression “in terminal position in the backbone” will be understood to mean a divalent unit or repeat unit that is linked at one side to such a terminal group and at the other side to another repeat unit. Such terminal groups include endcap groups, or reactive groups that are attached to a monomer forming the polymer backbone which did not participate in the polymerization reaction, like for example a group having the meaning of R³¹ or R³² as defined below.

As used herein, the term “endcap group” will be understood to mean a group that is attached to, or replacing, a terminal group of the polymer backbone. The endcap group can be introduced into the polymer by an endcapping process. Endcapping can be carried out for example by reacting the terminal groups of the polymer backbone with a monofunctional compound (“endcapper”) like for example an alkyl- or arylhalide, an alkyl- or arylstannane or an alkyl- or arylboronate. The endcapper can be added for example after the polymerization reaction. Alternatively the endcapper can be added in situ to the reaction mixture before or during the polymerization reaction. In situ addition of an endcapper can also be used to terminate the polymerization reaction and thus control the molecular weight of the forming polymer. Typical endcap groups are for example H, phenyl and lower alkyl.

As used herein, the term “small molecule” will be understood to mean a monomeric compound which typically does not contain a reactive group by which it can be reacted to form a polymer, and which is designated to be used in monomeric form. In contrast thereto, the term “monomer” unless stated otherwise will be understood to mean a monomeric compound that carries one or more reactive functional groups by which it can be reacted to form a polymer.

As used herein, the term “leaving group” will be understood to mean an atom or group (which may be charged or uncharged) that becomes detached from an atom in what is considered to be the residual or main part of the molecule taking part in a specified reaction (see also Pure Appl. Chem., 1994, 66, 1134).

As used herein, unless stated otherwise the molecular weight is given as the number average molecular weight Mₙ or weight average molecular weight M_{W}, which is determined by gel permeation chromatography (GPC) against polystyrene standards in eluent solvents such as tetrahydrofuran, trichloromethane (TCM, chloroform), chlorobenzene or 1,2,4-trichloro-benzene. Unless stated otherwise, chlorobenzene is used as solvent. The degree of polymerization, also referred to as total number of repeat units, n, will be understood to mean the number average degree of polymerization given as n=Mₙ/M_{U}, wherein Mₙ is the number average molecular weight and M_{U} is the molecular weight of the single repeat unit, see J. M. G. Cowie, Polymers: Chemistry & Physics of Modern Materials, Blackie, Glasgow, 1991.

As used herein, the term “carbyl group” will be understood to mean any monovalent or multivalent organic moiety which comprises at least one carbon atom either without any non-carbon atoms (like for example —C≡C—), or optionally combined with at least one non-carbon atom such as B, N, O, S, P, Si, Se, As, Te or Ge (for example carbonyl etc.).

As used herein, the term “hydrocarbyl group” will be understood to mean a carbyl group that does additionally contain one or more H atoms and optionally contains one or more hetero atoms like for example B, N, O, S, P, Si, Se, As, Te or Ge.

As used herein, the term “hetero atom” will be understood to mean an atom in an organic compound that is not a H- or C-atom, and preferably will be understood to mean B, N, O, S, P, Si, Se, Sn, As, Te or Ge.

A carbyl or hydrocarbyl group comprising a chain of 3 or more C atoms may be straight-chain, branched and/or cyclic, and may include spiro-connected and/or fused rings.

Preferred carbyl and hydrocarbyl groups include alkyl, alkoxy, thioalkyl, alkylcarbonyl, alkoxycarbonyl, alkylcarbonyloxy and alkoxycarbonyloxy, each of which is optionally substituted and has up to 40, preferably up to 25, very preferably up to 18 C atoms, furthermore optionally substituted aryl or aryloxy having 6 to 40, preferably 6 to 25 C atoms, furthermore alkylaryloxy, arylcarbonyl, aryloxycarbonyl, arylcarbonyloxy and aryloxycarbonyloxy, each of which is optionally substituted and has 1 to 40, preferably 6 to 40 C atoms, wherein each of these groups optionally contains one or more hetero atoms, preferably selected from B, N, O, S, P, Si, Se, As, Te and Ge.

Further preferred carbyl and hydrocarbyl group include for example: a C₁-C₄₀ alkyl group, a C₁-C₄₀ fluoroalkyl group, a C₁-C₄₀ alkoxy or oxaalkyl group, a C₂-C₄₀ alkenyl group, a C₂-C₄₀ alkynyl group, a C₃-C₄₀ allyl group, a C₄-C₄₀ alkyldienyl group, a C₄-C₄₀ polyenyl group, a C₂-C₄₀ ketone group, a C₂-C₄₀ ester group, a C₆-C₁₈ aryl group, a C₆-C₄₀ alkylaryl group, a C₆-C₄₀ arylalkyl group, a C₄-C₄₀ cycloalkyl group, a C₄-C₄₀ cycloalkenyl group, and the like. Preferred among the foregoing groups are a C₁-C₂₀ alkyl group, a C₁-C₂₀ fluoroalkyl group, a C₂-C₂₀ alkenyl group, a C₂-C₂₀ alkynyl group, a C₃-C₂₀ allyl group, a C₄-C₂₀ alkyldienyl group, a C₂-C₂₀ ketone group, a C₂-C₂₀ ester group, a C₆-C₁₂ aryl group, and a C₄-C₂₀ polyenyl group, respectively.

Also included are combinations of groups having carbon atoms and groups having hetero atoms, like e.g. an alkynyl group, preferably ethynyl, that is substituted with a silyl group, preferably a trialkylsilyl group.

The carbyl or hydrocarbyl group may be an acyclic group or a cyclic group. Where the carbyl or hydrocarbyl group is an acyclic group, it may be straight-chain or branched. Where the carbyl or hydrocarbyl group is a cyclic group, it may be a non-aromatic carbocyclic or heterocyclic group, or an aryl or heteroaryl group.

A non-aromatic carbocyclic group as referred to above and below is saturated or unsaturated and preferably has 4 to 30 ring C atoms. A non-aromatic heterocyclic group as referred to above and below preferably has 4 to 30 ring C atoms, wherein one or more of the C ring atoms are each optionally replaced by a hetero atom, preferably selected from N, O, P, S, Si and Se, or by a —S(O)— or —S(O)₂— group. The non-aromatic carbo- and heterocyclic groups are mono- or polycyclic, may also contain fused rings, preferably contain 1, 2, 3 or 4 fused or unfused rings, and are optionally substituted with one or more groups L.

L is selected from F, Cl, —CN, —NO₂, —NC, —NCO, —NCS, —OCN, —SCN, —R⁰, —OR⁰, —SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —CF₃, —SF₅, or optionally substituted silyl, or carbyl or hydrocarbyl with 1 to 30, preferably 1 to 20 C atoms that is optionally substituted and optionally comprises one or more hetero atoms, wherein X⁰ is halogen, preferably F or Cl, and R⁰, R⁰⁰ each independently denote H or straight-chain or branched alkyl with 1 to 20, preferably 1 to 12 C atoms that is optionally fluorinated.

Preferably L is selected from F, —CN, R⁰, —OR⁰, —SR⁰, —C(═O)—R⁰, —C(═O)—OR⁰, —O—C(═O)—R⁰, —O—C(═O)—OR⁰, —C(═O)—NHR⁰ and —C(═O)—NR⁰R⁰⁰.

Further preferably L is selected from F or alkyl, alkoxy, oxaalkyl, thioalkyl, fluoroalkyl, fluoroalkoxy, alkylcarbonyl, alkoxycarbonyl, with 1 to 16 C atoms, or alkenyl or alkynyl with 2 to 16 C atoms.

Preferred non-aromatic carbocyclic or heterocyclic groups are tetrahydrofuran, indane, pyran, pyrrolidine, piperidine, cyclopentane, cyclohexane, cycloheptane, cyclopentanone, cyclohexanone, dihydro-furan-2-one, tetrahydro-pyran-2-one and oxepan-2-one.

An aryl group as referred to above and below preferably has 4 to 30, very preferably 5 to 20, ring C atoms, is mono- or polycyclic and may also contain fused rings, preferably contains 1, 2, 3 or 4 fused or unfused rings, and is optionally substituted with one or more groups L as defined above.

A heteroaryl group as referred to above and below preferably has 4 to 30, very preferably 5 to 20, ring C atoms, wherein one or more of the ring C atoms are replaced by a hetero atom, preferably selected from N, O, S, Si and Se, is mono- or polycyclic and may also contain fused rings, preferably contains 1, 2, 3 or 4 fused or unfused rings, and is optionally substituted with one or more groups L as defined above.

An arylalkyl or heteroarylalkyl group as referred to above and below preferably denotes —(CH₂)ₐ-aryl or —(CH₂)ₐ-heteroaryl, wherein a is an integer from 1 to 6, preferably 1, and “aryl” and “heteroaryl” have the meanings given above and below. A preferred arylalkyl group is benzyl which is optionally substituted by L.

As used herein, “arylene” will be understood to mean a divalent aryl group, and “heteroarylene” will be understood to mean a divalent heteroaryl group, including all preferred meanings of aryl and heteroaryl as given above and below.

Preferred aryl and heteroaryl groups are phenyl in which, in addition, one or more CH groups may each be replaced by N, naphthalene, thiophene, selenophene, thienothiophene, dithienothiophene, fluorene and oxazole, all of which can be unsubstituted, mono- or polysubstituted with L as defined above. Very preferred aryl and heteroaryl groups are selected from phenyl, pyrrole, preferably N-pyrrole, furan, pyridine, preferably 2- or 3-pyridine, pyrimidine, pyridazine, pyrazine, triazole, tetrazole, pyrazole, imidazole, isothiazole, thiazole, thiadiazole, isoxazole, oxazole, oxadiazole, thiophene, preferably 2-thiophene, selenophene, preferably 2-selenophene, 2,5-dithiophene-2′,5′-diyl, thieno[3,2-b]thiophene, thieno[2,3-b]thiophene, furo[3,2-b]furan, furo[2,3-b]furan, seleno[3,2-b]selenophene, seleno[2,3-b]selenophene, thieno[3,2-b]selenophene, thieno[3,2-b]furan, indole, isoindole, benzo[b]furan, benzo[b]thiophene, benzo[1,2-b;4,5-b′]dithiophene, benzo[2,1-b;3,4-b′]dithiophene, quinole, 2-methylquinole, isoquinole, quinoxaline, quinazoline, benzotriazole, benzimidazole, benzothiazole, benzisothiazole, benzisoxazole, benzoxadiazole, benzoxazole, benzothiadiazole, 4H-cyclopenta[2,1-b;3,4-b′]dithiophene, 7H-3,4-dithia-7-sila-cyclopenta[a]pentalene, all of which can be unsubstituted, mono- or polysubstituted with L as defined above. Further examples of aryl and heteroaryl groups are those selected from the groups shown hereinafter.

An alkyl group or an alkoxy group, i.e., where the terminal CH₂ group is replaced by —O—, can be straight-chain or branched. Particularly preferred straight-chains have 2, 3, 4, 5, 6, 7, 8, 12 or 16 carbon atoms and accordingly denote preferably ethyl, propyl, butyl, pentyl, hexyl, heptyl, octyl, dodecyl or hexadecyl, ethoxy, propoxy, butoxy, pentoxy, hexoxy, heptoxy, octoxy, dodecoxy or hexadecoxy, furthermore methyl, nonyl, decyl, undecyl, tridecyl, tetradecyl, pentadecyl, nonoxy, decoxy, undecoxy, tridecoxy or tetradecoxy, for example.

An alkenyl group, i.e., wherein one or more CH₂ groups are each replaced by —CH═CH— can be straight-chain or branched. It is preferably straight-chain, has 2 to 10 C atoms and accordingly is preferably vinyl, prop-1-, or prop-2-enyl, but-1-, 2- or but-3-enyl, pent-1-, 2-, 3- or pent-4-enyl, hex-1-, 2-, 3-, 4- or hex-5-enyl, hept-1-, 2-, 3-, 4-, 5- or hept-6-enyl, oct-1-, 2-, 3-, 4-, 5-, 6- or oct-7-enyl, non-1-, 2-, 3-, 4-, 5-, 6-, 7- or non-8-enyl, dec-1-, 2-, 3-, 4-, 5-, 6-, 7-, 8- or dec-9-enyl.

Especially preferred alkenyl groups are C₂-C₇-1E-alkenyl, C₄-C₇-3E-alkenyl, C₅-C₇-4-alkenyl, C₆-C₇-5-alkenyl and C₇-6-alkenyl, in particular C₂-C₇-1E-alkenyl, C₄-C₇-3E-alkenyl and C₅-C₇-4-alkenyl. Examples for particularly preferred alkenyl groups are vinyl, 1E-propenyl, 1E-butenyl, 1E-pentenyl, 1E-hexenyl, 1E-heptenyl, 3-butenyl, 3E-pentenyl, 3E-hexenyl, 3E-heptenyl, 4-pentenyl, 4Z-hexenyl, 4E-hexenyl, 4Z-heptenyl, 5-hexenyl, 6-heptenyl and the like. Groups having up to 5 C atoms are generally preferred.

An oxaalkyl group, i.e., where one CH₂ group is replaced by —O—, can be straight-chain. Particularly preferred straight-chains are 2-oxapropyl (=methoxymethyl), 2-(=ethoxymethyl) or 3-oxabutyl (=2-methoxyethyl), 2-, 3-, or 4-oxapentyl, 2-, 3-, 4-, or 5-oxahexyl, 2-, 3-, 4-, 5-, or 6-oxaheptyl, 2-, 3-, 4-, 5-, 6- or 7-oxaoctyl, 2-, 3-, 4-, 5-, 6-, 7- or 8-oxanonyl or 2-, 3-, 4-, 5-, 6-, 7-, 8- or 9-oxadecyl, for example.

In an alkyl group wherein one CH₂ group is replaced by —O— and one CH₂ group is replaced by —C(O)—, these radicals are preferably neighboured. Accordingly these radicals together form a carbonyloxy group —C(O)—O— or an oxycarbonyl group —O—C(O)—. Preferably this group is straight-chain and has 2 to 6 C atoms. It is accordingly preferably acetyloxy, propionyloxy, butyryloxy, pentanoyloxy, hexanoyloxy, acetyloxymethyl, propionyloxymethyl, butyryloxymethyl, pentanoyloxymethyl, 2-acetyloxyethyl, 2-propionyloxyethyl, 2-butyryloxyethyl, 3-acetyloxypropyl, 3-propionyloxypropyl, 4-acetyloxybutyl, methoxycarbonyl, ethoxycarbonyl, propoxycarbonyl, butoxycarbonyl, pentoxycarbonyl, methoxycarbonylmethyl, ethoxycarbonylmethyl, propoxycarbonylmethyl, butoxycarbonylmethyl, 2-(methoxycarbonyl)ethyl, 2-(ethoxycarbonyl)ethyl, 2-(propoxycarbonyl)ethyl, 3-(methoxycarbonyl)propyl, 3-(ethoxycarbonyl)propyl or 4-(methoxycarbonyl)-butyl.

An alkyl group wherein two or more CH₂ groups are replaced by —O— and/or —C(O)O— can be straight-chain or branched. It is preferably straight-chain and has 3 to 12 C atoms. Accordingly, it is preferably bis-carboxy-methyl, 2,2-bis-carboxy-ethyl, 3,3-bis-carboxy-propyl, 4,4-bis-carboxy-butyl, 5,5-bis-carboxy-pentyl, 6,6-bis-carboxy-hexyl, 7,7-bis-carboxy-heptyl, 8,8-bis-carboxy-octyl, 9,9-bis-carboxy-nonyl, 10,10-bis-carboxy-decyl, bis-(methoxycarbonyl)-methyl, 2,2-bis-(methoxycarbonyl)-ethyl, 3,3-bis-(methoxycarbonyl)-propyl, 4,4-bis-(methoxycarbonyl)-butyl, 5,5-bis-(methoxycarbonyl)-pentyl, 6,6-bis-(methoxycarbonyl)-hexyl, 7,7-bis-(methoxycarbonyl)-heptyl, 8,8-bis-(methoxycarbonyl)-octyl, bis-(ethoxycarbonyl)-methyl, 2,2-bis-(ethoxycarbonyl)-ethyl, 3,3-bis-(ethoxycarbonyl)-propyl, 4,4-bis-(ethoxycarbonyl)-butyl or 5,5-bis-(ethoxycarbonyl)-hexyl.

A thioalkyl group, i.e., where one CH₂ group is replaced by —S—, is preferably straight-chain thiomethyl (—SCH₃), 1-thioethyl (—SCH₂CH₃), 1-thiopropyl (═—SCH₂CH₂CH₃), 1-(thiobutyl), 1-(thiopentyl), 1-(thiohexyl), 1-(thioheptyl), 1-(thiooctyl), 1-(thiononyl), 1-(thiodecyl), 1-(thioundecyl) or 1-(thiododecyl), wherein preferably the CH₂ group adjacent to the sp² hybridized vinyl carbon atom is replaced.

A fluoroalkyl group can be perfluoroalkyl CᵢF₂ᵢ₊₁, wherein i is an integer from 1 to 15, in particular CF₃, C₂F₅, C₃F₇, C₄F₉, C₅F₁₁, C₆F₁₃, C₇F₁₅ or C₈F₁₇, very preferably C₆F₁₃, or partially fluorinated alkyl, preferably with 1 to 15 C atoms, in particular 1,1-difluoroalkyl, all of the aforementioned being straight-chain or branched.

Preferably “fluoroalkyl” means a partially fluorinated (i.e. not perfluorinated) alkyl group.

Alkyl, alkoxy, alkenyl, oxaalkyl, thioalkyl, carbonyl and carbonyloxy groups can be achiral or chiral groups. Particularly preferred chiral groups are 2-butyl (=1-methylpropyl), 2-methylbutyl, 2-methylpentyl, 3-methylpentyl, 2-ethylhexyl, 2-butyloctyl, 2-hexyldecyl, 2-octyldodecyl, 3,7-dimethyloctyl, 3,7,11-trimethyldodecyl, 2-propylpentyl, in particular 2-methylbutyl, 2-methylbutoxy, 2-methylpentoxy, 3-methyl-pentoxy, 2-ethyl-hexoxy, 2-butyloctoxyo, 2-hexyldecoxy, 2-octyldodecoxy, 3,7-dimethyloctoxy, 3,7,11-trimethyldodecoxy, 1-methylhexoxy, 2-octyloxy, 2-oxa-3-methylbutyl, 3-oxa-4-methyl-pentyl, 4-methylhexyl, 2-hexyl, 2-octyl, 2-nonyl, 2-decyl, 2-dodecyl, 6-methoxy-octoxy, 6-methyloctoxy, 6-methyloctanoyloxy, 5-methylheptyloxy-carbonyl, 2-methylbutyryloxy, 3-methylvaleroyloxy, 4-methylhexanoyloxy, 2-chloro-propionyloxy, 2-chloro-3-methylbutyryloxy, 2-chloro-4-methyl-valeryl-oxy, 2-chloro-3-methylvaleryloxy, 2-methyl-3-oxapentyl, 2-methyl-3-oxa-hexyl, 1-methoxypropyl-2-oxy, 1-ethoxypropyl-2-oxy, 1-propoxypropyl-2-oxy, 1-butoxypropyl-2-oxy, 2-fluorooctyloxy, 2-fluorodecyloxy, 1,1,1-trifluoro-2-octyloxy, 1,1,1-trifluoro-2-octyl and 2-fluoromethyloctyloxy for example. Very preferred are 2-methylbutyl, 2-ethylhexyl, 2-butyloctyl, 2-hexyldecyl, 2-octyldodecyl, 3,7-dimethyloctyl, 3,7,11-trimethyldodecyl, 2-hexyl, 2-octyl, 2-octyloxy, 1,1,1-trifluoro-2-hexyl, 1,1,1-trifluoro-2-octyl and 1,1,1-trifluoro-2-octyloxy.

Preferred achiral branched groups are isopropyl, isobutyl (=methylpropyl), isopentyl (=3-methylbutyl), tert. butyl, isopropoxy, 2-methyl-propoxy and 3-methylbutoxy.

In a preferred embodiment, the substituents on an aryl or heteroaryl ring are independently of each other selected from primary, secondary or tertiary alkyl, alkoxy, oxaalkyl, thioalkyl, alkylcarbonyl or alkoxycarbonyl with 1 to 30 C atoms, wherein one or more H atoms are each optionally replaced by F, or aryl, aryloxy, heteroaryl or heteroaryloxy that is optionally alkylated, alkoxylated, alkylthiolated or esterified and has 4 to 30, preferably 5 to 20, ring atoms. Further preferred substituents are selected from the group consisting of the following formulae

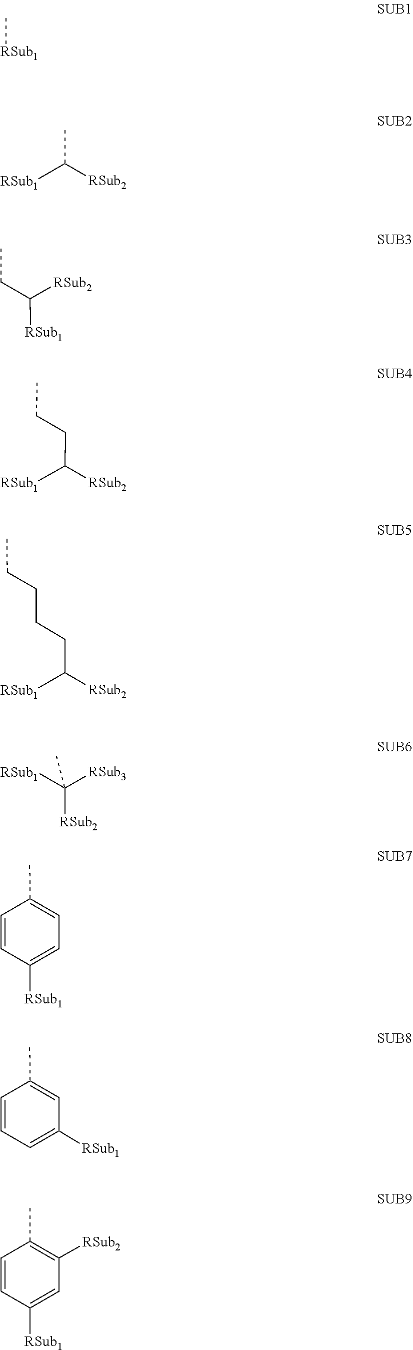

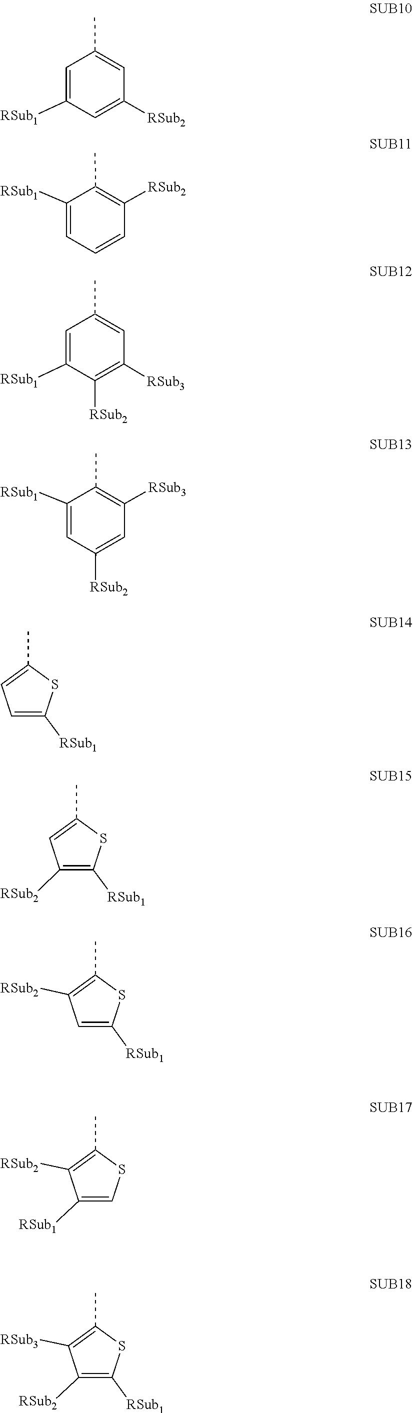

wherein RSub₁₋₃ each denote L as defined above and below and where at least, preferably all, of RSub₁₋₃ is alkyl, alkoxy, oxaalkyl, thioalkyl, alkylcarbonyl or alkoxycarbonyl with up to 24 C atoms, preferably up to 20 C atoms, that is optionally fluorinated, and wherein the dashed line denotes the link to the ring to which these groups are attached. Very preferred among these substituents are those wherein all RSub₁₋₃ subgroups are identical.

As used herein, if an aryl(oxy) or heteroaryl(oxy) group is “alkylated or alkoxylated”, this means that it is substituted with one or more alkyl or alkoxy groups having from 1 to 24 C-atoms and being straight-chain or branched and wherein one or more H atoms are each optionally substituted by an F atom.

Above and below, Y¹ and Y² are independently of each other H, F, Cl or CN.

As used herein, —CO—, —C(═O)— and —C(O)— will be understood to mean a carbonyl group, i.e. a group having the structure

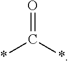

As used herein, C═CR¹R² will be understood to mean a group having the structure

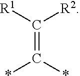

As used herein, “halogen” includes F, Cl, Br or I, preferably F, Cl or Br. A halogen atom that represents a substituent on a ring or chain is preferably F or Cl, very preferably F. A halogen atom that represents a reactive group in a monomer or an intermediate is preferably Br or I.

Above and below, the term “mirror image” means a moiety that can be obtained from another moiety by flipping it vertically or horizontally across an external symmetry plane or a symmetry plane extending through the moiety. For example the moiety

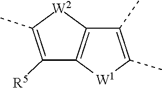

also includes the mirror images

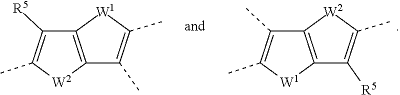

### DETAILED DESCRIPTION

The polymers of the present invention are easy to synthesize and exhibit advantageous properties. They show good processibility for the device manufacture process, high solubility in organic solvents, and are especially suitable for large scale production using solution processing methods.

The polymers of the present invention are especially suitable as (electron) acceptor or n-type semiconductor, and for the preparation of blends of n-type and p-type semiconductors which are suitable for use in OPD or BHJ OPV devices.

The polymers of the present invention are further suitable to replace the fullerene compounds that have hitherto been used as n-type semiconductor in OPV or OPD devices.

Besides, the polymers of the present invention show the following advantageous properties:
i) Optimization of the HOMO and LUMO levels of the polycyclic unit through substitution and/or careful selection of the heteroatoms may give improved light absorption.
ii) Further optimization of the HOMO and LUMO levels of the polycyclic unit through substitution and/or careful selection of the heteroatoms can increase the open circuit potential (V_{oc}).
iii) The choice of the co-monomers will influence HOMO and LUMO levels of the resulting polymer.
iv) The choice of the co-monomers will influence solubility of the resulting polymer, thus can improve morphology in the bulk, leading to higher PCE.

The synthesis of the repeating units of formula I and polymers comprising them can be achieved based on methods that are known to the skilled person and described in the literature, as will be further illustrated herein.

In the repeating units of formula I each pair of U¹ and U² is attached to the groups Ar¹ and Ar², or to the groups Ar¹ and Ar³ respectively, at directly adjacent C atoms, and in each of said pairs of U¹ and U² one of U¹ and U² is a single bond and the other is CR¹R², SiR¹R², GeR¹R², C═CR¹R², C═O or NR¹, so that each pair of U¹ and U² is forming a five-membered unsaturated ring together with the C atoms to which they are attached,

A first preferred embodiment of the present invention relates to repeating units of formula I wherein all indaceno-type groups have trans-configuration, i.e. wherein one of the two groups U¹, or one of the two groups U² respectively, which are attached to the same group Ar¹, is a single bond and the other is different from a single bond, as exemplarily illustrated below.

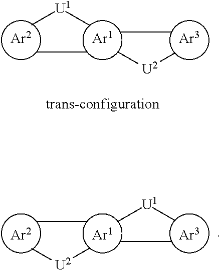

Preferred repeating units of formula I according to this first preferred embodiment are selected from the following subformulae

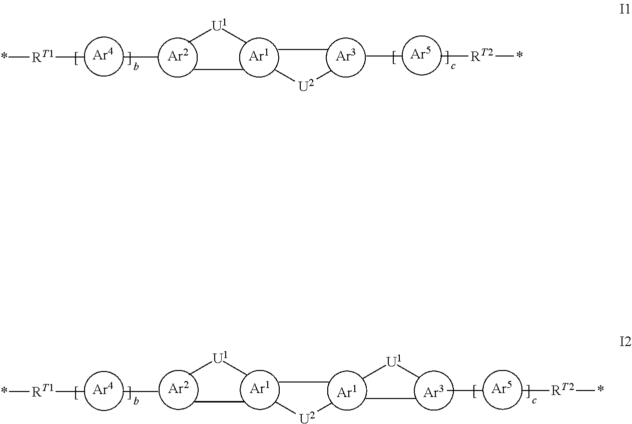

wherein U¹, U², Ar¹, Ar², Ar³, Ar⁴, R^{T1}, R^{T2}, b and c, independently of each other and on each occurrence identically or differently, have the meanings given in formula I or one of the preferred meanings given above, and U¹ and U² are different from a single bond.

Preferred repeating units of formula I1 and I2 are those wherein all of the groups U¹ and U² denote CR¹CR².

A second preferred embodiment of the present invention relates to repeating units of formula I wherein at least one, preferably all, indaceno-type groups have cis-configuration, i.e. wherein both groups U¹, or both U² respectively, which are attached to the same group Ar¹, are a single bond, as exemplarily illustrated below.

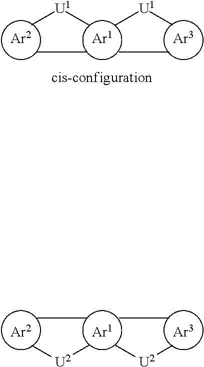

This second preferred embodiment includes repeating units of formula I having an “all-cis” configuration as exemplarily shown in formula I3 and I4 below, and repeating units of formula I including both trans-configuration and cis-configuration, as exemplarily shown in formula I5 below.

Preferred repeating units of formula I according to this second preferred embodiment are selected from the following subformulae

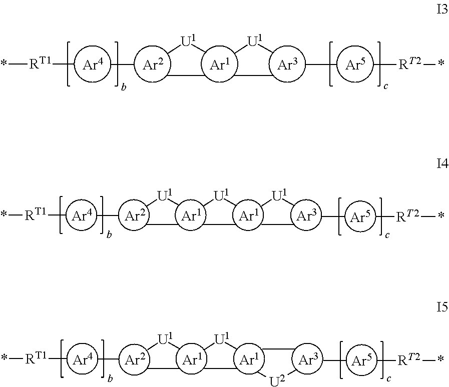

wherein U¹, U², Ar¹, Ar², Ar³, Ar⁴, R^{T1}, R^{T2}, b and c, independently of each other and on each occurrence identically or differently, have the meanings given in formula I or one of the preferred meanings given above and below, and U¹ and U² are different from a single bond.

Preferred repeating units of formula I3, I4 and I5 are those wherein all of the groups U¹ and U² denote CR¹CR².

Preferred groups Ar¹ in formula I, I1-I5 and their subformulae are on each occurrence identically or differently selected from the following formulae and their mirror images

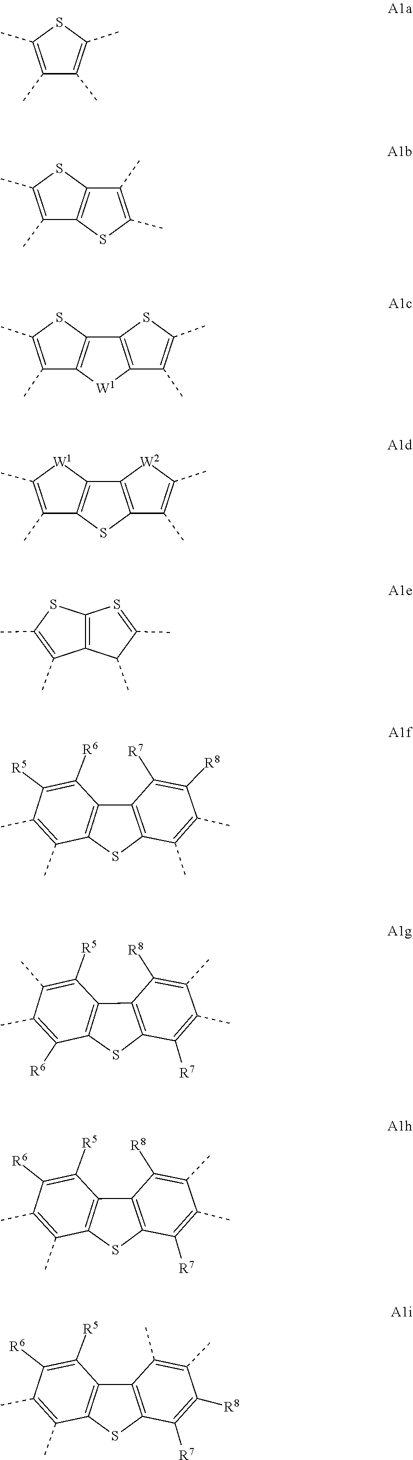

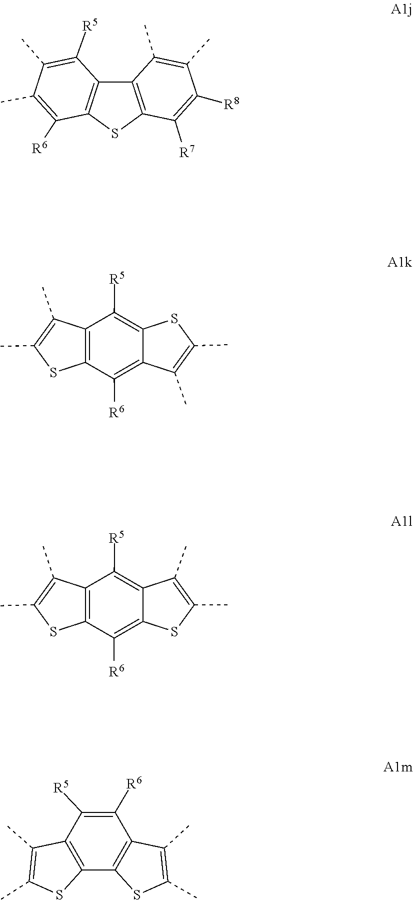

wherein the individual radicals, independently of each other and on each occurrence identically or differently, have the following meanings
W¹, W² S, O, Se or C═O,
R⁵⁻⁸ F, Cl, CN, or straight-chain, branched or cyclic alkyl with 1 to 30, preferably 1 to 20, C atoms, in which one or more CH₂ groups are each optionally replaced by —O—, —S—, —C(═O)—, —C(═S)—, —C(═O)—O—, —O—C(═O)—, —SiR⁰R⁰⁰—, —CF₂—, —CR⁰═CR⁰⁰—, —CY¹═CY²— or —C≡C— in such a manner that O and/or S atoms are not linked directly to one another, and in which one or more H atoms are each optionally replaced by F, Cl, Br, I or CN, and in which one or more CH₂ or CH₃ groups are each optionally replaced by a cationic or anionic group, or aryl, heteroaryl, arylalkyl, heteroarylalkyl, aryloxy or heteroaryloxy, wherein each of the aforementioned cyclic groups has 5 to 20 ring atoms, is mono- or polycyclic, optionally contains fused rings, and is unsubstituted or substituted by one or more identical or different groups L,
Y¹, Y², R⁰, R⁰⁰ one of the meanings given in formula I.

Preferred groups Ar¹ are those of formula A1a, A1b and A1k, especially those of formula A1a and A1b.

Preferred groups Ar¹ of formula A1c and A1d are those wherein W¹ and W² are S.

Preferred groups Ar² in formula I, I1-I5 and their subformulae are on each occurrence identically or differently selected from the following formulae and their mirror images

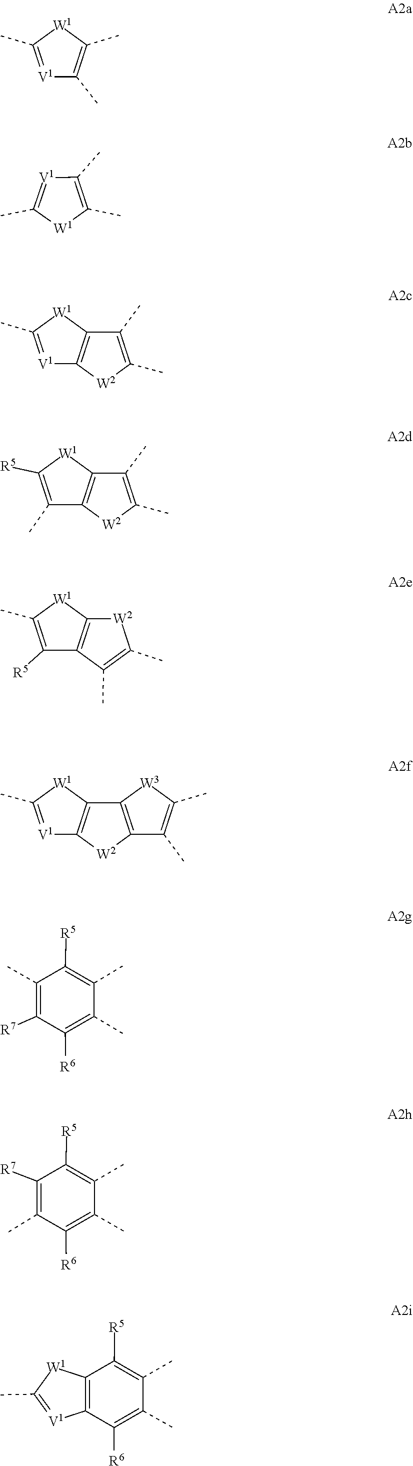

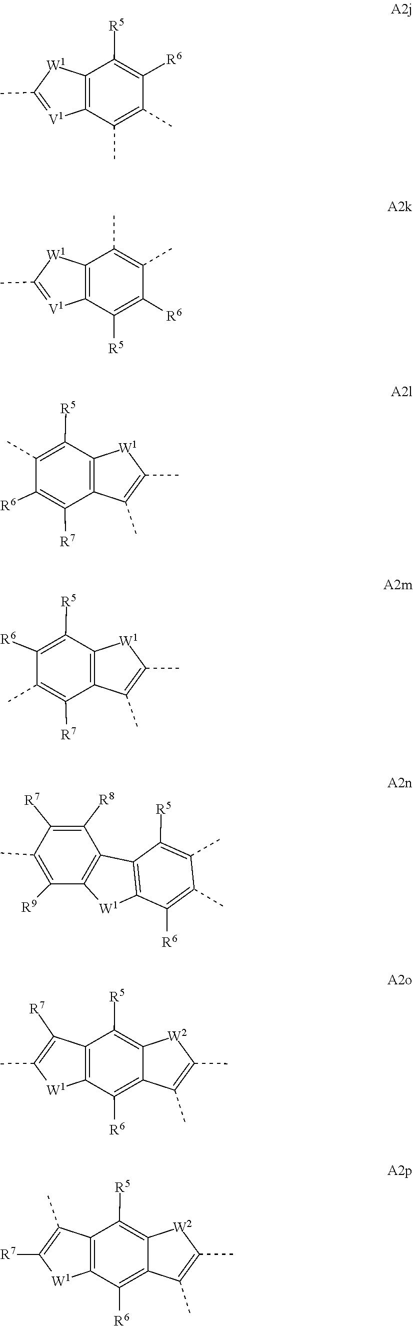

wherein, independently of each other and on each occurrence identically or differently, W¹, W² and R⁵⁻⁸ have the meanings given above and below, V¹ is CR³ or N, W³ has one of the meanings given for W¹, and R³ and R⁹ have one of the meanings given for R⁵.

Preferred groups Ar² are those of formula A2a, A2b, A2d, A2e, A2g and A2h, especially those of formula A2a and A2b.

Preferred groups Ar² of formula A2a-A2p are those wherein W¹, W² and W³ are S.

Very preferred groups Ar² in formula I, I1-I5 and their subformulae are on each occurrence identically or differently selected from the following formulae and their mirror images

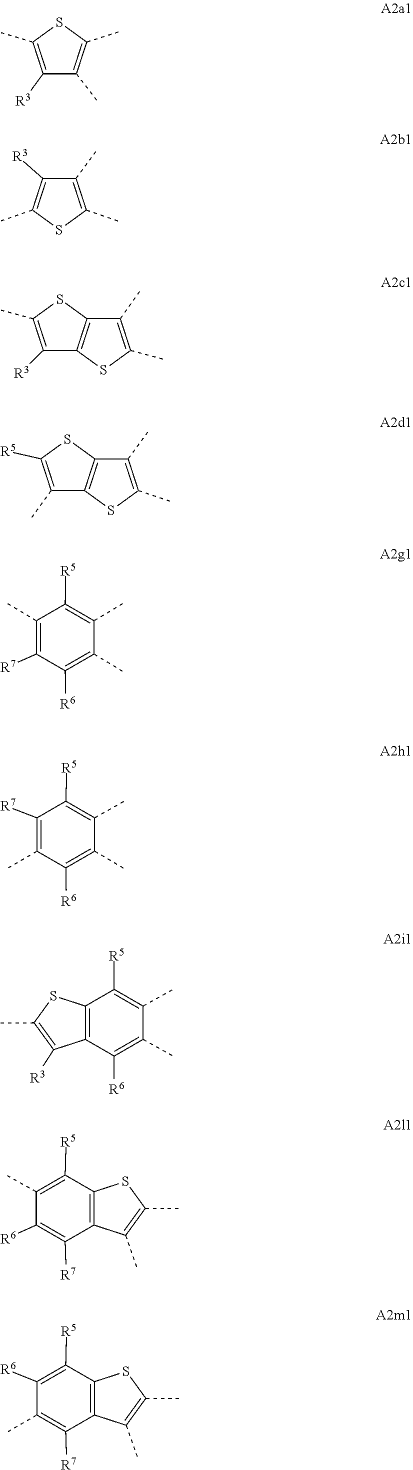

wherein R³⁻⁷ have the meanings given above and below.

Very preferred are the groups of formula A2a1 and A2b1.

Preferred groups Ar³ in formula I, I1-I5 and their subformulae are on each occurrence identically or differently selected from the following formulae and their mirror images

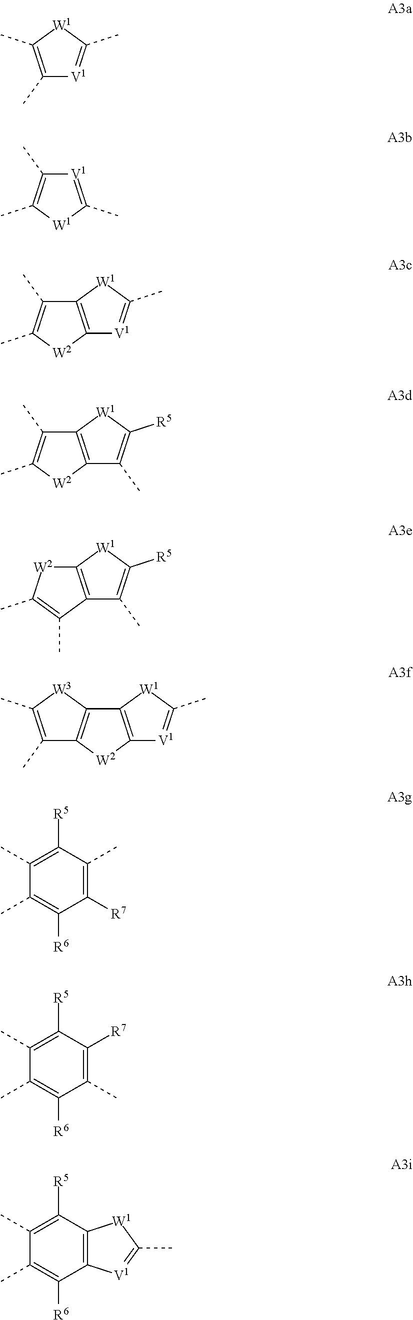

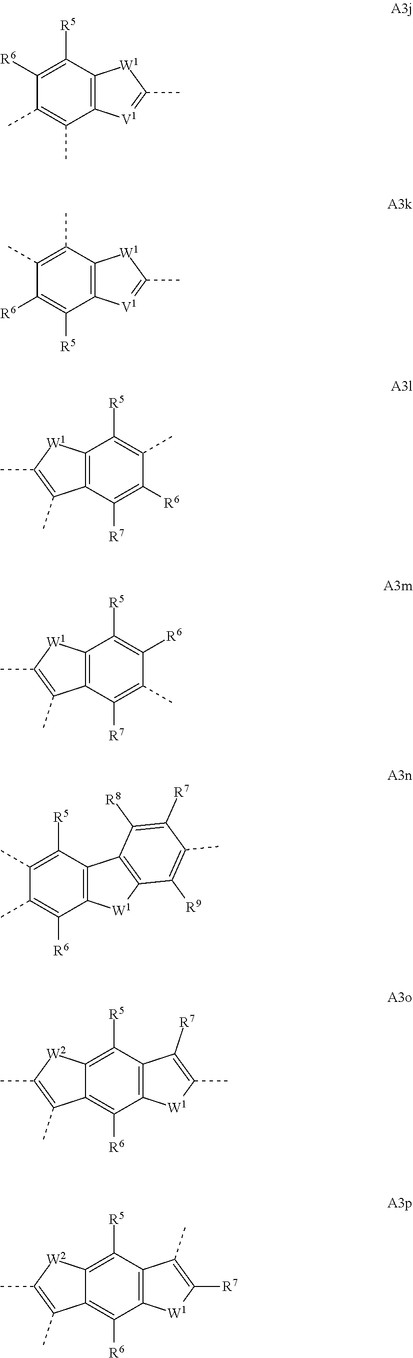

wherein V¹, W¹, W², W³ and R⁵⁻⁹ have the meanings given above and below.

Preferred groups Ar³ of formula A3a-A3p are those wherein W¹, W² and W³ are S.

Preferred groups Ar³ are those of formula A3a, A3b, A3c, A3d, A3g and A3h, especially those of formula A3a and A3b.

Very preferred groups Ar³ in formula I, I1-I5 and their subformulae are on each occurrence identically or differently selected from the following formulae and their mirror images

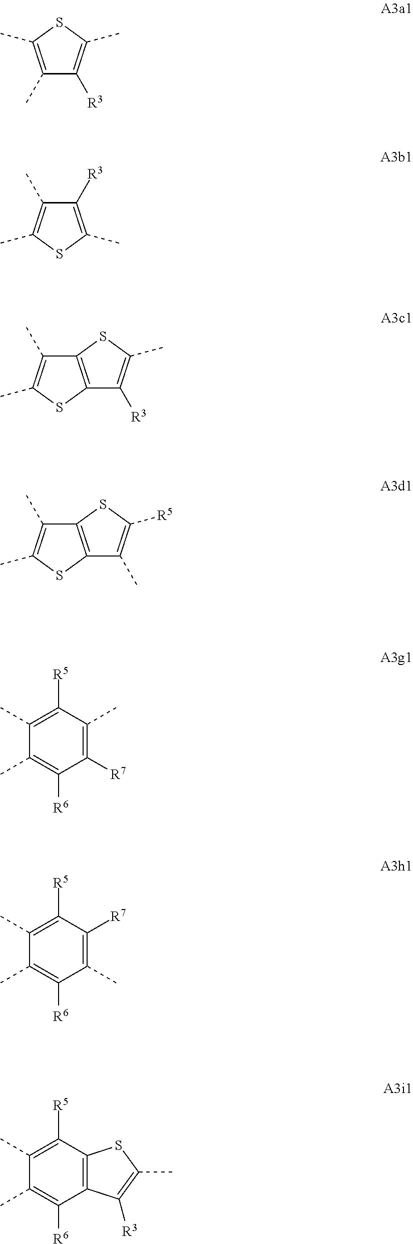

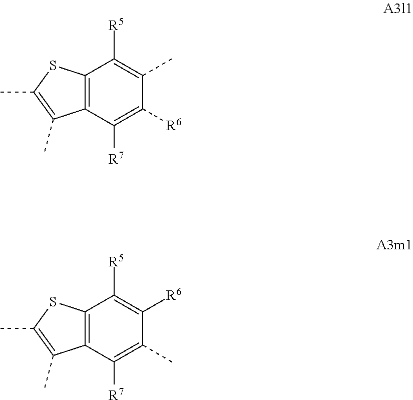

wherein R³⁻⁷ have the meanings given above and below.

Very preferred are the groups of formula A3a1 and A3b1.

In the repeating units of formula I and its subformulae, if a>0, Ar¹ contains at least one thiophene ring.

In the repeating units of formula I and its subformulae, if a=0, preferably at least one of Ar² and Ar³ contains at least one thiophene ring.

Preferred repeating units of formula I and I1-I5 are those wherein at least one of Ar¹, Ar² and Ar³, very preferably Ar¹, contains one or more thiophene rings.

Further preferred repeating units of formula I are those wherein a is 1 or 2, preferably selected from formulae I1 and I2, and Ar¹ is selected from formulae A1b and A1k.

Further preferred repeating units of formula I are those wherein a is 1 or 2, preferably selected from formulae I3, I4 and I5, and Ar¹ is selected from formulae A1a, A1c, A1d, A1e and A1m.

Further preferred repeating units of formula I are those wherein a is 0, and
Ar² is selected from formulae A2a-A2f and A2i-A2p, very preferably from formulae A2a, A2b, A2c, A2i, A2j, A2k, A2l and A2m, most preferably from formulae A2a1, A2b1, A2c1, A2i1, A2l1 and A2m1, and/or
Ar³ is selected from formulae A3a-A3f and A3i-A3p, very preferably from formulae A3a, A3b, A3c, A3i, A3j, A3k, A3l and A3m, most preferably from formulae A3a1, A3b1, A3c1, A3i1, A3l1 and A3m1.

Preferred groups Ar⁴ and Ar⁵ in formula I, I1-I5 and their subformulae are each independently and on each occurrence identically or differently selected from arylene or heteroarylene which has from 5 to 20 ring atoms, which is mono- or polycyclic, which optionally contains fused rings, and which is unsubstituted or substituted by one or more identical or different groups L, or from —CY¹═CY²—.

Very preferred groups Ar⁴ and Ar⁵ in formula I, I1-I5 and their subformulae are each independently and on each occurrence identically or differently selected from the following formulae and their mirror images:

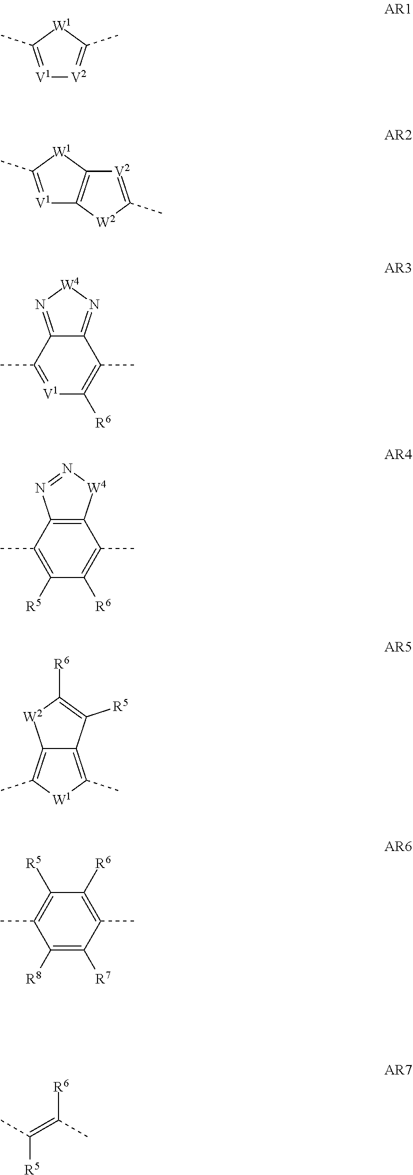

wherein the individual radicals, independently of each other and on each occurrence identically or differently, have the following meanings

V² CR⁴ or N,

W⁴ S, O, Se, NR⁰ or C═O,

R⁴ one of the meanings given for R³ above and below,

and V¹, W¹, W², R⁰, R⁵⁻⁸ are as defined above and below.

More preferred groups Ar⁴ and Ar⁵ in formula I, I1-I5 and their subformulae are each independently, and on each occurrence identically or differently, selected from the following formulae and their mirror images

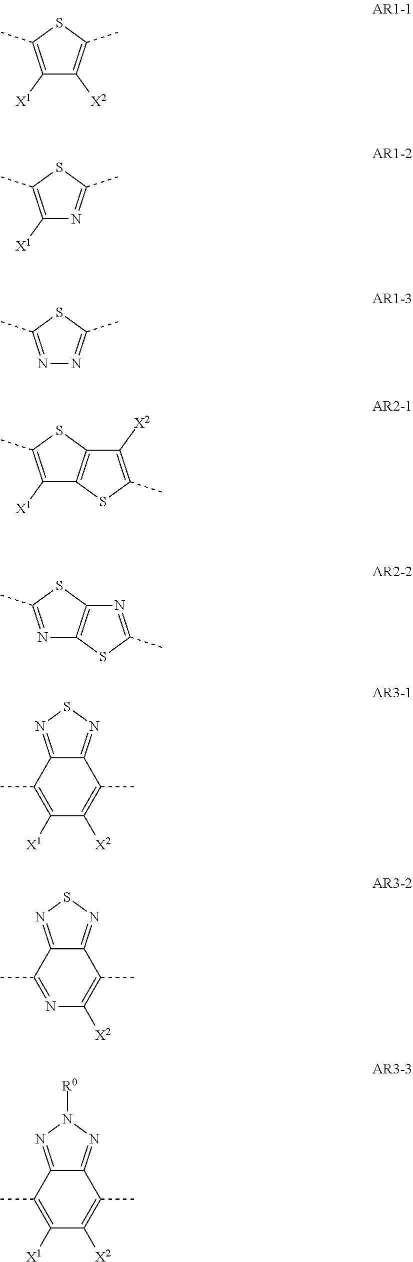

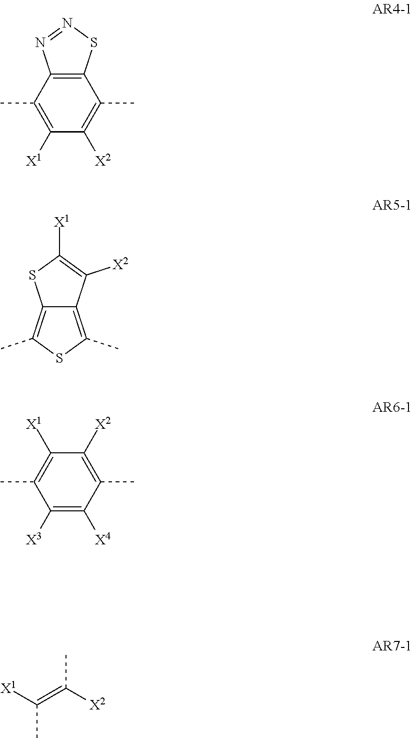

wherein X¹, X², X³ and X⁴ have one of the meanings given for R¹ above and below, and preferably denote H, F, Cl, —CN, R⁰, OR⁰ or C(═O)OR⁰, and R⁰ is as defined above and below.

Preferred formulae AR1-1 to AR7-1 are those containing at least one, preferably one, two or four substituents X¹⁻⁴ selected from F and Cl, very preferably F.

In formula AR6-1 preferably one or two, very preferably all of X¹⁻⁴ are F.

Preferred groups Ar⁴ and Ar⁵ are selected from formulae AR1, AR2, AR3, AR5 and AR7. Very preferred groups Ar³⁻⁴ are selected from formulae AR1-1, AR1-2, AR2-1, AR3-1, AR3-2, AR5-1 and AR7-1, most preferably from formulae AR1-1, AR2-1, AR3-1 and AR7-1.

The groups of formula T, and the units of formula I and its subformulae, as well as the repeating units, monomers and polymers comprising them, are understood to cover also isomeric mixtures. This is because in the electron withdrawing groups R^{T1} and R^{T2} the linkage of the ring Ar⁶ to the neighboured group (indicated in formula T by —*) may be located in different positions of Ar⁶, as shown e.g. in subformulae T1-T35 below, depending on the synthesis method. Thus, the synthesis of the groups of formula T and its subformulae may yield a mixture of isomers which is not necessarily separated before further reaction with the polycyclic core. As a consequence e.g. the further reaction of two moieties of formula T, each of which is a mixture of isomers, with the polycyclic core to form a monomer with a unit of formula I or its subformulae will yield another mixture of isomers.

In the groups of formula T, Ar⁶ is preferably selected from benzene, thiophene, naphthalene and benzothiophene, each of which is optionally substituted by one or more groups L as defined above.

In the groups of formula T, Z¹ and Z² are preferably selected from the group consisting of the formulae

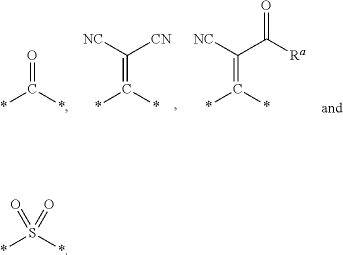

Preferred groups of formula T are those wherein one of Z¹ and Z² is

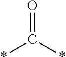 [SMILES: *C(*)=O]

and the other is

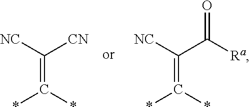

furthermore those wherein one of Z¹ and Z² is

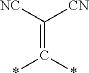 [SMILES: *C(*)=C(C#N)C#N]

and the other is

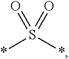

wherein R^{a} is as defined in formula T and preferably denotes C₁₋₁₂ alkyl.

In the groups of formula T preferably L′ is H.

Further preferably in the groups of formula T R^{a} denotes C₁-C₁₂-alkyl.

Preferably R^{T1} and R^{T2} in formula I, I1-I5 and their subformulae are each independently selected from the group consisting of the following formulae

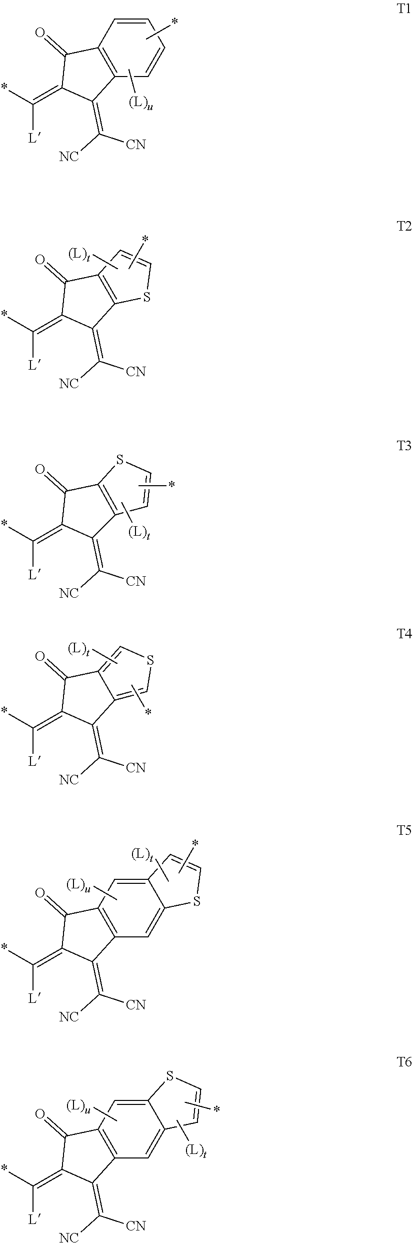

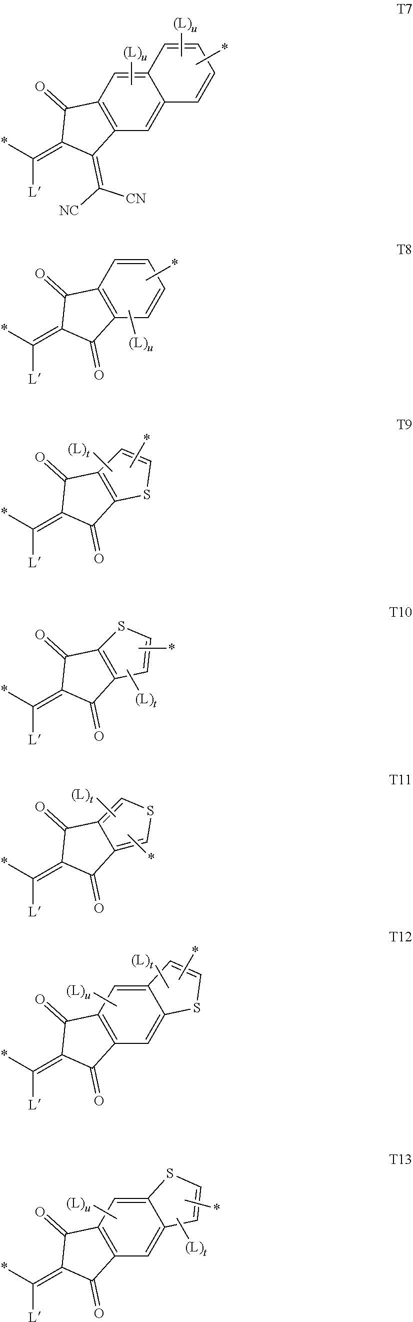

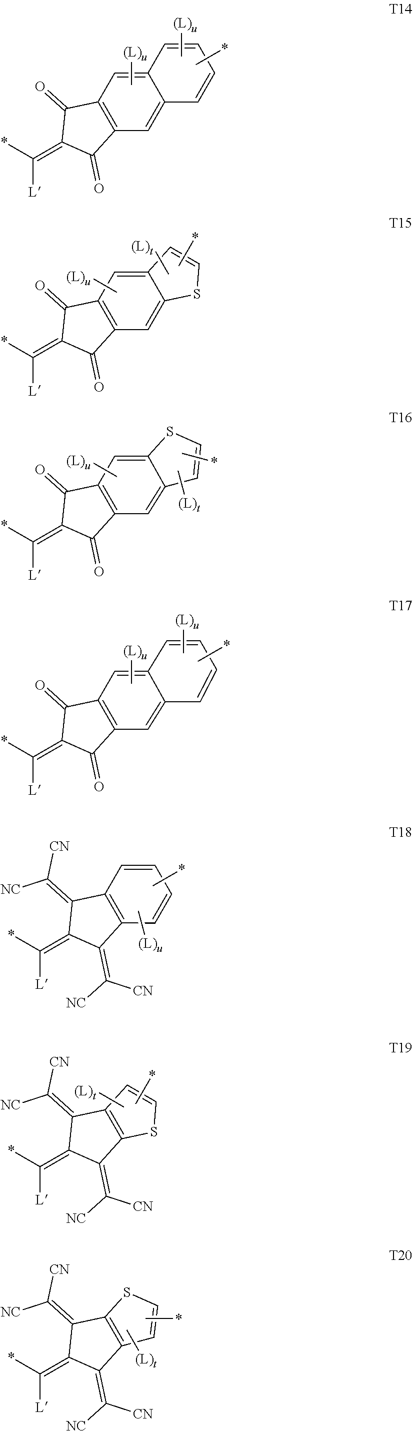

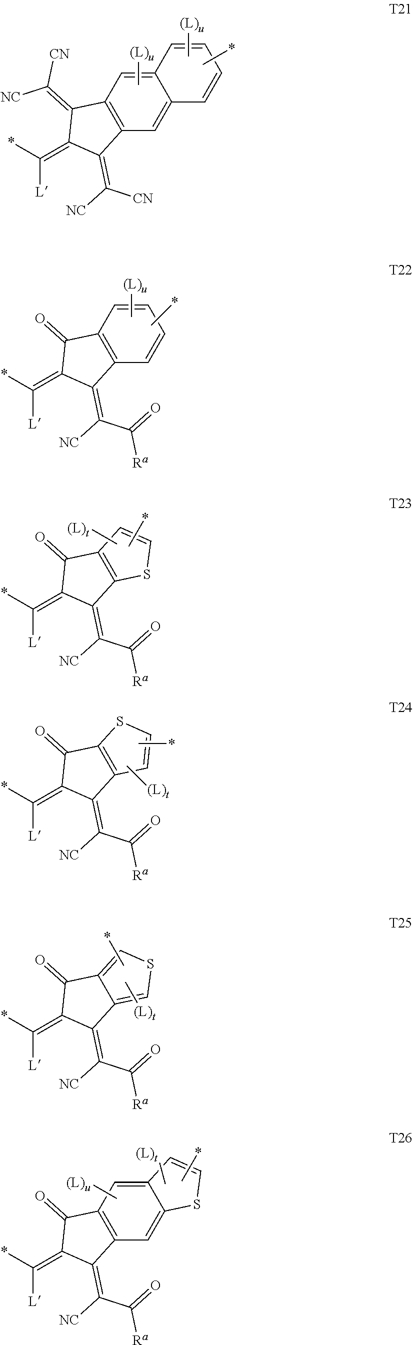

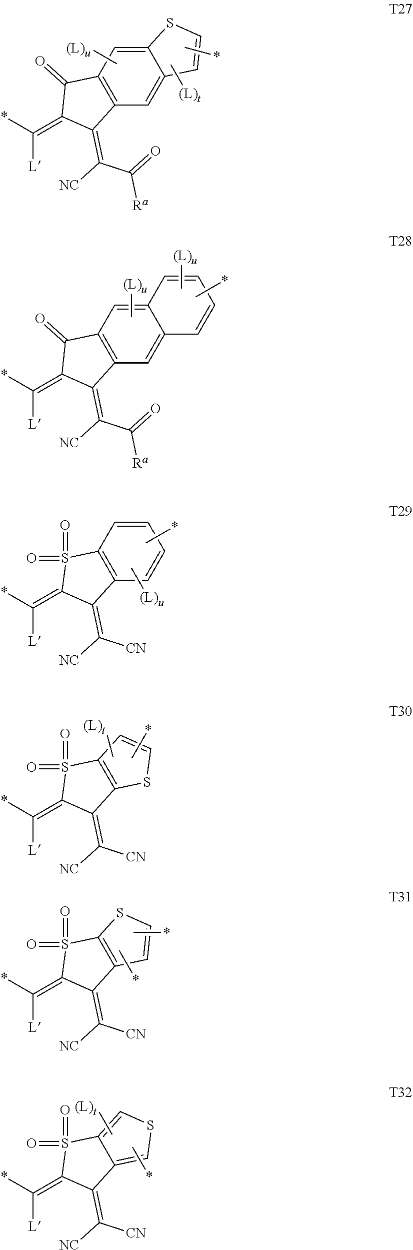

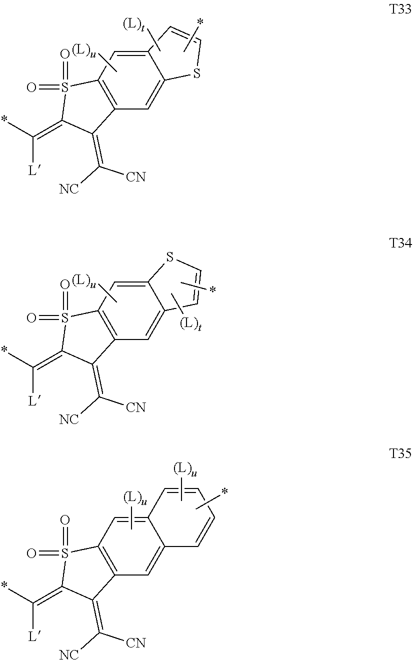

wherein R^{a}, L and L′ have the meanings given above and below, t is 0 or 1, and u is 0, 1 or 2.

Preferred groups T1-T35 are those wherein L′ is H, R^{a} denotes C₁-C₁₂-alkyl, t is 0 and u is 0.

Further preferred groups R^{T1} and R^{T2} are each independently selected from formulae T1, T2, T3, T7, T8, T14, T15, T21, T22, T28, T29 and T35, very preferably from formulae T1 and T7.

The above formulae T1-T35 are meant to also include their respective E- or Z-stereoisomer with respect to the C═C bond in α-position to the adjacent group Ar⁴ or Ar⁵, thus for example the group

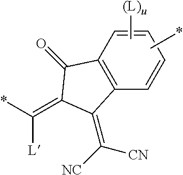

may also denote

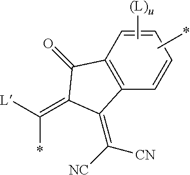

In the repeating units of formula I, I1-I5 and their subformulae R¹ and R² are preferably different from H.

In a preferred embodiment of the present invention, in the repeating units of formula I, I1-I5 and their subformulae R¹ and R² are selected from F, Cl, CN, or from straight-chain or branched alkyl, alkoxy, sulfanylalkyl, sulfonylalkyl, alkylcarbonyl, alkoxycarbonyl and alkylcarbonyloxy, each of which has 1 to 20 C atoms and is unsubstituted or substituted by one or more F atoms, most preferably from F, Cl or formulae SUB1-SUB6 above.

In another preferred embodiment of the present invention, in the repeating units of formula I, I1-I5 and their subformulae R¹ and R² are selected from mono- or polycyclic aryl or heteroaryl, each of which is optionally substituted with one or more groups L as defined in formula I and has 5 to 20 ring atoms, and wherein two or more rings may be fused to each other or connected with each other by a covalent bond, very preferably phenyl that is optionally substituted, preferably in 4-position, 2,4-positions, 2,4,6-positions or 3,5-positions, or thiophene that is optionally substituted, preferably in 5-position, 4,5-positions or 3,5-positions, with alkyl, alkoxy or thioalkyl having 1 to 16 C atoms, most preferably from formulae SUB7-SUB18 above.

In the repeating units of formula I, I1-I5 and their subformulae R³ and R⁴ are preferably H.

In another preferred embodiment of the present invention, in the repeating units of formula I, I1-I5 and their subformulae R³ and R⁴ are different from H.

In another preferred embodiment of the present invention, in the repeating units of formula I, I1-I5 and their subformulae R³ and R⁴ are selected from F, Cl, CN, or from straight-chain or branched alkyl, alkoxy, sulfanylalkyl, sulfonylalkyl, alkylcarbonyl, alkoxycarbonyl and alkylcarbonyloxy, each of which has 1 to 20 C atoms and is unsubstituted or substituted by one or more F atoms, most preferably from F, Cl or formulae SUB1-SUB6 above.

In another preferred embodiment of the present invention, in the repeating units of formula I, I1-I5 and their subformulae R³ and R⁴ are selected from mono- or polycyclic aryl or heteroaryl, each of which is optionally substituted with one or more groups L as defined in formula I and has 5 to 20 ring atoms, and wherein two or more rings may be fused to each other or connected with each other by a covalent bond, very preferably phenyl that is optionally substituted, preferably in 4-position, 2,4-positions, 2,4,6-positions or 3,5-positions, or thiophene that is optionally substituted, preferably in 5-position, 4,5-positions or 3,5-positions, with alkyl, alkoxy or thioalkyl having 1 to 16 C atoms, more preferably from formulae SUB7-SUB18 above, most preferably from formulae SUB14-SUB18 above.

In a preferred embodiment of the present invention, in the repeating units of formula I, I1-I5 and their subformulae R⁵⁻⁹ are H.

In another preferred embodiment of the present invention, in the repeating units of formula I, I1-I5 and their subformulae at least one of R⁵⁻⁹ is different from H.

In a preferred embodiment of the present invention, in the repeating units of formula I, I1-I5 and their subformulae R⁵⁻⁹, when being different from H, are each independently selected from F, Cl, CN, or from straight-chain or branched alkyl, alkoxy, sulfanylalkyl, sulfonylalkyl, alkylcarbonyl, alkoxycarbonyl and alkylcarbonyloxy, each of which has 1 to 20 C atoms and is unsubstituted or substituted by one or more F atoms, most preferably from F, Cl or formulae SUB1-SUB6 above.

In another preferred embodiment of the present invention, in the repeating units of formula I, I1-I5 and their subformulae R⁵⁻⁹, when being different from H, are each independently selected are selected from mono- or polycyclic aryl or heteroaryl, each of which is optionally substituted with one or more groups L as defined in formula I and has 5 to 20 ring atoms, and wherein two or more rings may be fused to each other or connected with each other by a covalent bond, very preferably phenyl that is optionally substituted, preferably in 4-position, 2,4-positions, 2,4,6-positions or 3,5-positions, or thiophene that is optionally substituted, preferably in 5-position, 4,5-positions or 3,5-positions, with alkyl, alkoxy or thioalkyl having 1 to 16 C atoms, more preferably from formulae SUB7-SUB18 above, most preferably from formulae SUB14-SUB18 above.

Preferred aryl and heteroaryl groups R¹⁻⁹, when being different from H, are each independently selected from the following formulae

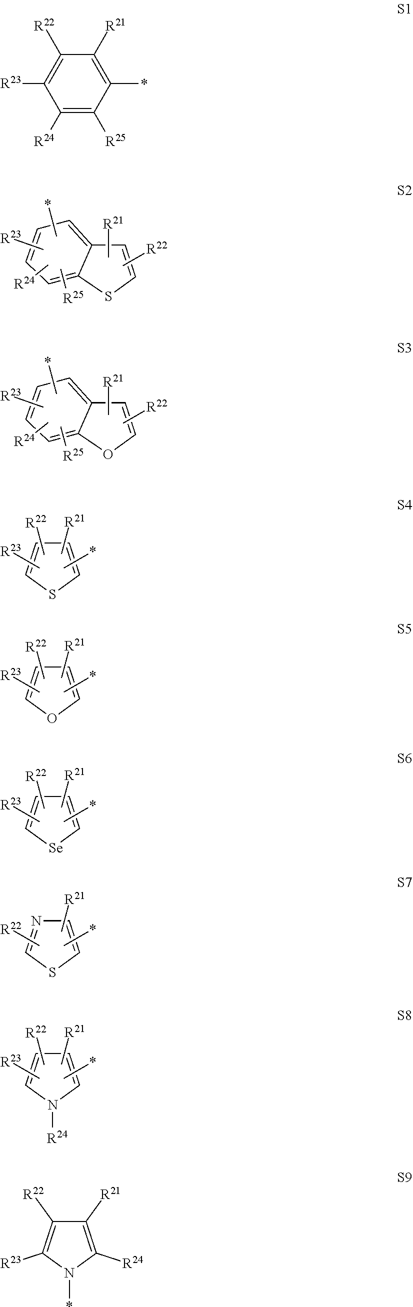

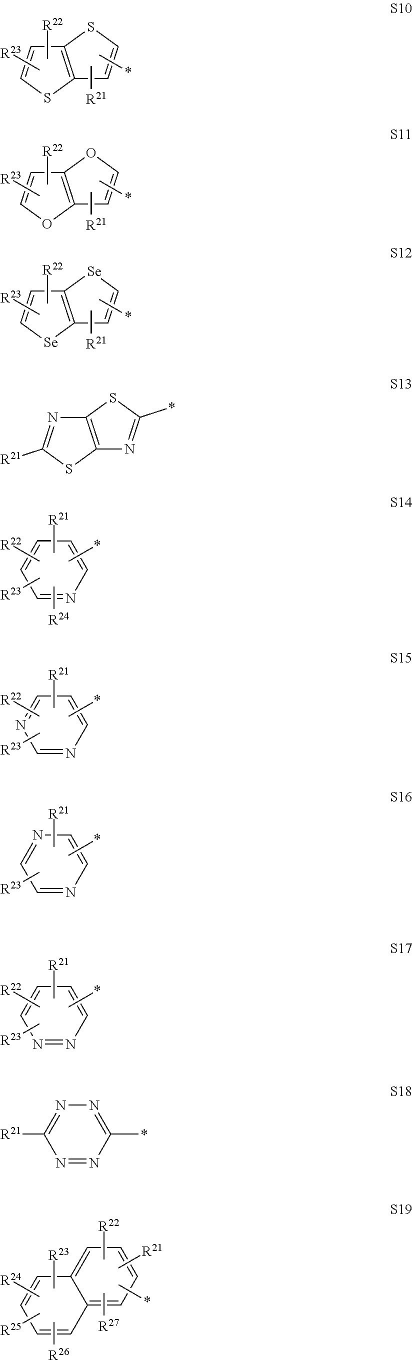

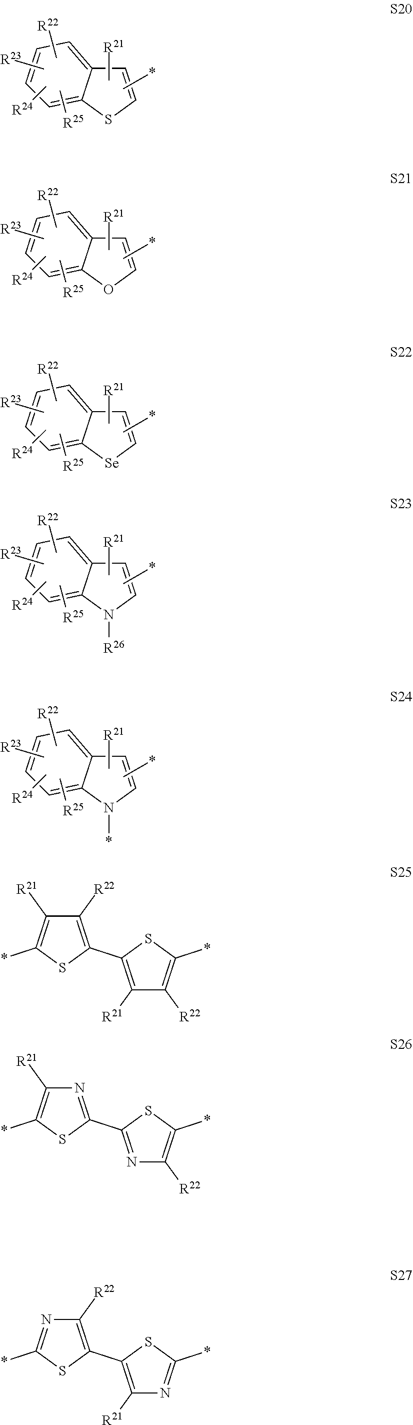

wherein R²¹⁻¹²⁷, independently of each other, and on each occurrence identically or differently, denote H, F, Cl, CN, or straight-chain, branched or cyclic alkyl with 1 to 30, preferably 1 to 20, C atoms, in which one or more CH₂ groups are each optionally replaced by —O—, —S—, —C(═O)—, —C(═S)—, —C(═O)—O—, —O—C(═O)—, —NR⁰—, —SiR⁰R⁰⁰—, —CF₂—, —CR⁰═CR⁰⁰—, —CY¹═CY²— or —C≡C— in such a manner that O and/or S atoms are not linked directly to one another, and in which one or more H atoms are each optionally replaced by F, Cl, Br, I or CN, and in which one or more CH₂ or CH₃ groups are each optionally replaced by a cationic or anionic group.

Very preferred aryl and heteroaryl groups R¹⁻⁸, when being different from H, are each independently selected from formulae S1, S4, S5, S7 and S10.

Most preferred aryl and heteroaryl groups R¹⁻⁹ are each independently selected from formulae SUB7-SUB16 as defined above.

In another preferred embodiment one or more of R¹⁻⁹ denote a straight-chain, branched or cyclic alkyl group with 1 to 50, preferably 2 to 50, very preferably 2 to 30, more preferably 2 to 24, most preferably 2 to 16 C atoms, in which one or more CH₂ or CH₃ groups are replaced by a cationic or anionic group.

The cationic group is preferably selected from the group consisting of phosphonium, sulfonium, ammonium, uronium, thiouronium, guanidinium or heterocyclic cations such as imidazolium, pyridinium, pyrrolidinium, triazolium, morpholinium or piperidinium cation.

Preferred cationic groups are selected from the group consisting of tetraalkylammonium, tetraalkylphosphonium, N-alkylpyridinium, N,N-dialkylpyrrolidinium, 1,3-dialkylimidazolium, wherein “alkyl” preferably denotes a straight-chain or branched alkyl group with 1 to 12 C atoms and very preferably is selected from formulae SUB1-6.

Further preferred cationic groups are selected from the group consisting of the following formulae

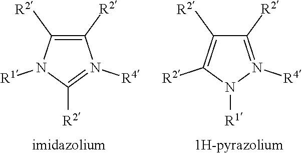

wherein R¹′, R²′, R³′ and R⁴′ denote, independently of each other, H, a straight-chain or branched alkyl group with 1 to 12 C atoms or non-aromatic carbo- or heterocyclic group or an aryl or heteroaryl group, each of the aforementioned groups having 3 to 20, preferably 5 to 15, ring atoms, being mono- or polycyclic, and optionally being substituted by one or more identical or different substituents L as defined above, or denote a link to the respective group R¹⁻⁹.

In the above cationic groups of the above-mentioned formulae any one of the groups R¹′, R²′, R³′ and R⁴′ (if they replace a CH₃ group) can denote a link to the respective group R¹⁻¹⁰, or two neighbored groups R¹′, R²′, R³′ or R⁴′ (if they replace a CH₂ group) can denote a link to the respective group R¹.

The anionic group is preferably selected from the group consisting of borate, imide, phosphate, sulfonate, sulfate, succinate, naphthenate or carboxylate, very preferably from phosphate, sulfonate or carboxylate.

Further preferred repeating units of formula I, I1-I5 and their subformulae are selected from the following preferred embodiments or any combination thereof:
a is 0,
a is 1 or 2, preferably 1,
b is 1 or 2, preferably 1,
cis 1 or 2, preferably 1,
b=c=0,
b=c=1 or 2,
U¹ and U² denote CR¹R²,
W¹, W² and W³ are S or Se, preferably S,
W⁴ is S or NR⁰, preferably S,
V¹ is CR³,
V² is CR⁴,
V¹ is N,
V² is N,
V¹ is CR³ and V² is CR⁴,
V¹ is CR³ and V² is N,
V¹ and V² are N,
Ar¹ is selected of formula A1a, A1b and A1k, preferably of formula A1a and A1b,
Ar² is selected from formulae A2a, A2b, A2d, A2e, A2g and A2h, preferably from formulae A2a and A2b, very preferably from formulae A2a1 and A2b1,
Ar³ is selected from formulae A3a, A3b, A3d, A3e, A3g and A3h, preferably from formulae A3a and A3b, very preferably from formulae A3a1 and A3b1,
in one or both of Ar² and Ar³ all substituents R⁵⁻⁸ are H,
in one or both of Ar² and Ar³ at least one, preferably one or two of R⁵⁻⁸ are different from H,
in one or both of Ar⁴ and Ar⁵ all substituents R⁵⁻⁸ are H,
in one or both of Ar⁴ and Ar⁵ at least one, preferably one or two of R⁵⁻⁸ are different from H,
Ar⁴ and Ar⁵ are selected from formulae AR1, AR2, AR3, AR5 and AR7,
Ar⁴ and Ar⁵ are selected from formulae AR1-1, AR1-2, AR2-1, AR3-1, AR3-2, AR5-1 and AR7-1, most preferably from formulae AR1-1, AR2-1, AR3-1 and AR7-1,
Ar⁴ and Ar⁵ denote thiophene, thiazole, thieno[3,2-b]thiophene, thiazolo[5,4-d]thiazole, benzene, 2,1,3-benzothiadiazole, 1,2,3-benzothiadiazole, thieno[3,4-b]thiophene, benzotriazole, thiadiazole[3,4-c]pyridine or vinyl, which are substituted by X¹, X², X³ and X⁴ as defined above,
Ar⁴ and Ar⁵ denote thiophene, thiazole, thieno[3,2-b]thiophene, thiazolo[5,4-d]thiazole, benzene, 2,1,3-benzothiadiazole, 1,2,3-benzothiadiazole, thieno[3,4-b]thiophene, benzotriazole, thiadiazole[3,4-c]pyridine or vinyl, wherein X¹, X², X³ and X⁴ are H,
Ar⁴ and Ar⁵ denote thiophene, thiazole, thieno[3,2-b]thiophene, thiazolothiazole, benzene, 2,1,3-benzothiadiazole, 1,2,3-benzothiadiazole, thieno[3,4-b]thiophene, benzotriazole, thiadiazole[3,4-c]pyridine or vinyl, wherein one or more of X¹, X², X³ and X⁴ are different from H,
Ar⁶ is selected from benzene, thiophene and naphthalene, each of which is optionally substituted by one or more groups L as defined above,
Z¹ and Z² are selected from the group consisting of the formulae

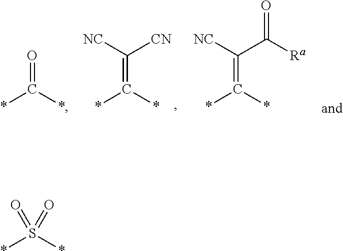

one of Z¹ and Z² is

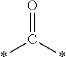 [SMILES: *C(*)=O]

and the other is

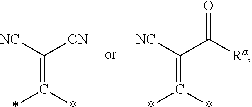

one of Z¹ and Z² is

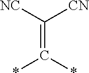 [SMILES: *C(*)=C(C#N)C#N]

and the other is

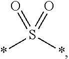

R^{a} denotes C₁₋₁₂ alkyl,
L′ is H,
R^{T1} and R^{T2} are selected from the formulae T1, T7, T8, T14, T15, T21, T22, T28, T29 and T35, very preferably from formulae T1 and T7, wherein preferably L′ is H, R^{a} is H or C₁-C₁₂-alkyl, t is 0 and u is 0,
L denotes F, Cl, CN, NO₂, or alkyl or alkoxy with 1 to 16 C atoms that is optionally fluorinated,
t is 1 and L is F, Cl, CN, NO₂, or alkyl or alkoxy with 1 to 16 C atoms that is optionally fluorinated,
u is 1 or 2 and L is F, Cl, CN, NO₂, or alkyl or alkoxy with 1 to 16 C atoms that is optionally fluorinated,
R¹ and R² are different from H,
R¹ and R², when being different from H, are each independently selected from F, Cl or straight-chain or branched alkyl, alkoxy, sulfanylalkyl, sulfonylalkyl, alkylcarbonyl, alkoxycarbonyl and alkylcarbonyloxy, each having 1 to 20 C atoms and being unsubstituted or substituted by one or more F atoms, or alkyl or alkoxy having 1 to 12 C atoms that is optionally fluorinated, more preferably from formulae SUB1-SUB6 above,
R¹ and R², when being different from H, and are each independently selected from phenyl that is substituted, preferably in 4-position, or in 2,4-positions, or in 2,4,6-positions or in 3,5-positions, with alkyl or alkoxy having 1 to 20 C atoms, preferably 1 to 16 C atoms, very preferably 4-alkylphenyl wherein alkyl is C1-16 alkyl, most preferably 4-methylphenyl, 4-hexylphenyl, 4-octylphenyl or 4-dodecylphenyl, or 4-alkoxyphenyl wherein alkoxy is C1-16 alkoxy, most preferably 4-hexyloxyphenyl, 4-octyloxyphenyl or 4-dodecyloxyphenyl or 2,4-dialkylphenyl wherein alkyl is C1-16 alkyl, most preferably 2,4-dihexylphenyl or 2,4-dioctylphenyl or 2,4-dialkoxyphenyl wherein alkoxy is C1-16 alkoxy, most preferably 2,4-dihexyloxyphenyl or 2,4-dioctyloxyphenyl or 3,5-dialkylphenyl wherein alkyl is C1-16 alkyl, most preferably 3,5-dihexylphenyl or 3,5-dioctylphenyl or 3,5-dialkoxyphenyl wherein alkoxy is C1-16 alkoxy, most preferably 3,5-dihexyloxyphenyl or 3,5-dioctyloxyphenyl, or 2,4,6-trialkylphenyl wherein alkyl is C1-16 alkyl, most preferably 2,4,6-trihexylphenyl or 2,4,6-trioctylphenyl or 2,4,6-trialkoxyphenyl wherein alkoxy is C1-16 alkoxy, most preferably 2,4,6-trihexyloxyphenyl or 2,4,6-trioctyloxyphenyl or 4-thioalkylphenyl wherein thioalkyl is C1-16 thioalkyl, most preferably 4-thiohexylphenyl, 4-thiooctylphenyl or 4-thiododecylphenyl, or 2,4-dithioalkylphenyl wherein thioalkyl is C1-16 thioalkyl, most preferably 2,4-dithiohexylphenyl or 2,4-dithiooctylphenyl, or 3,5-dithioalkylphenyl wherein thioalkyl is C1-16 thioalkyl, most preferably 3,5-dithiohexylphenyl or 3,5-dithiooctylphenyl, or 2,4,6-trithioalkylphenyl wherein thioalkyl is C1-16 thioalkyl, most preferably 2,4,6-trithiohexylphenyl or 2,4,6-trithiooctylphenyl, or from thiophene that is optionally substituted, preferably in 5-position, 4,5-positions or 3,5-positions, with alkyl, alkoxy or thioalkyl having 1 to 16 C atoms, most preferably from formulae SUB7-SUB18 above,
R³ and R⁴ are H,
R³ and R⁴ are different from H,
R³ and R⁴, when being different from H, are each independently selected from F, Cl or straight-chain or branched alkyl, alkoxy, sulfanylalkyl, sulfonylalkyl, alkylcarbonyl, alkoxycarbonyl and alkylcarbonyloxy, each having 1 to 20 C atoms and being unsubstituted or substituted by one or more F atoms, without being perfluorinated, or alkyl or alkoxy having 1 to 12 C atoms that is optionally fluorinated, more preferably from formulae SUB1-SUB6 above,
R³ and R⁴ are different from H, and are each independently selected from phenyl that is substituted, preferably in 4-position, or in 2,4-positions, or in 2,4,6-positions or in 3,5-positions, with alkyl or alkoxy having 1 to 20 C atoms, preferably 1 to 16 C atoms, very preferably 4-alkylphenyl wherein alkyl is C1-16 alkyl, most preferably 4-methylphenyl, 4-hexylphenyl, 4-octylphenyl or 4-dodecylphenyl, or 4-alkoxyphenyl wherein alkoxy is C1-16 alkoxy, most preferably 4-hexyloxyphenyl, 4-octyloxyphenyl or 4-dodecyloxyphenyl or 2,4-dialkylphenyl wherein alkyl is C1-16 alkyl, most preferably 2,4-dihexylphenyl or 2,4-dioctylphenyl or 2,4-dialkoxyphenyl wherein alkoxy is C1-16 alkoxy, most preferably 2,4-dihexyloxyphenyl or 2,4-dioctyloxyphenyl or 3,5-dialkylphenyl wherein alkyl is C1-16 alkyl, most preferably 3,5-dihexylphenyl or 3,5-dioctylphenyl or 3,5-dialkoxyphenyl wherein alkoxy is C1-16 alkoxy, most preferably 3,5-dihexyloxyphenyl or 3,5-dioctyloxyphenyl, or 2,4,6-trialkylphenyl wherein alkyl is C1-16 alkyl, most preferably 2,4,6-trihexylphenyl or 2,4,6-trioctylphenyl or 2,4,6-trialkoxyphenyl wherein alkoxy is C1-16 alkoxy, most preferably 2,4,6-trihexyloxyphenyl or 2,4,6-trioctyloxyphenyl or 4-thioalkylphenyl wherein thioalkyl is C1-16 thioalkyl, most preferably 4-thiohexylphenyl, 4-thiooctylphenyl or 4-thiododecylphenyl, or 2,4-dithioalkylphenyl wherein thioalkyl is C1-16 thioalkyl, most preferably 2,4-dithiohexylphenyl or 2,4-dithiooctylphenyl, or 3,5-dithioalkylphenyl wherein thioalkyl is C1-16 thioalkyl, most preferably 3,5-dithiohexylphenyl or 3,5-dithiooctylphenyl, or 2,4,6-trithioalkylphenyl wherein thioalkyl is C1-16 thioalkyl, most preferably 2,4,6-trithiohexylphenyl or 2,4,6-trithiooctylphenyl, or from thiophene that is optionally substituted, preferably in 5-position, 4,5-positions or 3,5-positions, with alkyl, alkoxy or thioalkyl having 1 to 16 C atoms, more preferably from formulae SUB7-SUB18 above, most preferably from subformulae SUB14-SUB18,
R⁵⁻⁹ are H,
at least one of R⁵⁻⁹ is different from H,
R⁵⁻⁹, when being different from H, are each independently selected from F, Cl, CN or straight-chain or branched alkyl, alkoxy, sulfanylalkyl, sulfonylalkyl, alkylcarbonyl, alkoxycarbonyl and alkylcarbonyloxy, each having up to 20 C atoms and being unsubstituted or substituted by one or more F atoms, preferably from F, or alkyl or alkoxy having up to 16 C atoms that is optionally fluorinated, more preferably from formulae SUB1-SUB6 above,
R⁵⁻⁹, when being different from H, are each independently selected from aryl or heteroaryl, preferably phenyl or thiophene, each of which is optionally substituted with one or more groups L as defined in formula IA and has 4 to 30 ring atoms, preferably from phenyl that is optionally substituted, preferably in 4-position, 2,4-positions, 2,4,6-positions or 3,5-positions, with alkyl or alkoxy having 1 to 20 C atoms, preferably 1 to 16 C atoms, more preferably from formulae SUB7-SUB18 above.

Preferred repeating units of formula I and I1-I5 are selected from the following subformula

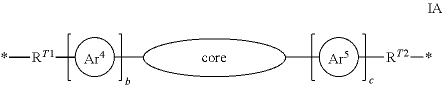

wherein Ar⁴, Ar⁵, R^{T1}, R^{T2}, b and c, independently of each other and on each occurrence identically or differently, have the meanings given in formula I or one of the preferred meanings given above and below, and “Core” is, on each occurrence identically or differently, a polycyclic divalent group selected from the following formulae

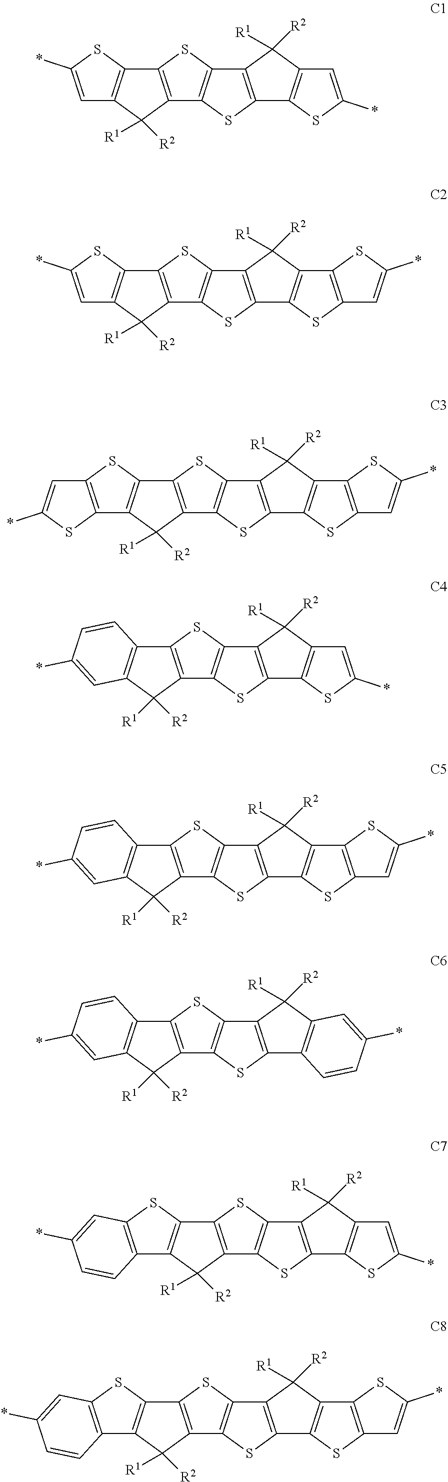

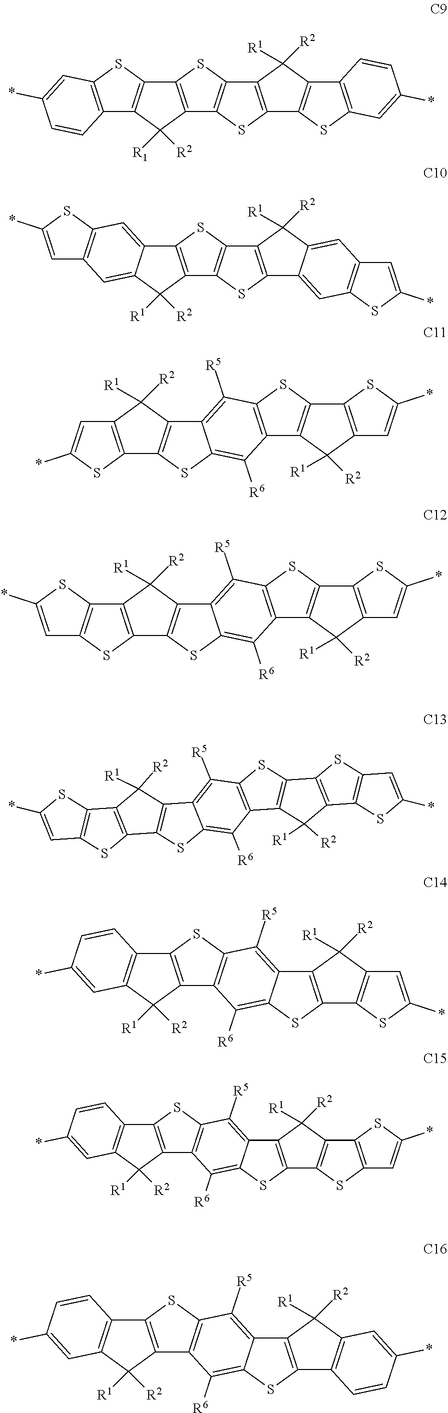

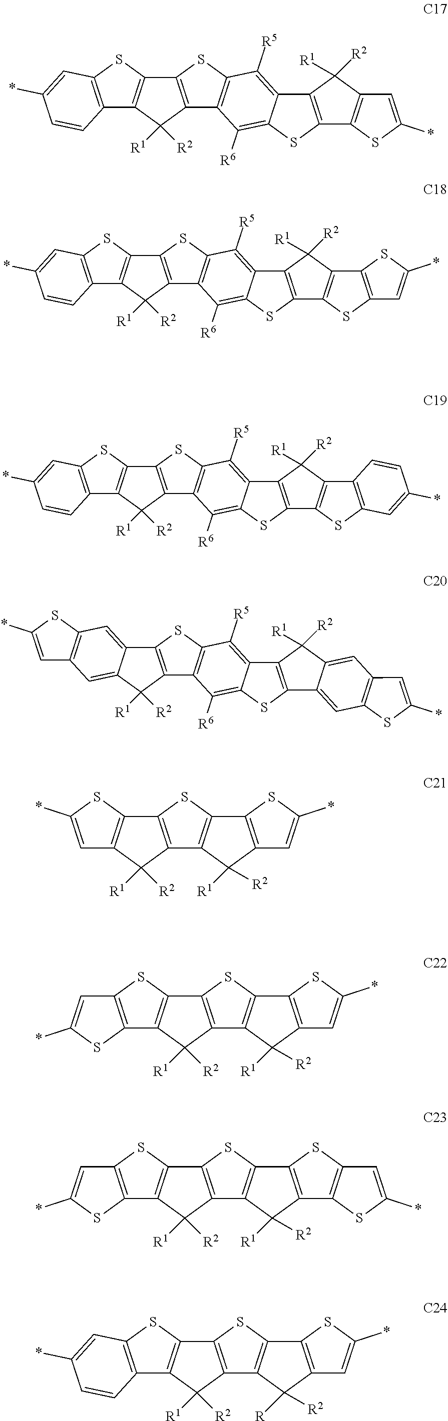

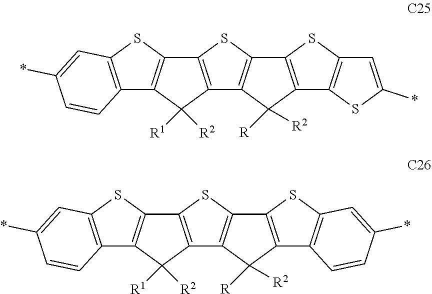

wherein R¹⁻⁶ have the meanings given above and below.

Preferred are the core groups of formula C1, C2, C3, C11, C12, C13, C21, C22 and C23; very preferred are C1, C2, C3, C11, C12, C13.

Preferred repeating units of formula I, I1-I5 and IA are selected from the following subformulae

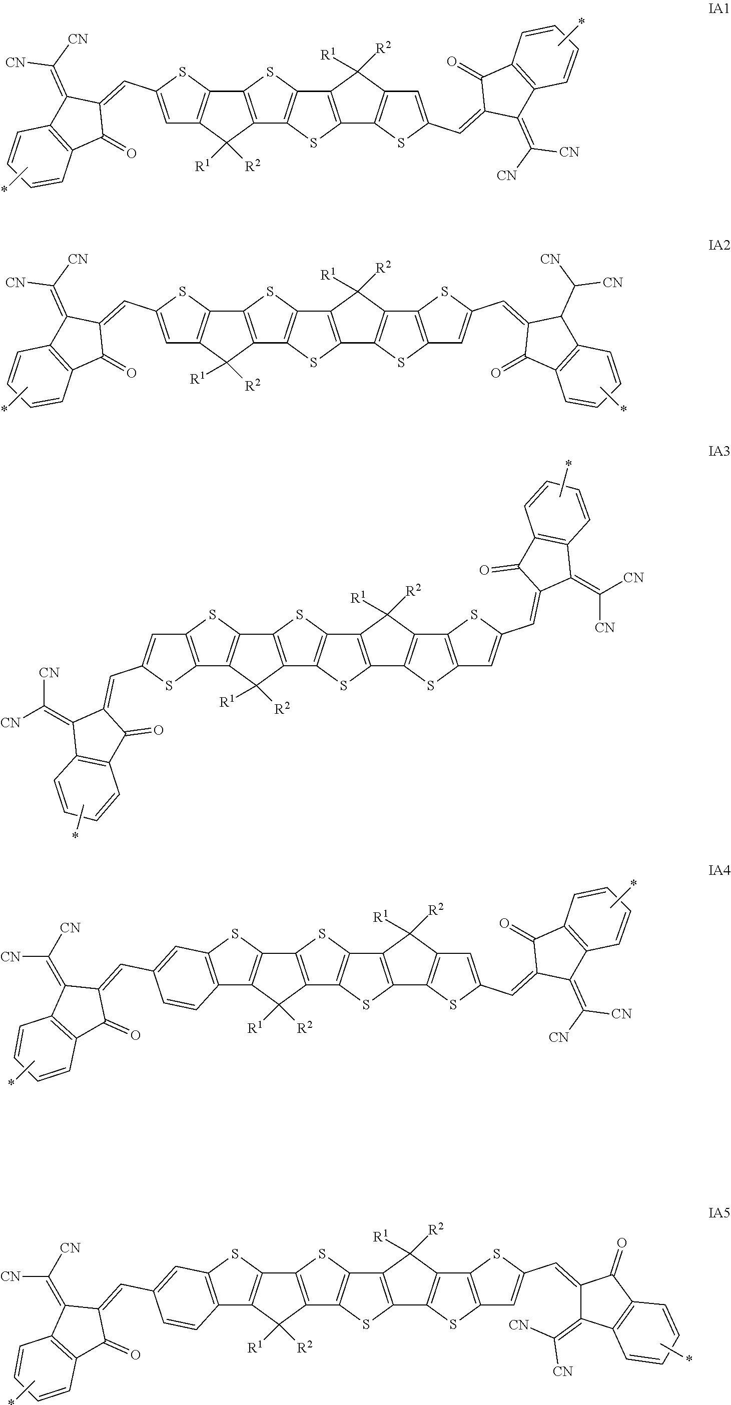

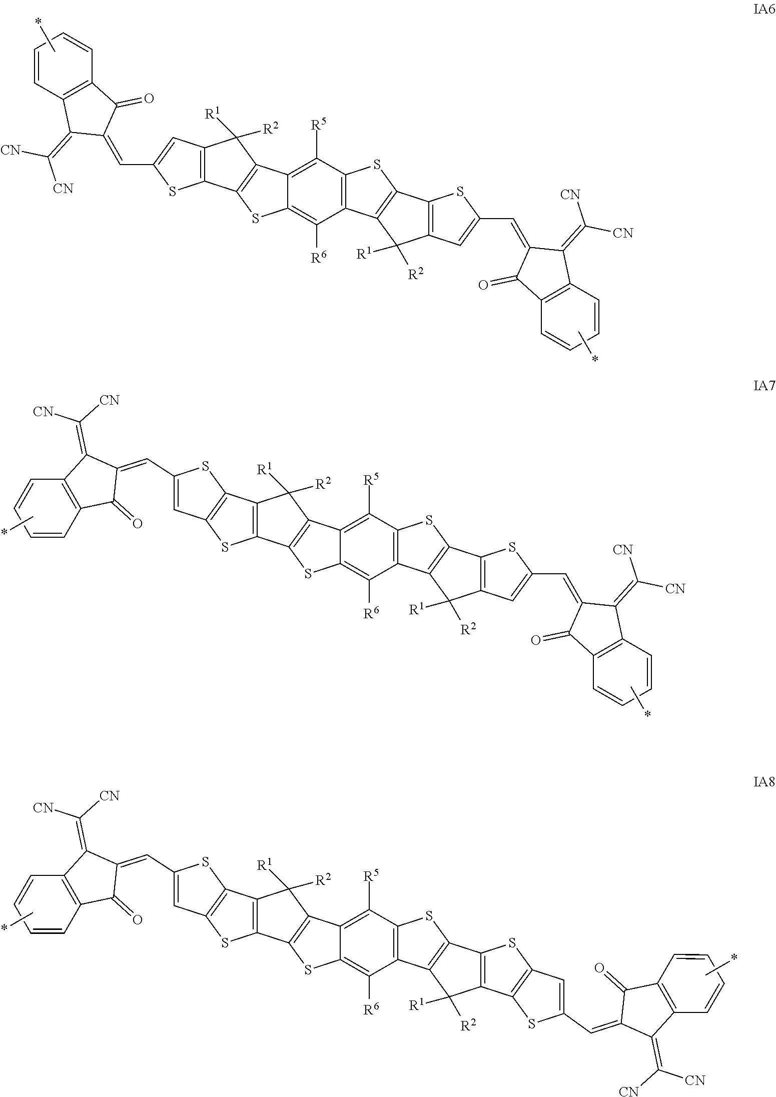

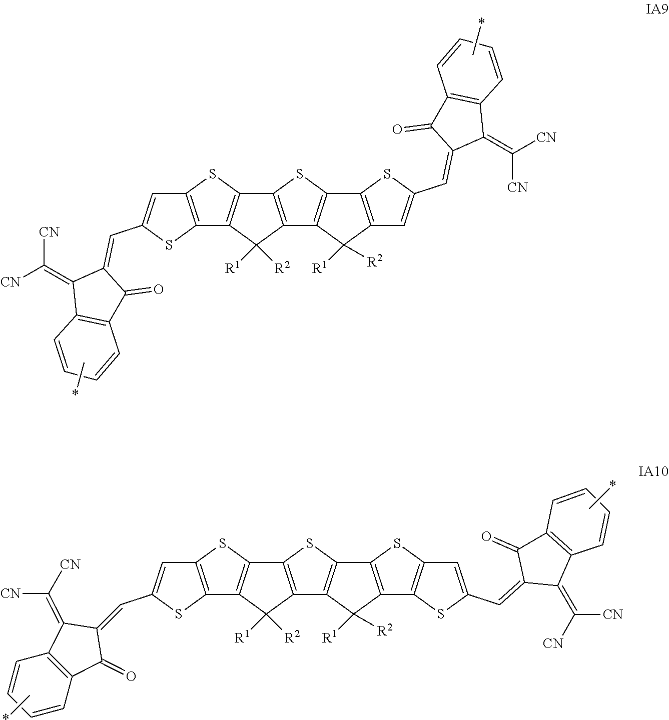

wherein R¹⁻⁶ have the meanings given in formula I or one of the preferred meanings as given above and below.

Another embodiment of the invention relates to a polymer comprising one or more repeating units of formula I, I1-I5, IA, IA1-IA10 or their subformulae.

Preferably, the polymer comprises, very preferably consists of, one or more, preferably two or more, units of formula I, I1-I5, IA, IA1-IA10 or their subformulae, and one or more units selected from the following groups
A1) the group consisting of arylene or heteroarylene units that are different from formula I and its subformulae, have from 5 to 20 ring atoms, are mono- or polycyclic, do optionally contain fused rings, and are unsubstituted or substituted by one or more identical or different groups L as defined in formula I,
B1) —CY¹═CY²— wherein Y¹ and Y² are independently of each other H, F, Cl or CN,
C1) —C≡C—.

Preferably one or more of the arylene or heteroarylene units of group A1 are selected from electron donor units, and/or one or more of the additional arylene or heteroarylene units of group A1 are selected from electron acceptor units.

Further preferably, the polymer comprises at least one acceptor unit and/or at least one donor unit, and further comprises one or more units selected from groups A1, B1 and C1 above, which are located between two units selected from the group consisting of U, A and D, hereinafter also referred to as “spacer units”.

In another preferred embodiment the polymer comprises, in addition or alternatively to the units selected from groups A1, B1 and C1, one or more units which interrupt conjugation, and which are preferably selected from the following groups:
D1) the group consisting of straight-chain, branched or cyclic alkylene with 1 to 30, preferably 2 to 16, C atoms, in which one or more CH₂ groups are each optionally replaced by —O—, —S—, —C(═O)—, —C(═S)—, —C(═O)—O—, —O—C(═O)—, —SiR⁰R⁰⁰—, —CF₂—, —CR⁰═CR⁰⁰—, —CY¹═CY²— or —C≡C— in such a manner that O and/or S atoms are not linked directly to one another, and in which one or more H atoms are each optionally replaced by F, Cl, Br, I or CN, but excluding fully conjugated unsaturated groups (like e.g. —CR⁰═CR⁰⁰—CR⁰═CR⁰⁰— or —C≡C—),
E1) the group consisting of units formed from one or more units selected from groups A1, B1 and C1 and one or more units of group D1.

Further preferably the polymer comprises, preferably consists of, one or more, preferably two or more, repeating units of formula II1 and/or II2, and optionally one or more repeating units of formula II3:

—(C¹)ₐ—U—(C²)_{b}—(C³)_{c}(C⁴)_{d}— II1

—(C¹)ₐ—(C²)_{b}—U—(C³)_{c}—(C⁴)_{d}— II2

—(C¹)ₐ—(C²)_{b}—(C³)_{c}—(C⁴)_{d}— II3

wherein the individual radicals, independently of each other and on each occurrence identically or differently, have the following meanings
U a unit selected from formula I, I1-I5, IA, IA1-IA10 or their subformulae as defined above and below,
C¹⁻⁴ a unit selected from groups A1, B1, C1, D1 and E1 as defined above, preferably from group A1 or E1,

a, b, c, d 0 or 1, wherein in formula II3 a+b+c+d≥1.

Preferably the polymer comprises one or more repeating units of formula II1 or II2 wherein a+b+c+d≥1.

Further preferably the polymer comprises one or more repeating units of formula II1 wherein b=1 and a=c=d=0 and one or more repeating units of formula II3 wherein a=b=0 and c=d=1.

Further preferably the polymer comprises two or more distinct repeating units of formula II1 wherein b=1 and a=c=d=0.

Further preferably the polymer comprises at least one unit of formula II1, II2 or II3 wherein at least one of C¹, C², C³ and C⁴ is a donor unit.

Further preferably the polymer is selected of formula III:

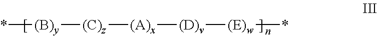

wherein the individual radicals, independently of each other and on each occurrence identically or differently, have the following meanings

A a unit of formula II1 or II12,

B, C, D, E a unit of formula II1, II2 or II3,

x>0 and ≤1,

v, w, y, z≥0 and <1,

v+w+x+y+z 1, and

n an integer >1, preferably ≥5.

Further preferably the polymer comprises, very preferably consists of, one or more units selected from the group consisting of the following formulae and their mirror images

—(U)— U1

—(U-Sp)- U2

-(Sp-U-Sp)- U3

—(U-D)- U4

—(U-A)- U5

-(D-Sp)- U6

-(A-Sp)- U7

-(A-D)- U8

-(D)- U9

-(Sp-D-Sp)- U10

-(A)- U11

-(Sp-A-Sp)- U12

wherein the individual radicals, independently of each other and on each occurrence identically or differently, have the following meanings
U the meaning given in formula II1,
D a unit selected from group A1 as defined above which is a donor unit,
A a unit selected from group A1 as defined above which is an acceptor unit,
Sp a spacer unit which is located between two units selected from the group consisting of U, D and A, and is selected from groups A1, B1, C1, D1 and E1 as defined above, preferably from groups A1 and E1,

and the polymer comprises at least one unit selected from formulae U1-U5.

Very preferred polymers are selected from formulae Pi-Px

—[(U-Sp]ₙ- Pi

—[(U¹-Sp)ₓ-(U²-Sp)_{y}]ₙ- Pii

—[(U-Sp¹)ₓ-(U-Sp2)_{y}]ₙ- Piii

—[(U-Sp)ₓ-(D-Sp)_{y}]ₙ- Piv

—[(U-D)ₓ-(U-Sp)_{y}]ₙ- Pv

-[(D)ₓ-(Sp-U-Sp)_{y}]ₙ- Pvi

—[(U)ₓ-(Sp-D-Sp)_{y}]ₙ- Pvi

-[D-U]ₙ— Pvii

-[D-Sp-U-Sp]ₙ- Pviii

-[D¹-U-D²-U]ₙ— Pix

-[D-U¹-D-U²]ₙ— Px

wherein A, D and Sp are as defined in formulae U2-U12, A, D and Sp can each, in case of multiple occurrence, also have different meanings, D¹ and D² have one of the meanings given for D and are different from each other, Sp¹ and Sp² have one of the meanings given for Sp and are different from each other, U¹ and U² have one of the meanings given for U and are different from each other, x and y denote the molar fractions of the corresponding units, x and y are each, independently of one another, a non-integer >0 and <1, with x+y=1, and n is an integer >1.

Especially preferred are polymers according to the present invention, repeating units of formulae II1, II2, II3 and U2-U12 and their subformulae, and polymers of formulae III, Pi-Px and their subformulae, wherein one or more of the units of group A1, or one or more of C¹, C², C³, C⁴, the donor units, or D, respectively, denote arylene or heteroarylene, which preferably has electron donor properties, and is selected from the group consisting of the following formulae and their mirror images

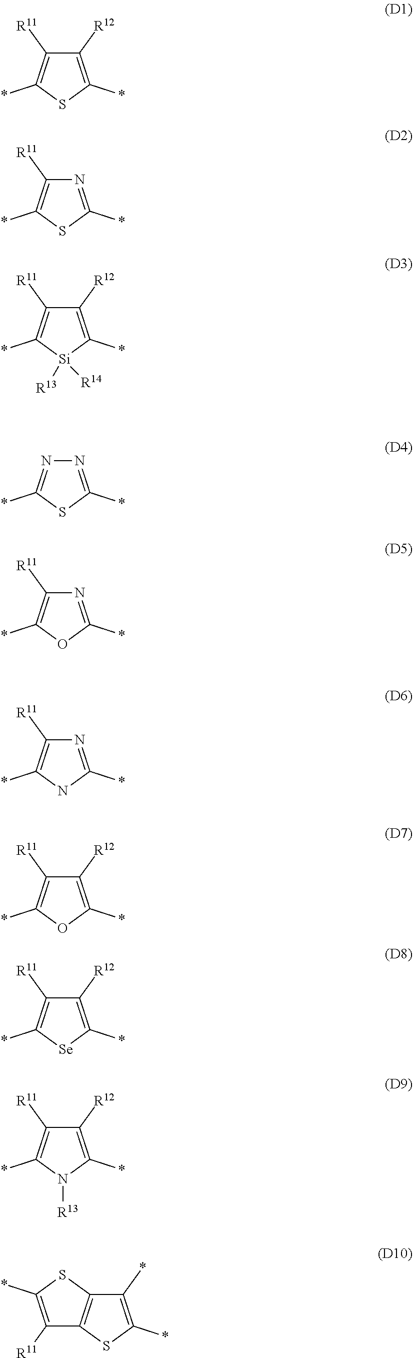

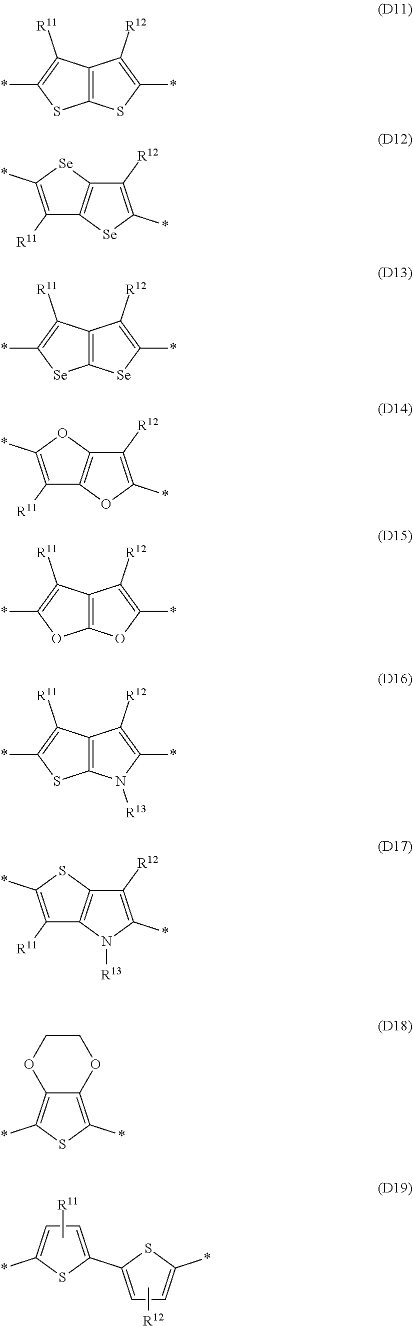

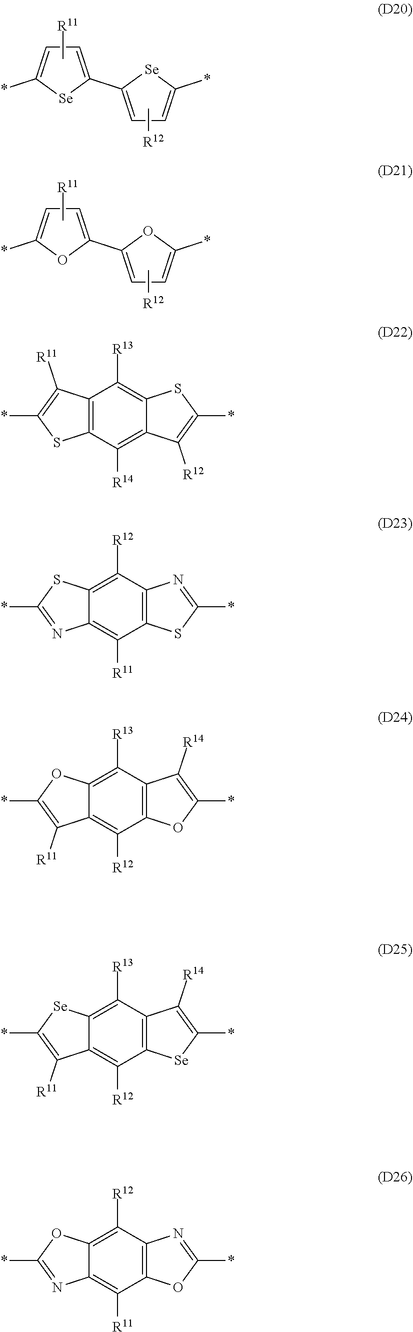

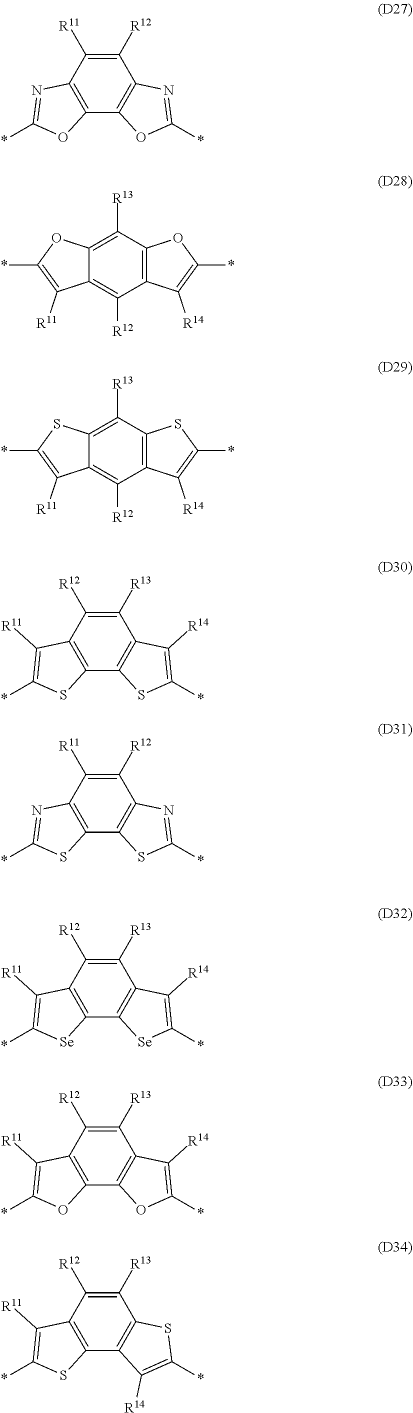

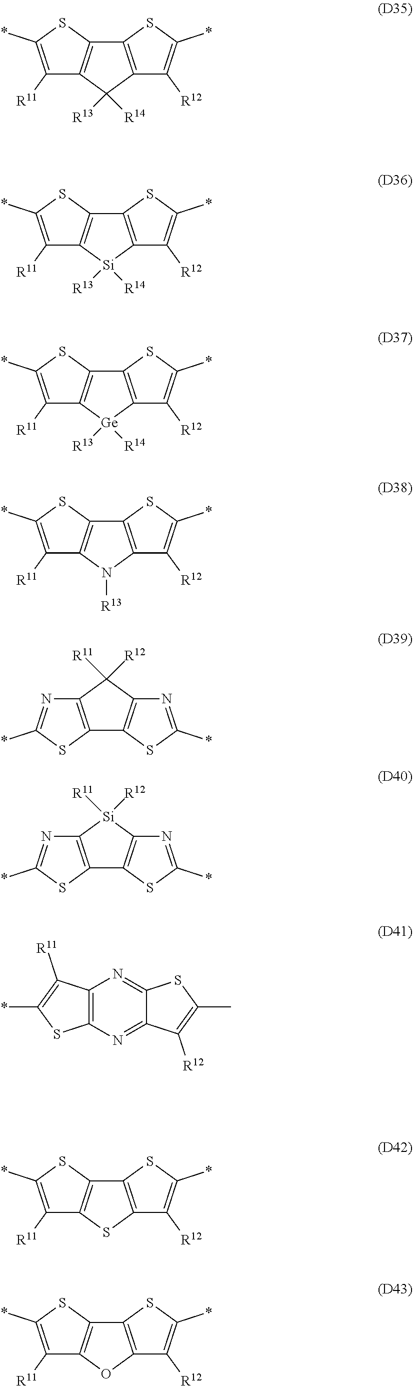

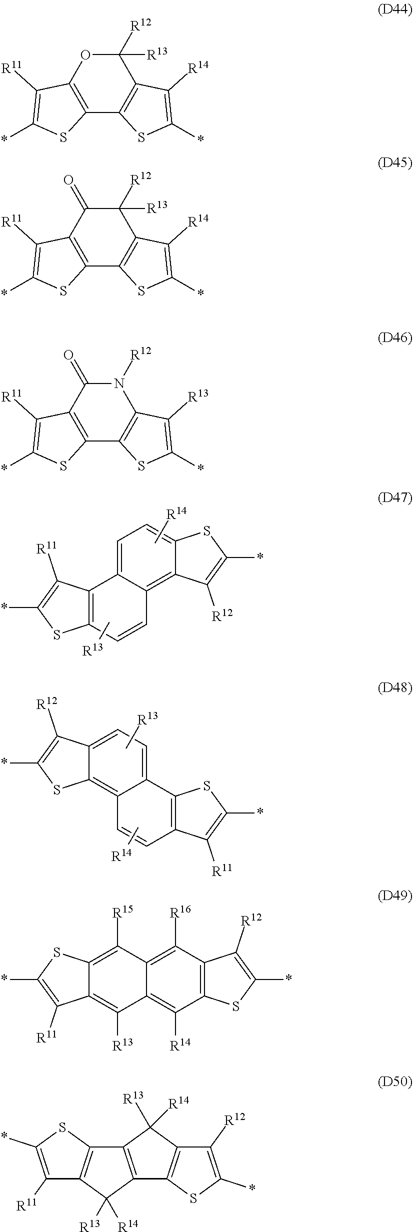

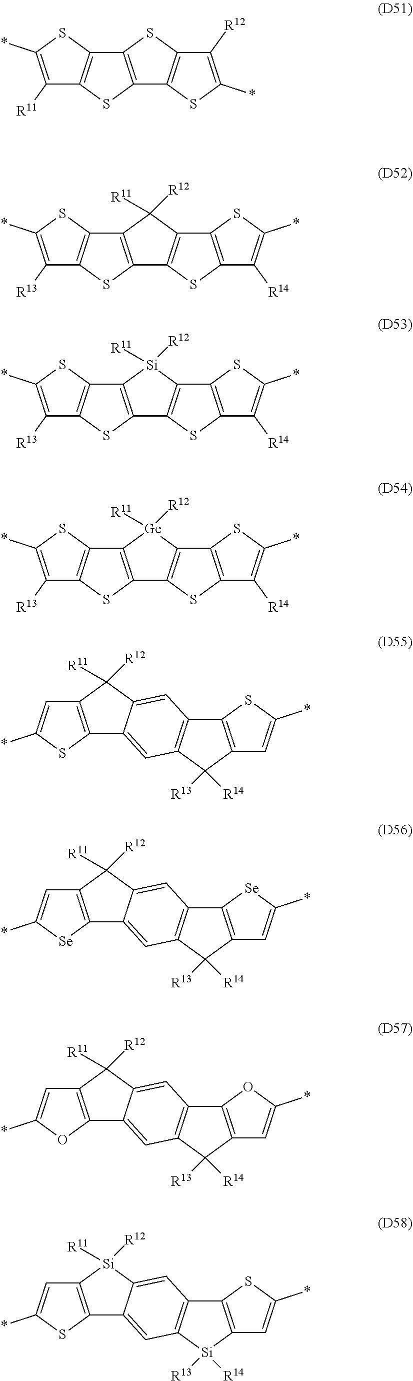

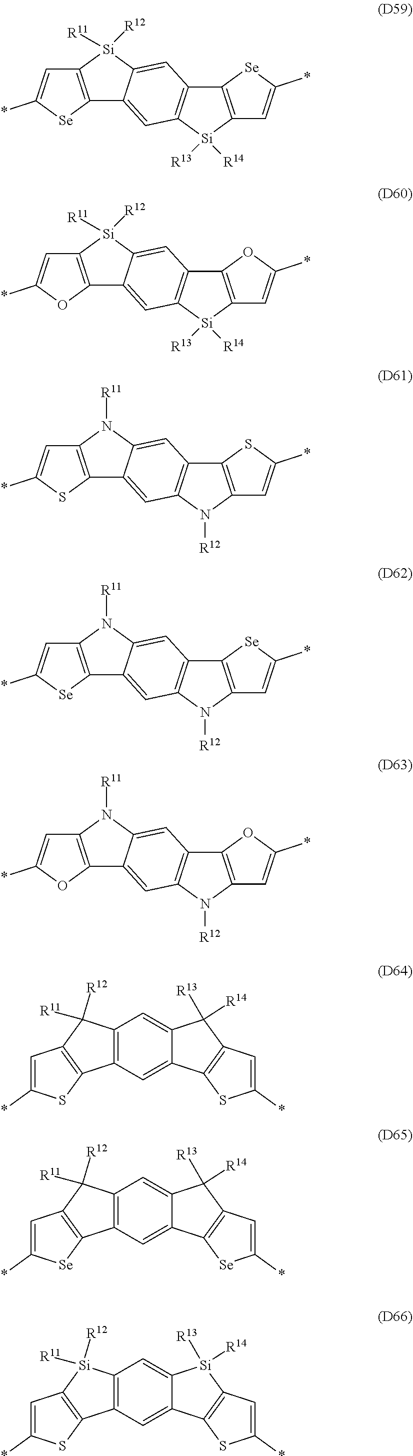

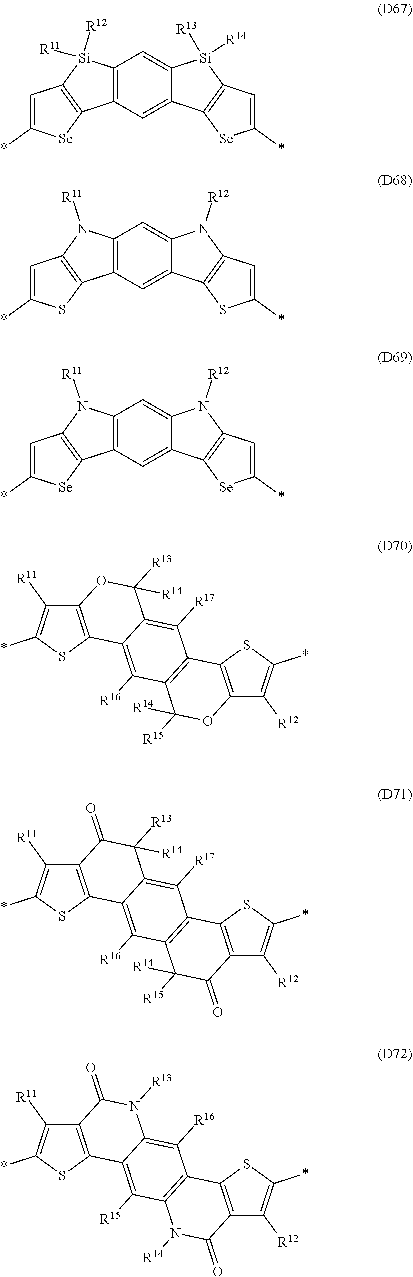

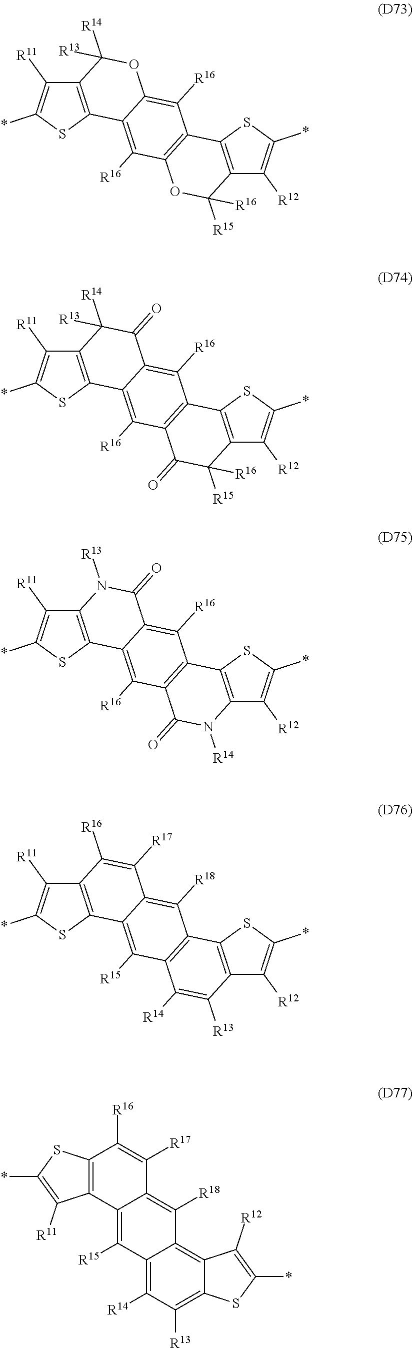

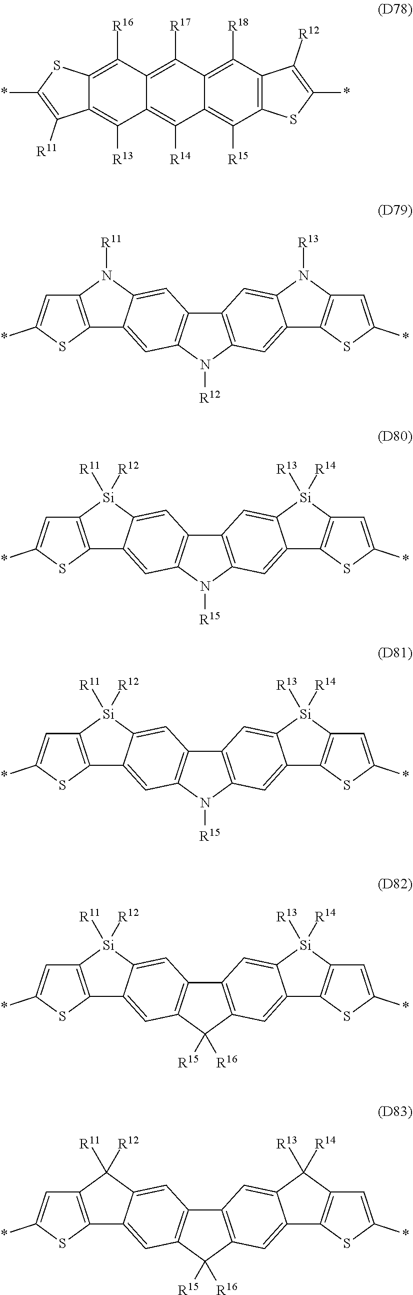

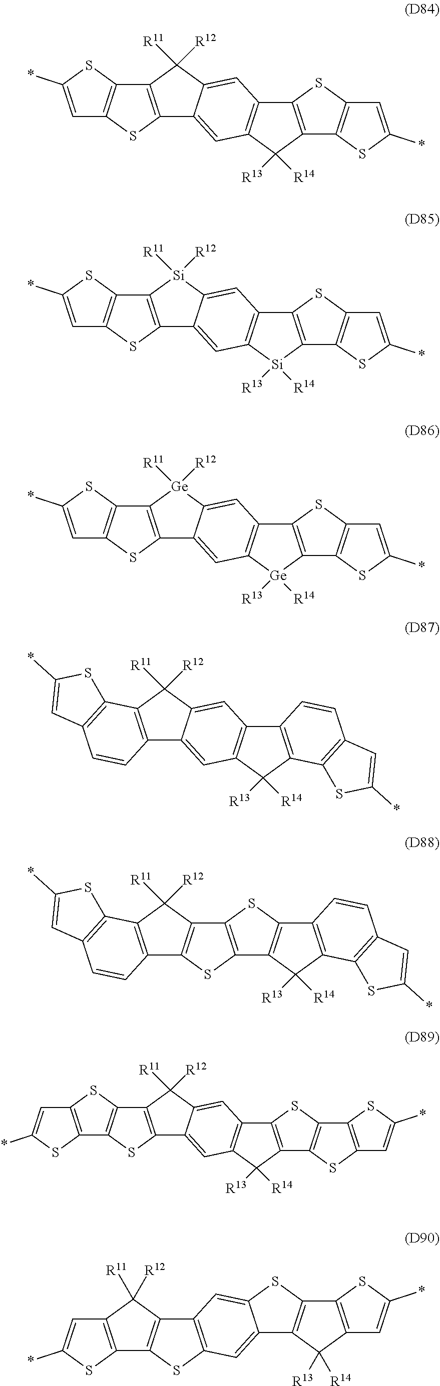

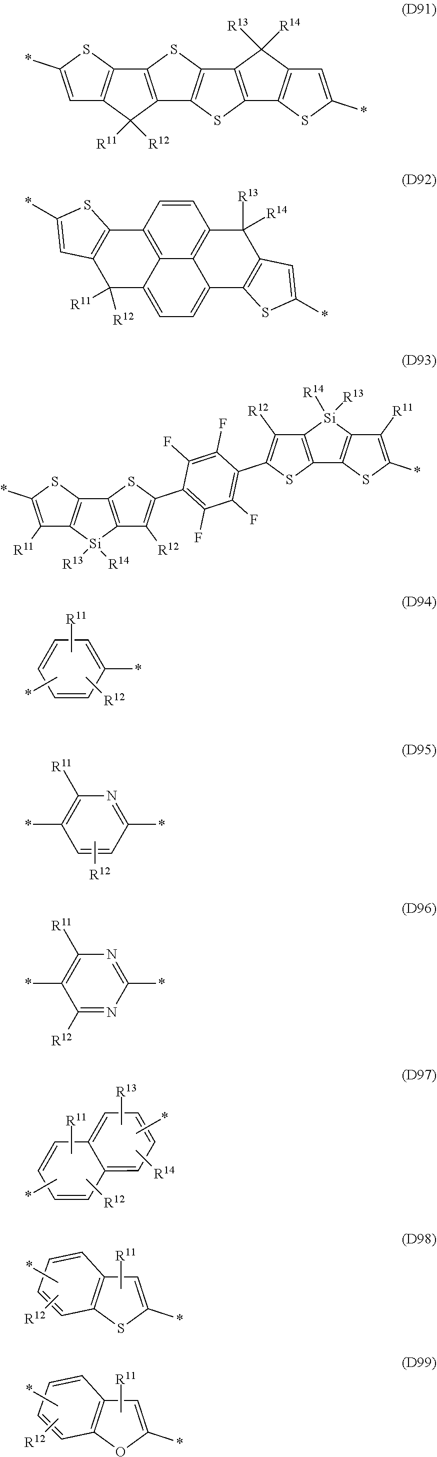

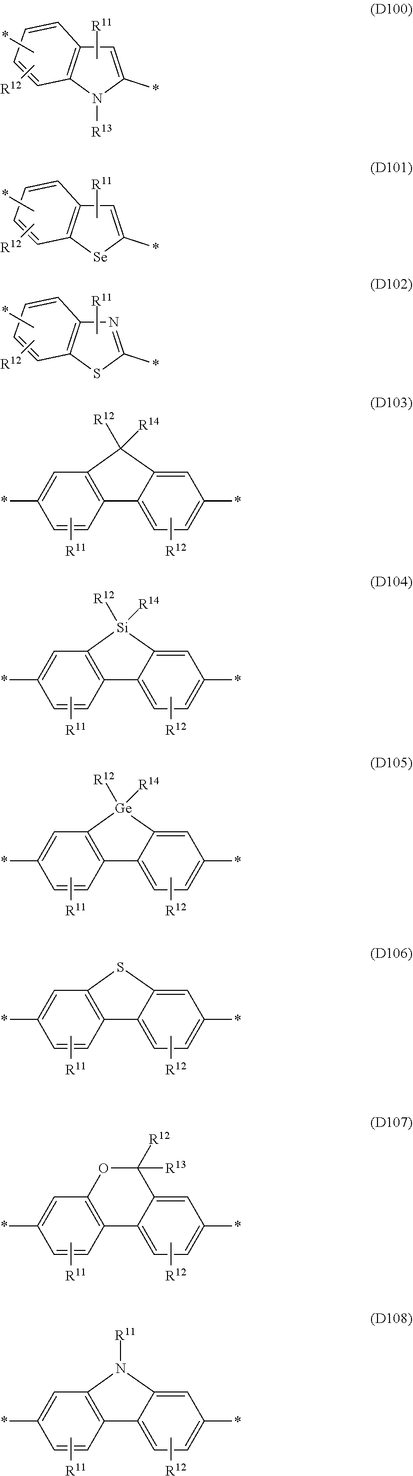

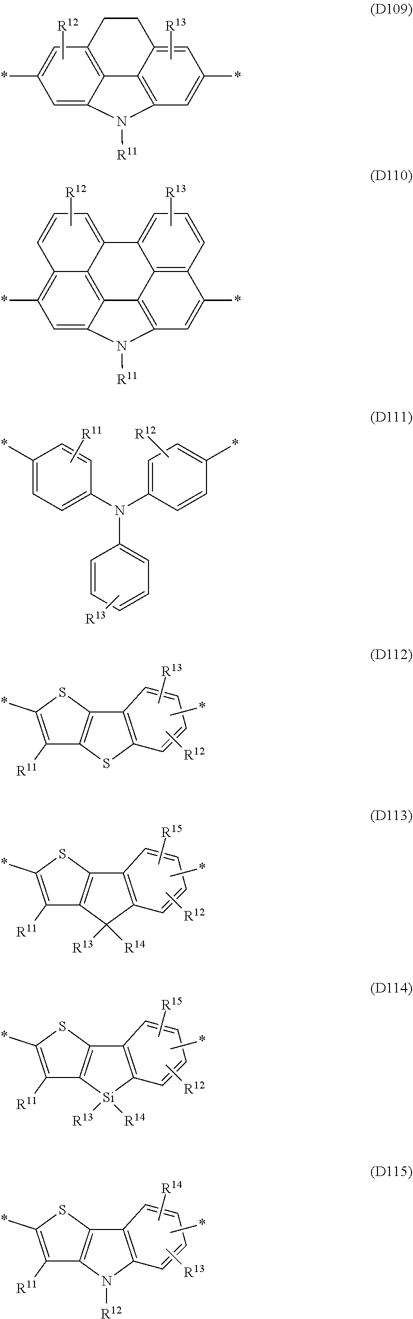

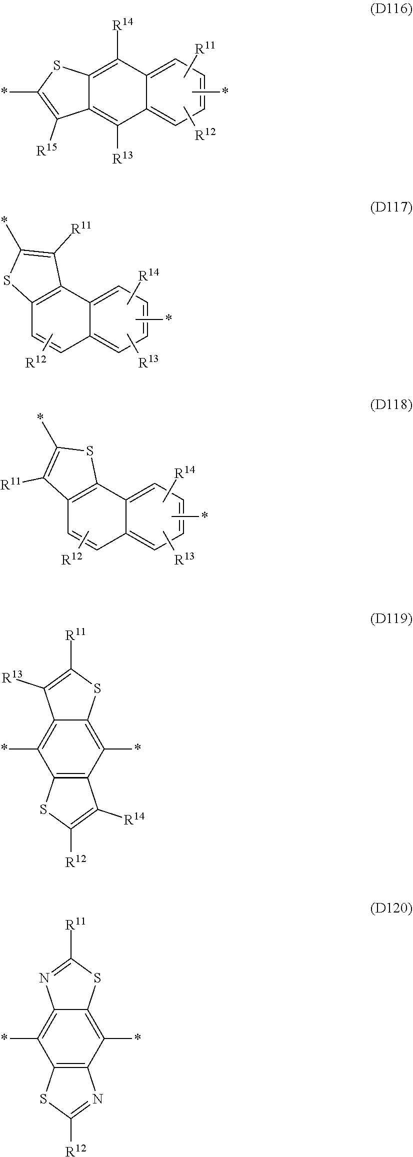

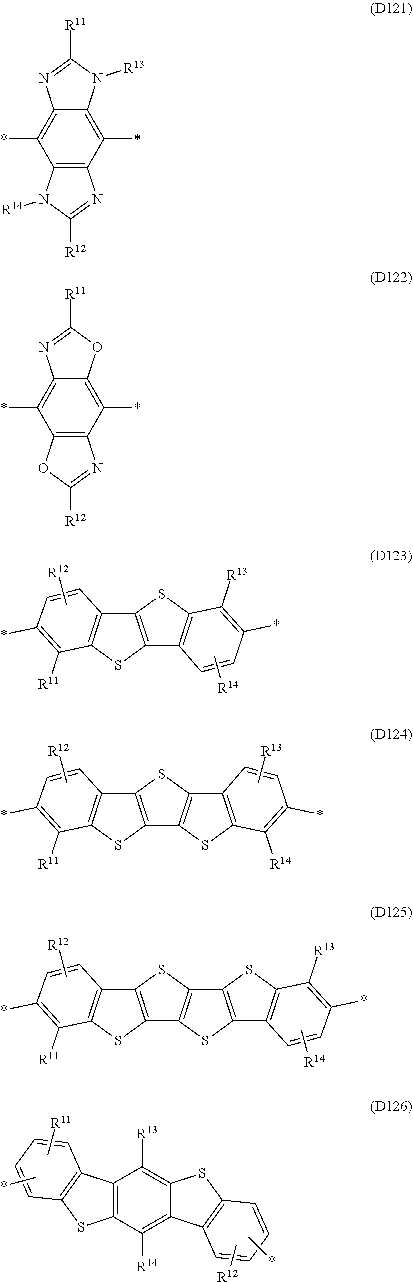

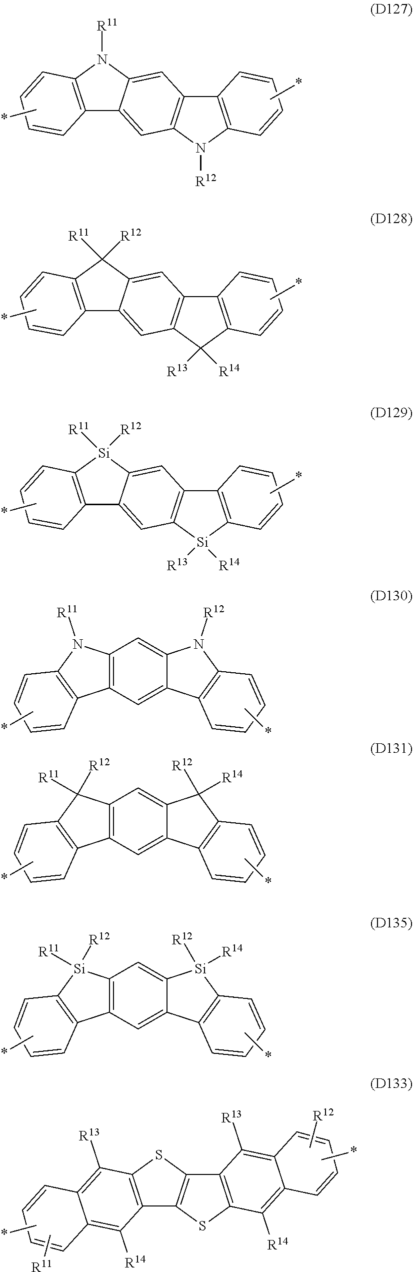

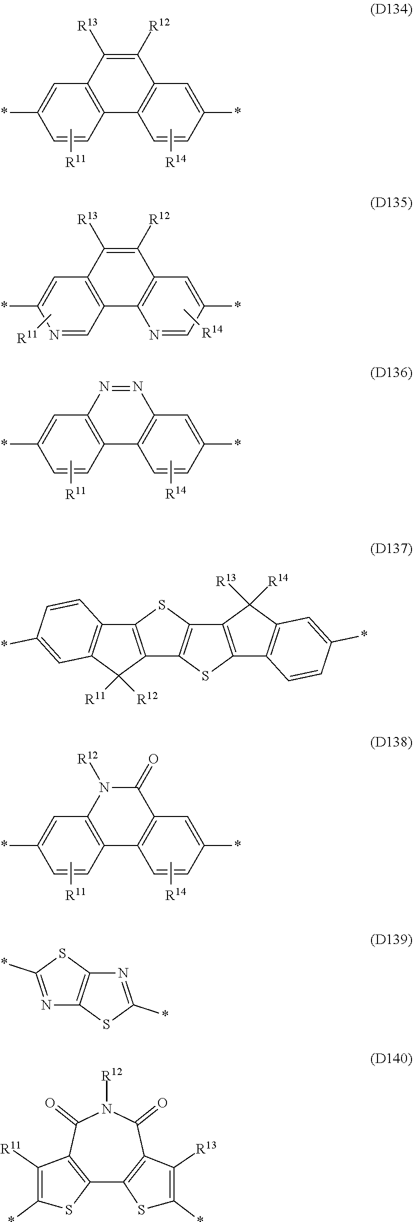

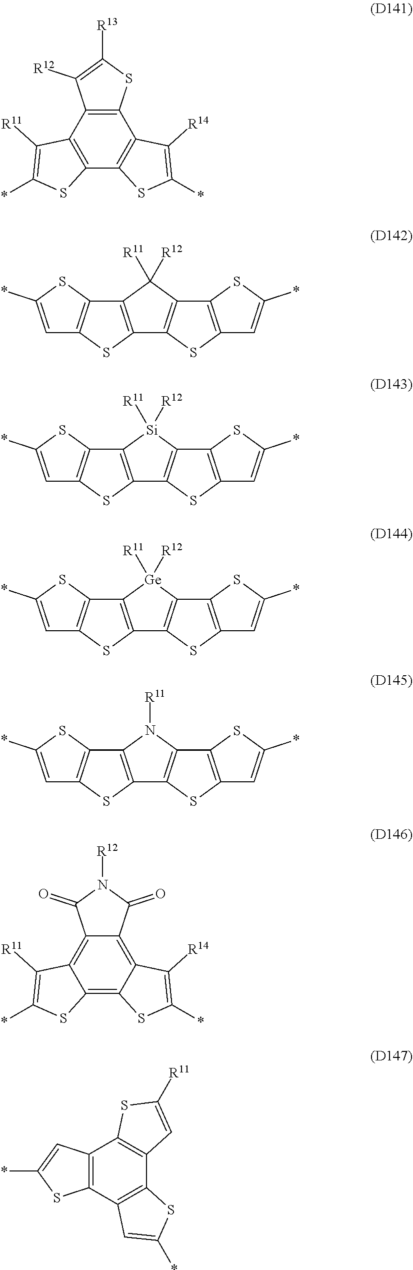

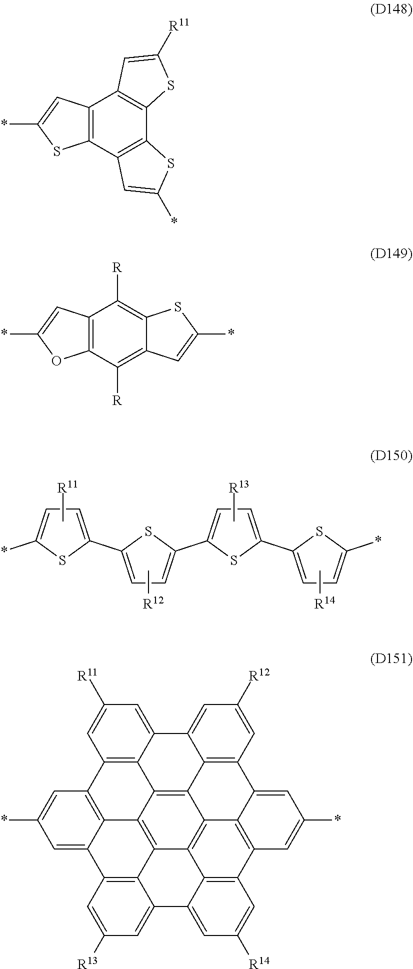

wherein R¹¹, R¹², R¹³, R¹⁴, R¹⁵, R¹⁶, R¹⁷ and R¹⁸ independently of each other have one of the meanings of R⁵ as given in formula I or one of its preferred meanings as given above and below.

Preferred donor units are selected from formulae D1, D7, D10, D11, D19, D22, D29, D30, D35, D36, D37, D44, D55, D84, D87, D88, D89, D93, D94, D106, D111, D139, D140, D141, D146 or D150 wherein preferably at least one of R¹¹, R¹², R¹³ and R¹⁴ is different from H, and in formula D150 preferably R¹² and R¹³ are F and R¹¹ and R¹⁴ are H or C1-C30 alkyl.

Further preferred are polymers according to the present invention, repeating units of formulae II1, II2, II3 and U2-U12 and their subformulae, and polymers of formulae III, Pi-Px and their subformulae, wherein one or more of the units of group A1, or one or more of C¹, C², C³, C⁴, the acceptor units, or A, respectively, denote arylene or respectively, denote arylene or heteroarylene, which preferably has electron acceptor properties and is selected from the group consisting of the following formulae and their mirror images

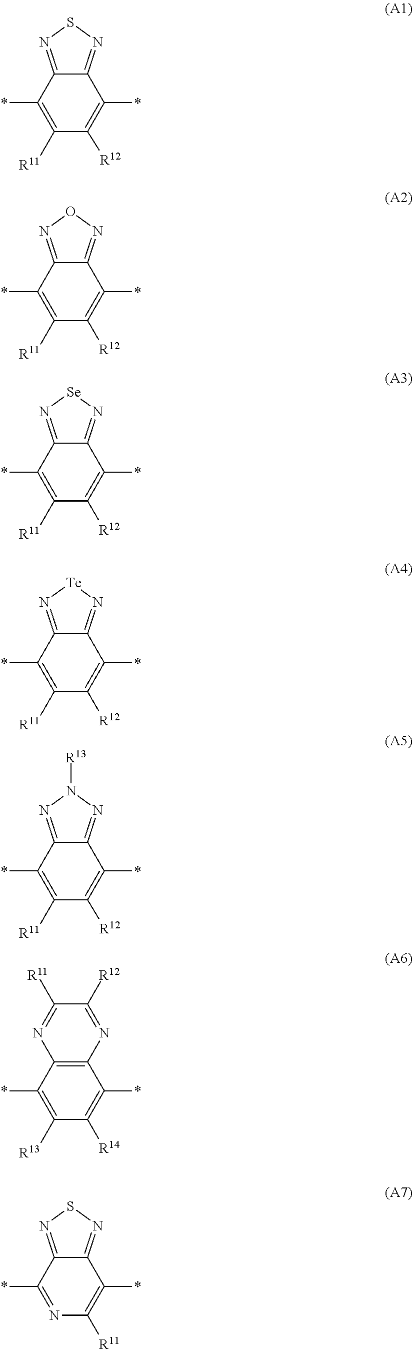

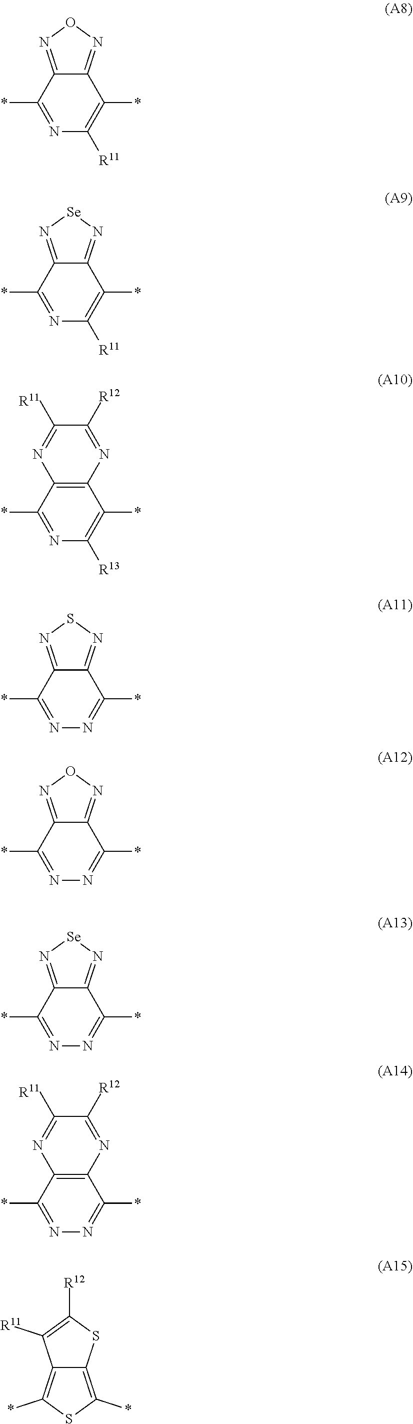

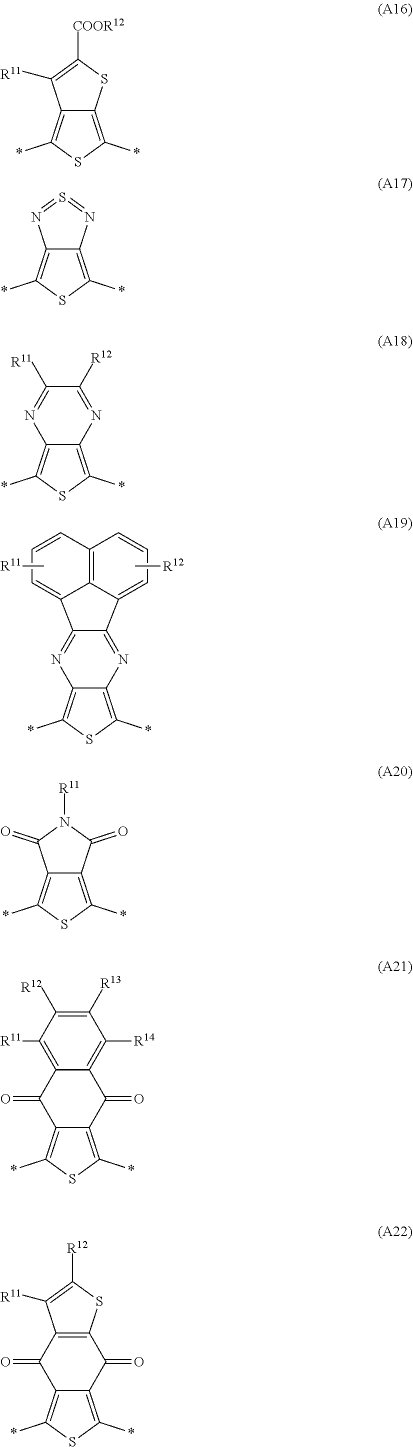

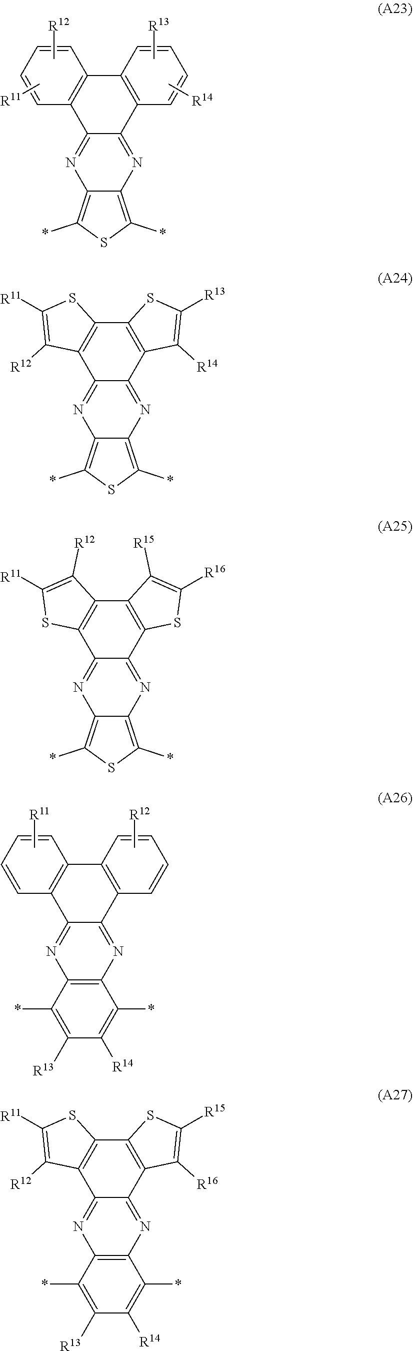

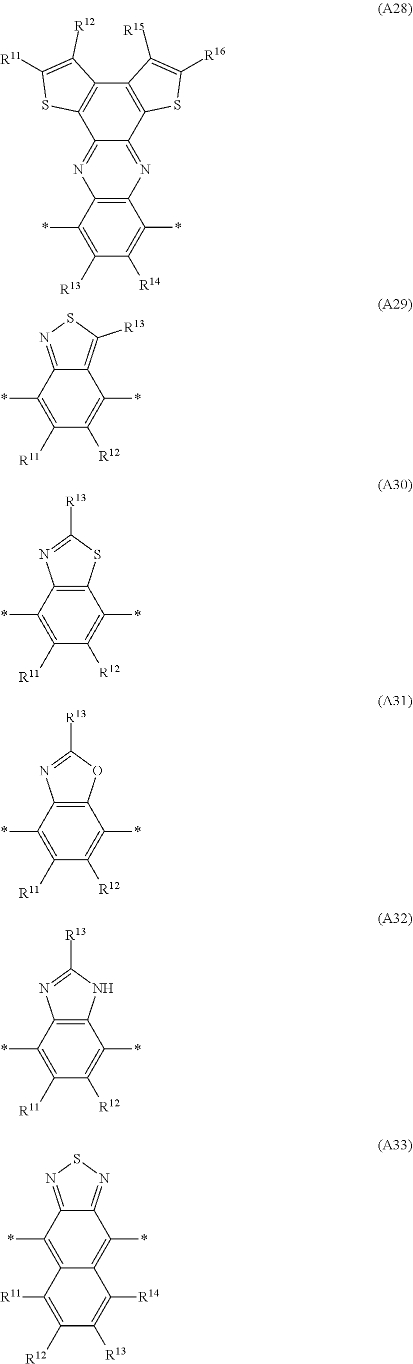

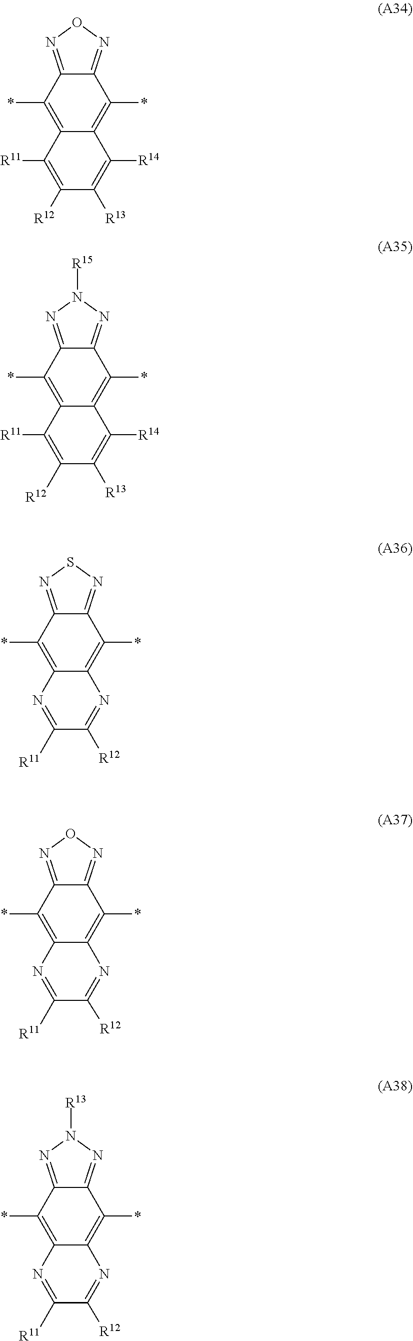

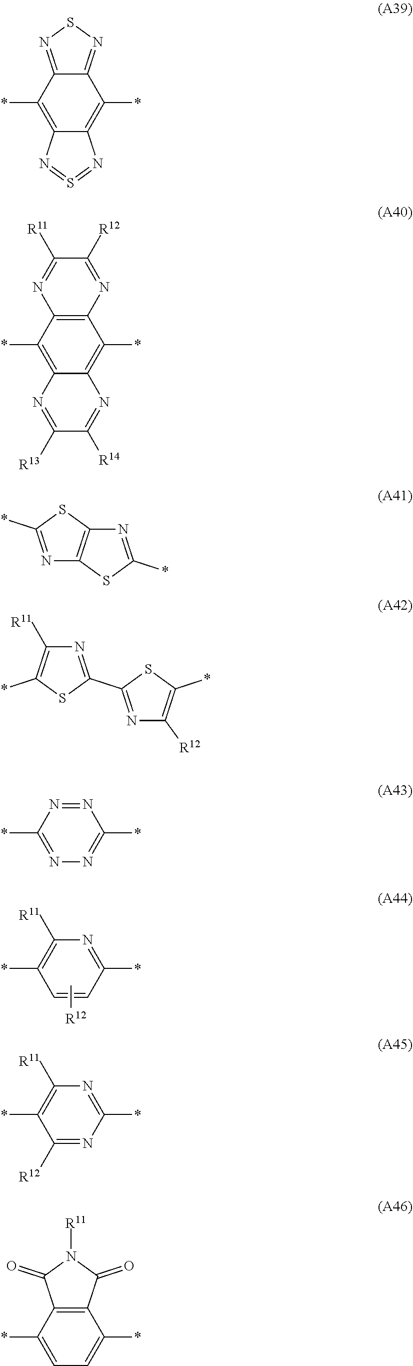

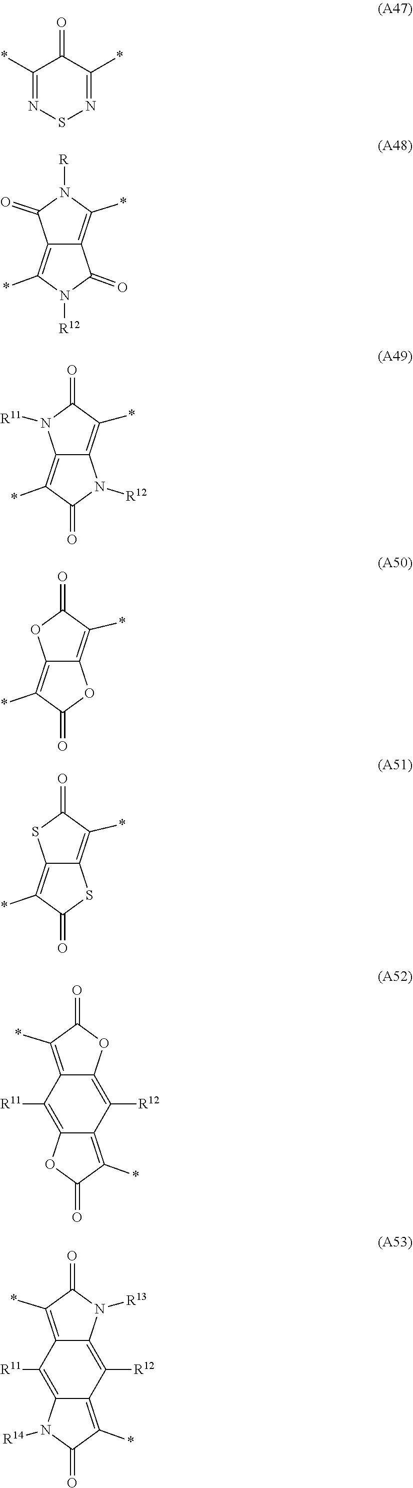

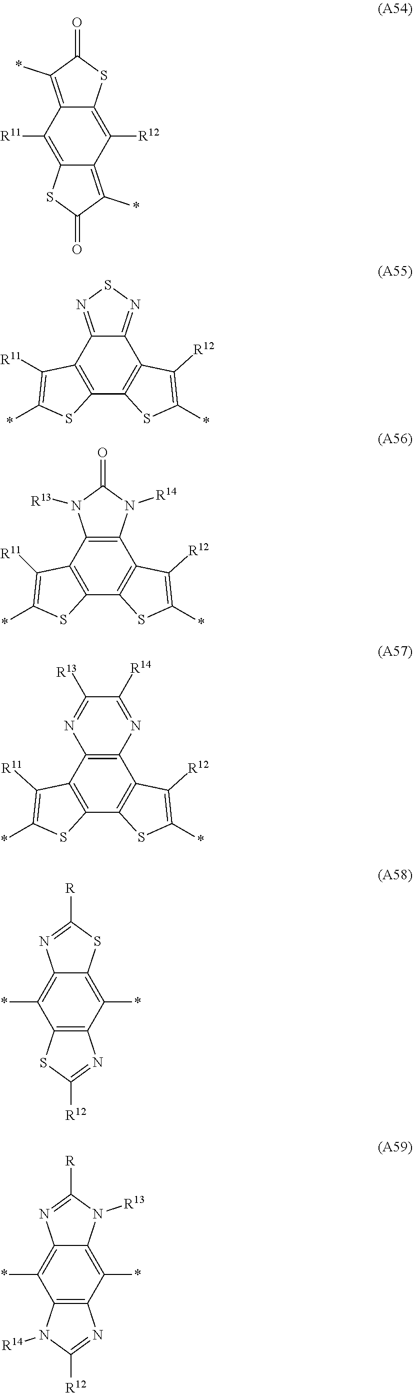

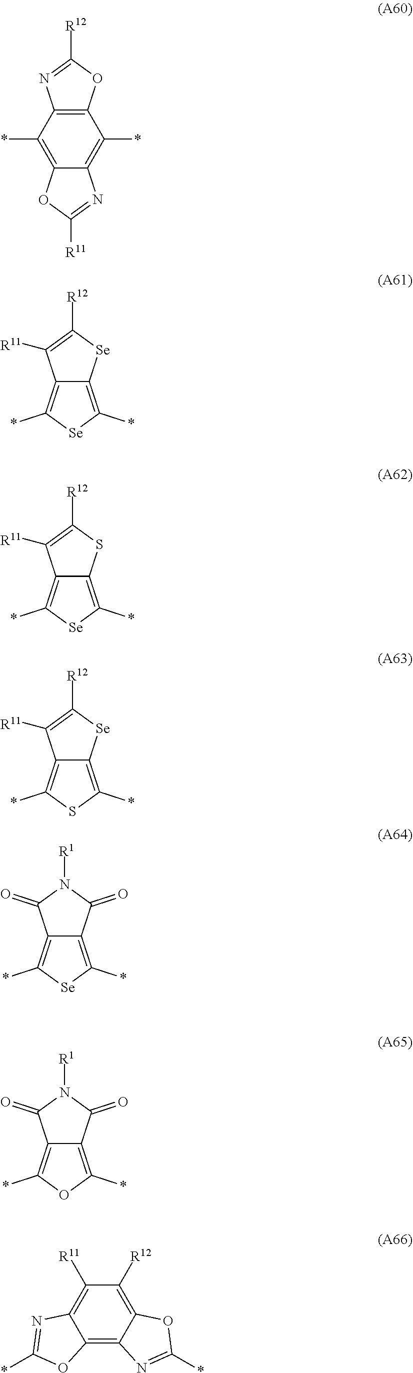

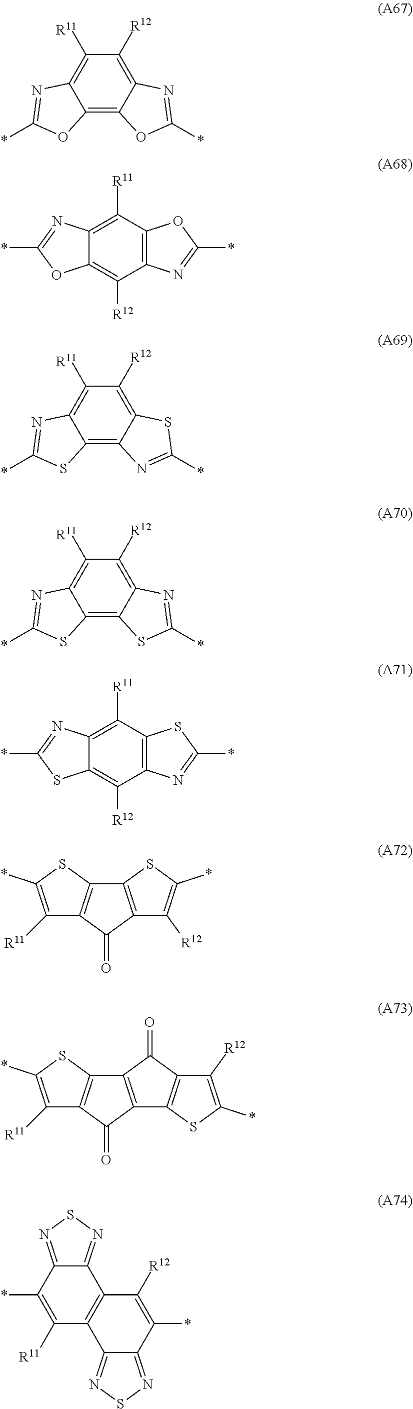

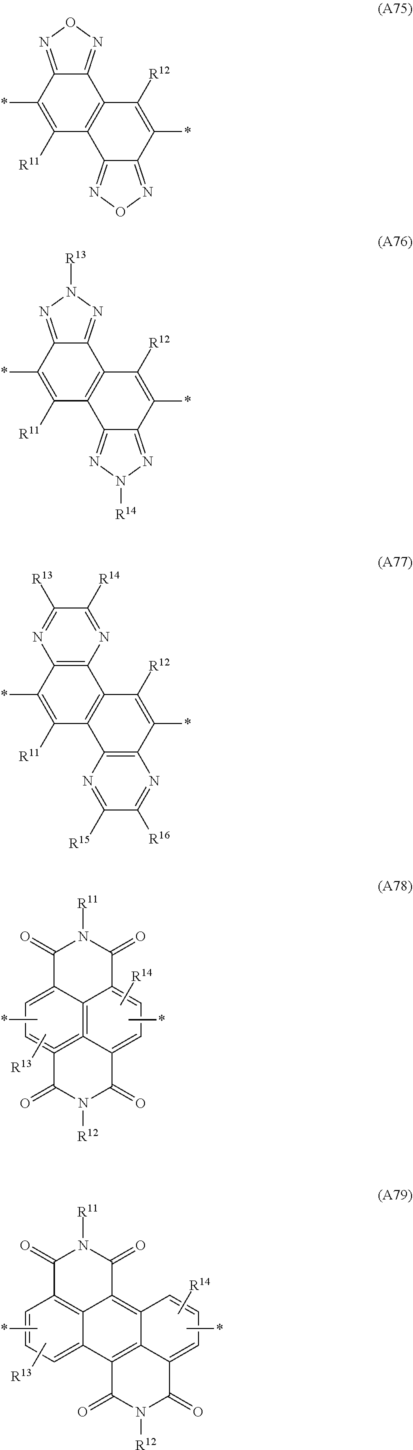

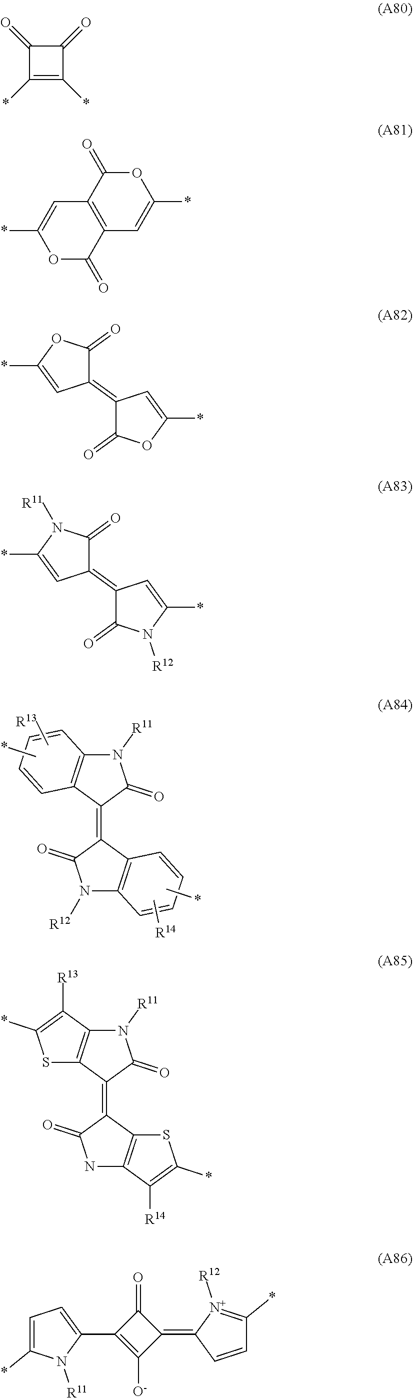

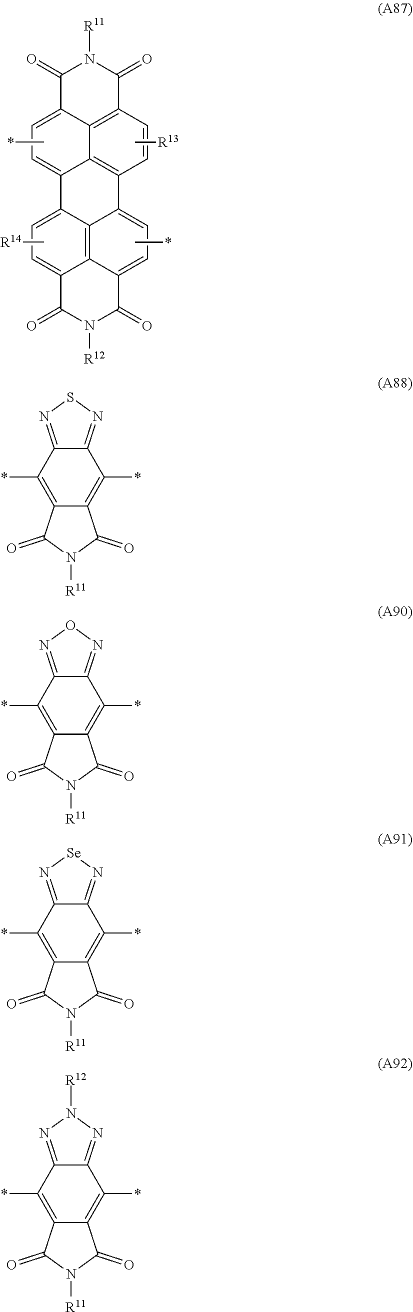

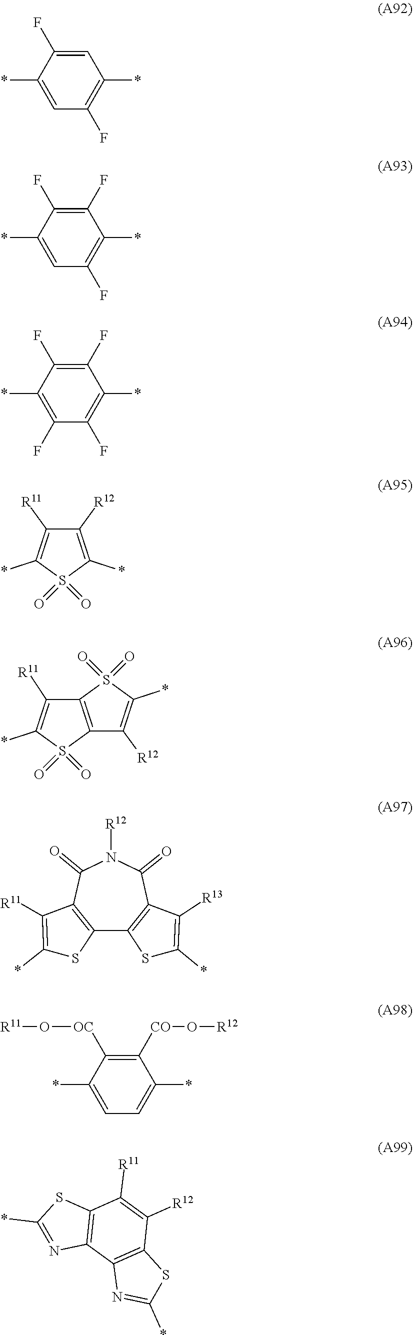

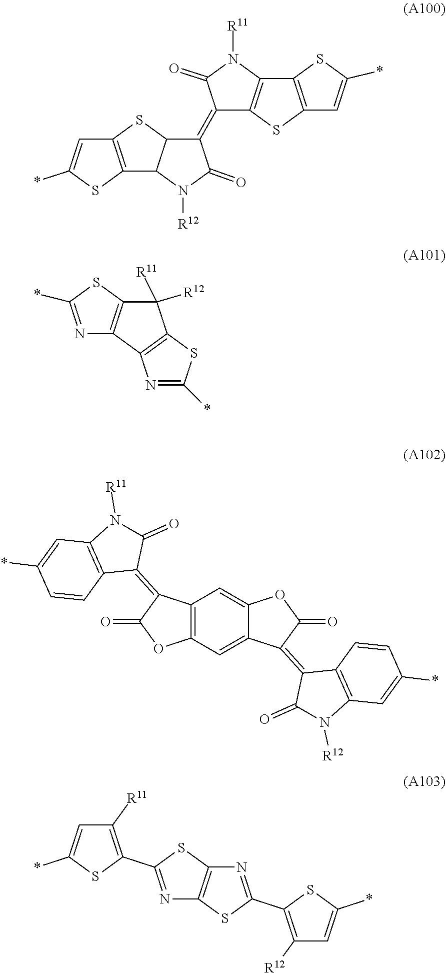

wherein R¹¹, R¹², R¹³, R¹⁴, R¹⁵ and R¹⁶ independently of each other have one of the meanings of R⁵ as given in formula I or one of its preferred meanings as given above and below.

Preferred acceptor units are selected from formulae A1, A6, A7, A15, A16, A20, A36, A74, A84, A88, A92, A94, A98 or A103 wherein preferably at least one of R¹¹, R¹², R¹³ and R¹⁴ is different from H.

Further preferred are polymers according to the present invention, repeating units of formulae II1, II2, II3 and U2-U12 and their subformulae, and polymers of formulae III, Pi-Px and their subformulae, wherein one or more of the units of group A1, or one or more of C¹, C², C³, C⁴, the spacer units, or Sp, respectively, denote arylene or heteroarylene selected from the group consisting of the following formulae and their mirror images

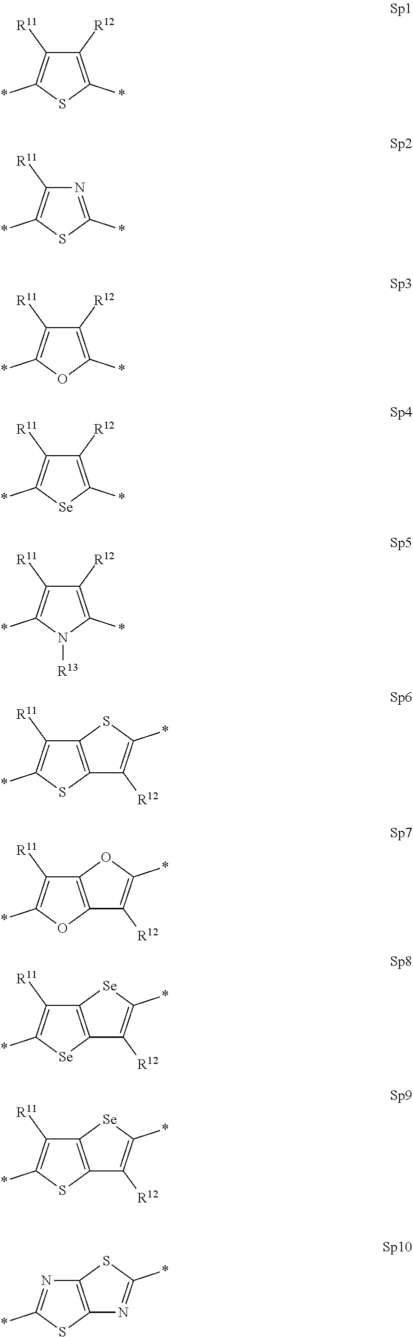

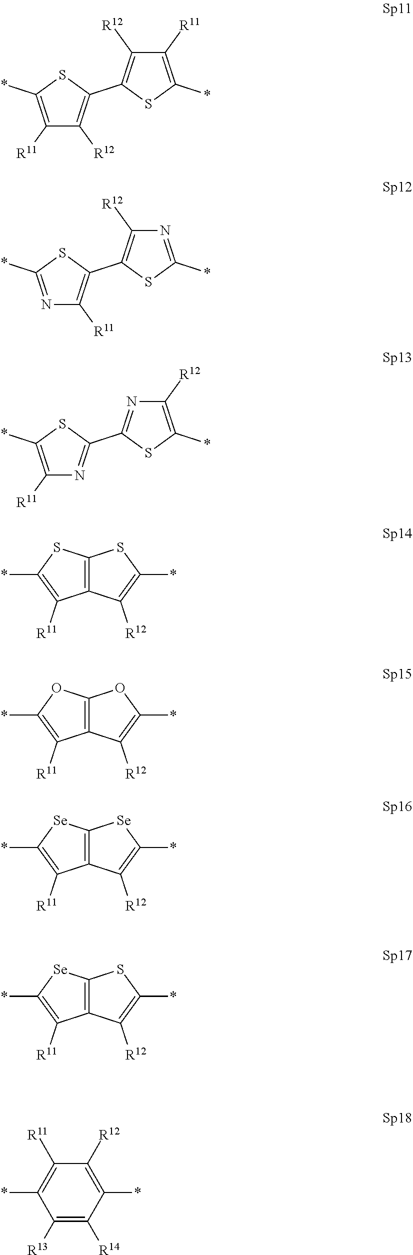

wherein R¹¹, R¹², R¹³, R¹⁴ independently of each other have one of the meanings of R⁵ as given in formula I or one of its preferred meanings as given above and below.

In the formulae Sp1 to Sp17 preferably R¹¹ and R¹² are H. In formula Sp18 preferably R¹¹⁻¹⁴ are H or F.

Very preferred are units selected from formulae Sp1, Sp2, Sp6, Sp10, Sp11, Sp12, Sp13 and Sp14, wherein preferably one of R¹¹ and R¹² is H or both R¹¹ and R¹² are H.

Further preferred are polymers according to the present invention, repeating units of formulae II1, II2, II3 and U2-U12 and their subformulae, and polymers of formulae III, Pi-Px and their subformulae, wherein one or more of the units of group D1, or one or more of C¹, C², C³, C⁴, the spacer units or Sp, respectively, are selected from the following formulae

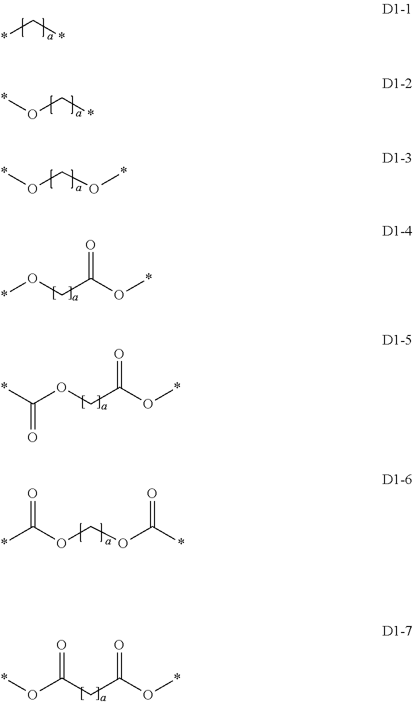

wherein a is an integer from 1 to 16, preferably from 2 to 6.

Further preferred are polymers according to the present invention, repeating units of formulae II1, II2, II3 and U2-U12 and their subformulae, and polymers of formulae III, Pi-Px and their subformulae, wherein one or more of the units of group E1, or one or more of C¹, C², C³, C⁴, the spacer units or Sp, respectively, are selected from formula E1a

A¹-B¹-(A²)ₐₐ E1a

wherein aa is 0 or 1, A¹ and A² have one of the meanings given for C¹ as defined in formula II1 or its preferred meanings as given above and below, and B¹ is selected from group D1, preferably from formulae D1-1 to D1-7.

Preferred units and groups of formula E1a are selected from the following formulae

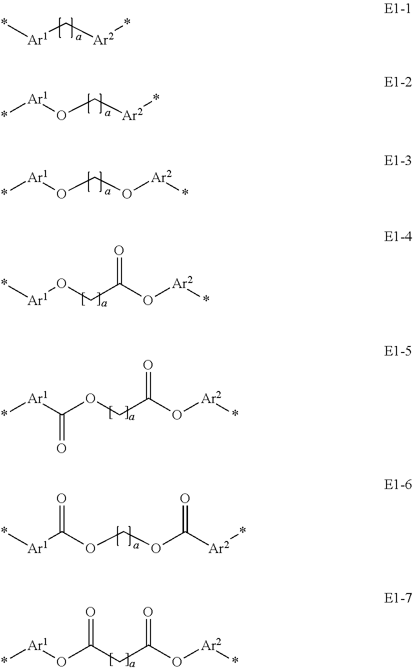

wherein a is an integer from 1 to 16, preferably from 2 to 6, and C¹ and C² have the meanings given in formula II1 or one of their preferred meanings given above and below.

Very preferred units and groups of formula E1a are selected from the following formulae

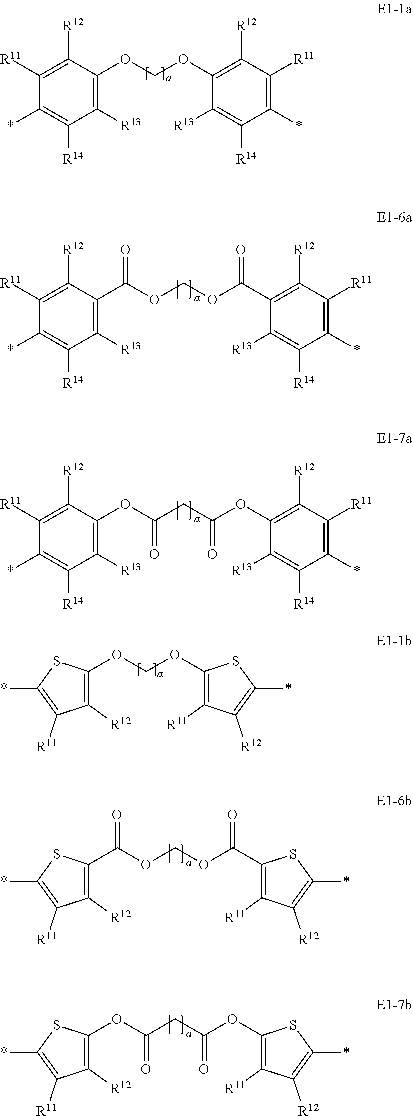

wherein a has the meanings given in formula D1-1 and R¹¹ to R¹⁴ have the meanings given above.

Further preferred are polymers comprising, preferably consisting of, one or more, preferably two or more, units of formula I, I1-I5, IA, IA1-IA10 or their subformulae, and one or more units selected from the following groups
A2) the group consisting of arylene or heteroarylene, preferably having electron donor properties, selected from the group consisting of the formulae D1-D151, very preferably of the formulae D1, D7, D10, D11, D19, D22, D29, D30, D35, D36, D37, D44, D55, D84, D87, D88, D89, D93, D94, D106, D111, D139, D140, D141, D146 and D150,
   and/or
A3) the group consisting of arylene or heteroarylene, preferably having electron acceptor properties, selected from the group consisting of the formulae A1-A103, very preferably of the formulae A1, A6, A7, A15, A16, A20, A36, A74, A84, A88, A92, A94, A98 and A103,
   and/or
A4) the group consisting of arylene or heteroarylene selected from the group consisting of the formulae Sp1-Sp18, very preferably of the formulae Sp1, Sp2, Sp6, Sp10, Sp11, Sp12, Sp13 and Sp14
   and/or
D2) the group consisting of formulae D1-1 to D1-7,
   and/or
E2) the group consisting of formulae E1a, E1-1 to E1-7 and E1-1a to E1-7b.

Further preferred are repeating units and polymers of formulae II1, II2, II3, III and their subformulae wherein C¹, C², C³ and C⁴ are selected from groups A2, A3, A4, D2 and E2 as defined above.

Further preferred are repeating units of formula U2-U12 and polymers of formulae Pi-Px wherein
a) the donor units D are selected from the group consisting of the formulae D1-D151, very preferably of the formulae D1, D7, D10, D11, D19, D22, D29, D30, D35, D36, D37, D44, D55, D84, D87, D88, D89, D93, D94, D106, D111, D139, D140, D141, D146 and D150,
b) the acceptor units A selected from the group consisting of the formulae A1-A103, very preferably of the formulae A1, A6, A7, A15, A16, A20, A36, A74, A84, A88, A92, A94, A98 and A103,
   and
c) the spacer units Sp selected from the group consisting of the formulae Sp1-Sp18, D1-1 to D1-7, E1a or E1-1 to E1-7, very preferably of the formulae Sp1, Sp2, Sp6, Sp10, Sp11, Sp12, Sp13 and Sp14, or E1-1a to E1-7b.

A preferred embodiment of the present invention relates to a polymer which consists of
one or more, preferably two or more, units of formula I, I1-I5, IA, IA1-IA10 or their subformulae, and
one or more units selected from groups A1, B1 and C1, preferably from group A1, very preferably from groups A2, A3 and A4, as defined above.

Another preferred embodiment of the present invention relates to a polymer which consists of
one or more, preferably two or more, units of formula I, I1-I5, IA, IA1-IA10 or their subformulae, and
one or more units selected from groups D1 and E1, preferably from group D1, very preferably from groups D2 and E2, most preferably from group E2, as defined above,
and optionally one or more units selected from groups A1, B1 and C1, preferably from group A1, very preferably from groups A2, B2 and C2, as defined above.

Preferred polymers of formula III and Pi-Px are selected from the following subformulae

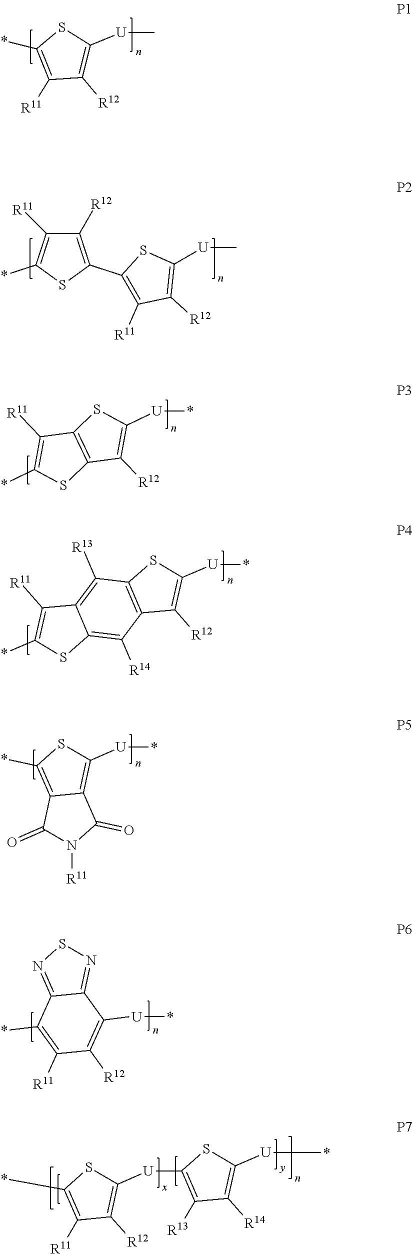

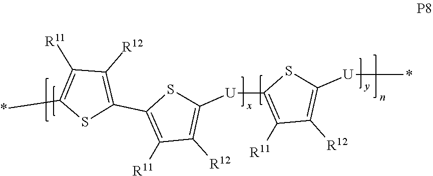

wherein R¹¹⁻¹⁴, n, x and y have the meanings given above and below, and U is a unit selected from formula I, I1-I5, IA, IA1-IA10 or their subformulae as defined above and below, preferably selected from formulae IA1-IA10.

Further preferred polymers of formula III and Pi-Px are selected from the following subformulae

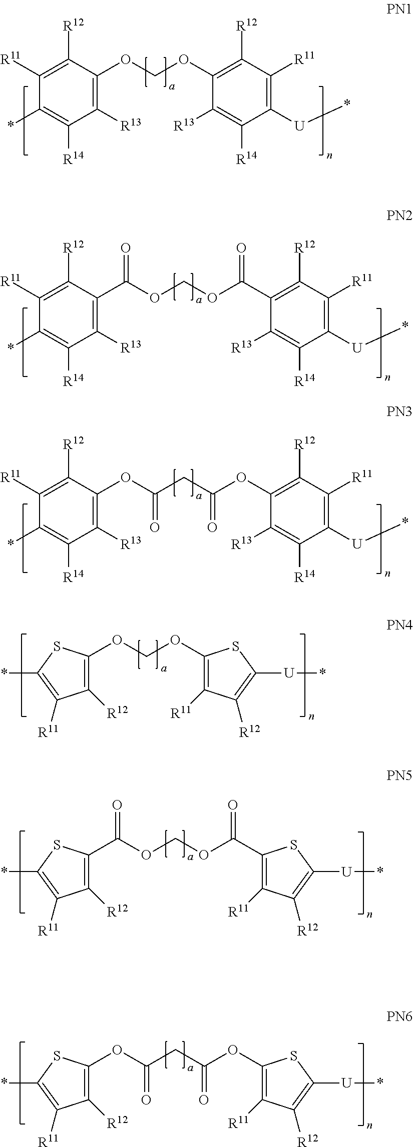

wherein R¹¹⁻¹⁴, U, a and n have the meanings given above and below.

Further preferably the polymer is selected of formula IV

R^{E1}-chain-R^{E2} IV

wherein “chain” denotes a polymer chain selected from formulae III, Pi-Px, P1-P8 or PN1-PN6, and R^{E1} and R^{E2} have independently of each other one of the meanings of L as defined above, or denote, independently of each other, H, F, Br, Cl, I, —CH₂Cl, —CHO, —CR′═CR″₂, —SiR′R″R′″, —SiR′X′X″, —SiR′R″X′, —SnR′R″R′″, —BR′R″, —B(OR′)(OR″), —B(OH)₂, —O—SO₂—R′, —C≡CH, —C≡C—SiR′₃, —ZnX′ or an endcap group, X′ and X″ denote halogen, R′, R″ and R′″ have independently of each other one of the meanings of R⁰ given in formula I, and preferably denote alkyl with 1 to 12 C atoms, and two of R′, R″ and R′″ may also form a cyclosilyl, cyclostannyl, cycloborane or cycloboronate group with 2 to 20 C atoms together with the respective hetero atom to which they are attached.

Preferred endcap groups R^{E1} and R^{E2} are H, C₁₋₂₀ alkyl, or optionally substituted C₆₋₁₂ aryl or C₂₋₁₀ heteroaryl, very preferably H or phenyl.

In the polymers according to the present invention, including but not limited to those of formulae III, Pi-Px, P1-P8, PN1-PN6 and their subformulae, the indices v, w, x, y and z denote the mole fraction of the corresponding repeating units, such as units A-E in formula III, and n denotes the degree of polymerisation or total number of repeating units. These formulae include block copolymers, random or statistical copolymers and alternating copolymers, as well as homopolymers for the case when x>0 and v=w=y=z=0.

In the polymers according to the present invention, including but not limited to those of formulae III, Pi-Px, P1-P8, PN1-PN6 and their subformulae, wherein one of v, w, y and z is not 0 and the others of v, w, y and z are 0, x and the one of v, w, y and z which is not 0 are each preferably from 0.1 to 0.9, very preferably from 0.3 to 0.7.

In the polymers according to the present invention, including but not limited to those of formulae III, Pi-Px, P1-P8, PN1-PN6 and their subformulae, wherein two of v, w, y and z are not 0 and the others of v, w, y and z are 0, x and those of v, w, y and z which are not 0 are each preferably from 0.1 to 0.8, very preferably from 0.2 to 0.6.

In the polymers according to the present invention, including but not limited to those of formulae III, Pi-Px, P1-P8, PN1-PN6 and their subformulae, wherein three of v, w, y and z are not 0 and the others of v, w, y and z are 0, x and those of v, w, y and z which are not 0 are each preferably from 0.1 to 0.7, very preferably from 0.2 to 0.5.

In the polymers according to the present invention, including but not limited to those of formulae III, Pi-Px, P1-P8, PN1-PN6 and their subformulae, wherein all of v, w, y and z are not 0, x, v, w, y and z are each preferably from 0.1 to 0.6, very preferably from 0.2 to 0.4.

In the polymers according to the present invention, the total number of repeating units n is preferably from 2 to 10,000, very preferably from 5 to 10,000. The total number of repeating units n is preferably ≥5, very preferably ≥10, most preferably ≥50, and preferably ≤500, very preferably ≤1,000, most preferably ≤2,000, including any combination of the aforementioned lower and upper limits of n.

The polymers of the present invention include homopolymers and copolymers, like statistical or random copolymers, alternating copolymers and block copolymers, as well as combinations thereof.

The invention further relates to monomers of formula V1 or V2

R^{R1}—(C¹)ₐ—U—(C²)_{b}—(C³)_{c}—(C⁴)_{d}—R^{R2} V1

R^{R1}—(C¹)ₐ—(C²)_{b}—U—(C³)_{c}—(C⁴)_{d}—R^{R2} V2

wherein U, C¹⁻⁴, a, b, c and d have the meanings of formula II1, or one of the preferred meanings as described above and below, and R^{R1} and R^{R2} are independently of each other selected from the group consisting of H, which is preferably an activated C—H bond, Cl, Br, I, O-tosylate, O-triflate, O-mesylate, O-nonaflate, —SiMe₂F, —SiMeF₂, —O—SO₂Z¹, —B(OZ²)₂, —CZ³═C(Z³)₂, —C≡CH, —C≡CSi(Z¹)₃, —ZnX⁰ and —Sn(Z⁴)₃, wherein X⁰ is halogen, Z¹⁻⁴ are selected from the group consisting of alkyl and aryl, preferably C₁₋₁₀ alkyl and C₆₋₁₂ aryl, each being optionally substituted, and two groups Z² may also form a cycloboronate group having 2 to 20 C atoms together with the B- and O-atoms, and wherein at least one of R^{R1} and R^{R2} is different from H, and preferably both of R^{R1} and R^{R2} are different from H.

Very preferred are monomers of formula V1 and V2 wherein a+b+c+d≥1.

Further preferred are monomers of formula V1 wherein a+b+c+d=0.

Further preferred are monomers selected from the following subformulae

R^{R1}—U—R^{R2} V1a

R^{R1}—C¹—U—C²—R^{R2} V1b

R^{R1}—C¹—U—R^{R2} V1c

R^{R1}—U—C²—R^{R2} V1d

wherein U, C¹, C², R^{R1} and R^{R2} are as defined in formula V1.

Further preferred are monomers of formula V1, V2 and V1a-d wherein R^{R1} and R^{R2} are selected from Br, —B(OZ²)₂ and Sn(Z⁴)₃.

Further preferred are monomers of formulae V1, V2 and V1a-V1d wherein C¹ and/or C² are selected from groups A2, A3, A4, D2 and E2 as defined above.

Further preferred are monomers of formula V3

R^{R1}—U*—R^{R2} V3

wherein R^{R1} and R^{R2} have the meanings given above and below, and preferably denote Br, B(OZ²)₂ or Sn(Z⁴)₃, and U* is a unit selected from formulae P1-P8 or PN1-PN6 wherein n is 1.

Further preferred units, monomers and polymers of formulae I, I1-I5, II1, II2, II3, III, Pi-Px, P1-P8, PN1-PN6, IV, V1-V3, V1a-d and their subformulae are selected from the following embodiments, including any combination thereof:
n≥5,
n is from 5 to 1,000, most preferably from 10 to 2,000,
a1=b1=1 and c1 and d1 are independently of each other 0, 1 or 2, preferably 0 or 1, very preferably 0,
a1 is 2, b1 is 1 or 2, c1 is 0 or 1, preferably 0, and d1 is 0, 1 or 2, preferably 0 or 1, very preferably 0,
one or more of R¹¹⁻¹⁸ is different from H and is selected from alkyl, alkoxy or thiaalkyl, all of which are straight-chain or branched, have 1 to 25, preferably 1 to 18 C atoms, and are optionally fluorinated,
one or more of R¹¹⁻¹⁸ is different from H and is selected from F, Cl, CN, —C(═O)—Rⁿ, —C(═O)—ORⁿ, —C(═O)—NHRⁿ and —C(═O)—NRⁿR^{m}, wherein R^{m} and Rⁿ are independently of each other straight-chain or branched alkyl with 1 to 25, preferably 1 to 18 C atoms that is optionally fluorinated,
one or more of R¹¹⁻¹⁸ is different from H and is selected from the group consisting of aryl, heteroaryl, aryloxy, heteroaryloxy, arylalkyl and heteroarylalkyl, each of which has 4 to 20 ring atoms and optionally contains fused rings and is unsubstituted or substituted by one or more groups L as defined in formula I,
R^{E1} and R^{E2} are selected from H, C₁₋₂₀ alkyl, or optionally substituted C₆₋₁₂ aryl or E1₋₁₀ heteroaryl, very preferably H or phenyl,
R^{R1} and R^{R2} denote Br, B(OZ²)₂ or Sn(Z⁴)₃, wherein Z² and Z⁴ are as defined in formula V1.

The polymers according to the present invention can be synthesized according to or in analogy to methods that are known to the skilled person and are described in the literature. Other methods of preparation can be taken from the examples.

The polymers of the present invention can be prepared from the corresponding monomers, which are preferably selected from formula V1-V3 or V1a-d, for example by copolymerising one or more monomers of formula V1-V3 or V1a-d with each other or with one or monomers of the following formulae in an aryl-aryl coupling reaction

R^{R1}—C¹—R^{R2} MI

R^{R1}—C²—R^{R2} MII

R^{R1}—C³—R^{R2} MIII

R^{R1}—C⁴—R^{R2} MIV

wherein C¹⁻⁴, R^{R1} and R^{R2} have the meanings given in formula II2 and V1 or one of the preferred meanings given above and below.

For example, the polymers can be suitably prepared by aryl-aryl coupling reactions, such as Yamamoto coupling, C—H activation coupling, Suzuki coupling, Stille coupling, Sonogashira coupling, Heck coupling or Buchwald coupling. Suzuki coupling, Stille coupling and Yamamoto coupling are especially preferred. The monomers which are polymerised to form the repeat units of the polymers can be prepared according to methods which are known to the person skilled in the art.

Preferably the polymers are prepared from monomers selected from formulae V1-V3, V1a-d and MI-MIV as described above.

Another aspect of the invention is a process for preparing an polymer by coupling one or more identical or different monomers selected from formulae V1. V2, V5 and V1a-d with each other and/or with one or more co-monomers, preferably selected from formulae MI-MIV, in a polymerisation reaction, preferably in an aryl-aryl coupling reaction.

Preferred aryl-aryl coupling methods used in the synthesis methods as described above and below are Yamamoto coupling, Kumada coupling, Negishi coupling, Suzuki coupling, Stille coupling, Sonogashira coupling, Heck coupling, C—H activation coupling, Ullmann coupling or Buchwald coupling. Especially preferred are Suzuki coupling, Negishi coupling, Stille coupling and Yamamoto coupling. Suzuki coupling is described for example in WO 00/53656 A1. Negishi coupling is described for example in J. Chem. Soc., Chem. Commun., 1977, 683-684. Yamamoto coupling is described in for example in T. Yamamoto et al., Prog. Polym. Sci., 1993, 17, 1153-1205, or WO 2004/022626 A1. Stille coupling is described for example in Z. Bao et al., J. Am. Chem. Soc., 1995, 117, 12426-12435 and C—H activation is described for example in M. Leclerc et al, Angew. Chem. Int. Ed., 2012, 51, 2068-2071. For example, when using Yamamoto coupling, educts having two reactive halide groups are preferably used. When using Suzuki coupling, educts having two reactive boronic acid or boronic acid ester groups or two reactive halide groups are preferably used. When using Stille coupling, educts having two reactive stannane groups or two reactive halide groups are preferably used. When using Negishi coupling, educts having two reactive organozinc groups or two reactive halide groups are preferably used.

Preferred catalysts, especially for Suzuki, Negishi or Stille coupling, are selected from Pd(0) complexes or Pd(II) salts. Preferred Pd(0) complexes are those bearing at least one phosphine ligand such as Pd(Ph₃P)₄. Another preferred phosphine ligand is tris(ortho-tolyl)phosphine, i.e. Pd(o-Tol₃P)₄. Preferred Pd(II) salts include palladium acetate, i.e. Pd(OAc)₂. Alternatively the Pd(0) complex can be prepared by mixing a Pd(0) dibenzylideneacetone complex, for example tris(dibenzyl-ideneacetone)dipalladium(0), bis(dibenzylideneacetone)palladium(0), or Pd(II) salts e.g. palladium acetate, with a phosphine ligand, for example triphenylphosphine, tris(ortho-tolyl)phosphine or tri(tert-butyl)phosphine. Suzuki coupling is performed in the presence of a base, for example sodium carbonate, potassium carbonate, cesium carbonate, lithium hydroxide, potassium phosphate or an organic base such as tetraethylammonium carbonate or tetraethylammonium hydroxide. Yamamoto coupling employs a Ni(0) complex, for example bis(1,5-cyclooctadienyl) nickel(0).

As alternatives to halogens as described above, leaving groups of formula —O—SO₂Z⁰ can be used wherein Z⁰ is an alkyl or aryl group, preferably C₁₋₁₀ alkyl or C₆₋₁₂ aryl. Particular examples of such leaving groups are tosylate, mesylate and triflate.

Especially suitable and preferred synthesis methods of the repeating units of formula I and the conjugated polymers comprising them are illustrated in the synthesis schemes shown hereinafter, wherein “core”, C¹⁻⁴, R¹⁻⁴, L and n have the meanings as described above and below, Z¹ and Z² denote C, Si or Ge, and i is 0, 1 or 2.

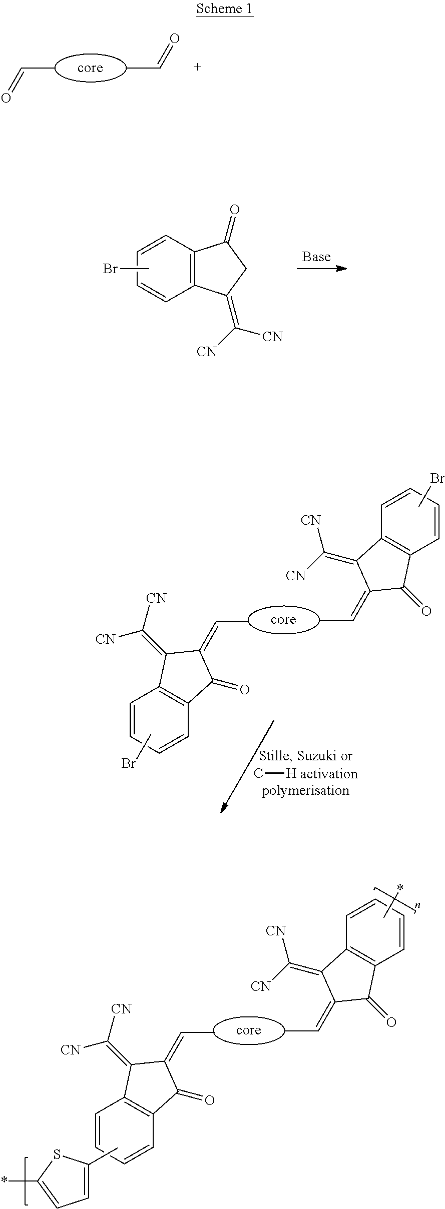

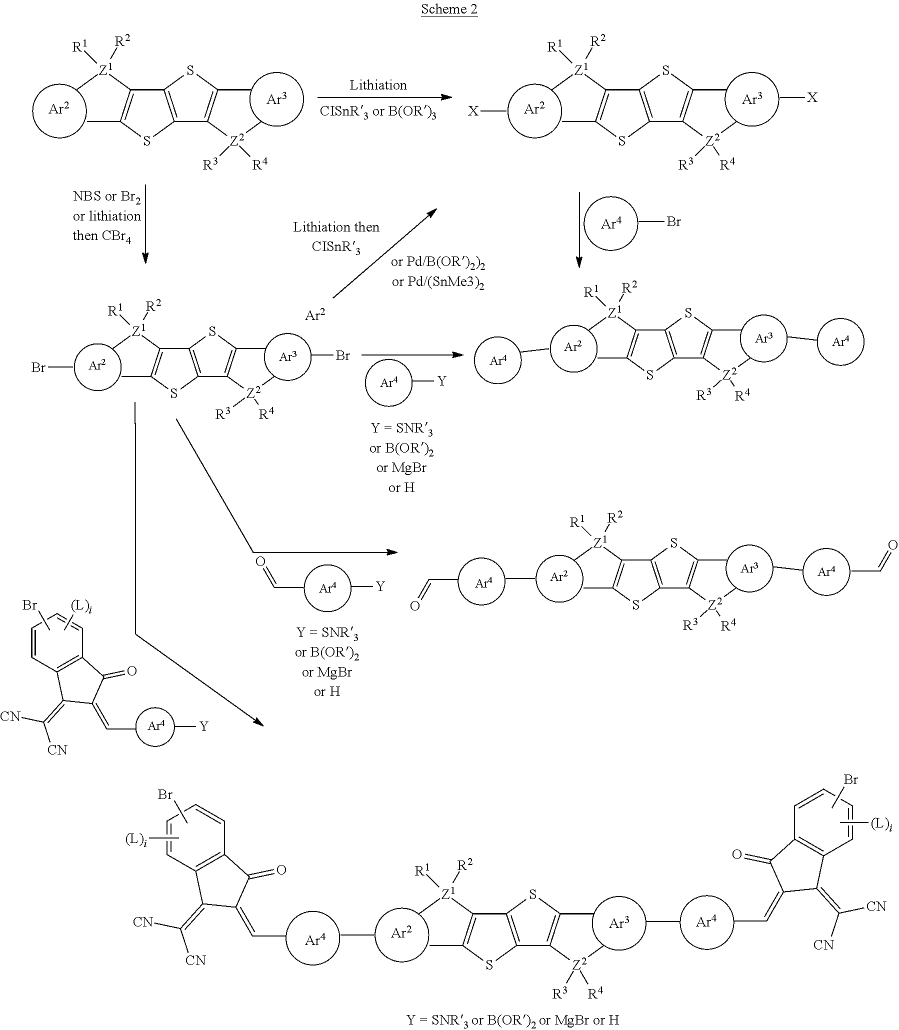

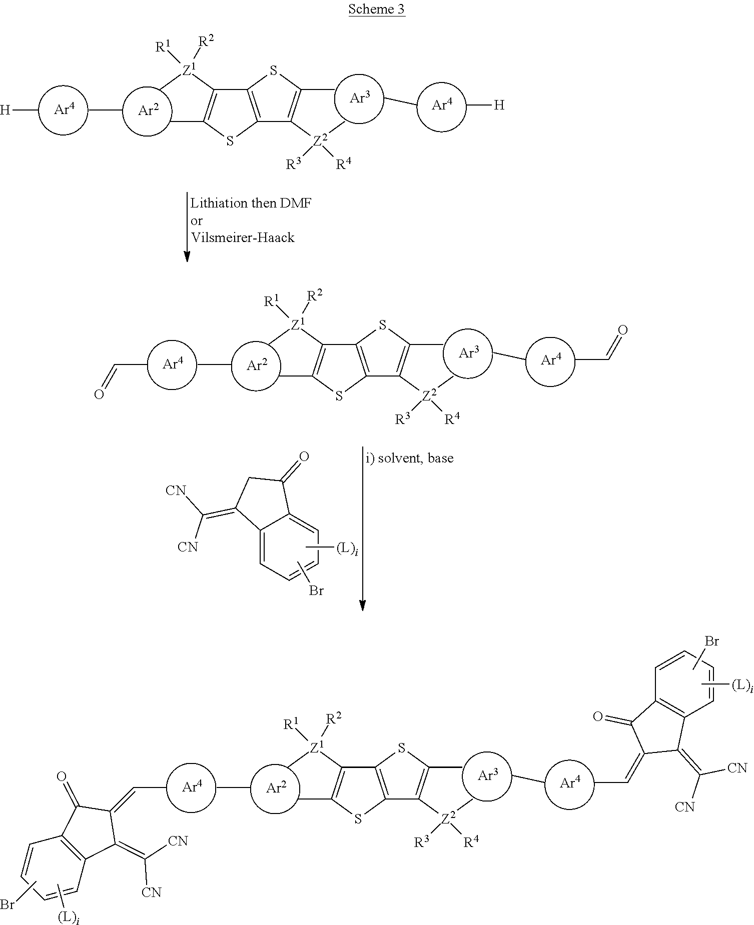

Novel methods of preparing repeating units of formula I and monomers and polymers comprising them as described above and below are another aspect of the invention.

The polymer according to the present invention can also be used in compositions, for example together with monomeric or polymeric compounds having charge-transport, semiconducting, electrically conducting, photoconducting and/or light emitting semiconducting properties, or for example with compounds having hole blocking or electron blocking properties for use as interlayers or charge blocking layers in PSCs or OLEDs.

Thus, another aspect of the invention relates to a composition comprising one or more polymers according to the present invention and one or more small molecule compounds and/or polymers having one or more of a charge-transport, semiconducting, electrically conducting, photoconducting, hole blocking and electron blocking property.

The invention further relates to a composition comprising one or more polymers according to the present invention, and further comprising one or more p-type organic semiconductors, preferably selected from conjugated polymers.

The invention further relates to a composition comprising a first n-type semiconductor which is a polymer according to the present invention, a second n-type semiconductor, which is preferably a fullerene or fullerene derivative, a non-fullerene acceptor small molecule, or an n-type conjugated polymer, and a p-type semiconductor, which is preferably a conjugated polymer.

In a preferred embodiment the second n-type OSC compound is a non-fullerene acceptor (NFA) small molecule having an A-D-A structure as described above with an electron donating polycyclic core and two terminal electron withdrawing groups attached thereto.

Suitable and preferred NFA small molecules for use as second n-type OSC in this preferred embodiment are for example those disclosed in Y. Lin et al., Adv. Mater., 2015, 27, 1170; H. Lin et al., Adv. Mater., 2015, 27, 7299; N. Qiu et al., Adv. Mater., 2017, 29, 1604964; CN104557968 A and CN105315298 A, furthermore those disclosed in WO 2018/007479 A1.

In another preferred embodiment the second n-type OSC compound is a fullerene or substituted fullerene.

The fullerene is for example an indene-C₆₀-fullerene bisadduct like ICBA, or a (6,6)-phenyl-butyric acid methyl ester derivatized methano C₆₀ fullerene, also known as “PCBM-C₆₀” or “C₆₀PCBM”, as disclosed for example in G. Yu, J. Gao, J. C. Hummelen, F. Wudl, A. J. Heeger, Science 1995, Vol. 270, p. 1789 ff and having the structure shown below, or structural analogous compounds with e.g. a C₆₁ fullerene group, a C₇₀ fullerene group, or a C₇₁ fullerene group, or an organic polymer (see for example Coakley, K. M. and McGehee, M. D. Chem. Mater. 2004, 16, 4533).

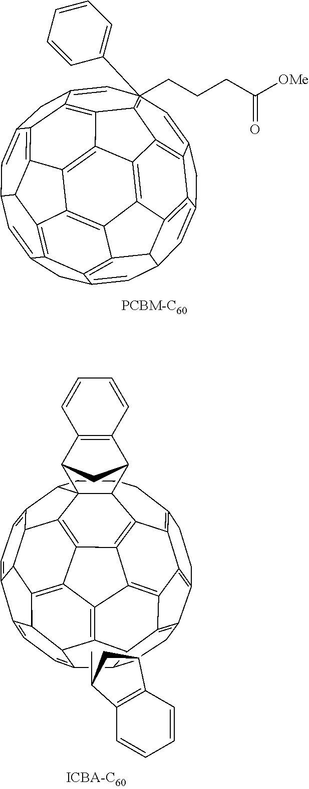

Preferably the polymer according to the present invention is blended with an n-type semiconductor such as a fullerene or substituted fullerene of formula Full-I to form the active layer in an OPV or OPD device,

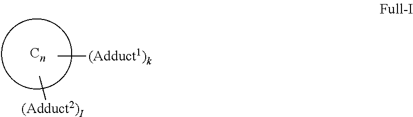

wherein
Cₙ denotes a fullerene composed of n carbon atoms, optionally with one or more atoms trapped inside,
Adduct¹ is a primary adduct appended to the fullerene Cₙ with any connectivity,
Adduct² is a secondary adduct, or a combination of secondary adducts, appended to the fullerene Cₙ with any connectivity,
k is an integer ≥1,
and
l is 0, an integer ≥1, or a non-integer >0.

In the formula Full-I and its subformulae, k preferably denotes 1, 2, 3 or, 4, very preferably 1 or 2.

The fullerene Cₙ in formula Full-I and its subformulae may be composed of any number n of carbon atoms Preferably, in the compounds of formula XII and its subformulae the number of carbon atoms n of which the fullerene Cₙ is composed is 60, 70, 76, 78, 82, 84, 90, 94 or 96, very preferably 60 or 70.

The fullerene Cₙ in formula Full-I and its subformulae is preferably selected from carbon based fullerenes, endohedral fullerenes, or mixtures thereof, very preferably from carbon based fullerenes.

Suitable and preferred carbon based fullerenes include, without limitation, (C₆₀₋ₗₕ)[5,6]fullerene, (C_{70-D5h})[5,6]fullerene, (C_{76-D2*})[5,6]fullerene, (C_{84-D2*})[5,6]fullerene, (C_{84-D2d})[5,6]fullerene, or a mixture of two or more of the aforementioned carbon based fullerenes.

The endohedral fullerenes are preferably metallofullerenes. Suitable and preferred metallofullerenes include, without limitation, La@C₆₀, La@C₈₂, Y@C₈₂, Sc₃N@C₈₀, Y₃N@C₈₀, Sc₃C₂@C₈₀ or a mixture of two or more of the aforementioned metallofullerenes.

Preferably the fullerene Cₙ is substituted at a [6,6] and/or [5,6] bond, preferably substituted on at least one [6,6] bond.

Primary and secondary adducts, named “Adduct1” and “Adduct 2” in formula Full-I and its subformulae, are each preferably selected from the following formulae

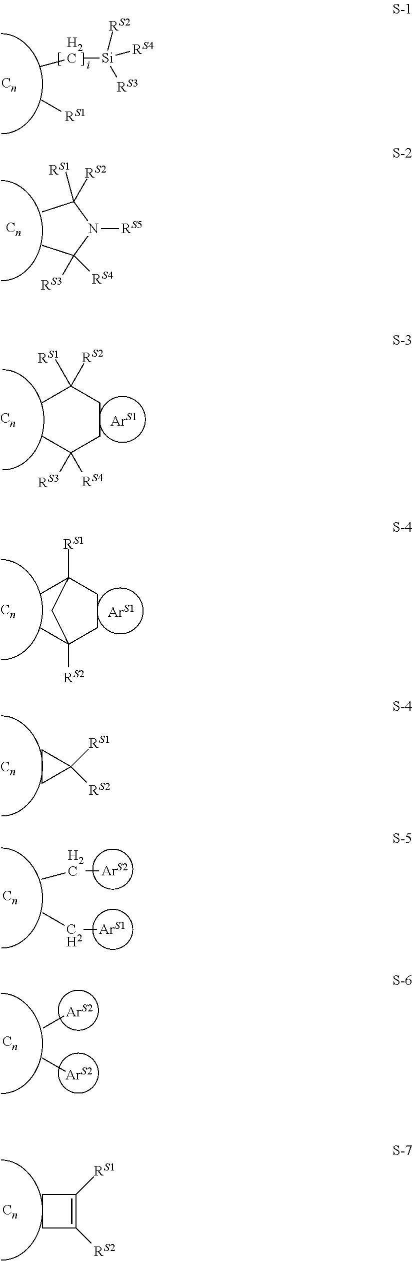

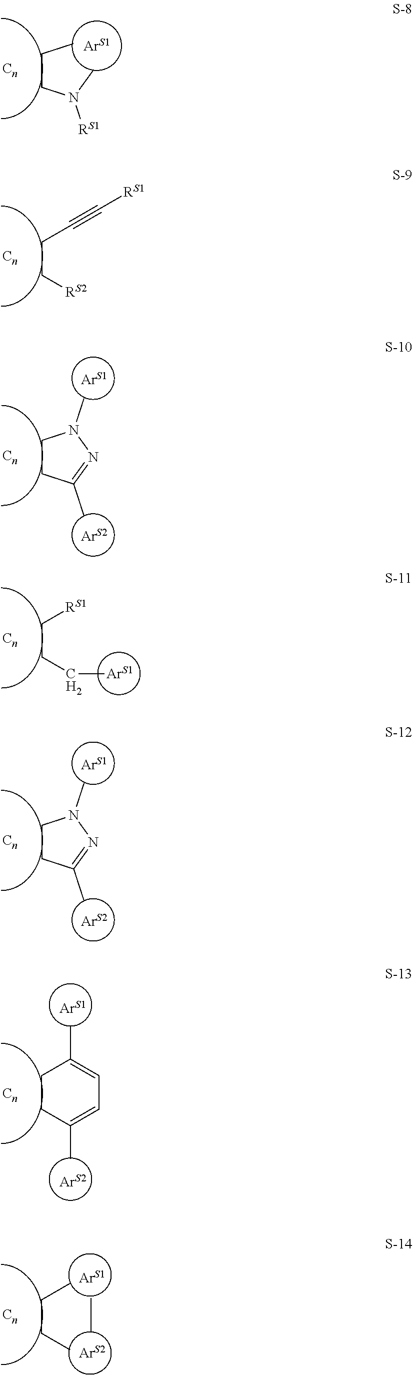

wherein
Ar^{S1}, Ar^{S2} denote, independently of each other, an aryl or heteroaryl group with 5 to 20, preferably 5 to 15, ring atoms, which is mono- or polycyclic, and which is optionally substituted by one or more identical or different substituents having one of the meanings of L as defined above and below,

R^{S1}, R^{S2}, R^{S3}, R^{S4} and R^{S5} independently of each other denote H, CN or have one of the meanings of L as defined above and below, and

i is an integer from 1 to 20, preferably from 1 to 12.

Preferred compounds of formula Full-I are selected from the following subformulae:

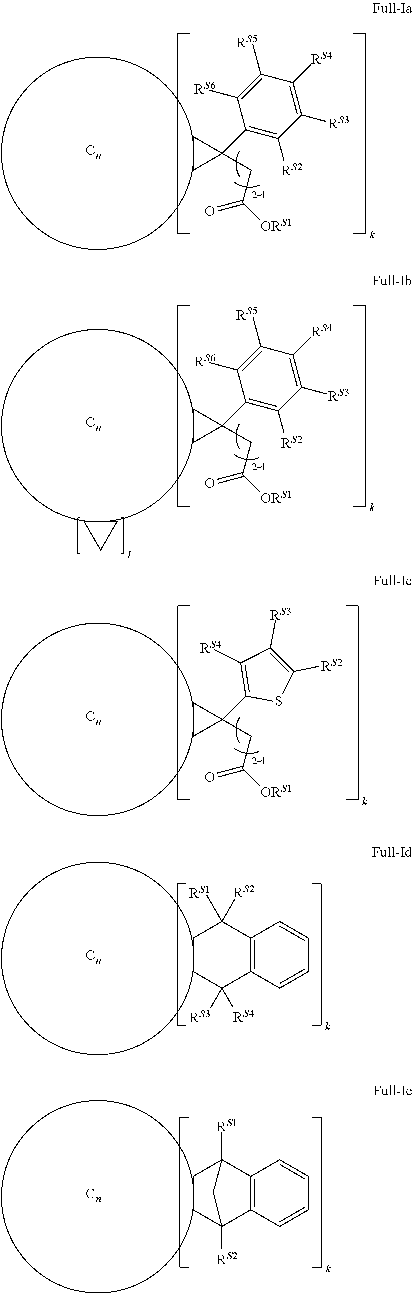

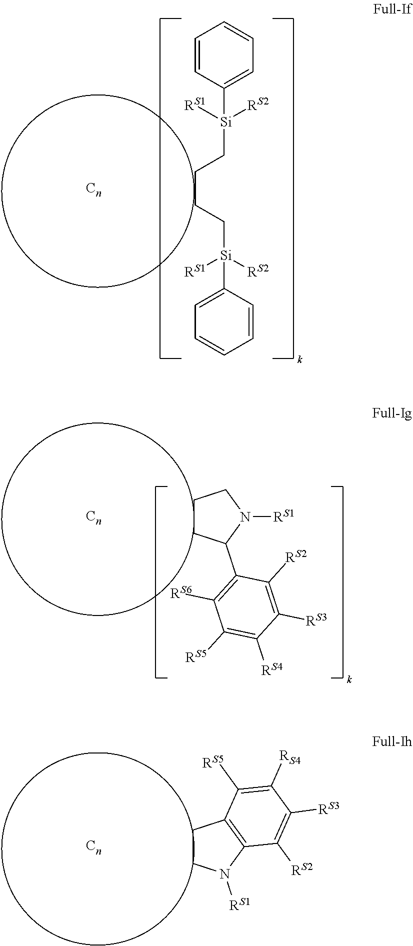

wherein

R^{S1}, R^{S2}, R^{S3}, R^{S4} R^{S5} and R^{S6} independently of each other denote H or have one of the meanings of R^{S} as defined above and below.

Most preferably the fullerene is PCBM-C60, PCBM-C70, bis-PCBM-C60, bis-PCBM-C70, ICMA-c60 (1′,4′-dihydro-naphtho[2′,3′:1,2][5,6]fullerene-C60), ICBA, oQDM-C60 (1′,4′-dihydro-naphtho[2′,3′1,9][5,6]fullerene-C60-lh), or bis-oQDM-C60.

In another preferred embodiment the second n-type OSC compound is a small molecule which does not contain a fullerene moiety, and which is selected from naphthalene or perylene carboximide derivatives.

Preferred naphthalene or perylene carboximide derivatives for use as n-type OSC compounds are described for example in Adv. Sci. 2016, 3, 1600117, Adv. Mater. 2016, 28, 8546-8551, J. Am. Chem. Soc., 2016, 138, 7248-7251 and J. Mater. Chem. A, 2016, 4, 17604.

Preferred n-type OSC compounds of this preferred embodiment are selected from the following formulae

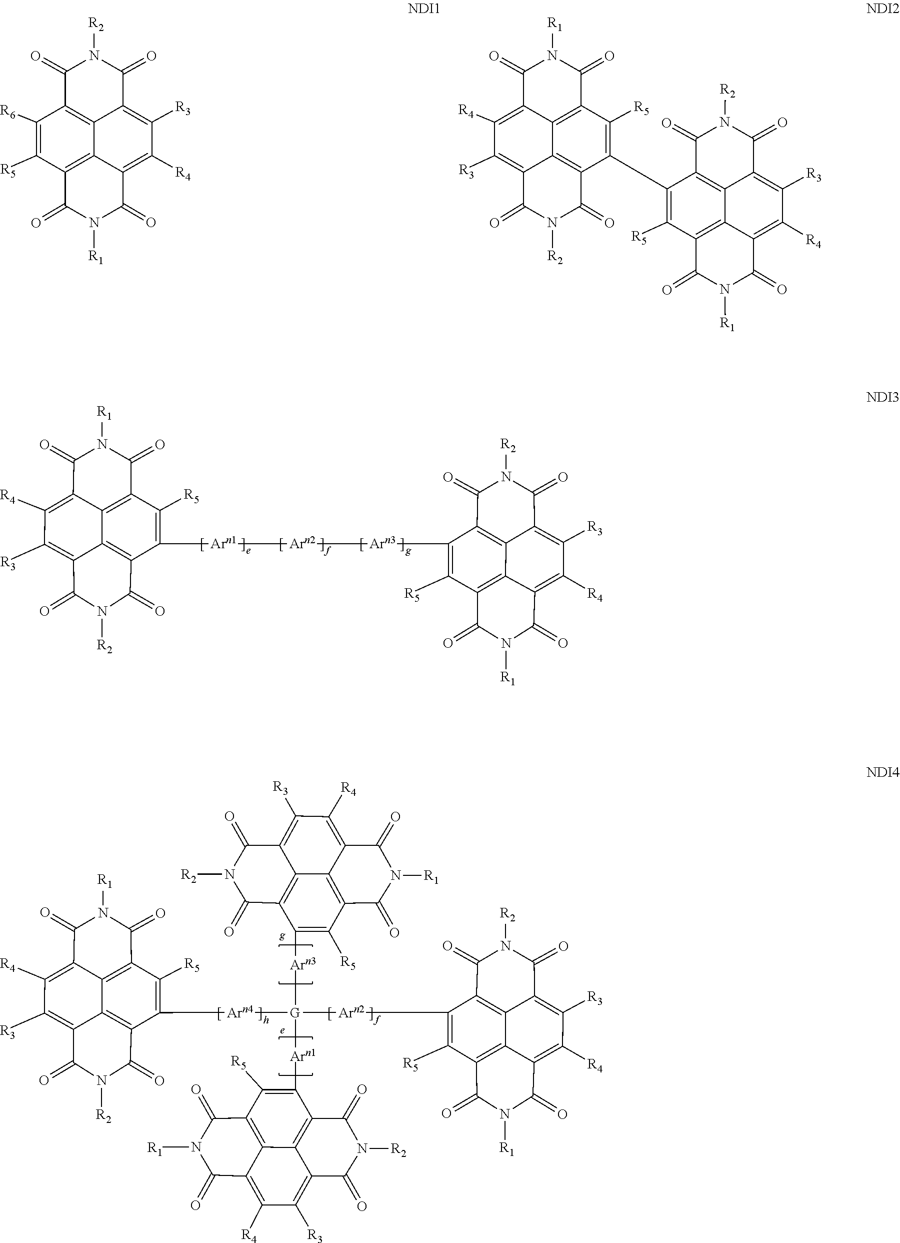

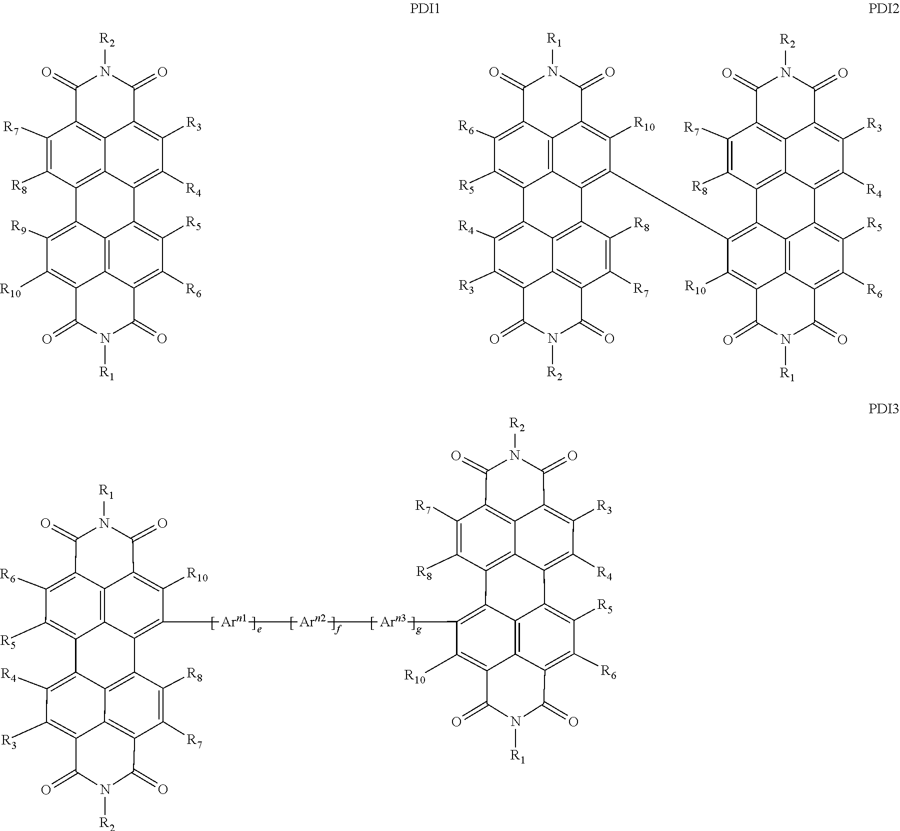

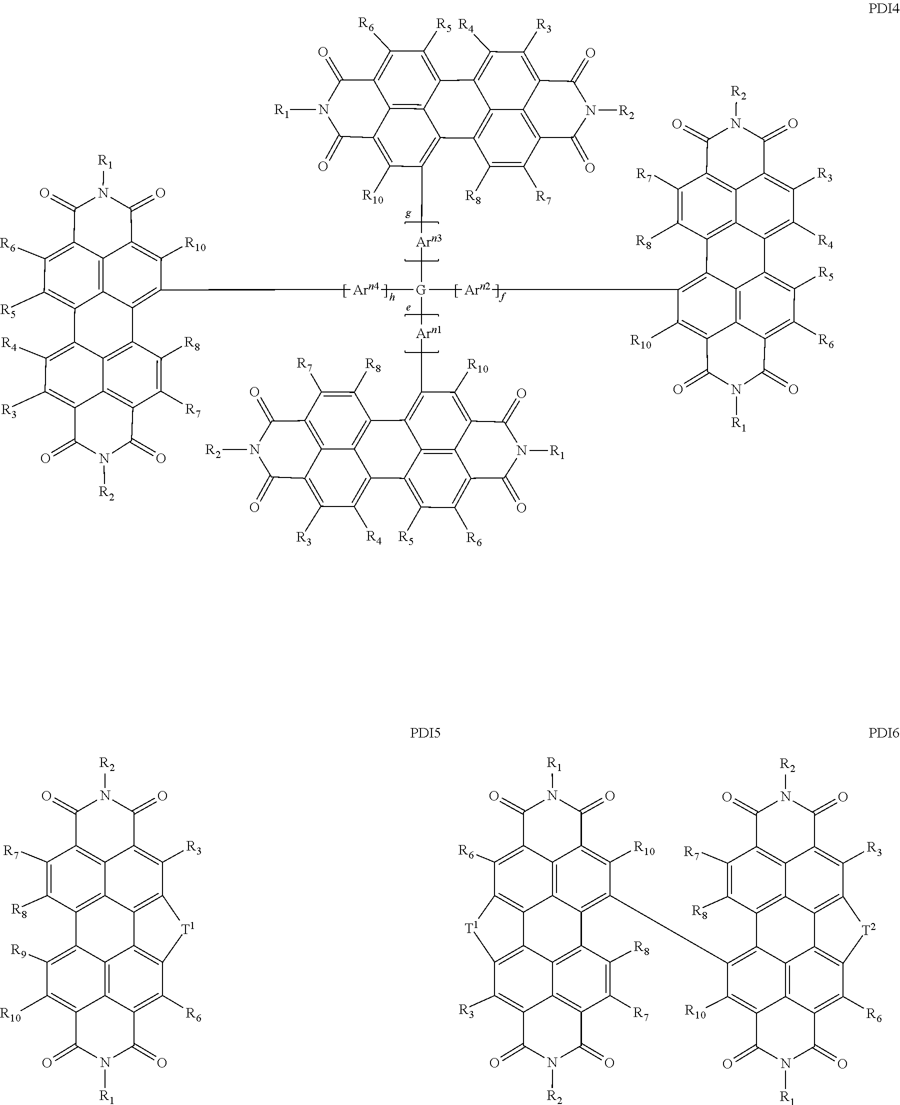

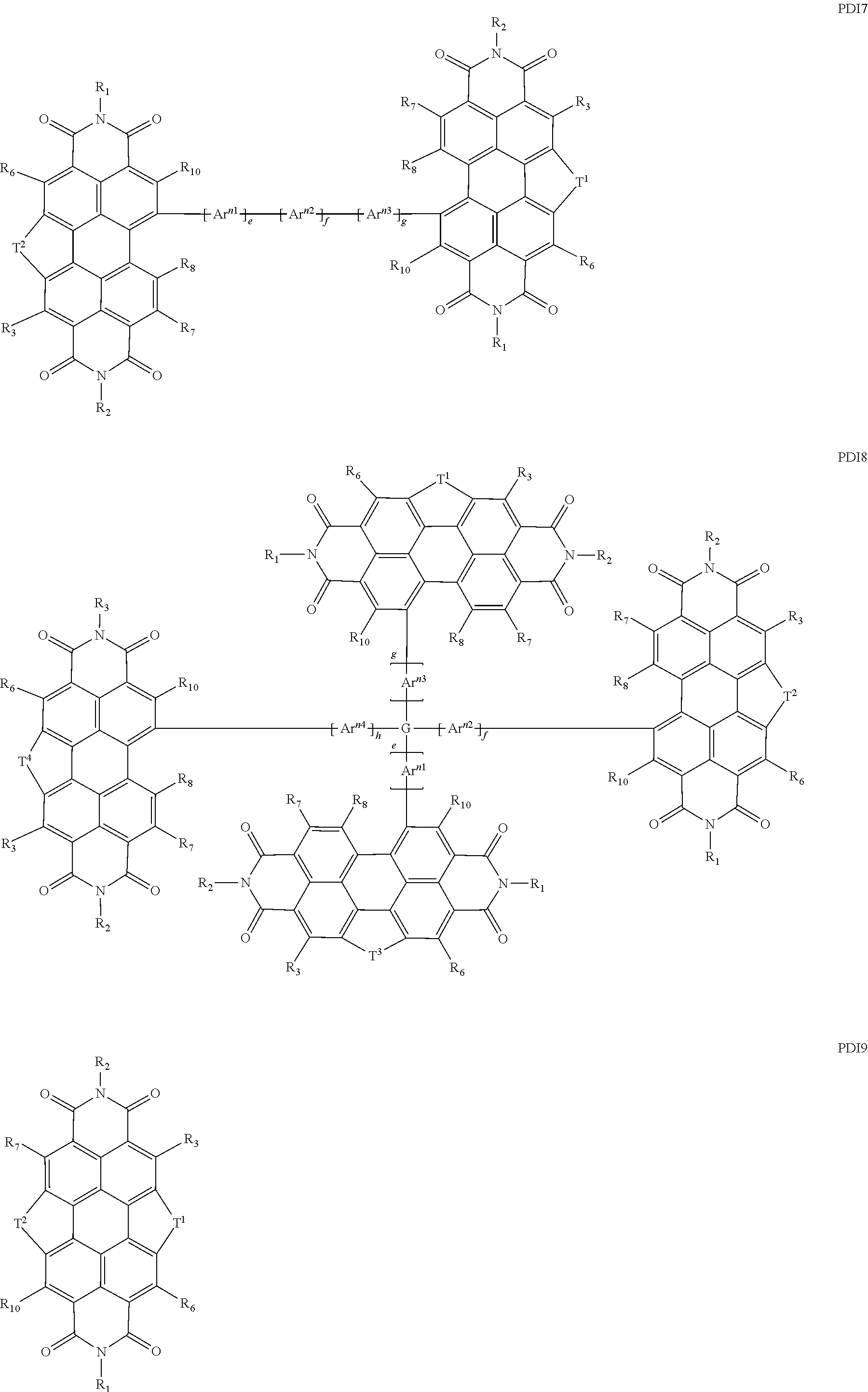

wherein the individual radicals, independently of each other and on each occurrence identically or differently, have the following meanings

R¹⁻¹⁰ Z¹, H, F, Cl, or straight-chain, branched or cyclic alkyl with 1 to 30, preferably 1 to 20, C atoms, in which one or more CH₂ groups are optionally replaced by —O—, —S—, —C(═O)—, —C(═S)—, —C(═O)—O—, —O—C(═O)—, —NR⁰—, —SiR⁰R⁰⁰—, —CF₂—, —CR⁰═CR⁰⁰—, —CY¹═CY²— or —C≡C— in such a manner that O and/or S atoms are not linked directly to one another, and in which one or more H atoms are optionally replaced by F, Cl, Br, I or CN, and in which one or more CH₂ or CH₃ groups are optionally replaced by a cationic or anionic group, or aryl, heteroaryl, arylalkyl, heteroarylalkyl, aryloxy or heteroaryloxy, wherein each of the aforementioned cyclic groups has 5 to 20 ring atoms, is mono- or polycyclic, does optionally contain fused rings, and is unsubstituted or substituted by one or more identical or different groups L,
Z¹ an electron withdrawing group, preferably having one of the preferred meanings as given above for formula T, very preferably CN,
Y¹, Y²H, F, Cl or CN,
L F, Cl, —NO₂, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30, preferably 1 to 20 C atoms that is optionally substituted and optionally comprises one or more hetero atoms, preferably F, —CN, R⁰, —OR⁰, —SR⁰, —C(═O)—R⁰, —C(═O)—OR⁰, —O—C(═O)—R⁰, —O—C(═O)—OR⁰, —C(═O)—NHR⁰, or —C(═O)—NR⁰R⁰⁰,

T¹⁻⁴ —O—, —S—, —C(═O)—, —C(═S)—, —CR⁰R⁰⁰—, —SiR⁰R⁰⁰—, —NR⁰—, —CR⁰═CR⁰⁰— or —C≡C—,
G C, Si, Ge, C═C or a four-valent aryl or heteroaryl group that has from 5 to 20 ring atoms, is mono- or polycyclic, optionally contains fused rings, and is unsubstituted or substituted by one or more identical or different groups R¹ or L,
Arⁿ¹⁻ⁿ⁴ independently of each other, and on each occurrence identically or differently arylene or heteroarylene that has from 5 to 20 ring atoms, is mono- or polycyclic, optionally contains fused rings, and is unsubstituted or substituted by one or more identical or different groups R¹ or L, or CY¹═CY² or —C≡C—,
e, f, g, h 0 or an integer from 1 to 10.

In another preferred embodiment the second n-type OSC compound is a conjugated OSC polymer. Preferred n-type OSC polymers are described, for example, in Acc. Chem. Res., 2016, 49 (11), pp 2424-2434 and WO 2013/142841 A1.

In another preferred embodiment, the second n-type OSC compound is selected from small molecules that do not contain a fullerene moiety, (hereinafter also referred to as “non-fullerene acceptors” or NFAs), and which comprise a polycyclic core and attached thereto two terminal groups which are electron withdrawing relative to the polycyclic core, and optionally further comprise one or more aromatic or heteroaromatic spacer groups, which are located between the polycyclic core and the terminal groups and which can be electron withdrawing or electron donating relative to the polycyclic core. As a result these preferred NFAs have an acceptor-donor-acceptor (A-D-A) structure, wherein the polycyclic core acts as donor and the terminal groups, optionally together with the spacer groups, act as acceptor.

Examples for suitable and preferred NFAs are the compound ITIC shown below, as disclosed by Y. Lin, J. Wang, Z.-G. Zhang, H. Bai, Y. Li, D. Zhu and X. Zhan, Adv. Mater. 2015, 27, 1170-1174, and the compound IEIC shown below, as disclosed by H. Lin, S. Chen, Z. Li, J. Y. L. Lai, G. Yang, T. McAfee, K. Jiang, Y. Li, Y. Liu, H. Hu, J. Zhao, W. Ma, H. Ade and H. Yan, Zhan, Adv. Mater., 2015, 27, 7299.

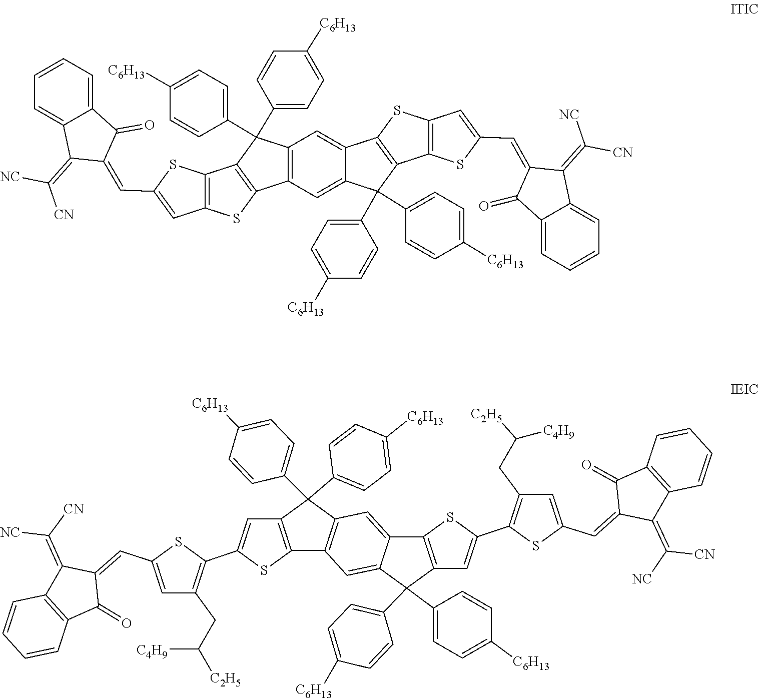

Further examples for suitable and preferred NFAs are disclosed in WO 2018/007479 A1, WO 2018/036914 A1, WO 2018/065350 A1, WO 2018/065352 A1, WO 2018/065356 A1 and EP 3306690 A1.

Preferred NFAs are selected from formula N

R^{T1}—(Ar¹¹)ₙ₁₁-core-(Ar¹²)ₙ₁₂—R^{T2} N

wherein, independently of each other and on each occurrence identically or differently, Ar¹¹ and Ar¹² have one of the meanings given for Ar⁴ above or one of its preferred meanings, “core” has the meaning given in formula IA or one of its preferred meanings, R^{T1} and R^{T2} are electron withdrawing groups, and n11, n12 denote 0, 1, 2 or 3.

Preferred compounds of formula N are selected from the following embodiments or any combination thereof:
the groups R^{T1} and R^{T2} are selected from the group consisting of monovalent groups of formulae T1 to T35 above which do not contain the second linkage via the benzene or thiophene ring (i.e. they only contain the linkage via the vinyl group), very preferably from formulae T1, T2, T3, T7, T8, T14, T15, T21, T22, T28, T29 and T35,
n11 and n12 are 0 or 1, preferably 0,
the group “core” is selected from the following formulae

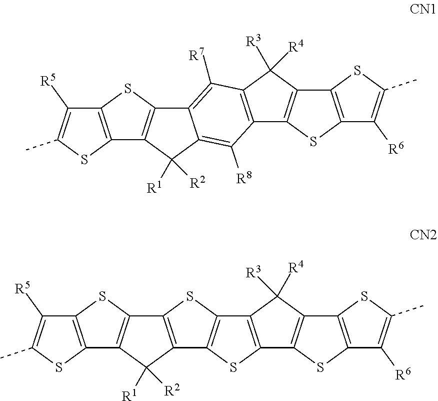

wherein R¹⁻⁸ have the meanings given above, and wherein preferably R⁷ and Fe denote H or F, preferably R⁵ and R⁶ denote H, and preferably R¹⁻⁴ are selected from 4-alkylphenyl, 4-alkoxyphenyl, 3,5-dialkylphenyl or 3,5-dialkoxyphenyl, wherein alkyl is C1-16 alkyl, and alkoxy is C1-16 alkoxy.

Preferred n-type conjugated OSC polymers for use as second n-type OSC compound in this preferred embodiment comprise one or more units derived from perylene or naphthalene are poly[[N,N′-bis(2-octyldodecyl)naphthalene-1,4,5,8-bis(dicarboximide)-2,6-diyl]-alt-5,5′-(2,2′-bithiophene)], poly[[N,N′-bis(2-hexyldecyl)naphthalene-1,4,5,8-bis(dicarboximide)-2,6-diyl]-alt-5,5′-thiophene].

The composition according to the present invention can be prepared by conventional methods that are described in prior art and known to the skilled person. Typically the compounds and/or polymers are mixed with each other or dissolved in suitable solvents and the solutions combined.

Another aspect of the invention relates to a formulation comprising one or more polymers according to the present invention or compositions as described above and below and one or more organic solvents.

Preferred solvents are aliphatic hydrocarbons, chlorinated hydrocarbons, aromatic hydrocarbons, ketones, ethers and mixtures thereof. Further suitable and preferred solvents used include 1,2,4-trimethylbenzene, 1,2,3,4-tetra-methyl benzene, pentylbenzene, mesitylene, cumene, cymene, cyclohexylbenzene, diethylbenzene, tetralin, indane, 1,5-dimethyltetraline, decalin, 1-methylnaphthalene, 2,6-lutidine, 2-chlorobenzotrifluoride, N,N-dimethylformamide, 2-chloro-6-fluorotoluene, 2-fluoroanisole, anisole, 2,3-dimethylpyrazine, 4-fluoroanisole, 3-fluoroanisole, 3-trifluoro-methylanisole, 2-methylanisole, phenetol, 4-methylanisole, 3-methylanisole, 4-fluoro-3-methylanisole, 2-fluorobenzonitrile, 4-fluoroveratrol, 2,6-dimethylanisole, 3-fluorobenzonitrile, 2,5-dimethylanisole, 2,4-dimethylanisole, benzonitrile, 3,5-dimethylanisole, N,N-dimethylaniline, ethyl benzoate, 1-fluoro-3,5-dimethoxy-benzene, N-methylpyrrolidinone, 3-fluorobenzo-trifluoride, benzotrifluoride, dioxane, trifluoromethoxy-benzene, 4-fluorobenzotrifluoride, 3-fluoropyridine, toluene, 2-fluoro-toluene, 2-fluorobenzotrifluoride, 3-fluorotoluene, 4-isopropylbiphenyl, phenyl ether, pyridine, 4-fluorotoluene, 2,5-difluorotoluene, 1-chloro-2,4-difluorobenzene, 2-fluoropyridine, 3-chlorofluoro-benzene, 1-chloro-2,5-difluorobenzene, 4-chlorofluorobenzene, chloro-benzene, o-dichlorobenzene, 2-chlorofluorobenzene, p-xylene, m-xylene, o-xylene, a mixture of o-, m-, and p-xylene, 2-fluoro-m-xylene, 3-fluoro-o-xylene, tetrahydrofuran, morpholine, 1,4-dioxane, 2-methylthiophene, 3-methylthiophene, chloroform, 1,2-dichloroethane, dichloromethane, carbon tetrachloride, 1,2-dichloroethane, 1,1,1-trichloroethane, 1,1,2,2-tetrachloroethane, acetone, methylethylketone, propiophenone, acetophenone, cyclohexanone, ethyl acetate, n-butyl acetate, ethyl benzoate, ethyl benzoate, dimethylacetamide, dimethylsulfoxide, or mixtures of the aforementioned. Solvents with relatively low polarity are generally preferred.

Examples of especially preferred solvents include, without limitation, dichloromethane, trichloromethane, chlorobenzene, o-dichlorobenzene, tetrahydrofuran, anisole, 2,4-dimethylanisole, 1-methylnaphthalene, morpholine, toluene, o-xylene, m-xylene, p-xylene, 1,4-dioxane, acetone, methylethylketone, 1,2-dichloroethane, 1,1,1-trichloroethane, 1,1,2,2-tetrachloroethane, ethyl acetate, n-butyl acetate, N,N-dimethylformamide, dimethylacetamide, dimethylsulfoxide, 1,5-dimethyltetraline, propiophenone, acetophenone, tetralin, 2-methylthiophene, 3-methylthiophene, decaline, indane, methyl benzoate, ethyl benzoate, mesitylene, or mixtures thereof.

The concentration of the compounds or polymers in the solution is preferably 0.1 to 10% by weight, more preferably 0.5 to 5% by weight.

Optionally, the solution also comprises one or more binders to adjust the rheological properties, as described for example in WO 2005/055248 A1.

After the appropriate mixing and ageing, solutions are evaluated as one of the following categories: complete solution, borderline solution or insoluble. The contour line is drawn to outline the solubility parameter-hydrogen bonding limits dividing solubility and insolubility. ‘Complete’ solvents falling within the solubility area can be chosen from literature values such as published in “Crowley, J. D., Teague, G. S. Jr and Lowe, J. W. Jr., Journal of Paint Technology, 1966, 38 (496), 296”. Solvent blends may also be used and can be identified as described in “Solvents, W. H. Ellis, Federation of Societies for Coatings Technology, p 9-10, 1986”. Such a procedure may lead to a blend of ‘non’ solvents that will dissolve both the polymers of the present invention, although it is desirable to have at least one true solvent in a blend.

The compositions and formulations according to the present invention can additionally comprise one or more further components or additives selected for example from surface-active compounds, lubricating agents, wetting agents, dispersing agents, hydrophobing agents, adhesive agents, flow improvers, defoaming agents, deaerators, diluents which may be reactive or non-reactive, auxiliaries, colourants, dyes or pigments, sensitizers, stabilizers, nanoparticles or inhibitors.

In a composition according to the present invention comprising a first polymer, which is polymer according to the present invention, and a second conjugated polymer, the ratio 1^{st} polymer:2^{nd} polymer is preferably from 5:1 to 1:5 by weight, more preferably from 3:1 to 1:3 by weight, most preferably 2:1 to 1:2 by weight.

The composition according to the present invention may also comprise a polymeric binder, preferably from 0.001 to 95% by weight. Examples of binder include polystyrene (PS), polydimethylsilane (PDMS), polypropylene (PP) and polymethylmethacrylate (PMMA).

A binder to be used in the formulation as described before, which is preferably a polymer, may comprise either an insulating binder or a semiconducting binder, or mixtures thereof, may be referred to herein as the organic binder, the polymeric binder or simply the binder.

Preferably, the polymeric binder comprises a weight average molecular weight in the range of 1,000 to 5,000,000 g/mol, especially 1,500 to 1,000,000 g/mol and more preferable 2,000 to 500,000 g/mol. Surprising effects can be achieved with polymers having a weight average molecular weight of at least 10,000 g/mol, more preferably at least 100,000 g/mol.

In particular, the polymer can have a polydispersity index M_{w}/Mₙ in the range of 1.0 to 10.0, more preferably in the range of 1.1 to 5.0 and most preferably in the range of 1.2 to 3.

Preferably, the inert binder is a polymer having a glass transition temperature in the range of −70 to 160° C., preferably 0 to 150° C., more preferably 50 to 140° C. and most preferably 70 to 130° C. The glass transition temperature can be determined by measuring the DSC of the polymer (DIN EN ISO 11357, heating rate 10° C. per minute).

The weight ratio of the polymeric binder to the OSC polymer according to the present invention is preferably in the range of 30:1 to 1:30, particularly in the range of 5:1 to 1:20 and more preferably in the range of 1:2 to 1:10.

According to a preferred embodiment the binder preferably comprises repeating units derived from styrene monomers and/or olefin monomers. Preferred polymeric binders can comprise at least 80%, preferably 90% and more preferably 99% by weight of repeating units derived from styrene monomers and/or olefins.

Styrene monomers are well known in the art. These monomers include styrene, substituted styrenes with an alkyl substituent in the side chain, such as a-methylstyrene and a-ethylstyrene, substituted styrenes with an alkyl substituent on the ring such as vinyltoluene and p-methylstyrene, halogenated styrenes such as monochlorostyrenes, dichlorostyrenes, tribromostyrenes and tetrabromostyrenes.

Olefin monomers consist of hydrogen and carbon atoms. These monomers include ethylene, propylene, butylenes, isoprene and 1,3-butadiene.

According to a preferred embodiment of the present invention, the polymeric binder is polystyrene having a weight average molecular weight in the range of 50,000 to 2,000,000 g/mol, preferably 100,000 to 750,000 g/mol, more preferably in the range of 150,000 to 600,000 g/mol and most preferably in the range of 200,000 to 500,000 g/mol.

Further examples of suitable binders are disclosed for example in US 2007/0102696 A1. Especially suitable and preferred binders are described in the following.

The binder should preferably be capable of forming a film, more preferably a flexible film.

Suitable polymers as binders include poly(1,3-butadiene), polyphenylene, polystyrene, poly(α-methylstyrene), poly(α-vinylnaphtalene), poly(vinyltoluene), polyethylene, cis-polybutadiene, polypropylene, polyisoprene, poly(4-methyl-1-pentene), poly (4-methylstyrene), poly(chorotrifluoroethylene), poly(2-methyl-1,3-butadiene), poly(p-xylylene), poly(α-α-α′-α′ tetrafluoro-p-xylylene), poly[1,1-(2-methyl propane)bis(4-phenyl)carbonate], poly(cyclohexyl methacrylate), poly(chlorostyrene), poly(2,6-dimethyl-1,4-phenylene ether), polyisobutylene, poly(vinyl cyclohexane), poly(vinylcinnamate), poly(4-vinylbiphenyl), 1,4-polyisoprene, polynorbornene, poly(styrene-block-butadiene); 31% wt styrene, poly(styrene-block-butadiene-block-styrene); 30% wt styrene, poly(styrene-co-maleic anhydride) (and ethylene/butylene) 1-1.7% maleic anhydride, poly(styrene-block-ethylene/butylene-block-styrene) triblock polymer 13% styrene, poly(styrene-block-ethylene-propylene-block-styrene) triblock polymer 37% wt styrene, poly(styrene-block-ethylene/butylene-block-styrene) triblock polymer 29% wt styrene, poly(l-vinylnaphthalene), poly(l-vinylpyrrolidone-co-styrene) 64% styrene, poly(l-vinylpyrrolidone-co-vinyl acetate) 1.3:1, poly(2-chlorostyrene), poly(2-vinylnaphthalene), poly(2-vinylpyridine-co-styrene) 1:1, poly(4,5-Difluoro-2,2-bis(CF3)-1,3-dioxole-co-tetrafluoroethylene) Teflon, poly(4-chlorostyrene), poly(4-methyl-1-pentene), poly(4-methylstyrene), poly(4-vinylpyridine-co-styrene) 1:1, poly(alpha-methylstyrene), poly(butadiene-graft-poly(methyl acrylate-co-acrylonitrile)) 1:1:1, poly(butyl methacrylate-co-isobutyl methacrylate) 1:1, poly(butyl methacrylate-co-methyl methacrylate) 1:1, poly(cyclohexylmethacrylate), poly(ethylene-co-1-butene-co-1-hexene) 1:1:1, poly(ethylene-co-ethylacrylate-co-maleic anhydride); 2% anhydride, 32% ethyl acrylate, poly(ethylene-co-glycidyl methacrylate) 8% glycidyl methacrylate, poly(ethylene-co-methyl acrylate-co-glycidyl meth-acrylate) 8% glycidyl metha-crylate 25% methyl acrylate, poly(ethylene-co-octene) 1:1, poly(ethylene-co-propylene-co-5-methylene-2-norbornene) 50% ethylene, poly(ethylene-co-tetrafluoroethylene) 1:1, poly(isobutyl methacrylate), poly(isobutylene), poly(methyl methacrylate)-co-(fluorescein O-methacrylate) 80% methyl methacrylate, poly(methyl methacrylate-co-butyl methacrylate) 85% methyl methacrylate, poly(methyl methacrylate-co-ethyl acrylate) 5% ethyl acrylate, poly(propylene-co-butene) 12% 1-butene, poly(styrene-co-allyl alcohol) 40% allyl alcohol, poly(styrene-co-maleic anhydride) 7% maleic anhydride, poly(styrene-co-maleic anhydride) cumene terminated (1.3:1), poly(styrene-co-methyl methacrylate) 40% styrene, poly(vinyltoluene-co-alpha-methylstyrene) 1:1, poly-2-vinylpyridine, poly-4-vinylpyridine, poly-alpha-pinene, polymethylmethacrylate, polybenzylmethacrylate, polyethylmethacrylate, polyethylene, polyethylene terephthalate, polyethylene-co-ethylacrylate 18% ethyl acrylate, polyethylene-co-vinylacetate 12% vinyl acetate, polyethylene-graft-maleic anhydride 0.5% maleic anhydride, polypropylene, polypropylene-graft-maleic anhydride 8-10% maleic anhydride, polystyrene poly(styrene-block-ethylene/butylene-block-styrene) graft maleic anhydride 2% maleic anhydride 1:1:1 others, poly(styrene-block-butadiene) branched 1:1, poly(styrene-block-butadiene-block-styrene), 30% styrene, poly(styrene-block-isoprene) 10% wt styrene, poly(styrene-block-isoprene-block-styrene) 17% wt styrene, poly(styrene-co-4-chloromethylstyrene-co-4-methoxymethylstyrene 2:1:1, polystyrene-co-acrylonitrile 25% acrylonitrile, polystyrene-co-alpha-methylstyrene 1:1, polystyrene-co-butadiene 4% butadiene, polystyrene-co-butadiene 45% styrene, polystyrene-co-chloromethylstyrene 1:1, polyvinylchloride, polyvinylcinnamate, polyvinylcyclohexane, polyvinylidenefluoride, polyvinylidenefluoride-co-hexafluoropropylene assume 1:1, poly(styrene-block-ethylene/propylene-block-styrene) 30% styrene, poly(styrene-block-ethylene/propylene-block-styrene) 18% styrene, poly(styrene-block-ethylene/propylene-block-styrene) 13% styrene, poly(styrene-block ethylene block-ethylene/propylene-block styrene) 32% styrene, poly(styrene-block ethylene block-ethylene/propylene-block styrene) 30% styrene, poly(styrene-block-ethylene/butylene-block-styrene) 31 styrene, poly(styrene-block-ethylene/butylene-block-styrene) 34% styrene, poly(styrene-block-ethylene/butylene-block-styrene) 30% styrene, poly(styrene-block-ethylene/butylene-block-styrene) 60%, styrene, branched or non-branched polystyrene-block-polybutadiene, polystyrene-block(polyethylene-ran-butylene)-block-polystyrene, polystyrene-block-polybutadiene-block-polystyrene, polystyrene-(ethylene-propylene)-diblock-copolymers (e.g. KRATON®-G1701E, Shell), poly(propylene-co-ethylene) and poly(styrene-co-methylmethacrylate).

Preferred insulating binders to be used in the formulations as described before are polystryrene, poly(α-methylstyrene), polyvinylcinnamate, poly(4-vinylbiphenyl), poly(4-methylstyrene), and polymethyl methacrylate. Most preferred insulating binders are polystyrene and polymethyl methacrylate.

The binder can also be selected from crosslinkable binders, like e.g. acrylates, epoxies, vinylethers, thiolenes etc. The binder can also be mesogenic or liquid crystalline.

The organic binder may itself be a semiconductor, in which case it will be referred to herein as a semiconducting binder. The semiconducting binder is still preferably a binder of low permittivity as herein defined. Semiconducting binders for use in the present invention preferably have a number average molecular weight (Mₙ) of at least 1500-2000, more preferably at least 3000, even more preferably at least 4000 and most preferably at least 5000. The semiconducting binder preferably has a charge carrier mobility of at least 10⁻⁵ cm²V⁻¹s⁻¹, more preferably at least 10⁻⁴ cm²V⁻¹s⁻¹.

A preferred semiconducting binder comprises a homo-polymer or copolymer (including block-copolymer) containing arylamine (preferably triarylamine).

The polymers and compositions according to the present invention are useful as charge transport, semiconducting, electrically conducting, photoconducting or light emitting materials in optical, electronic, optoelectronic, electroluminescent or photoluminescent components or devices. In these devices, the polymers and compositions of the present invention are typically applied as thin layers or films.

Thus, the present invention also provides the use of the polymer or composition or layer in an electronic device. The polymer or composition may be used as a high mobility semiconducting material in various devices and apparatus. The polymer or composition may be used, for example, in the form of a semiconducting layer or film. Accordingly, in another aspect, the present invention provides a semiconducting layer for use in an electronic device, the layer comprising a polymer or composition according to the invention. The layer or film may be less than about 30 microns. For various electronic device applications, the thickness may be less than about 1 micron thick. The layer may be deposited, for example on a part of an electronic device, by any of the aforementioned solution coating or printing techniques.

The polymers according to the present invention can also be used in patterned OSC layers in the devices as described above and below. For applications in modern microelectronics it is generally desirable to generate small structures or patterns to reduce cost (more devices/unit area), and power consumption. Patterning of thin layers comprising a compound according to the present invention can be carried out for example by photolithography, electron beam lithography or laser patterning.

For use as thin layers in electronic or optoelectronic devices the compounds, compositions or formulations of the present invention may be deposited by any suitable method. Liquid coating of devices is more desirable than vacuum deposition techniques. Solution deposition methods are especially preferred. The formulations of the present invention enable the use of a number of liquid coating techniques. Preferred deposition techniques include, without limitation, dip coating, spin coating, ink jet printing, nozzle printing, letter-press printing, screen printing, gravure printing, doctor blade coating, roller printing, reverse-roller printing, offset lithography printing, dry offset lithography printing, flexographic printing, web printing, spray coating, curtain coating, brush coating, slot dye coating or pad printing.

For the fabrication of OPV devices and modules area printing method compatible with flexible substrates are preferred, for example slot dye coating, spray coating and the like.

Ink jet printing is particularly preferred when high resolution layers and devices needs to be prepared. Selected formulations of the present invention may be applied to prefabricated device substrates by ink jet printing or microdispensing. Preferably industrial piezoelectric print heads such as but not limited to those supplied by Aprion, Hitachi-Koki, InkJet Technology, On Target Technology, Picojet, Spectra, Trident, Xaar may be used to apply the organic semiconductor layer to a substrate. Additionally semi-industrial heads such as those manufactured by Brother, Epson, Konica, Seiko Instruments Toshiba TEC or single nozzle microdispensers such as those produced by Microdrop and Microfab may be used.

In order to be applied by ink jet printing or microdispensing, the compounds or polymers should be first dissolved in a suitable solvent.

Suitable solvents should be selected to ensure full dissolution of all components, like p-type and n-type OSCs, and take into account the boundary conditions (for example rheological properties) introduced by the chosen printing method. For inkjet printing solvents and solvent mixtures with high boiling temperatures are preferred. For spin coating alkylated benzenes like xylene and toluene are preferred.

Apart from the requirements stated above the solvents should not have any detrimental effect on the chosen print head. Additionally, the solvents should preferably have boiling points >100° C., preferably >140° C. and more preferably >150° C. in order to prevent operability problems caused by the solution drying out inside the print head.

Apart from the solvents mentioned above, suitable solvents include substituted and non-substituted xylene derivatives, di-C₁₋₂-alkyl formamide, substituted and non-substituted anisoles and other phenol-ether derivatives, substituted heterocycles such as substituted pyridines, pyrazines, pyrimidines, pyrrolidinones, substituted and non-substituted N,N-di-C₁₋₂-alkylanilines and other fluorinated or chlorinated aromatics.

A preferred solvent for depositing a polymer by ink jet printing comprises a benzene derivative which has a benzene ring substituted by one or more substituents wherein the total number of carbon atoms among the one or more substituents is at least three. For example, the benzene derivative may be substituted with a propyl group or three methyl groups, in either case there being at least three carbon atoms in total. Such a solvent enables an ink jet fluid to be formed comprising the solvent with the polymer, which reduces or prevents clogging of the jets and separation of the components during spraying. The solvent(s) may include those selected from the following list of examples: dodecylbenzene, 1-methyl-4-tert-butylbenzene, terpineol, limonene, isodurene, terpinolene, cymene, and diethylbenzene. The solvent may be a solvent mixture, that is a combination of two or more solvents, each solvent preferably having a boiling point >100° C., more preferably >140° C. Such solvent(s) also enhance film formation in the layer deposited and reduce defects in the layer.

The ink jet fluid (that is mixture of solvent, binder and semiconducting compound) preferably has a viscosity at 20° C. of 1-100 mPa·s, more preferably 1-50 mPa·s and most preferably 1-30 mPa·s.

The invention additionally provides an OE device comprising a polymer or composition or organic semiconducting layer according to the present invention.

Preferred OE devices are OFETs, TFTs, ICs, logic circuits, capacitors, RFID tags, OLEDs, OLETs, OPEDs, OPVs, PSCs, OPDs, solar cells, laser diodes, photoconductors, photodetectors, electrophotographic devices, electrophotographic recording devices, organic memory devices, sensor devices, charge injection layers, Schottky diodes, planarizing layers, antistatic films, conducting substrates and conducting patterns.

Very preferred OE devices are OPV, PSC and OPD devices, OFETs, and OLEDs, in particular OPD, PSC and bulk heterojunction (BHJ) OPV devices. In an OFET, for example, the active semiconductor channel between the drain and source may comprise the polymer or composition of the invention. As another example, in an OLED device, the charge (hole or electron) injection or transport layer may comprise the polymer or composition of the invention.

An OPV or OPD device according to the present invention preferably further comprises a first transparent or semi-transparent electrode on a transparent or semi-transparent substrate on one side of the photoactive layer, and a second metallic or semi-transparent electrode on the other side of the photoactive layer.

Further preferably the OPV or OPD device comprises, between the photoactive layer and the first or second electrode, one or more additional buffer layers acting as hole transporting layer and/or electron blocking layer, which comprise a material such as metal oxide, like for example, ZTO, MoOₓ, NiOₓ a conjugated polymer electrolyte, like for example PEDOT:PSS, a conjugated polymer, like for example polytriarylamine (PTAA), an insulating polymer, like for example nafion, polyethyleneimine or polystyrenesulphonate, an organic compound, like for example N,N′-diphenyl-N, N′-bis(1-naphthyl)(1,1′-biphenyl)-4,4′diamine (NPB), N,N′-diphenyl-N,N′-(3-methylphenyl)-1,1′-biphenyl-4,4′-diamine (TPD), or alternatively as hole blocking layer and/or electron transporting layer, which comprise a material such as metal oxide, like for example, ZnOₓ, TiOₓ, a salt, like for example LiF, NaF, CsF, a conjugated polymer electrolyte, like for example poly[3-(6-trimethylammoniumhexyl)thiophene], poly(9,9-bis(2-ethylhexyl)-fluorene]-b-poly[3-(6-trimethylammoniumhexyl)thiophene], or poly [(9,9-bis(3′-(N,N-dimethylamino)propyl)-2,7-fluorene)-alt-2,7-(9,9-dioctylfluorene)] or an organic compound, like for example tris(8-quinolinolato)-aluminum(III) (Alq₃), 4,7-diphenyl-1,10-phenanthroline.

The OPV device can for example be of any type known from the literature (see e.g. Waldauf et al., Appl. Phys. Left., 2006, 89, 233517).

A first preferred OPV device according to the invention comprises the following layers (in the sequence from bottom to top):
optionally a substrate,
a high work function electrode, preferably comprising a metal oxide, like for example ITO, serving as anode,
an optional conducting polymer layer or hole transport layer, preferably comprising an organic polymer or polymer blend, for example of PEDOT:PSS (poly(3,4-ethylenedioxythiophene): poly(styrene-sulfonate), or TBD (N,N′-dyphenyl-N—N′-bis(3-methylphenyl)-1,1′biphenyl-4,4′-diamine) or NBD (N,N′-dyphenyl-N—N′-bis(1-napthylphenyl)-1,1′biphenyl-4,4′-diamine),
a layer, also referred to as “photoactive layer”, comprising a p-type and an n-type organic semiconductor, which can exist for example as a p-type/n-type bilayer or as distinct p-type and n-type layers, or as blend or p-type and n-type semiconductor, forming a BHJ,
optionally a layer having electron transport properties, for example comprising LiF or PFN,
a low work function electrode, preferably comprising a metal like for example aluminum, serving as cathode,
wherein at least one of the electrodes, preferably the anode, is transparent to visible light, and
wherein the n-type semiconductor is a polymer according to the present invention.

A second preferred OPV device according to the invention is an inverted OPV device and comprises the following layers (in the sequence from bottom to top):
optionally a substrate,
a high work function metal or metal oxide electrode, comprising for example ITO, serving as cathode,
a layer having hole blocking properties, preferably comprising an organic polymer, polymer blend, metal or metal oxide like TiOₓ, ZnOₓ, Ca, Mg, poly(ethyleneimine), poly(ethyleneimine) ethoxylated or poly [(9,9-bis(3′-(N,N-dimethylamino)propyl)-2,7-fluorene)-alt-2,7-(9,9-dioctylfluorene)],
a photoactive layer comprising a p-type and an n-type organic semiconductor, situated between the electrodes, which can exist for example as a p-type/n-type bilayer or as distinct p-type and n-type layers, or as blend or p-type and n-type semiconductor, forming a BHJ,
an optional conducting polymer layer or hole transport layer, preferably comprising an organic polymer or polymer blend, metal or metal oxide, for example PEDOT:PSS, nafion, a substituted triaryl amine derivative like for example TBD or NBD, or WOₓ, MoOₓ, NiOₓ, Pd or Au,
an electrode comprising a high work function metal like for example silver, serving as anode,
wherein at least one of the electrodes, preferably the cathode, is transparent to visible light, and
wherein the n-type semiconductor is a polymer according to the present invention.

In the OPV devices of the present invention the p-type and n-type semiconductor materials are preferably selected from the materials, like the polymer/polymer/fullerene systems, as described above.

When the photoactive layer is deposited on the substrate, it forms a BHJ that phase separates at nanoscale level. For discussion on nanoscale phase separation see Dennler et al, Proceedings of the IEEE, 2005, 93 (8), 1429 or Hoppe et al, Adv. Func. Mater, 2004, 14(10), 1005. An optional annealing step may be then necessary to optimize blend morphology and consequently OPV device performance.

Another method to optimize device performance is to prepare formulations for the fabrication of OPV(BHJ) devices that may include high boiling point additives to promote phase separation in the right way. 1,8-Octanedithiol, 1,8-diiodooctane, nitrobenzene, chloronaphthalene, and other additives have been used to obtain high-efficiency solar cells. Examples are disclosed in J. Peet, et al, Nat. Mater., 2007, 6, 497 or Fréchet et al. J. Am. Chem. Soc., 2010, 132, 7595-7597.

Another preferred embodiment of the present invention relates to the use of a polymer or composition according to the present invention as dye, hole transport layer, hole blocking layer, electron transport layer and/or electron blocking layer in a DSSC or a perovskite-based solar cell (PSC), and to a DSSC or PSC comprising a polymer or composition according to the present invention.

DSSCs and PSCs can be manufactured as described in the literature, for example in Chem. Rev. 2010, 110, 6595-6663, Angew. Chem. Int. Ed. 2014, 53, 2-15 or in WO2013171520A1

A preferred OE device according to the invention is a solar cell, preferably a PSC, comprising a light absorber which is at least in part inorganic as described below.

In a solar cell comprising the light absorber according to the invention there are no restrictions per se with respect to the choice of the light absorber material which is at least in part inorganic.

The term “at least in part inorganic” means that the light absorber material may be selected from metalorganic complexes or materials which are substantially inorganic and possess preferably a crystalline structure where single positions in the crystalline structure may be allocated by organic ions.

Preferably, the light absorber comprised in the solar cell according to the invention has an optical band-gap ≤2.8 eV and ≥0.8 eV.

Very preferably, the light absorber in the solar cell according to the invention has an optical band-gap ≤2.2 eV and ≥1.0 eV.

The light absorber used in the solar cell according to the invention does preferably not contain a fullerene. The chemistry of fullerenes belongs to the field of organic chemistry. Therefore fullerenes do not fulfil the definition of being “at least in part inorganic” according to the invention.

Preferably, the light absorber which is at least in part inorganic is a material having perovskite structure or a material having 2D crystalline perovskite structure.

The term “perovskite” as used above and below denotes generally a material having a perovskite crystalline structure or a 2D crystalline perovskite structure.

The term perovskite solar cell (PSC) means a solar cell comprising a light absorber which is a material having perovskite structure or a material having 2D crystalline perovskite structure.

The light absorber which is at least in part inorganic is without limitation composed of a material having perovskite crystalline structure, a material having 2D crystalline perovskite structure (e.g. CrystEngComm, 2010, 12, 2646-2662), Sb₂S₃ (stibnite), Sb₂(SₓSe₍ₓ₋₁₎)₃, PbSₓSe₍ₓ₋₁₎, CdSₓSe₍ₓ₋₁₎, ZnTe, CdTe, ZnSₓSe₍ₓ₋₁₎, InP, FeS, FeS₂, Fe₂S₃, Fe₂SiS₄, Fe₂GeS₄, Cu₂S, CuInGa, CuIn(SeₓS₍₁₋ₓ₎)₂, Cu₃SbₓBi₍ₓ₋₁₎, (S_{y}Se_{(y-1)})₃, Cu₂SnS₃, SnSₓSe₍ₓ₋₁₎, Ag₂S, AgBiS₂, BiSI, BiSeI, Bi₂(SₓSe₍ₓ₋₁₎)₃, BiS₍₁₋ₓ₎SeₓI, WSe₂, AlSb, metal halides (e.g. BiI₃, Cs₂SnI₆), chalcopyrite (e.g. CuInₓGa₍₁₋ₓ₎(S_{y}Se₍₁₋ₓ₎)₂), kesterite (e.g. Cu₂ZnSnS₄, Cu₂ZnSn(SeₓS₍₁₋ₓ₎)₄, Cu₂Zn(Sn₁₋ₓGeₓ)S₄) and metal oxide (e.g. CuO, Cu₂O) or a mixture thereof.

Preferably, the light absorber which is at least in part inorganic is a perovskite.

In the above definition for light absorber, x and y are each independently defined as follows: (0≤x≤1) and (0≤y≤1).

Very preferably, the light absorber is a special perovskite namely a metal halide perovskite as described in detail above and below. Most preferably, the light absorber is an organic-inorganic hybrid metal halide perovskite contained in the perovskite solar cell (PSC).

In one particularly preferred embodiment of the invention, the perovskite denotes a metal halide perovskite with the formula ABX₃,

where
A is a monovalent organic cation, a metal cation or a mixture of two or more of these cations
B is a divalent cation and
X is F, Cl, Br, I, BF₄ or a combination thereof.

Preferably, the monovalent organic cation of the perovskite is selected from alkylammonium, wherein the alkyl group is straight chain or branched having 1 to 6 C atoms, formamidinium or guanidinium or wherein the metal cation is selected from K⁺, Cs⁺ or Rb⁺.

Suitable and preferred divalent cations B are Ge²⁺, Sn²⁺ or Pb²⁺.

Suitable and preferred perovskite materials are CsSnI₃, CH₃NH₃Pb(I₁₋ₓClₓ)₃, CH₃NH₃PbI₃, CH₃NH₃Pb(I₁₋ₓBrₓ)₃, CH₃NH₃Pb(I₁₋ₓ(BF₄)ₓ)₃, CH₃NH₃Sn(I₁₋ₓClₓ)₃, CH₃NH₃SnI₃ or CH₃NH₃Sn(I₁₋ₓBrₓ)₃ wherein x is each independently defined as follows: (0<x≤1).

Further suitable and preferred perovskites may comprise two halides corresponding to formula Xa₍₃₋ₓ₎Xb₍ₓ₎, wherein Xa and Xb are each independently selected from Cl, Br, or I, and x is greater than 0 and less than 3.

Suitable and preferred perovskites are also disclosed in WO 2013/171517, claims 52 to 71 and claims 72 to 79, which is entirely incorporated herein by reference. The materials are defined as mixed-anion perovskites comprising two or more different anions selected from halide anions and chalcogenide anions. Preferred perovskites are disclosed on page 18, lines 5 to 17. As described, the perovskite is usually selected from CH₃NH₃PbBrI₂, CH₃NH₃PbBrCl₂, CH₃NH₃PbIBr₂, CH₃NH₃PbICl₂, CH₃NH₃SnF₂Br, CH₃NH₃SnF₂I and (H₂N═CH—NH₂)PbI_{3z}Br_{3(1-z)}, wherein z is greater than 0 and less than 1.

The invention further relates to a solar cell comprising the light absorber, preferably a PSC, as described above and below, wherein the polymer according to the present invention is employed as a layer between one electrode and the light absorber layer.

The invention further relates to a solar cell comprising the light absorber, preferably a PSC, as described above and below, wherein the polymer according to the present invention is comprised in an electron-selective layer.

The electron selective layer is defined as a layer providing a high electron conductivity and a low hole conductivity favoring electron-charge transport.

The invention further relates to a solar cell comprising the light absorber, preferably a PSC, as described above and below, wherein the polymer according to the present invention is employed as electron transport material (ETM) or as hole blocking material as part of the electron selective layer.

Preferably, the polymer according to the present invention is employed as electron transport material (ETM).

In an alternative preferred embodiment, the polymer according to the present invention is employed as hole blocking material.

The device architecture of a PSC device according to the invention can be of any type known from the literature.

A first preferred device architecture of a PSC device according to the invention comprises the following layers (in the sequence from bottom to top):
optionally a substrate which, in any combination, can be flexible or rigid and transparent, semi-transparent or non-transparent and electrically conductive or non-conductive;
a high work function electrode, preferably comprising a doped metal oxide, for example fluorine-doped tin oxide (FTO), tin-doped indium oxide (ITO), or aluminum-doped zinc oxide;
an electron-selective layer which comprises one or more electron-transporting materials, at least one of which is a polymer according to the present invention, and which, in some cases, can also be a dense layer and/or be composed of nanoparticles, and which preferably comprises a metal oxide such as TiO₂, ZnO₂, SnO₂, Y₂O₅, Ga₂O₃, SrTiO₃, BaTiO₃ or combinations thereof;
optionally a porous scaffold which can be conducting, semi-conducting or insulating, and which preferably comprises a metal oxide such as TiO₂, ZnO₂, SnO₂, Y₂O₅, Ga₂O₃, SrTiO₃, BaTiO₃, Al₂O₃, ZrO₂, SiO₂ or combinations thereof, and which is preferably composed of nanoparticles, nanorods, nanoflakes, nanotubes or nanocolumns;
a layer comprising a light absorber which is at least in part inorganic, particularly preferably a metal halide perovskite as described above which, in some cases, can also be a dense or porous layer and which optionally partly or fully infiltrates into the underlying layer;
optionally a hole selective layer, which comprises one or more hole-transporting materials, and which, in some cases, can also comprise additives such as lithium salts, for example LiY, where Y is a monovalent organic anion, preferably bis(trifluoromethylsulfonyl)imide, tertiary amines such as 4-tert-butylpyridine, or any other covalent or ionic compounds, for example tris(2-(1H-pyrazol-1-yl)-4-tert-butylpyridine)-cobalt(III) tris(bis(trifluoromethylsulfonyl)imide)), which can enhance the properties of the hole selective layer, for example the electrical conductivity, and/or facilitate its processing;

and a back electrode which can be metallic, for example made of Au, Ag, Al, Cu, Ca, Ni or combinations thereof, or non-metallic and transparent, semi-transparent or non-transparent.

A second preferred device architecture of a PSC device according to the invention comprises the following layers (in the sequence from bottom to top):
optionally a substrate which, in any combination, can be flexible or rigid and transparent, semi-transparent or non-transparent and electrically conductive or non-conductive;
a high work function electrode, preferably comprising a doped metal oxide, for example fluorine-doped tin oxide (FTO), tin-doped indium oxide (ITO), or aluminum-doped zinc oxide;
optionally a hole injection layer which, for example, changes the work function of the underlying electrode, and/or modifies the surface of the underlying layer and/or helps to planarize the rough surface of the underlying layer and which, in some cases, can also be a monolayer;
optionally a hole selective layer, which comprises one or more hole-transporting materials and which, in some cases, can also comprise additives such as lithium salts, for example LiY, where Y is a monovalent organic anion, preferably bis(trifluoromethylsulfonyl)imide, tertiary amines such as 4-tert-butylpyridine, or any other covalent or ionic compounds, for example tris(2-(1H-pyrazol-1-yl)-4-tert-butylpyridine)-cobalt(III) tris(bis(trifluoromethylsulfonyl)imide)), which can enhance the properties of the hole selective layer, for example the electrical conductivity, and/or facilitate its processing;
a layer comprising a light absorber which is at least in part inorganic, particularly preferably a metal halide perovskite as described or preferably described above;
an electron-selective layer, which comprises one or more electron-transporting materials, at least one of which is a polymer according to the present invention and which, in some cases, can also be a dense layer and/or be composed of nanoparticles, and which, for example, can comprise a metal oxide such as TiO₂, ZnO₂, SnO₂, Y₂O₅, Ga₂O₃, SrTiO₃, BaTiO₃ or combinations thereof, and/or which can comprise a substituted fullerene, for example [6,6]-phenyl C61-butyric acid methyl ester, and/or which can comprise a molecular, oligomeric or polymeric electron-transport material, for example 2,9-Dimethyl-4,7-diphenyl-1,10-phenanthroline, or a mixture thereof;

and a back electrode which can be metallic, for example made of Au, Ag, Al, Cu, Ca, Ni or combinations thereof, or non-metallic and transparent, semi-transparent or non-transparent.

To produce electron selective layers in PSC devices according to the invention, the polymers according to the present invention, optionally together with other compounds or additives in the form of blends or mixtures, may be deposited by any suitable method. Liquid coating of devices is more desirable than vacuum deposition techniques. Solution deposition methods are especially preferred. Formulations comprising the polymers according to the present invention enable the use of a number of liquid coating techniques. Preferred deposition techniques include, without limitation, dip coating, spin coating, ink jet printing, nozzle printing, letter-press printing, screen printing, gravure printing, doctor blade coating, roller printing, reverse-roller printing, offset lithography printing, dry offset lithography printing, flexographic printing, web printing, spray coating, curtain coating, brush coating, slot die coating or pad printing. For the fabrication of PSC devices and modules, deposition techniques for large area coating are preferred, for example slot die coating or spray coating.

Formulations that can be used to produce electron selective layers in optoelectronic devices according to the invention, preferably in PSC devices comprise one or more polymers according to the present invention or preferred embodiments as described above in the form of blends or mixtures optionally together with one or more further electron transport materials and/or hole blocking materials and/or binders and/or other additives as described above and below, and one or more solvents.

The formulation may include or comprise, essentially consist of or consist of the said necessary or optional constituents as described above or below. All compounds or components which can be used in the formulations are either known or commercially available, or can be synthesized by known processes.

The formulation as described before may be prepared by a process which comprises:
(i) first mixing a polymer according to the present invention, optionally a binder or a precursor of a binder as described before, optionally a further electron transport material, optionally one or more further additives as described above and below and a solvent or solvent mixture as described above and below and
(ii) applying such mixture to a substrate; and optionally evaporating the solvent(s) to form an electron selective layer according to the present invention.

In step (i) the solvent may be a single solvent for the polymer according to the present invention and the organic binder and/or further electron transport material may each be dissolved in a separate solvent followed by mixing the resultant solutions to mix the compounds.

Alternatively, the binder may be formed in situ by mixing or dissolving a polymer according to the present invention in a precursor of a binder, for example a liquid monomer, oligomer or crosslinkable polymer, optionally in the presence of a solvent, and depositing the mixture or solution, for example by dipping, spraying, painting or printing it, on a substrate to form a liquid layer and then curing the liquid monomer, oligomer or crosslinkable polymer, for example by exposure to radiation, heat or electron beams, to produce a solid layer. If a preformed binder is used it may be dissolved together with the polymer in a suitable solvent as described before, and the solution deposited for example by dipping, spraying, painting or printing it on a substrate to form a liquid layer and then removing the solvent to leave a solid layer. It will be appreciated that solvents are chosen which are able to dissolve all ingredients of the formulation, and which upon evaporation from the solution blend give a coherent defect free layer.

Besides the said components, the formulation as described before may comprise further additives and processing assistants. These include, inter alia, surface-active substances (surfactants), lubricants and greases, additives which modify the viscosity, additives which increase the conductivity, dispersants, hydrophobicizing agents, adhesion promoters, flow improvers, antifoams, deaerating agents, diluents, which may be reactive or unreactive, fillers, assistants, processing assistants, dyes, pigments, stabilizers, sensitizers, nanoparticles and inhibitors.

Additives can be used to enhance the properties of the electron selective layer and/or the properties of any of the neighbouring layers and/or the performance of the optoelectronic device according to the invention. Additives can also be used to facilitate the deposition, the processing or the formation of the electron selective layer and/or the deposition, the processing or the formation of any of the neighbouring layers. Preferably, one or more additives are used which enhance the electrical conductivity of the electron selective layer and/or passivate the surface of any of the neighbouring layers.

Suitable methods to incorporate one or more additives include, for example exposure to a vapor of the additive at atmospheric pressure or at reduced pressure, mixing a solution or solid containing one or more additives and a material or a formulation as described or preferably described before, bringing one or more additives into contact with a material or a formulation as described before, by thermal diffusion of one or more additives into a material or a formulation as described before, or by ion-implantation of one or more additives into a material or a formulation as described before.

Additives used for this purpose can be organic, inorganic, metallic or hybrid materials. Additives can be molecular compounds, for example organic molecules, salts, ionic liquids, coordination complexes or organometallic compounds, polymers or mixtures thereof. Additives can also be particles, for example hybrid or inorganic particles, preferably nanoparticles, or carbon based materials such as fullerenes, carbon nanotubes or graphene flakes.

Examples for additives that can enhance the electrical conductivity are for example halogens (e.g. I₂, Cl₂, Br₂, ICl, ICl₃, IBr and IF), Lewis acids (e.g. PF₅, AsF₅, SbF₅, BF₃, BCl₃, SbCl₅, BBr₃ and SO₃), protonic acids, organic acids, or amino acids (e.g. HF, HCl, HNO₃, H₂SO₄, HClO₄, FSO₃H and ClSO₃H), transition metal compounds (e.g. FeCl₃, FeOCl, Fe(ClO₄)₃, Fe(4-CH₃C₆H₄SO₃)₃, TiCl₄, ZrCl₄, HfCl₄, NbF₅, NbCl₅, TaCl₅, MoF₅, MoCl₅, WF₅, WCl₆, UF₆ and LnCl₃ (wherein Ln is a lanthanoid)), anions (e.g. Cl⁻, Br⁻, I⁻, I₃⁻, HSO₄⁻, SO₄²⁻, NO₃⁻, ClO₄⁻, BF₄⁻, PF₆⁻, AsF₆⁻, SbF₆⁻, FeCl₄⁻, Fe(CN)₆³⁻, and anions of various sulfonic acids, such as aryl-SO₃⁻), cations (e.g. H⁺, Li⁺, Na⁺, K⁺, Rb⁺, Cs⁺, Co³⁺ and Fe³⁺), O₂, redox active salts (e.g. XeOF₄, (NO₂⁺) (SbF₆⁻), (NO₂⁺) (SbCl₆⁻), (NO₂⁺) (BF₄⁻), NOBF₄, NOPF₆, AgClO₄, H₂IrCl₆ and La(NO₃)₃.6H₂O), strongly electron-accepting organic molecules (e.g. 2,3,5,6-tetrafluoro-7,7,8,8-tetracyanoquinodimethane (F4-TCNQ)), transition metal oxides (e.g. WO₃, Re₂O₇ and MoO₃), metalorganic complexes of cobalt, iron, bismuth and molybdenum, (p-BrC₆H₄)₃NSbCl₆, bismuth(III) tris(trifluoroacetate), FSO₂OOSO₂F, acetylcholine, R₄N⁺, (R is an alkyl group), R₄P⁺ (R is a straight-chain or branched alkyl group 1 to 20), R₆As⁺ (R is an alkyl group), R₃S⁺ (R is an alkyl group) and ionic liquids (e.g. 1-Ethyl-3-methylimidazolium bis(trifluoromethylsulfonyl)imide). Suitable cobalt complexes beside of tris(2-(1H-pyrazol-1-yl)-4-tert-butylpyridine)-cobalt(III) tris(bis(trifluoromethylsulfonyl)imide)) are cobalt complex salts as described in WO 2012/114315, WO 2012/114316, WO 2014/082706, WO 2014/082704, EP 2883881 or JP 2013-131477.

Suitable lithium salts are beside of lithium bis(trifluoromethylsulfonyl)imide, lithium tris(pentafluoroethyl)trifluorophosphate, lithium dicyanamide, lithium methylsulfate, lithium trifluormethanesulfonate, lithium tetracyanoborate, lithium dicyanamide, lithium tricyanomethide, lithium thiocyanate, lithium chloride, lithium bromide, lithium iodide, lithium hexafluorophosphate, lithium tetrafluoroborate, lithium perchlorate, lithium hexafluoroantimonate, lithium hexafluoroarsenate or a combination of two or more. A preferred lithium salt is lithium bis(trifluoromethylsulfonyl)imide.

Preferably, the formulation comprises from 0.1 mM to 50 mM, preferably from 5 to 20 mM of the lithium salt.

Suitable device structures for PSCs comprising a polymer according to the present invention and a mixed halide perovskite are described in WO 2013/171517, claims 52 to 71 and claims 72 to 79, which is entirely incorporated herein by reference.

Suitable device structures for PSCs comprising a polymer according to the present invention and a dielectric scaffold together with a perovskite are described in WO 2013/171518, claims 1 to 90 or WO 2013/171520, claims 1 to 94 which are entirely incorporated herein by reference.

Suitable device structures for PSCs comprising a polymer according to the present invention, a semiconductor and a perovskite are described in WO 2014/020499, claims 1 and 3 to 14, which is entirely incorporated herein by reference The surface-increasing scaffold structure described therein comprises nanoparticles which are applied and/or fixed on a support layer, e.g. porous TiO₂.

Suitable device structures for PSCs comprising a polymer according to the present invention and comprising a planar heterojunction are described in WO 2014/045021, claims 1 to 39, which is entirely incorporated herein by reference. Such a device is characterized in having a thin film of a light-absorbing or light-emitting perovskite disposed between n-type (electron conducting) and p-type (hole-conducting) layers. Preferably, the thin film is a compact thin film.

The invention further relates to a method of preparing a PSC as described above or below, the method comprising the steps of:
providing a first and a second electrode;
providing an electron selective layer comprising a polymer according to the present invention.

The invention relates furthermore to a tandem device comprising at least one device according to the invention as described above and below. Preferably, the tandem device is a tandem solar cell.

The tandem device or tandem solar cell according to the invention may have two semi-cells wherein one of the semi cells comprises the compounds, oligomers or polymers in the active layer as described or preferably described above. There exists no restriction for the choice of the other type of semi cell which may be any other type of device or solar cell known in the art.

There are two different types of tandem solar cells known in the art. The so called 2-terminal or monolithic tandem solar cells have only two connections. The two subcells (or synonymously semi cells) are connected in series. Therefore, the current generated in both subcells is identical (current matching). The gain in power conversion efficiency is due to an increase in voltage as the voltages of the two subcells add up. The other type of tandem solar cells is the so called 4-terminal or stacked tandem solar cell. In this case, both subcells are operated independently. Therefore, both subcells can be operated at different voltages and can also generate different currents. The power conversion efficiency of the tandem solar cell is the sum of the power conversion efficiencies of the two subcells.

The invention furthermore relates to a module comprising a device according to the invention as described before or preferably described before.

The polymers and compositions according to the present invention can also be used as dye or pigment in other applications, for example as an ink dye, laser dye, fluorescent marker, solvent dye, food dye, contrast dye or pigment in coloring paints, inks, plastics, fabrics, cosmetics, food and other materials.

The polymers and compositions of the present invention are also suitable for use in the semiconducting channel of an OFET. Accordingly, the invention also provides an OFET comprising a gate electrode, an insulating (or gate insulator) layer, a source electrode, a drain electrode and an organic semiconducting channel connecting the source and drain electrodes, wherein the organic semiconducting channel comprises a polymer or a composition according to the present invention. Other features of the OFET are well known to those skilled in the art.

OFETs where an OSC material is arranged as a thin film between a gate dielectric and a drain and a source electrode, are generally known, and are described for example in U.S. Pat. Nos. 5,892,244, 5,998,804, 6,723,394 and in the references cited in the background section. Due to the advantages, like low cost production using the solubility properties of the polymers according to the invention and thus the processibility of large surfaces, preferred applications of these OFETs are such as integrated circuitry, TFT displays and security applications.

The gate, source and drain electrodes and the insulating and semiconducting layer in the OFET device may be arranged in any sequence, provided that the source and drain electrode are separated from the gate electrode by the insulating layer, the gate electrode and the semiconductor layer both contact the insulating layer, and the source electrode and the drain electrode both contact the semiconducting layer.

An OFET device according to the present invention preferably comprises:
a source electrode,
a drain electrode,
a gate electrode,
a semiconducting layer,
one or more gate insulator layers,
optionally a substrate.

wherein the semiconductor layer preferably comprises a polymer according to the present invention.

The OFET device can be a top gate device or a bottom gate device. Suitable structures and manufacturing methods of an OFET device are known to the skilled in the art and are described in the literature, for example in US 2007/0102696 A1.

The gate insulator layer preferably comprises a fluoropolymer, like e.g. the commercially available Cytop 809M® or Cytop 107M® (from Asahi Glass). Preferably the gate insulator layer is deposited, e.g. by spin-coating, doctor blading, wire bar coating, spray or dip coating or other known methods, from a formulation comprising an insulator material and one or more solvents with one or more fluoro atoms (fluorosolvents), preferably a perfluorosolvent. A suitable perfluorosolvent is e.g. FC75® (available from Acros, catalogue number 12380). Other suitable fluoropolymers and fluorosolvents are known in prior art, like for example the perfluoropolymers Teflon AF® 1600 or 2400 (from DuPont) or Fluoropel® (from Cytonix) or the perfluorosolvent FC 43® (Acros, No. 12377). Especially preferred are organic dielectric materials having a low permittivity (or dielectric constant) from 1.0 to 5.0, very preferably from 1.8 to 4.0 (“low k materials”), as disclosed for example in US 2007/0102696 A1 or U.S. Pat. No. 7,095,044.

In security applications, OFETs and other devices with semiconducting materials according to the present invention, like transistors or diodes, can be used for RFID tags or security markings to authenticate and prevent counterfeiting of documents of value like banknotes, credit cards or ID cards, national ID documents, licenses or any product with monetary value, like stamps, tickets, shares, cheques etc.

Alternatively, the polymers and compositions (hereinafter referred to as “materials”) according to the present invention can be used in OLEDs, e.g. as the active display material in a flat panel display applications, or as backlight of a flat panel display like e.g. a liquid crystal display. Common OLEDs are realized using multilayer structures. An emission layer is generally sandwiched between one or more electron-transport and/or hole-transport layers. By applying an electric voltage electrons and holes as charge carriers move towards the emission layer where their recombination leads to the excitation and hence luminescence of the lumophor units contained in the emission layer. The materials according to the present invention may be employed in one or more of the charge transport layers and/or in the emission layer, corresponding to their electrical and/or optical properties. Furthermore their use within the emission layer is especially advantageous, if the materials according to the present invention show electroluminescent properties themselves or comprise electroluminescent groups or compounds. The selection, characterization as well as the processing of suitable monomeric, oligomeric and polymeric compounds or materials for the use in OLEDs is generally known by a person skilled in the art, see, e.g., Müller et al, Synth. Metals, 2000, 111-112, 31-34, Alcala, J. Appl. Phys., 2000, 88, 7124-7128 and the literature cited therein.

According to another use, the materials according to the present invention, especially those showing photoluminescent properties, may be employed as materials of light sources, e.g. in display devices, as described in EP 0 889 350 A1 or by C. Weder et al., Science, 1998, 279, 835-837.

A further aspect of the invention relates to both the oxidized and reduced form of the materials according to the present invention. Either loss or gain of electrons results in formation of a highly delocalized ionic form, which is of high conductivity. This can occur on exposure to common dopants. Suitable dopants and methods of doping are known to those skilled in the art, e.g. from EP 0 528 662, U.S. Pat. No. 5,198,153 or WO 96/21659.

The doping process typically implies treatment of the semiconductor material with an oxidating or reducing agent in a redox reaction to form delocalized ionic centers in the material, with the corresponding counterions derived from the applied dopants. Suitable doping methods comprise for example exposure to a doping vapor in the atmospheric pressure or at a reduced pressure, electrochemical doping in a solution containing a dopant, bringing a dopant into contact with the semiconductor material to be thermally diffused, and ion-implantation of the dopant into the semiconductor material.

When electrons are used as carriers, suitable dopants are for example halogens (e.g., I₂, Cl₂, Br₂, ICl, ICl₃, IBr and IF), Lewis acids (e.g., PF₅, AsF₅, SbF₅, BF₃, BCl₃, SbCl₅, BBr₃ and SO₃), protonic acids, organic acids, or amino acids (e.g., HF, HCl, HNO₃, H₂SO₄, HClO₄, FSO₃H and ClSO₃H), transition metal compounds (e.g., FeCl₃, FeOCl, Fe(ClO₄)₃, Fe(4-CH₃C₆H₄SO₃)₃, TiCl₄, ZrCl₄, HfCl₄, NbF₅, NbCl₅, TaCl₅, MoF₅, MoCl₅, WF₅, WCl₆, UF₆ and LnCl₃ (wherein Ln is a lanthanoid), anions (e.g., Cl⁻, Br⁻, I⁻, I₃⁻, HSO₄⁻, SO₄²⁻, NO₃⁻, ClO₄⁻, BF₄⁻, PF₆⁻, AsF₆⁻, SbF₆⁻, FeCl₄⁻, Fe(CN)₆³⁻, and anions of various sulfonic acids, such as aryl-SO₃⁻). When holes are used as carriers, examples of dopants are cations (e.g., H⁺, Li⁺, Na⁺, K⁺, Rb⁺ and Cs⁺), alkali metals (e.g., Li, Na, K, Rb, and Cs), alkaline-earth metals (e.g., Ca, Sr, and Ba), O₂, XeOF₄, (NO₂⁺) (SbF₆⁻), (NO₂⁺) (SbCl₆⁻), (NO₂⁺) (BF₄ AgClO₄, H₂IrCl₆, La(NO₃)₃.6H₂O, FSO₂OOSO₂F, Eu, acetylcholine, R₄N⁺, (R is an alkyl group), R₄P⁺ (R is an alkyl group), R₆As⁺ (R is an alkyl group), and R₃S⁺ (R is an alkyl group).

The conducting form of the materials according to the present invention can be used as an organic “metal” in applications including, but not limited to, charge injection layers and ITO planarizing layers in OLED applications, films for flat panel displays and touch screens, antistatic films, printed conductive substrates, patterns or tracts in electronic applications such as printed circuit boards and condensers.

The materials according to the present invention may also be suitable for use in organic plasmon-emitting diodes (OPEDs), as described for example in Koller et al., Nat. Photonics, 2008, 2, 684.

According to another use, the materials according to the present invention can be used alone or together with other materials in or as alignment layers in LCD or OLED devices, as described for example in US 2003/0021913. The use of charge transport compounds according to the present invention can increase the electrical conductivity of the alignment layer. When used in an LCD, this increased electrical conductivity can reduce adverse residual dc effects in the switchable LCD cell and suppress image sticking or, for example in ferroelectric LCDs, reduce the residual charge produced by the switching of the spontaneous polarization charge of the ferroelectric LCs. When used in an OLED device comprising a light emitting material provided onto the alignment layer, this increased electrical conductivity can enhance the electroluminescence of the light emitting material.

The materials according to the present invention having mesogenic or liquid crystalline properties can form oriented anisotropic films as described above, which are especially useful as alignment layers to induce or enhance alignment in a liquid crystal medium provided onto said anisotropic film.

According to another use, the materials according to the present invention are suitable for use in liquid crystal (LC) windows, also known as smart windows.

The materials according to the present invention may also be combined with photoisomerizable compounds and/or chromophores for use in or as photoalignment layers, as described in US 2003/0021913 A1.

According to another use, the materials according to the present invention, especially their water-soluble derivatives (for example with polar or ionic side groups) or ionically doped forms, can be employed as chemical sensors or materials for detecting and discriminating DNA sequences. Such uses are described for example in L. Chen, D. W. McBranch, H. Wang, R. Helgeson, F. Wudl and D. G. Whitten, Proc. Natl. Acad. Sci. U.S.A., 1999, 96, 12287; D. Wang, X. Gong, P. S. Heeger, F. Rininsland, G. C. Bazan and A. J. Heeger, Proc. Natl. Acad. Sci. U.S.A., 2002, 99, 49; N. DiCesare, M. R. Pinot, K. S. Schanze and J. R. Lakowicz, Langmuir, 2002, 18, 7785; D. T. McQuade, A. E. Pullen, T. M. Swager, Chem. Rev., 2000, 100, 2537.

Unless the context clearly indicates otherwise, as used herein plural forms of the terms herein are to be construed as including the singular form and vice versa.

Throughout the description and claims of this specification, the words “comprise” and “contain” and variations of the words, for example “comprising” and “comprises”, mean “including but not limited to”, and are not intended to (and do not) exclude other components.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

Above and below, unless stated otherwise percentages are percent by weight and temperatures are given in degrees Celsius.

The invention will now be described in more detail by reference to the following examples, which are illustrative only and do not limit the scope of the invention.

### Example 1

Intermediate 2 is prepared as follows

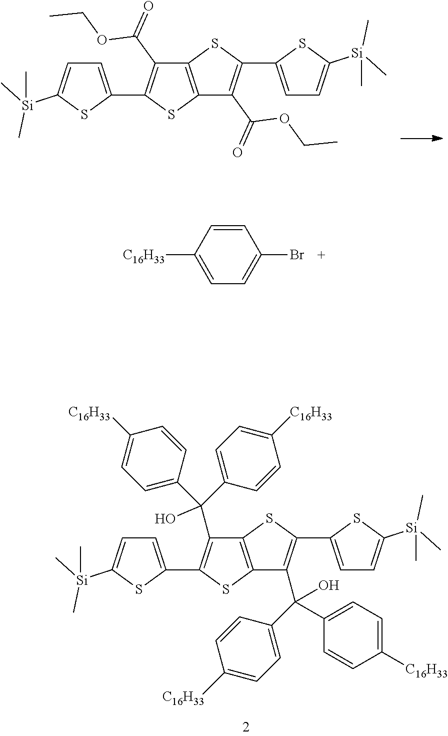

To a solution of 1-bromo-4-hexadecyl-benzene (19.3 g; 50.59 mmol) suspended in anhydrous tetrahydrofuran (400.00 cm³) at −35° C. is added tert-butyllithium (59.5 cm³; 101 mmol; 1.7 M in pentane) over 30 minutes. The suspension is warmed to −25° C. to provide a solution which is then re-cooled to −40° C. and stirred for an hour. 2,5-Bis-(5-trimethylsilanyl-thiophen-2-yl)-thieno[3,2-b]thiophene-3,6-dicarboxylic acid diethyl ester (5.00 g; 8.43 mmol) is dissolved in tetrahydrofuran (50 m³) and added to the mixture. The resulting suspension is warmed to 23° C. and stirred for 16 hours. Water (20 cm³) is added slowly followed by water (300 cm³). The biphasic solution is extracted with diethyl ether (300 cm³) and the organic phase washed with water (2×250 cm³), brine (100 cm³), dried over magnesium sulphate and concentrated in vacou to give an orange oil. The crude product is dissolved in acetone (200 cm³) and cooled in an ice bath. Methanol (200 cm³) is added portion wise, the precipitated product is filtered off and washed with methanol. The product is isolated as an off white solid (14 g, 97%). ¹H NMR (400 MHz, CDCl₃) 7.01-6.94 (8H, m), 6.94-6.84 (8H, m), 6.61 (2H, d, J 3.4), 6.22 (2H, d, J 3.4), 3.19 (2H, s), 2.38 (8H, t, J 7.7), 1.43-1.36 (8H, m), 1.15-0.97 (104H, m), 0.67 (12H, t, J 6.8), 0.00 (18H, s).

Intermediate 3 is prepared as follows

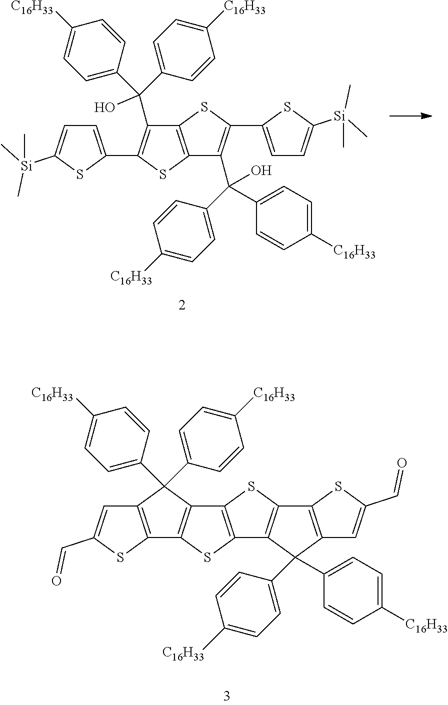

To a nitrogen flushed solution of intermediate 2 (5.0 g; 2.9 mmol) in toluene (200.00 cm³) at 55° C. is added p-toluenesulfonic acid (1.67 g; 8.8 mmol), the reaction is further flushed with nitrogen for 10 minutes. The reaction is stirred 55° C. for 16 hours. Upon cooling to 23° C. the reaction is filtered, concentrated and purified by column chromatography eluting with 40-60 petrol:dichloromethane (9:1).

To an ice bath cooled solution of the intermediate material in chloroform (100 cm³) and N,N-dimethylformamide (12 g; 47 mmol) is added phosphorus oxychloride (6.72 g; 43.8 mmol). The ice bath is removed and the reaction is heated externally to 65° C. for 16 hours. Water (25 cm³) is slowly added to the mixture followed by a solution of sodium acetate (5 M; 250 cm³). The biphasic solution is stirred for 1 hour and the aqeuous phase is extracted with dichloromethane (2×30 cm³). The combined organic phases are washed with water (2×50 cm³), dried over magnesium sulphate and concentrated in vacuo. The product is triturated in acetone and filtered off. The solid is purified by column chromatography eluting with 40-60 petrol:dichloromethane (4:1). The resulting solid is triturated in boiling 40-60 petrol to afford intermediate 3 as an orange solid (1.59 g, 34%). ¹H NMR (400 MHz, CDCl₃) 9.82 (2H, s), 7.70 (2H, s), 7.18-7.08 (16H, m), 2.57 (8H, t), 1.65-1.53 (8H, m), 1.26 (104H, d, J 3.4), 0.89 (12H, t, J 6.8).

Compound 4 is prepared as follows

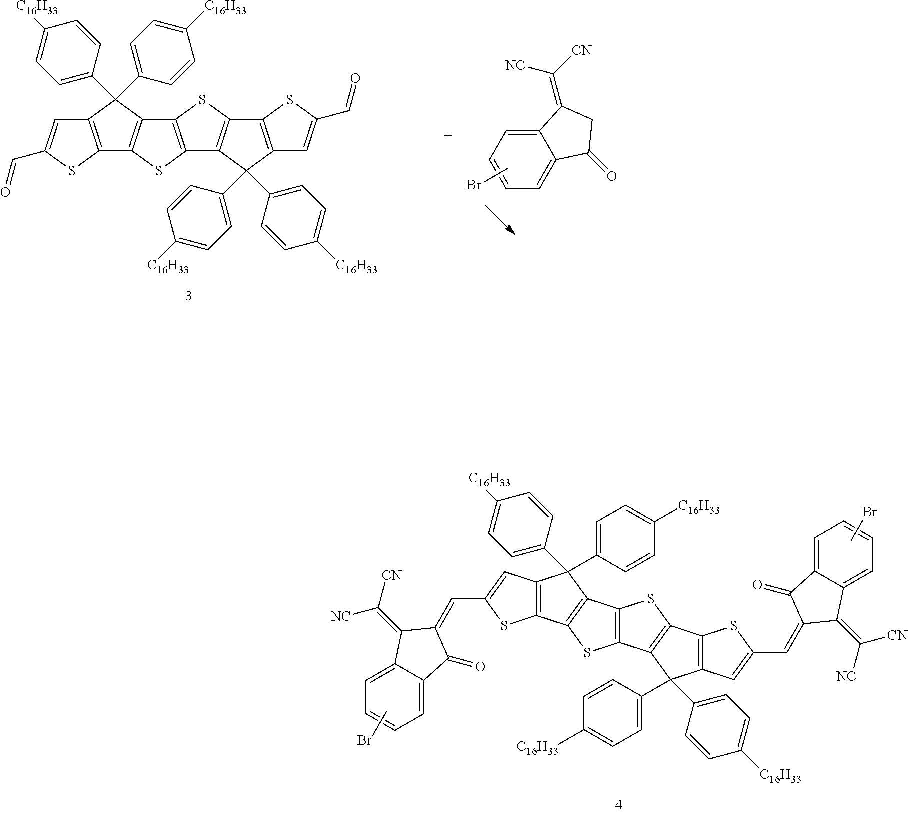

To a solution of intermediate 3 (600 mg; 0.38 mmol) in anhydrous chloroform (25 cm³) is added pyridine (5 cm³). The mixture is then degassed before a mixture of 2-(6-bromo-3-oxo-indan-1-ylidene)-malononitrile and 2-(5-bromo-3-oxo-indan-1-ylidene)-malononitrile (227 mg; 0.83 mmol; 2.20 eq.) is added. The mixture is stirred at 50° C. for 4 hours. Methanol (20 cm³) is added dropwise and the mixture is stirred for 15 minutes while cooling down. The precipitate is collected by filtration and washed with methanol (100 cm³). The solid is dissolved in dichloromethane:40-60 petrol 1:1 mixture and filtered through a pad of silica. The blue fraction is collected and concentrated in vacuo to dryness. The resulting solid is triturated in a mixture of chloroform (50 cm³) and acetone (150 cm³) at 50° C. for 30 minutes. Filtration affords 590 mg (74%) of the expected mixture of isomers as a dark blue solid. ¹H NMR (400 MHz, CDCl₃) 8.91-8.86 (2H, m), 8.85-8.50 (2H, m), 8.03-7.75 (4H, m), 7.73 (2H, s), 7.15 (16H, s), 2.64-2.55 (8H, m), 1.65-1.56 (8H, m), 1.41-1.19 (104H, m), 0.93-0.84 (12H, m).

Polymer 1 is prepared as follows:

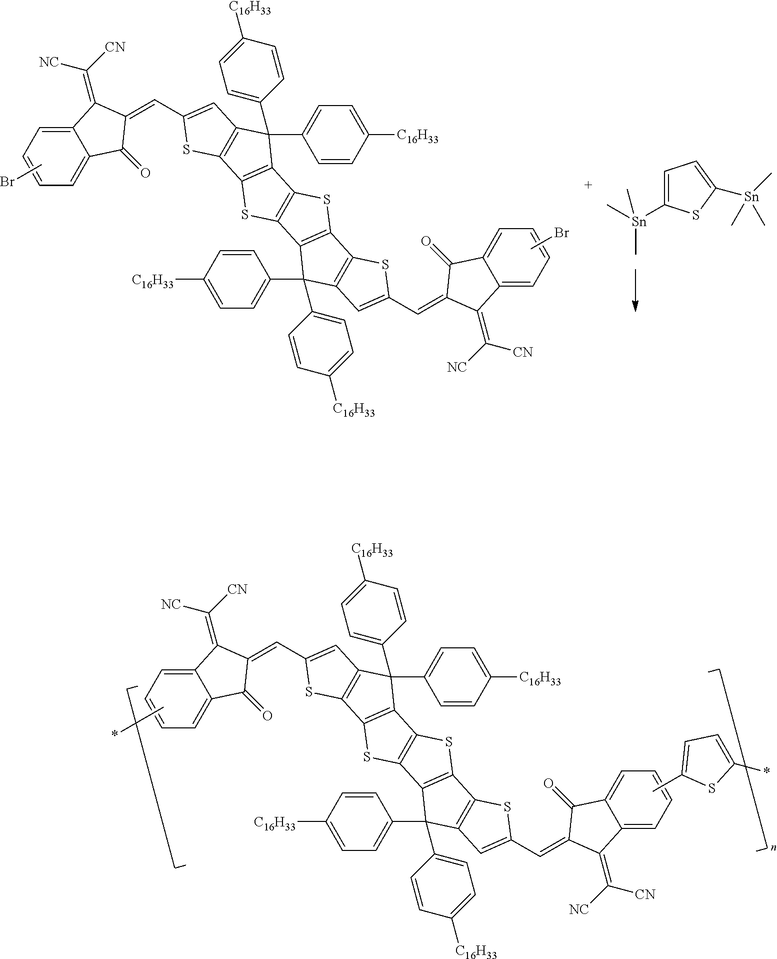

A solution of compound 4 (218.1 mg; 0.10 mmol), 2,5-bis-trimethylstannanyl-thiophene (42.6 mg; 0.10 mmol), tris(dibenzylideneacetone)dipalladium(0) (1.9 mg; 0.005 mmol.) and tri(o-tolyl)phosphine (5.1 mg; 0.02 mmol.) in anhydrous toluene (10 cm³) is degassed for 10 minutes with nitrogen. The mixture is then heated at 95° C. for 3 hours. Phenyl-tributylstannane (0.17 cm³; 0.52 mmol) is then added and the reaction mixture is stirred at the same temperature for 2 hours. Bromo-benzene (0.22 cm³; 2.1 mmol) is added and the mixture stirred at 95° C. for 16 hours. The reaction mixture is poured on methanol (50 cm³). The polymer is collected by filtration and washed with methanol (100 cm³) to give a solid. The polymer is subjected to sequential Soxhlet extraction with acetone, 40-60 petrol, cyclohexane and chloroform. The chloroform fraction is concentrated in vacuo to 20 cm³, precipitated into stirred methanol (150 cm³) and collected by filtration to give a black solid (177 mg, 78%). GPC (50° C., chlorobenzene) Mₙ=35.5 kg mol⁻¹; M_{w}=126.38 kg mol⁻¹; PDI=3.56.

### Example 2

Polymer 2 is prepared as follows:

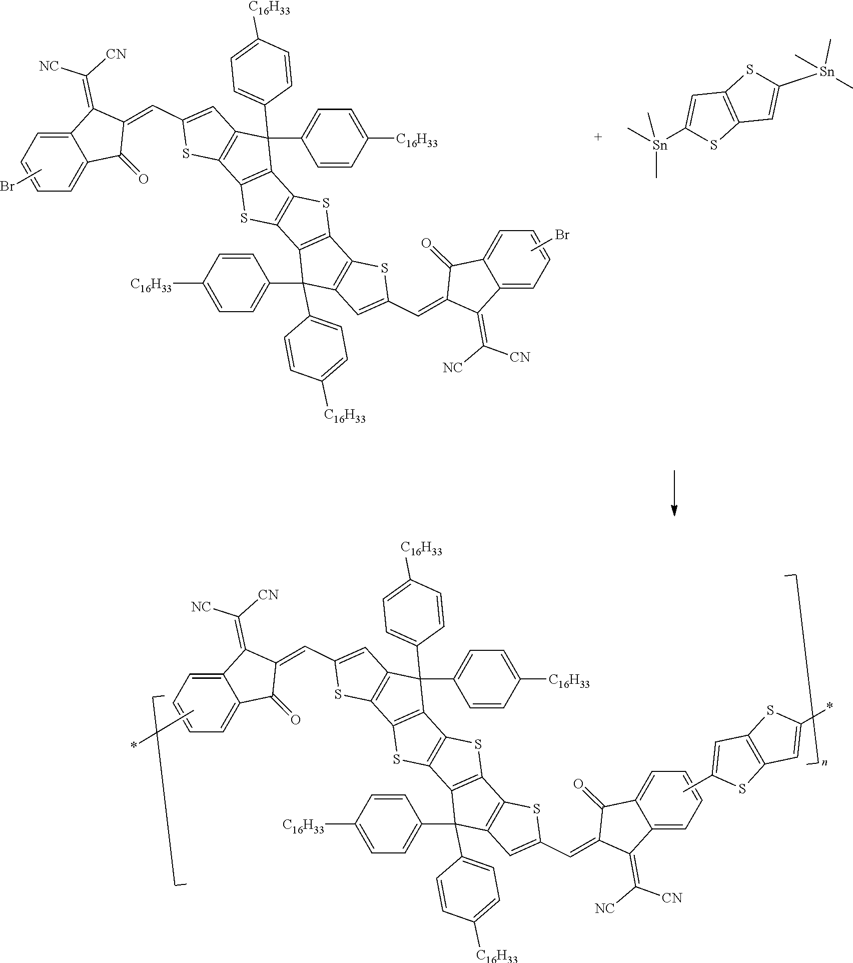

A solution of compound 4 (225.1 mg; 0.11 mmol), 2,5-bis-trimethylstannanyl-thieno[3,2-b]thiophene (50.0 mg; 0.11 mmol), tris(dibenzylideneacetone)dipalladium(0) (2.0 mg; 0.005 mmol.) and tri(o-tolyl)phosphine (5.2 mg; 0.02 mmol.) in anhydrous toluene (10 cm³) is degassed for 10 minutes with nitrogen. The mixture is then heated at 95° C. for 90 minutes. Toluene (10 cm³) and phenyl-tributylstannane (0.07 cm³; 0.21 mmol) are then added and the reaction mixture is stirred at the same temp for 1 hour. Bromo-benzene (0.22 cm³; 2.1 mmol) is added and the mixture stirred at 95° C. for 16 hours. The reaction mixture is poured on methanol (50 cm³). The polymer is collected by filtration and washed with methanol (100 cm³) to give a solid. The polymer is subjected to sequential Soxhlet extraction with acetone, 40-60 petrol, cyclohexane, chloroform and chlorobenzene. The chlorobenzene fraction is concentrated in vacuo to 20 cm³, precipitated into stirred methanol (150 cm³) and collected by filtration to give a black solid (46 mg, 19%).

### Use Example A

### A1: Inverted Bulk Heterojunction Organic Photovoltaic Devices

Organic photovoltaic (OPV) devices are fabricated on pre-patterned ITO-glass substrates (13 Ω/sq.). Substrates are cleaned using common solvents (acetone, iso-propanol, deionized-water) in an ultrasonic bath. A layer of commercially available aluminium zinc oxide (AlZnO, Nanograde) was applied as a uniform coating by doctor blade at 40° C. The AlZnO films are then annealed at 100° C. for 10 minutes in air. Active material solutions are prepared to fully dissolve the solutes at a 23 mg·cm⁻³ solution concentration. Thin films are blade-coated in air atmosphere to achieve active layer thicknesses between 50 and 800 nm as measured using a profilometer. A short drying period follows to ensure removal of any residual solvent.

Typically, blade-coated films are dried at 60° C. for 2 minutes on a hotplate. On top of the active layer 0.1 mL of a conducting polymer poly(ethylene dioxythiophene) doped with poly(styrene sulfonic acid) [PEDOT:PSS HTL 4083 (Heraeus)] was spread and uniformly coated by doctor blade at 70° C. Afterwards Ag (100 nm) cathodes are thermally evaporated through a shadow mask to define the cells.

Table 1 shows the formulation characteristics of the photoactive material solutions, comprising polymer P3, respectively, as electron donor component, and polymer P1 as electron acceptor component. The solvent is o-xylene.

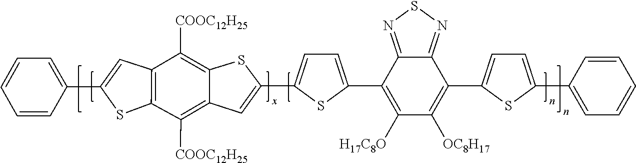

Polymer 3 (x=y=1) and its preparation are disclosed in WO 2011/131280 A1.

TABLE 1
Formulation characteristics

| | | | Ratio | Concentra- | |
|---|---|---|---|---|---|
| No. | Donor | Acceptor | Polymer:Acceptor | tion g/L | Solvent |
| 1 | P3 | P1 | 1:1.3 | 23 mg/ml | o-xylene |

Current-voltage characteristics are measured using a Keithley 2400 SMU while the solar cells are illuminated by a Newport Solar Simulator at 100 mW·cm⁻² white light. The solar simulator is equipped with AM1.5G filters. The illumination intensity is calibrated using a Si photodiode. All the device characterization is done in a dry-nitrogen atmosphere.

Power conversion efficiency is calculated using the following expression

$$η = \frac{{V}_{oc} \times {J}_{sc} \times FF}{{P}_{in}}$$

where FF is defined as

$$FF = \frac{{V}_{max} \times {J}_{max}}{{V}_{oc} \times {J}_{sc}}$$

OPV device characteristics were measured for a blend which contains Polymer P3 as a donor and Polymer P1 as an acceptor, and is coated from an organic solution. Details of the solution composition are shown in Table 1.

Table 2 shows the device characteristics for the individual OPV devices comprising a photoactive layer with a BHJ formed from the active material (acceptor/polymer) solutions of Table 2.

TABLE 2
Photovoltaic cell characteristics under simulated
solar irradiation at 1 sun (AM 1.5G)

| | | | Average Performance | | | |
|---|---|---|---|---|---|---|
| | | | Voc | Jsc | FF | PCE |
| No. | Acceptor | Polymer | mV | mA cm⁻² | % | % |
| 1 | P1 | P3 | 663 | 10.4 | 34.5 | 2.38 |

1. A polymer comprising one or more repeating units of formula I 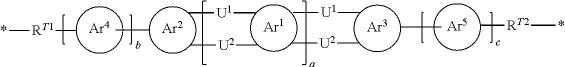
wherein the individual radicals, independently of each other and on each occurrence identically or differently, have the following meanings,
each pair of U¹ and U² is attached to two directly adjacent C atoms in a ring Ar¹, Ar² or Ar³ respectively, and in each of said pairs of U¹ and U² one of U¹ and U² is a single bond and the other is CR¹R², SiR¹R², GeR¹R², C═CR¹R², C═O or NR¹, so that each pair of U¹ and U² is forming a five-membered unsaturated ring together with the C atoms to which they are attached,
R^{T1}, R^{T2} a group of formula T 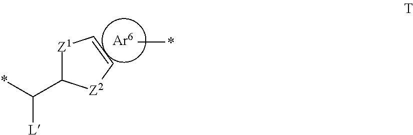
Z¹, Z² C═O, C═CX¹X², SO₂,
X¹, X² CN, C(═O)R^{a},
Ar¹, Ar², Ar³ arylene or heteroarylene which has from 5 to 20 ring atoms, is mono- or polycyclic, optionally contains fused rings, and is unsubstituted or substituted by one or more identical or different groups L,
Ar⁴, Ar⁵ —CY¹═CY²—, —C≡C—, or arylene or heteroarylene which has from 5 to 20 ring atoms, is mono- or polycyclic, optionally contains fused rings, and is unsubstituted or substituted by one or more identical or different groups L,
Ar⁶ arylene or heteroarylene which has from 5 to 20 ring atoms, is mono- or polycyclic, optionally contains fused rings, and is unsubstituted or substituted by one or more identical or different groups L,
Y¹, Y²H, F, Cl or CN,
R¹, R²H, F, Cl, CN, or straight-chain, branched or cyclic alkyl with 1 to 30, preferably 1 to 20, C atoms, in which one or more CH₂ groups are each optionally replaced by —O—, —S—, —C(═O)—, —C(═S)—, —C(═O)—O—, —O—C(═O)—, —NR⁰—, —SiR⁰R⁰⁰—, —CF₂—, —CR⁰═CR⁰⁰—, —CY¹═CY²— or —C≡C— in such a manner that O and/or S atoms are not linked directly to one another, and in which one or more H atoms are each optionally replaced by F, Cl, Br, I or CN, and in which one or more CH₂ or CH₃ groups are each optionally replaced by a cationic or anionic group, or aryl, heteroaryl, arylalkyl, heteroarylalkyl, aryloxy or heteroaryloxy, wherein each of the aforementioned cyclic groups has 5 to 20 ring atoms, is mono- or polycyclic, optionally contains fused rings, and is unsubstituted or substituted by one or more identical or different groups L,
and the pair of R¹ and R², together with the C, Si or Ge atom to which they are attached, may also form a spiro group with 5 to 20 ring atoms which is mono- or polycyclic, optionally contains fused rings, and is unsubstituted or substituted by one or more identical or different groups L,
R^{a} R⁰ or aryl or heteroaryl, each having from 4 to 30 ring atoms, optionally containing fused rings and being unsubstituted or substituted with one or more groups L,
L F, Cl, —NO₂, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —CF₃, —SF₅, or optionally substituted silyl, or carbyl or hydrocarbyl with 1 to 30, preferably 1 to 20 C atoms that is optionally substituted and optionally comprises one or more hetero atoms, preferably F, —CN, R⁰, —OR⁰, —SR⁰, —C(═O)—R⁰, —C(═O)—OR⁰, —O—C(═O)—R⁰, —O—C(═O)—OR⁰, —C(═O)—NHR⁰, or —C(═O)—NR⁰R⁰⁰,
L′ H or one of the meanings given for L,
R⁰, R⁰⁰H or straight-chain or branched alkyl with 1 to 20, preferably 1 to 12, C atoms that is optionally fluorinated,
X⁰ halogen, preferably F or Cl,
a 0, 1, 2 or 3,
b, c 0, 1, 2 or 3,
characterized in that, if a>0, Ar¹ contains at least one thiophene ring.

2. The polymer according to claim 1, characterized in that the repeating units of formula I are selected from the following subformulae 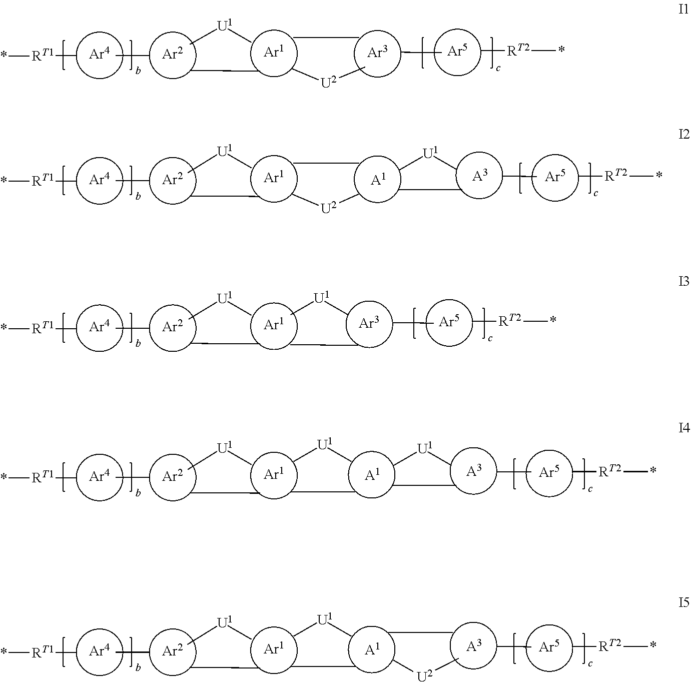
wherein Ar¹, Ar², Ar³, Ar⁴, R^{T1}, R^{T2}, b and c have the meanings given in claim 1, and U¹ and U² have one of the meanings given in claim 1 which is different from a single bond.

3. The polymer according to claim 1, characterized in that the groups Ar¹ are on each occurrence identically or differently selected from the following formulae and their mirror images 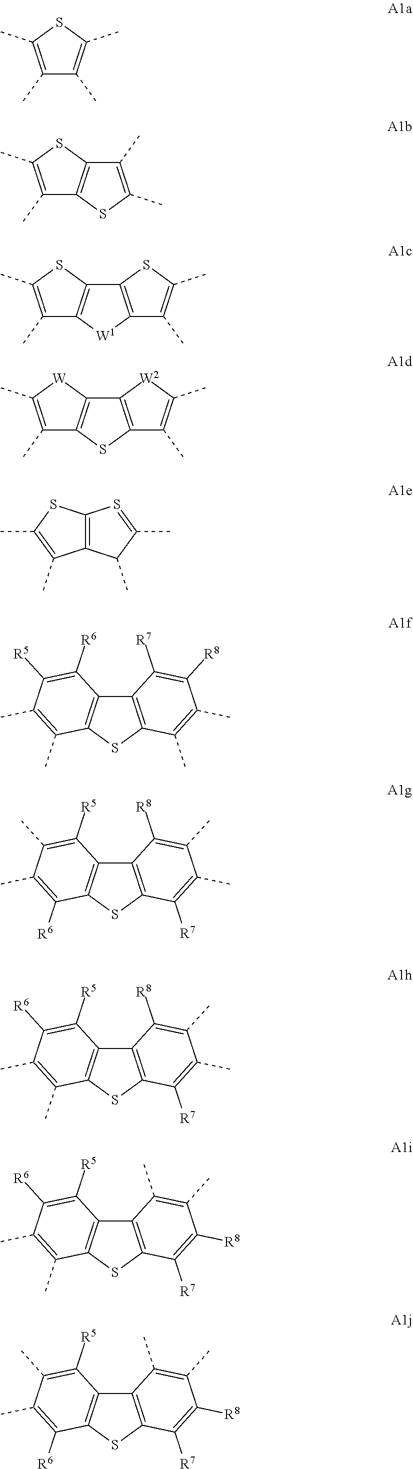 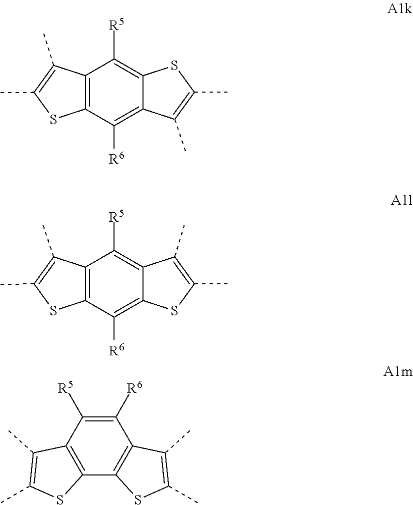
wherein the individual radicals, independently of each other and on each occurrence identically or differently, have the following meanings
W¹, W² S, O, Se or C═O,
R⁵⁻⁸ H, F, Cl, CN, or straight-chain, branched or cyclic alkyl with 1 to 30, preferably 1 to 20, C atoms, in which one or more CH₂ groups are each optionally replaced by —O—, —S—, —C(═O)—, —C(═S)—, —C(═O)—O—, —O—C(═O)—, —NR⁰—, —SiR⁰R⁰⁰—, —CF₂—, —CR⁰═CR⁰⁰—, —CY¹═CY²— or —C≡C— in such a manner that O and/or S atoms are not linked directly to one another, and in which one or more H atoms are each optionally replaced by F, Cl, Br, I or CN, and in which one or more CH₂ or CH₃ groups are each optionally replaced by a cationic or anionic group, or aryl, heteroaryl, arylalkyl, heteroarylalkyl, aryloxy or heteroaryloxy, wherein each of the aforementioned cyclic groups has 5 to 20 ring atoms, is mono- or polycyclic, optionally contains fused rings, and is unsubstituted or substituted by one or more identical or different groups L,
Y¹, Y², R⁰, R⁰⁰ one of the meanings given in claim 1.

4. The polymer according to claim 3, characterized in that the groups Ar² are on each occurrence identically or differently selected from the following formulae and their mirror images 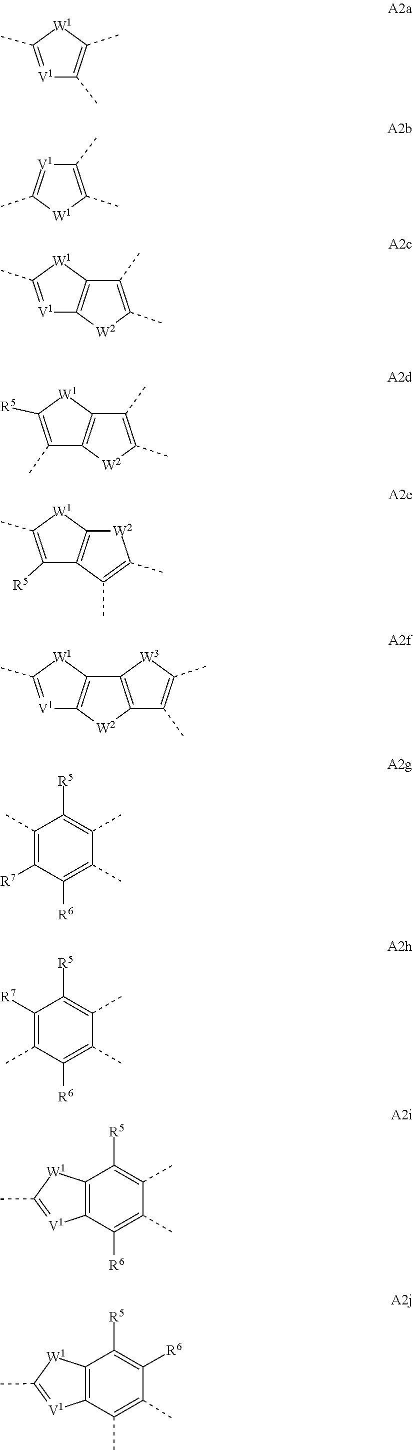 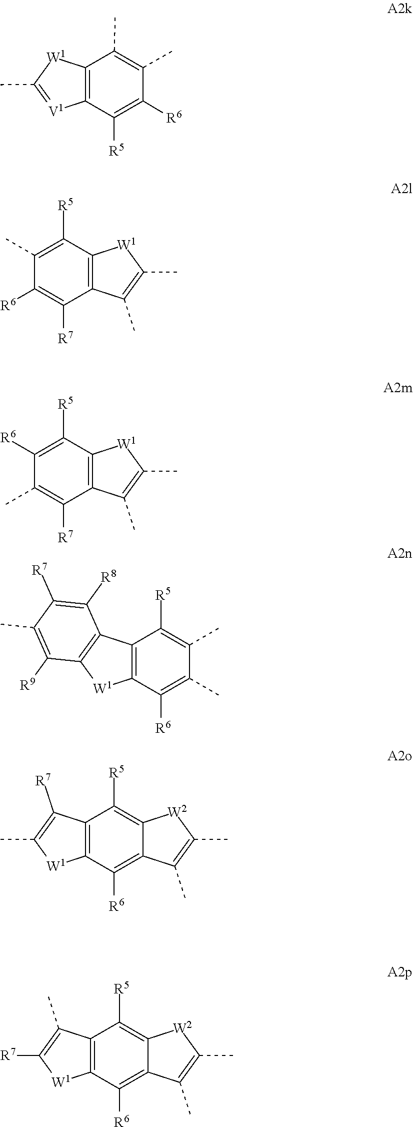
wherein, independently of each other and on each occurrence identically or differently, W¹, W² and R⁵⁻⁸ have the meanings given in claim 3, V¹ is CR³ or N, W³ has one of the meanings given for W¹, and R³ and R⁹ have one of the meanings given for R⁵.

5. The polymer according to claim 4, characterized in that the groups Ar² are on each occurrence identically or differently selected from the following formulae and their mirror images 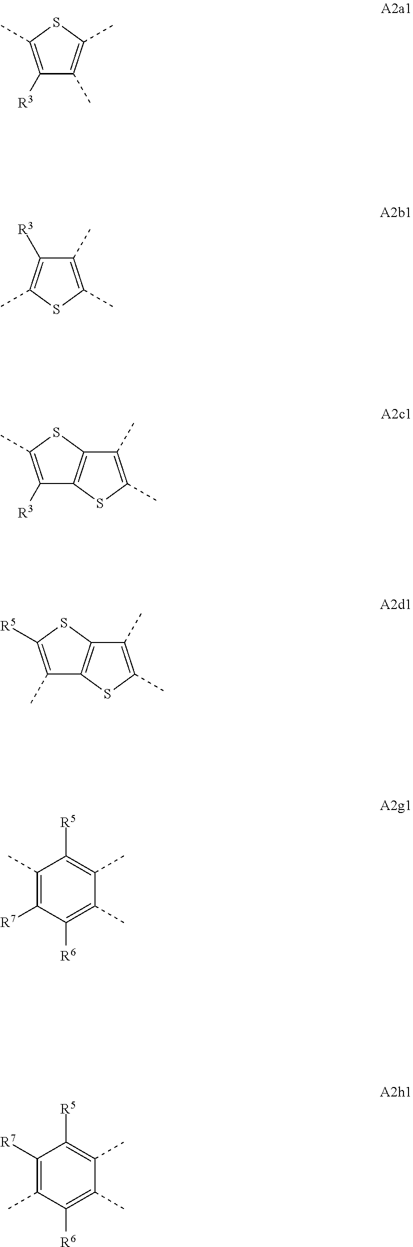 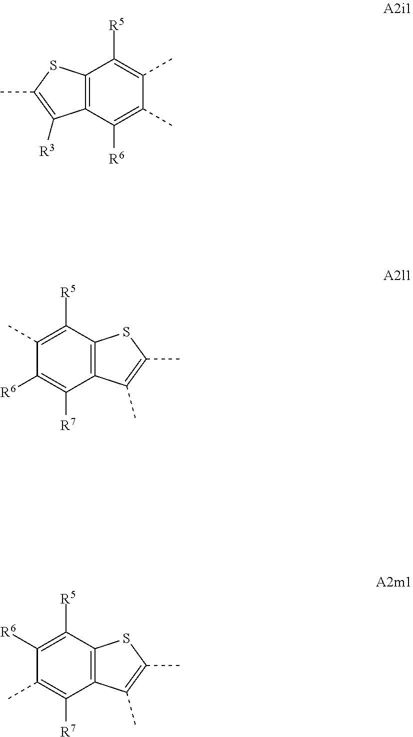
wherein R³⁻⁷ have the meanings given in claim 4.

6. The polymer according to claim 4, characterized in that the groups Ar³ are on each occurrence identically or differently selected from the following formulae and their mirror images 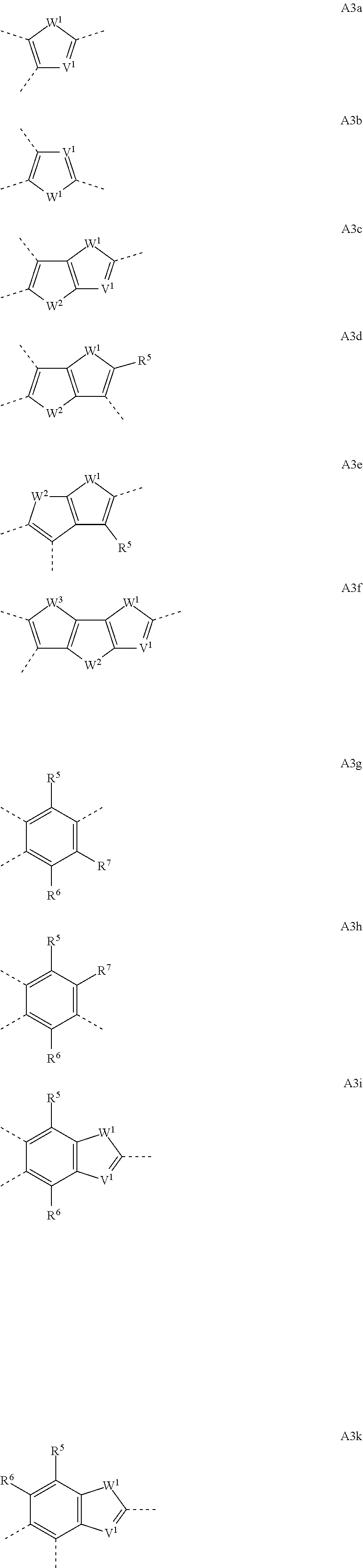 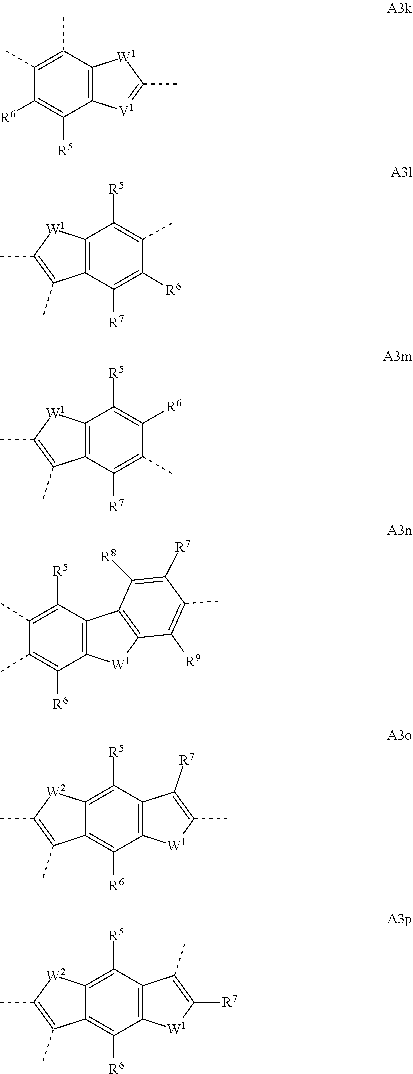
wherein V¹, W¹, W², W³ and R⁵⁻⁹ have the meanings given in claim 4.

7. The polymer according to 4, characterized in that the groups Ar³ are on each occurrence identically or differently selected from the following formulae and their mirror images 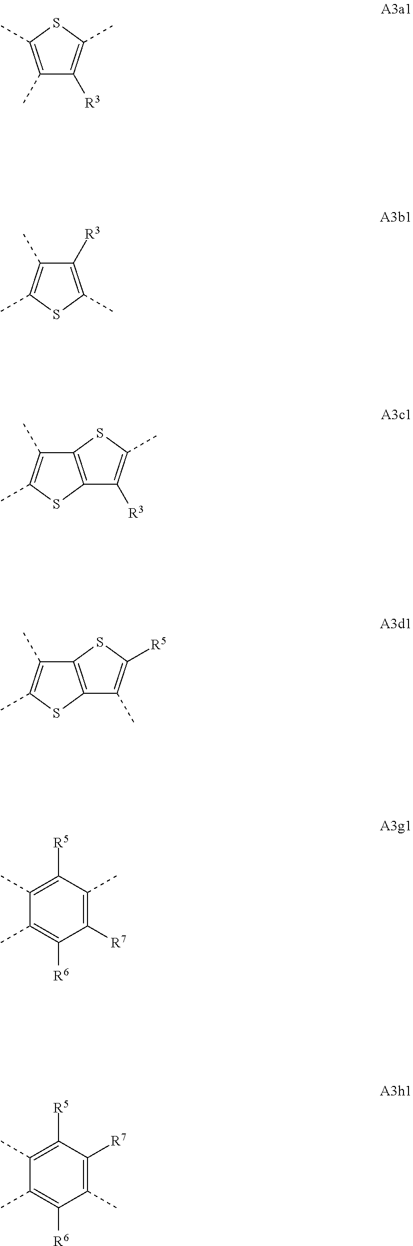 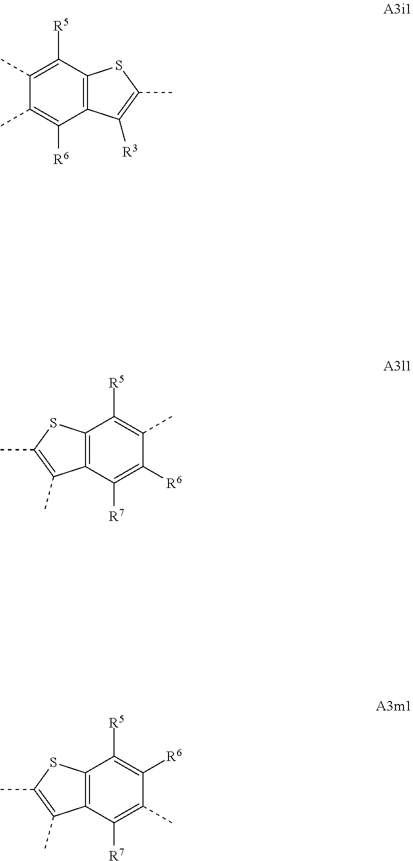
wherein R³⁻⁷ have the meanings given in claim 4.

8. The polymer according to claim 4, characterized in that the groups Ar⁴ and Ar⁵ are independently of each other, and on each occurrence identically or differently, selected from the following formulae and their mirror images 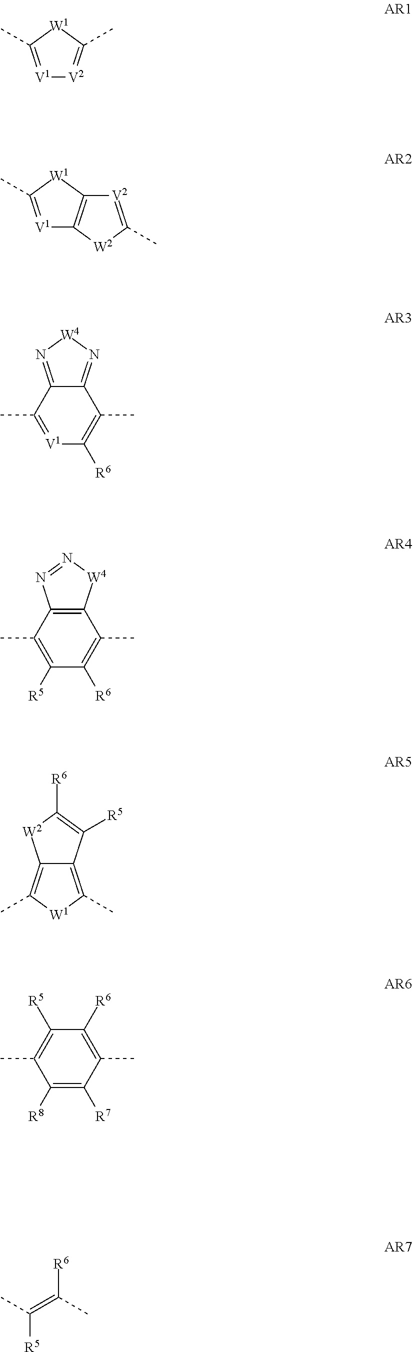
wherein the individual radicals, independently of each other and on each occurrence identically or differently, have the following meanings
V² CR⁴ or N,
W⁴ S, O, Se, NR⁰ or C═O,
R⁴ one of the meanings given for R³ above and below,
and V¹, W¹, W², R⁰, R⁵⁻⁸ are as defined in claim 4.

9. The polymer according to claim 1, characterized in that the groups Ar⁴ and Ar⁵ are independently of each other, and on each occurrence identically or differently, selected from the following formulae and their mirror images 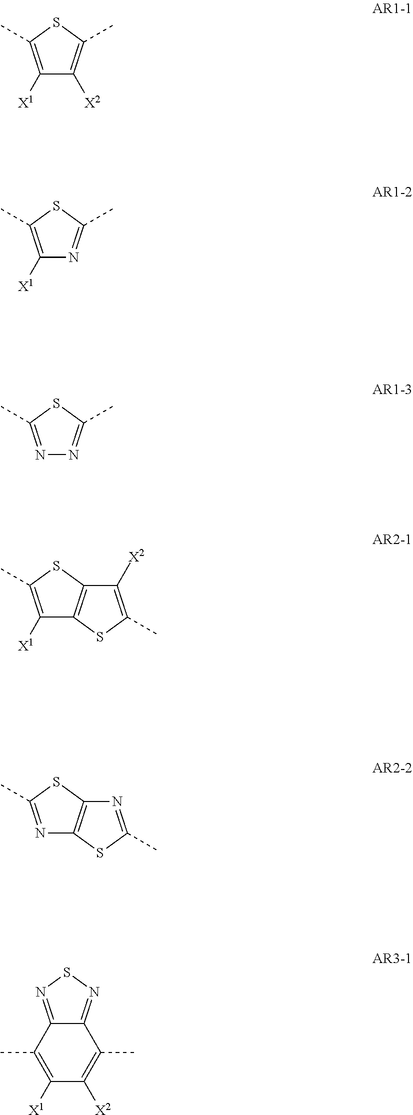 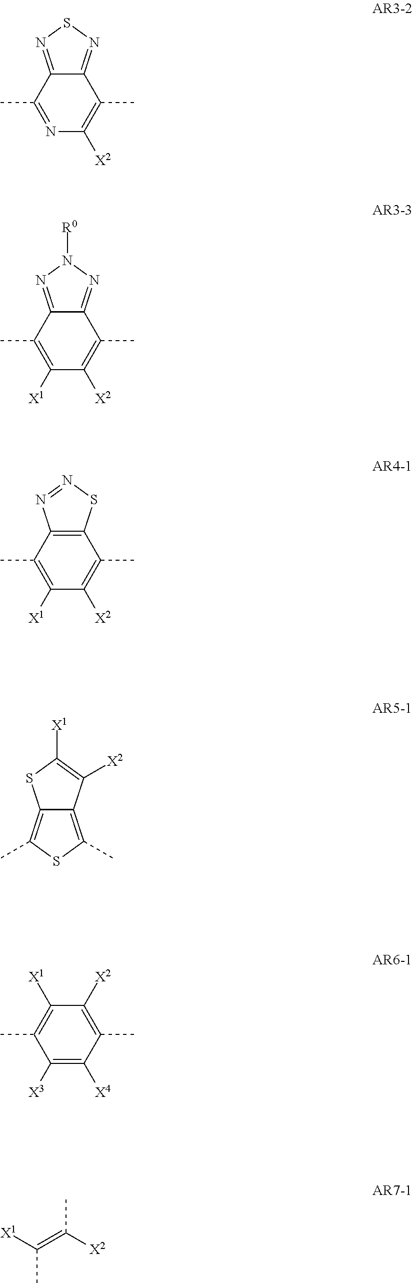
wherein X¹, X², X³ and X⁴ have one of the meanings given for R¹ in claim 1.

10. The polymer according to claim 1, characterized in that R^{T1} and R^{T2} are independently of each other selected from the following formulae 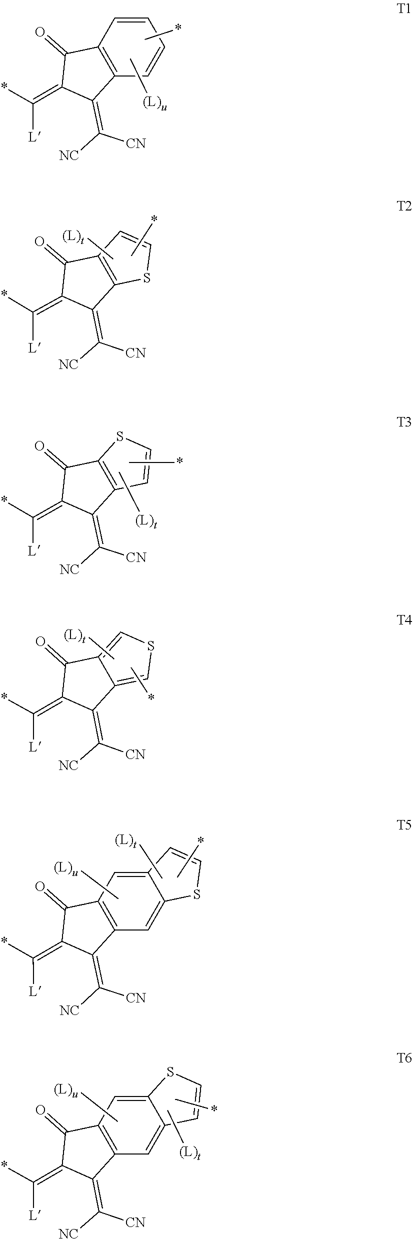 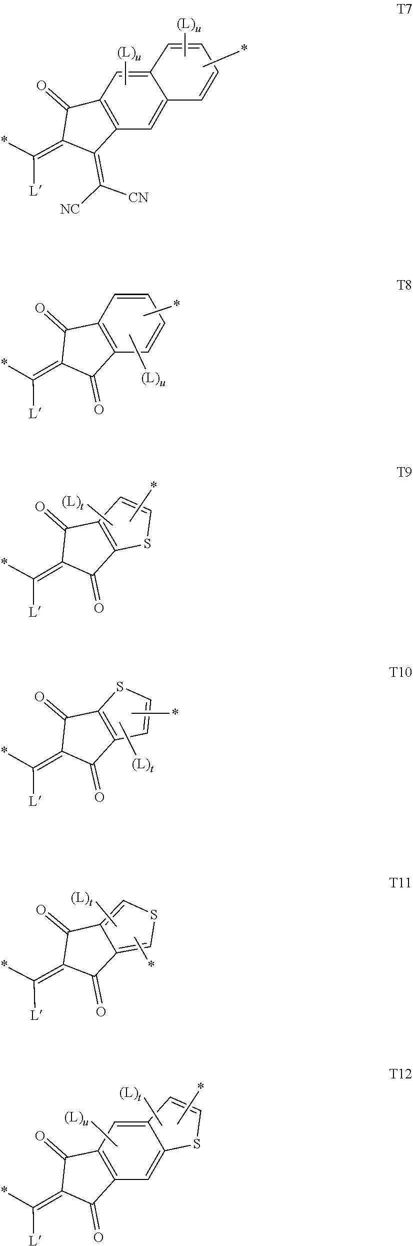 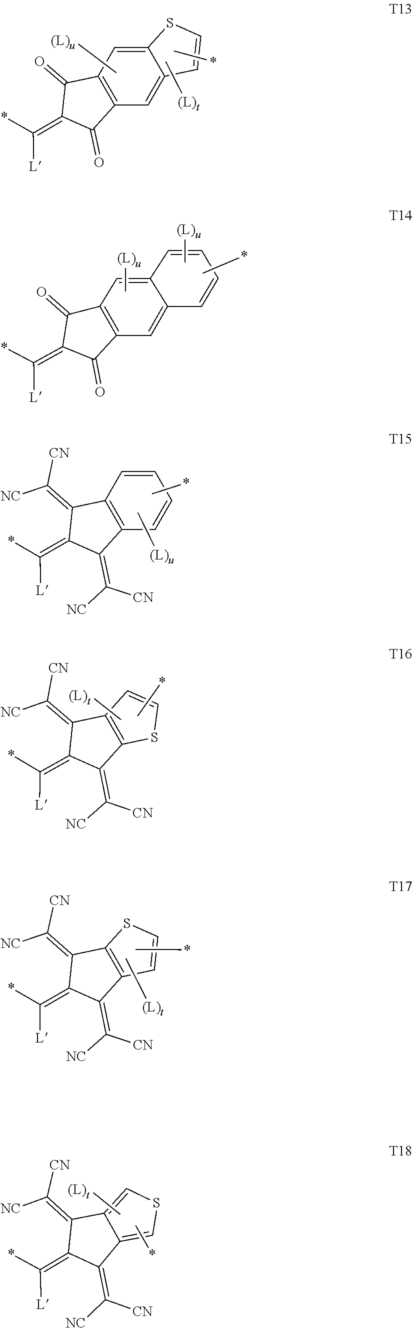 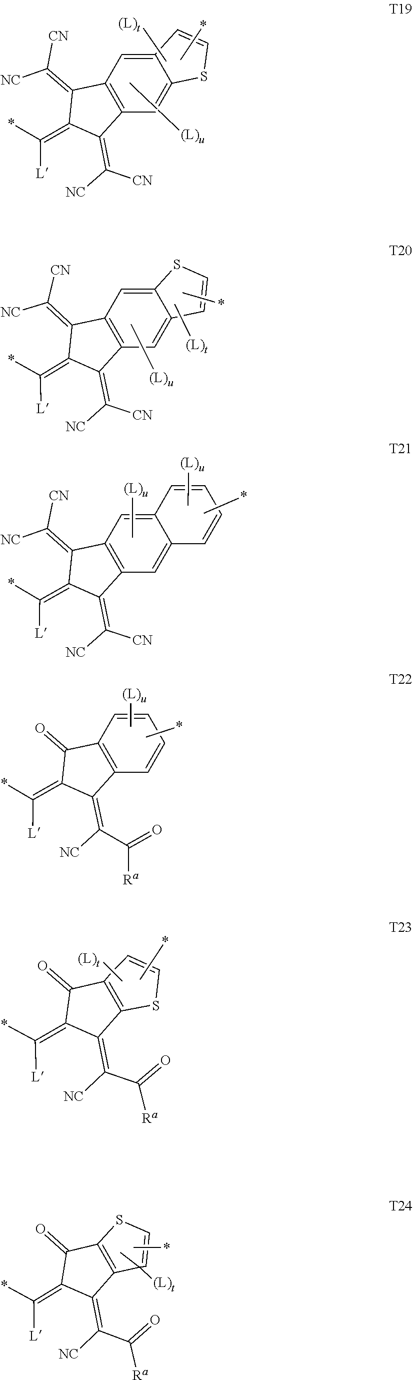 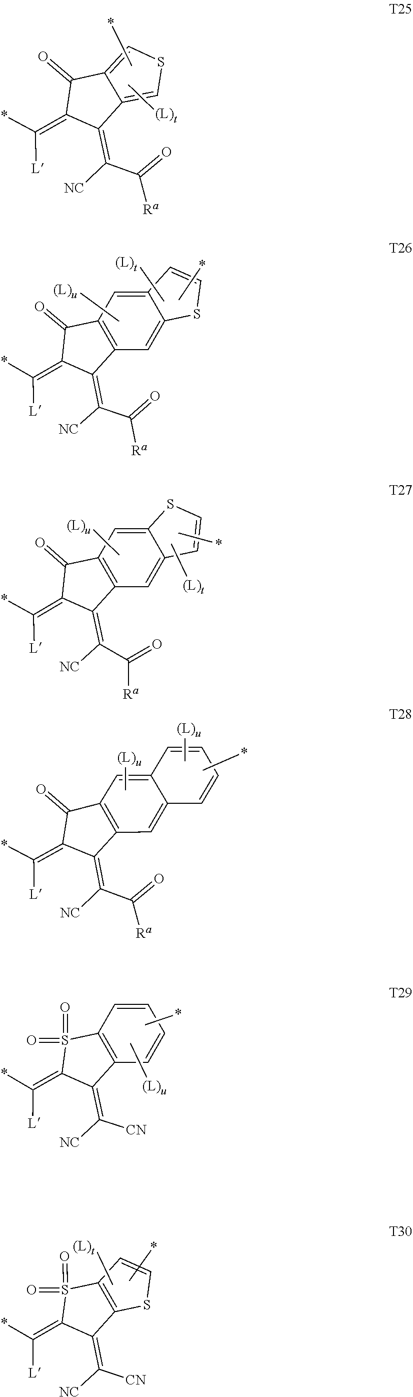 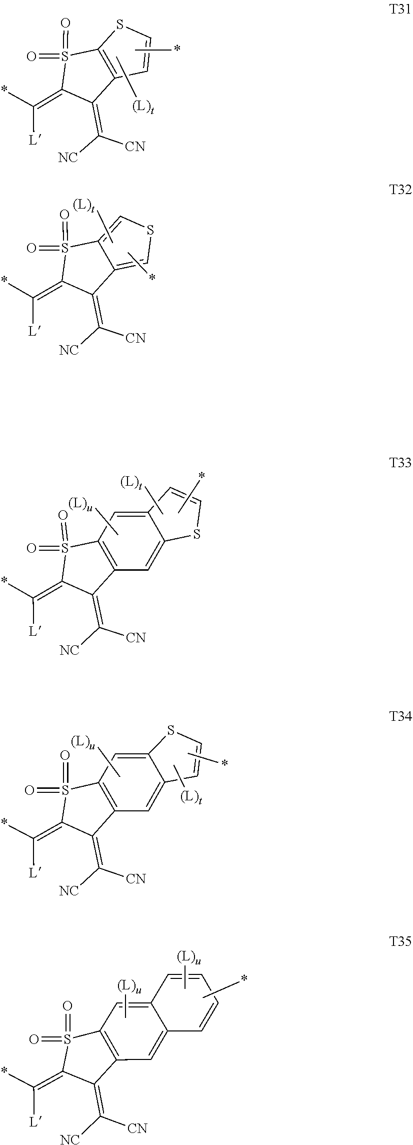
wherein R^{a}, L and L′ have the meanings given in claim 1, t is 0 or 1, and u is 0, 1 or 2.

11. The polymer according to claim 3, characterized in that the repeating units of formula I are selected of formula IA 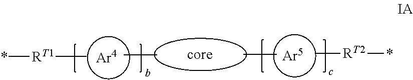
wherein Ar⁴, Ar⁵, R^{T1}, R^{T2}, b and c, independently of each other and on each occurrence identically or differently, have the meanings given in claim 1, and “Core” is, on each occurrence identically or differently, a polycyclic divalent group selected from the following formulae 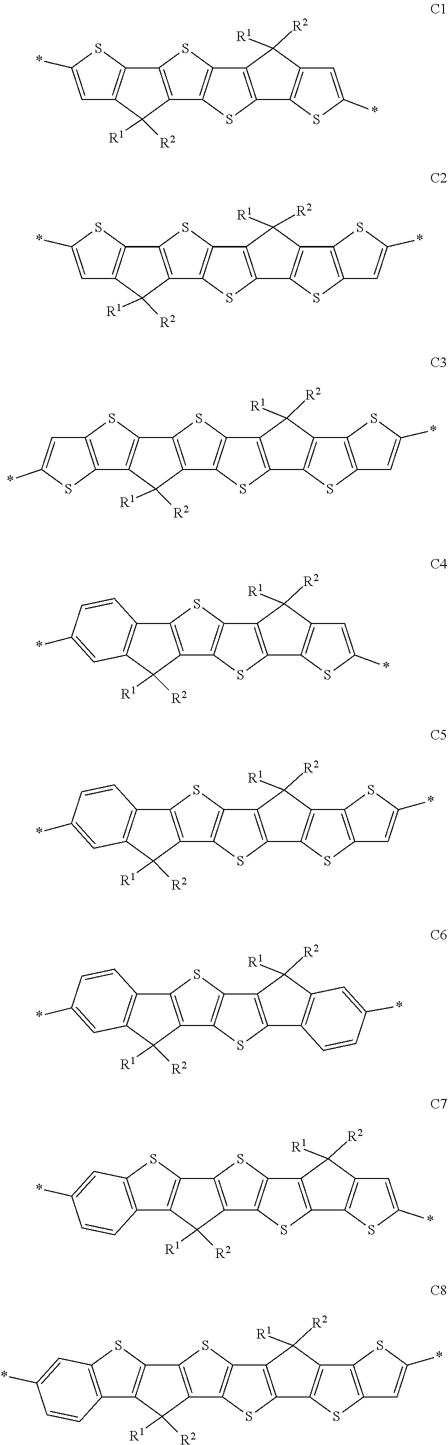 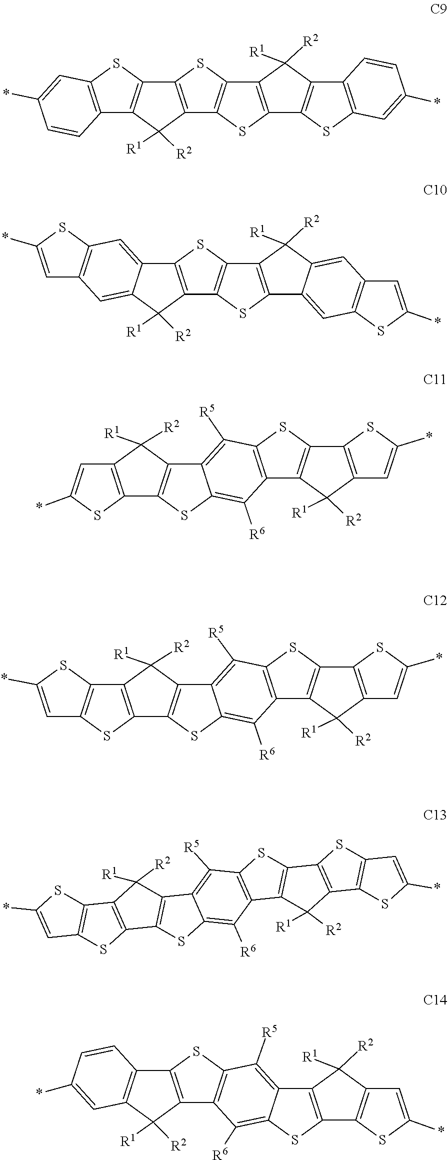 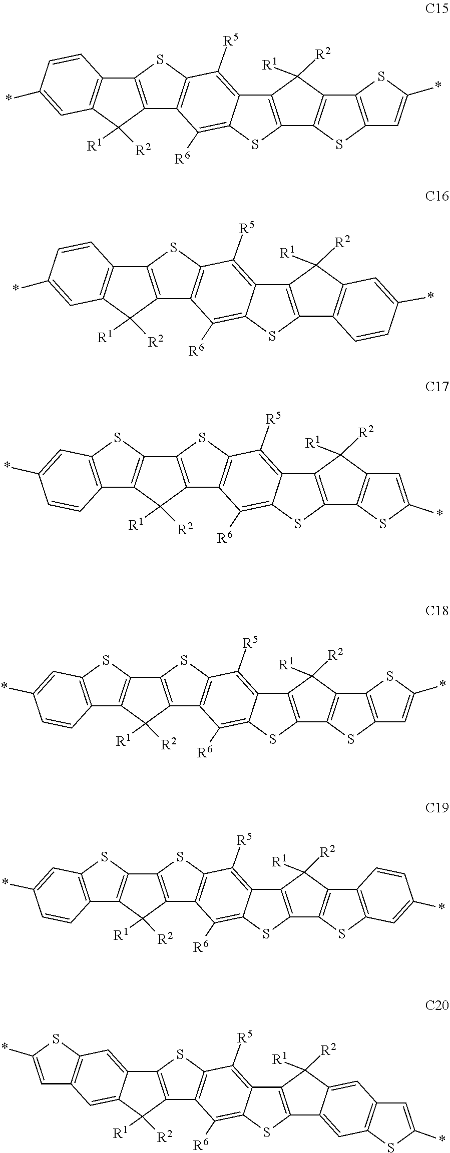 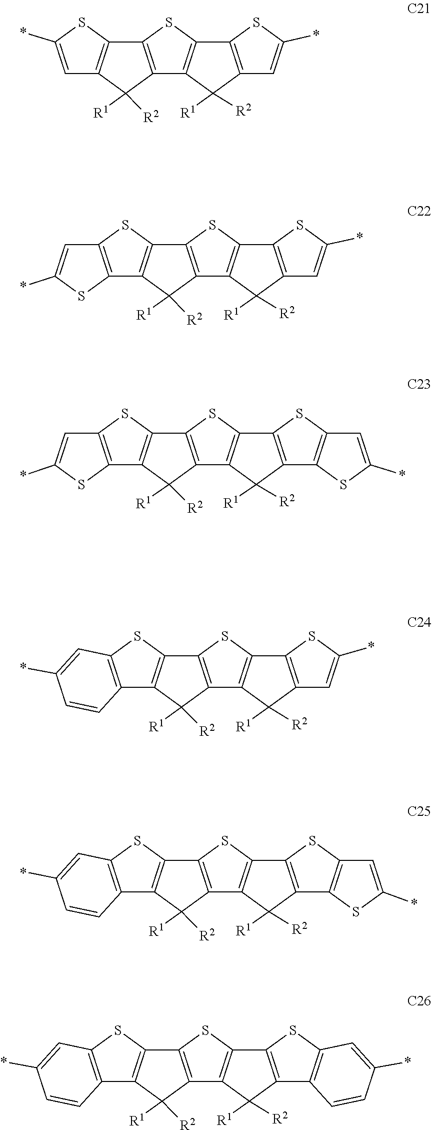
wherein R¹⁻⁶ have the meanings given in claims 1 and 3.

12. The polymer according to claim 3, characterized in that the repeating units of formula I are selected from the following subformulae 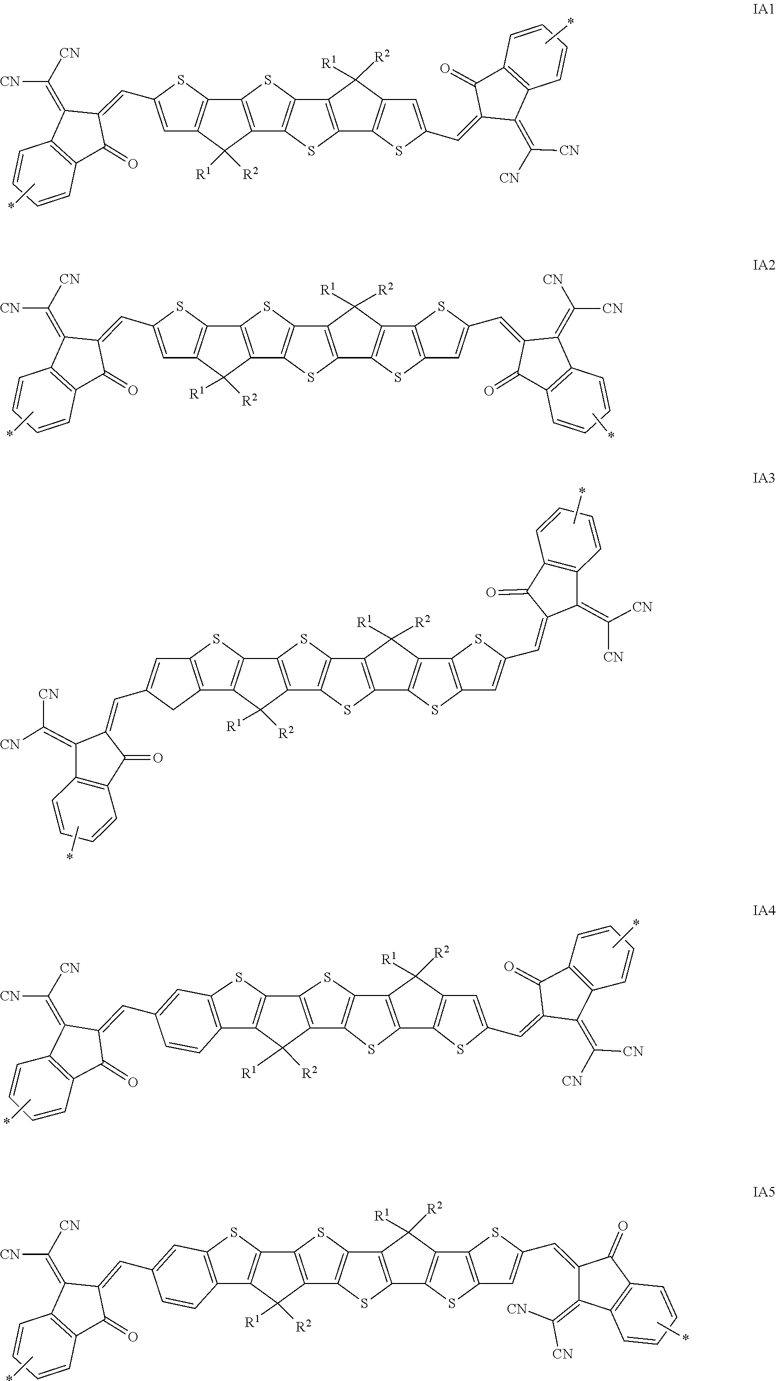 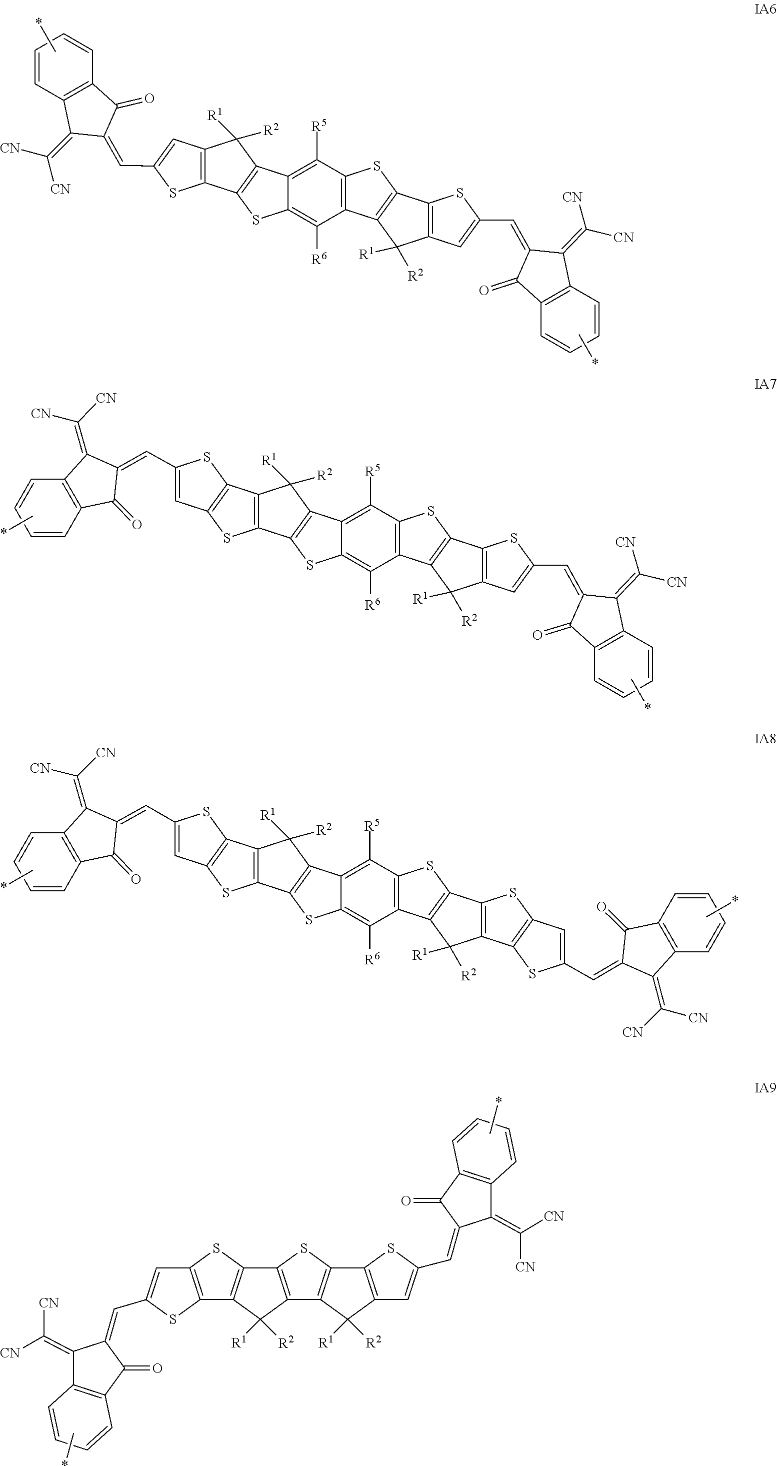 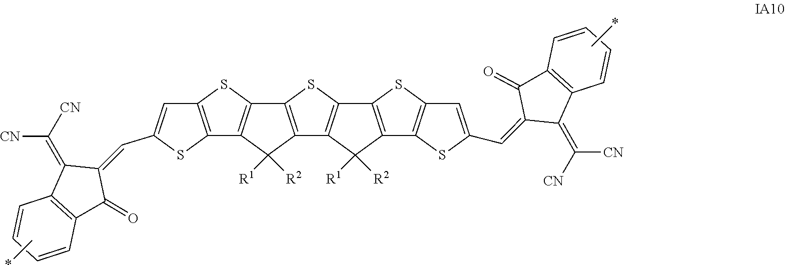
wherein R¹⁻⁶ have the meanings given in claims 1 and 3.

13. The polymer according to claim 1, characterized in that R¹ and R² are selected from F, Cl, CN, straight-chain or branched alkyl, alkoxy, sulfanylalkyl, sulfonylalkyl, alkylcarbonyl, alkoxycarbonyl and alkylcarbonyloxy, each having 1 to 20 C atoms and being unsubstituted or substituted by one or more F atoms.

14. The polymer according to claim 1, characterized in that R¹ and R² are selected from mono- or polycyclic aryl or heteroaryl, each of which is optionally substituted with one or more groups L as defined in formula I and has 5 to 20 ring atoms, and wherein two or more rings may be fused to each other or connected with each other by a covalent bond.

15. The polymer according to claim 1, characterized in that R⁵⁻⁹, when being different from H, are each independently selected from F, Cl, CN, or from straight-chain or branched alkyl, alkoxy, sulfanylalkyl, sulfonylalkyl, alkylcarbonyl, alkoxycarbonyl and alkylcarbonyloxy, each of which has 1 to 20 C atoms and is unsubstituted or substituted by one or more F atoms, or from mono- or polycyclic aryl or heteroaryl, each of which is optionally substituted with one or more groups L as defined in formula I and has 5 to 20 ring atoms, and wherein two or more rings may be fused to each other or connected with each other by a covalent bond.

16. The polymer according to claim 1, characterized in that it comprises one or more units of formula I or its subformulae as defined in claim 1, and one or more units selected from the following groups
A1) the group consisting of arylene or heteroarylene units that are different from formula I and its subformulae, have from 5 to 20 ring atoms, are mono- or polycyclic, do optionally contain fused rings, and are unsubstituted or substituted by one or more identical or different groups L as defined in formula I,
B1) —CY¹═CY²— wherein Y¹ and Y² are independently of each other H, F, Cl or CN,
C1) —C≡C—,
D1) the group consisting of straight-chain, branched or cyclic alkylene with 1 to 30, preferably 2 to 16, C atoms, in which one or more CH₂ groups are each optionally replaced by —O—, —S—, —C(═O)—, —C(═S)—, —C(═O)—O—, —O—C(═O)—, —NR⁰—, —SiR⁰R⁰⁰—, —CF₂—, —CR⁰═CR⁰⁰—, —CY¹═CY²— or —C≡C— in such a manner that O and/or S atoms are not linked directly to one another, and in which one or more H atoms are each optionally replaced by F, Cl, Br, I or CN, but excluding fully conjugated unsaturated groups (like e.g. —CR⁰═CR⁰⁰—CR⁰═CR⁰⁰— or —C≡C—),
E1) the group consisting of units formed from one or more units selected from groups A1, B1 and 01 and one or more units of group D1.

17. The polymer according to claim 16, characterized in that it comprises one or more repeating units of formula II1 and/or II2, and optionally one or more repeating units of formula II3:
—(C¹)ₐ—U—(C²)_{b}—(C³)_{c}—(C⁴)_{d}— II1
—(C¹)ₐ—(C²)_{b}—U—(C³)_{c}—(C⁴)_{d}— II2
—(C¹)ₐ—(C²)_{b}—(C³)_{c}—(C⁴)_{d}— II3
wherein the individual radicals, independently of each other and on each occurrence identically or differently, have the following meanings
U a unit selected from formula I or its subformulae as defined in claim 1,
C¹⁻⁴ a unit selected from groups A1, B1, C1, D1 and E1 as defined in claim 16,
a, b, c, d 0 or 1, wherein in formula II3 a+b+c+d≥1.

18. The polymer according to claim 17, characterized in that it comprises one or more repeating units selected from the group consisting of the following formulae and their mirror images
—(U)— U1
—(U-Sp)- U2
-(Sp-U-Sp)- U3
—(U-D)- U4
—(U-A)- U5
-(D-Sp)- U6
-(A-Sp)- U7
-(A-D)- U8
-(D)- U9
-(Sp-D-Sp)- U10
-(A)- U11
-(Sp-A-Sp)- U12
wherein the individual radicals, independently of each other and on each occurrence identically or differently, have the following meanings
U the meaning given in claim 17,
D a unit of group A1 as defined in claim 17 which is a donor unit,
A a unit of group A1 as defined in claim 17 which is an acceptor unit,
Sp a spacer unit which is located between two units selected from the group consisting of U, D and A, and is selected from groups A1, B1, C1, D1 and E1 as defined in claim 17,
and the polymer comprises at least one unit selected from formulae U1-U5.

19. The polymer according to claim 17, characterized in that it is selected of formula III: 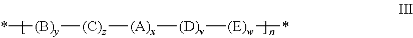
wherein the individual radicals, independently of each other and on each occurrence identically or differently, have the following meanings
A a unit of formula II1 or II2 as defined in claim 17,
B, C, D, E a unit of formula II1, II2 or II3 as defined in claim 17,
x>0 and ≤1,
v, w, y, z≥0 and <1,
v+w+x+y+z 1, and
n an integer >1.

20. The polymer according to claim 18, characterized in that it is selected from formulae Pi-Px
—[(U-Sp]ₙ- Pi
—[(U¹-Sp)ₓ-(U²—Sp)_{y}]ₙ- Pii
—[(U-Sp¹)ₓ-(U-Sp2)_{y}]ₙ- Piii
—[(U-Sp)ₓ-(D-Sp)_{y}]ₙ- Piv
—[(U-D)ₓ-(U-Sp)_{y}]ₙ- Pv
-[(D)ₓ-(Sp-U-Sp)_{y}]ₙ- Pvi
—[(U)ₓ-(Sp-D-Sp)_{y}]ₙ- Pvi
-[D-U]ₙ— Pvii
-[D-Sp-U-Sp]ₙ- Pviii
-[D¹-U-D²-U]ₙ— Pix
-[D-U¹-D-U²]ₙ— Px
wherein A, D and Sp are as defined in claim 18, A, D and Sp can each, in case of multiple occurrence, also have different meanings, D¹ and D² have one of the meanings given for D and are different from each other, Sp¹ and Sp² have one of the meanings given for Sp and are different from each other, U¹ and U² have one of the meanings given for U and are different from each other, x and y denote the molar fractions of the corresponding units, x and y are each, independently of one another, a non-integer >0 and <1, with x+y=1, and n is an integer >1.

21. The polymer according to claim 3, characterized in that one or more of the units of group A1, or one or more of C¹, C², C³, C⁴, or D, respectively, denote arylene or heteroarylene selected from the group consisting of the following formulae and their mirror images 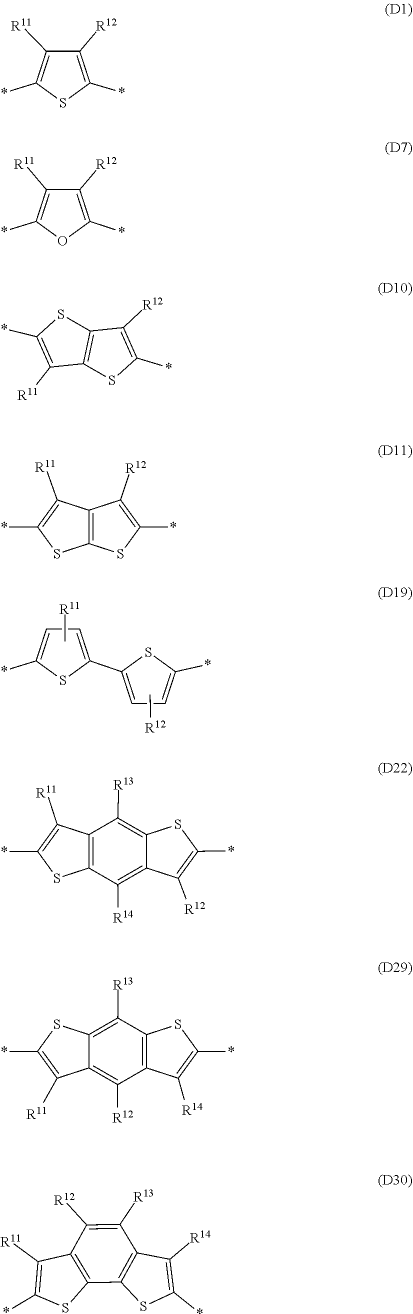 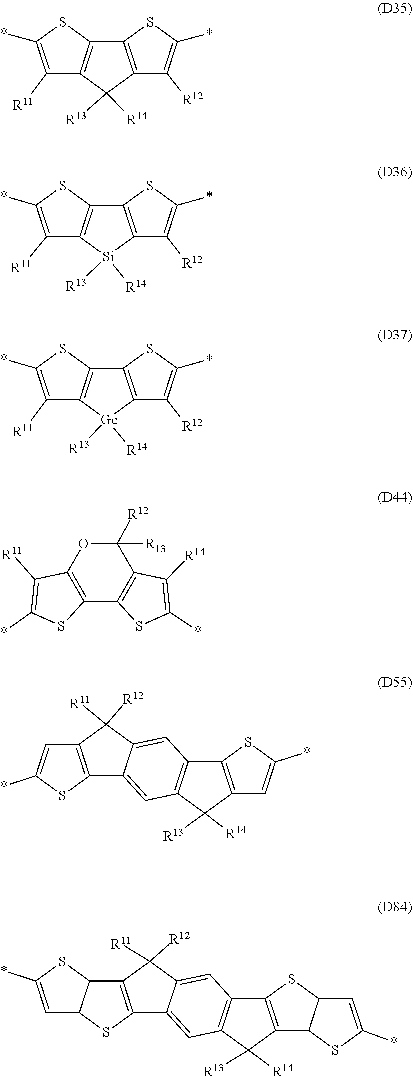 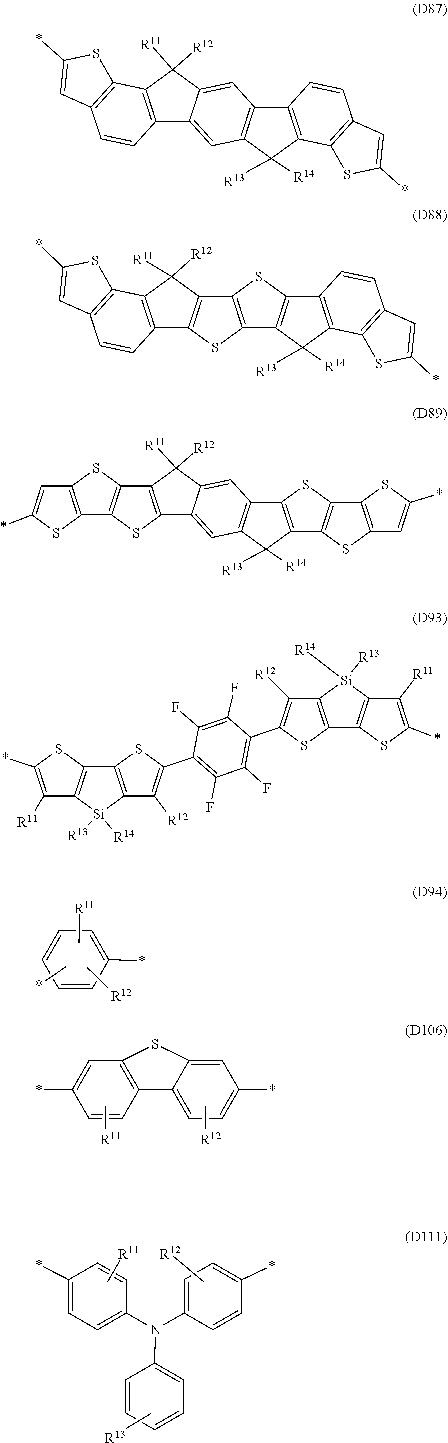 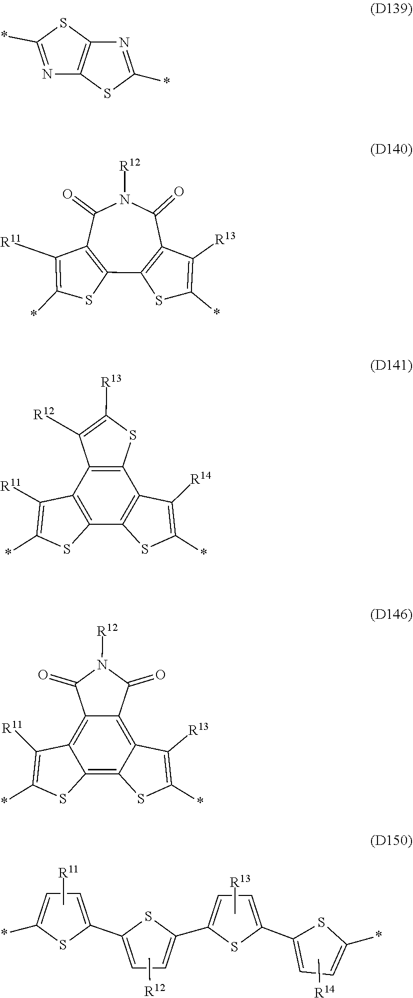
wherein R¹¹, R¹², R¹³, R¹⁴, R¹⁵, R¹⁶, R¹⁷ and R¹⁸ independently of each other have one of the meanings of R⁵ as given in claim 3.

22. The polymer according to claim 21, characterized in that one or more of the units of group A1, or one or more of C¹, C², C³, C⁴, or A, respectively, denote arylene or heteroarylene selected from the group consisting of the following formulae and their mirror images 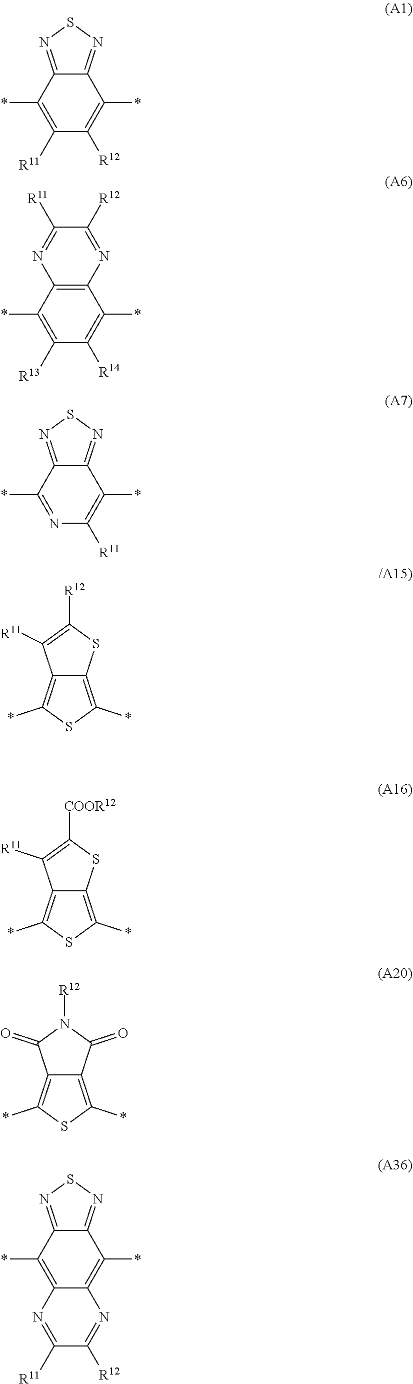 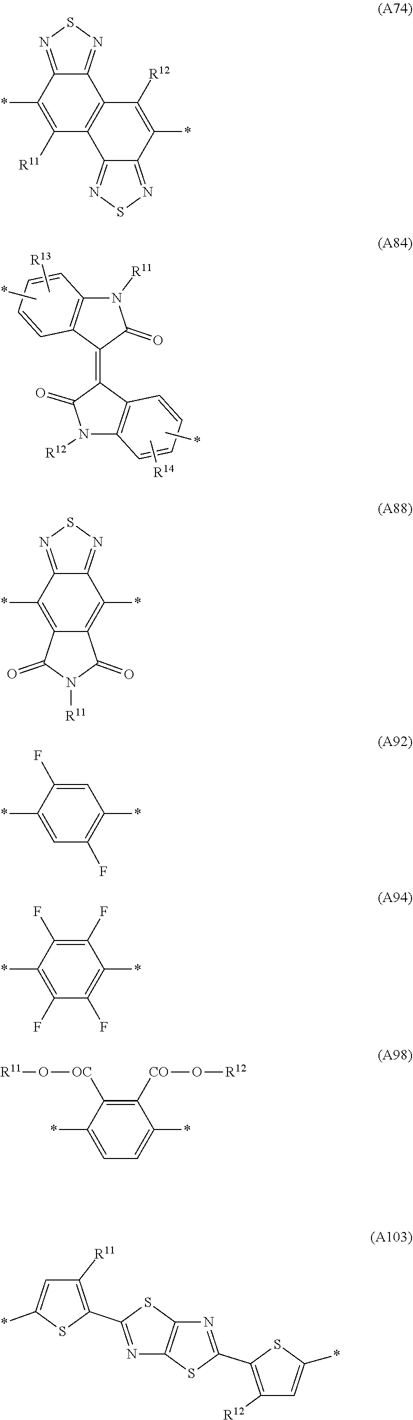
wherein R¹¹, R¹², R¹³, R¹⁴, R¹⁵, R¹⁶, R¹⁷ and R¹⁸ are as defined in claim 21.

23. The polymer according to claim 22, characterized in that one or more of the units of group A1, or one or more of C¹, C², C³, C⁴, or Sp, respectively, denote arylene or heteroarylene selected from the group consisting of the following formulae and their mirror images 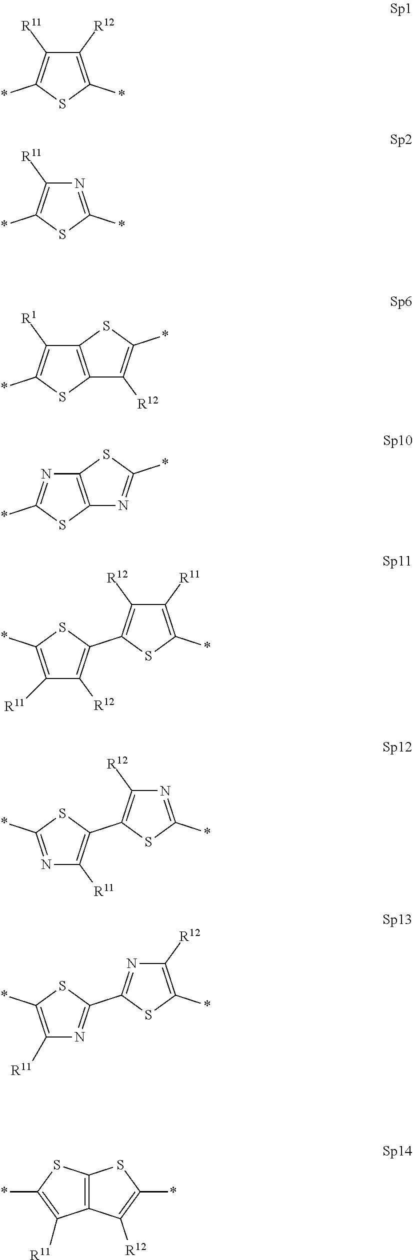
wherein R¹¹, R¹², R¹³, R¹⁴ are as defined in claim 22.

24. The polymer according to claim 17, characterized in that it comprises one or more units of group D1 as defined in claim 17, which are selected from the following formulae 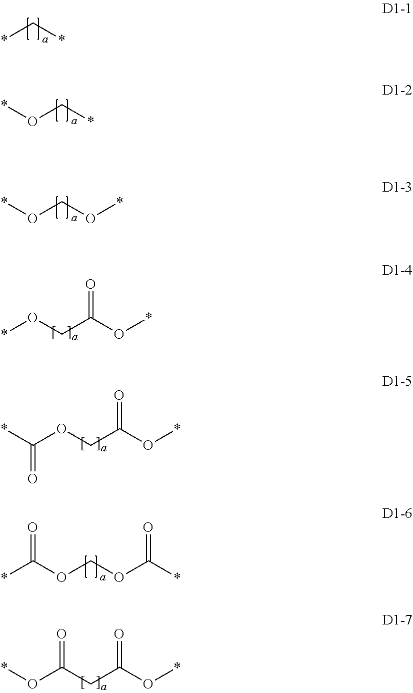
wherein a is an integer from 1 to 16.

25. The polymer according to claim 17, characterized in that it comprises one or more units of group E1 as defined in claim 17, which are selected from the following formula
A¹-B¹-(A²)ₐₐ E1a
wherein aa is 0 or 1, A¹ and A² have one of the meanings given for Ar¹ in claim 17, and B¹ is selected from group D1 as defined in claim 17.

26. The polymer according to claim 17, characterized in that it comprises one or more units of group E1 as defined in claim 17, which are selected from the following formulae 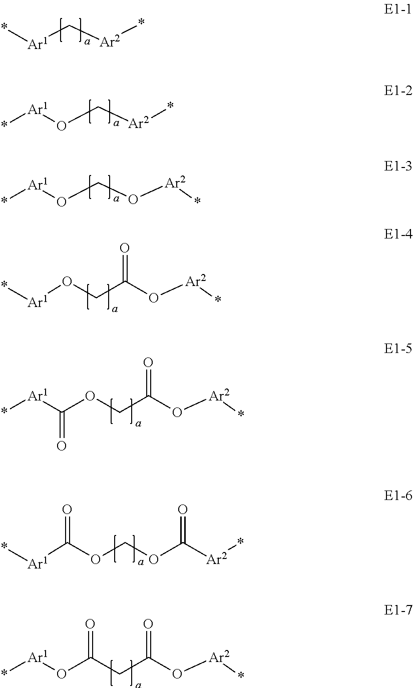
wherein a is an integer from 1 to 16, and C¹ and C² have the meanings given in claim 17.

27. The polymer according to claim 17, characterized in that it comprises one or more units of group E1 as defined in claim 17, which are selected from the following formulae 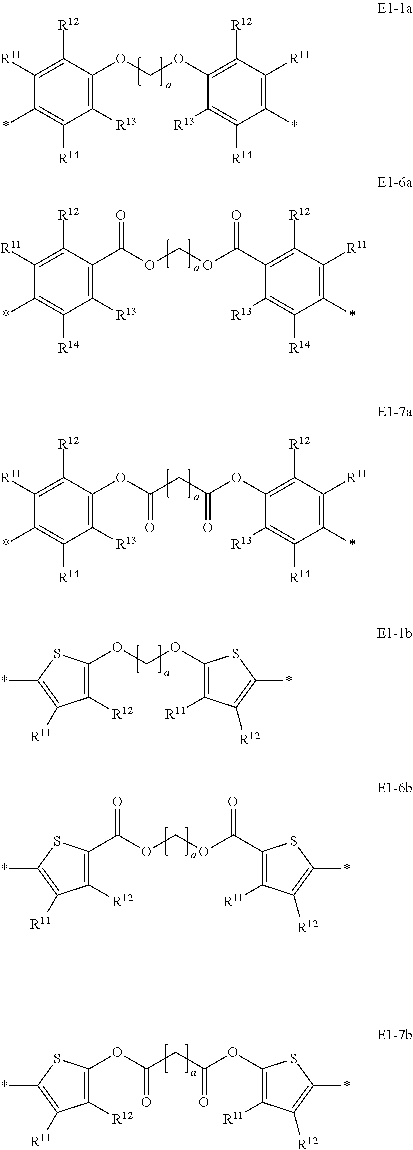
wherein a is an integer from 1 to 16 and R¹¹⁻¹⁴ independently of each other have one of the meanings of R⁵ as given in claim 3.

28. The polymer according to claim 1, characterized in that it comprises one or more units of formula I as defined in claim 1, and one or more units selected from the following groups
A2) the group consisting of arylene or heteroarylene selected from the group consisting of the formulae D1, D7, D10, D11, D19, D22, D29, D30, D35, D36, D37, D44, D55, D84, D87, D88, D89, D93, D94, D106, D111, D139, D140, D141, D146 and D150 as defined in claim 21,
and/or
A3) the group consisting of arylene or heteroarylene selected from the group consisting of the formulae A1, A6, A7, A15, A16, A20, A36, A74, A84, A88, A92, A94, A98 and A103 as defined in claim 22,
and/or
A4) the group consisting of arylene or heteroarylene selected from the group consisting of the formulae Sp1, Sp2, Sp6, Sp10, Sp11, Sp12, Sp13 and Sp14 s defined in claim 23,
and/or
D2) the group consisting of formulae D1-1 to D1-7 as defined in claim 24,
and/or
E2) the group consisting of formulae E1a, E1-1 to E1-7 and E1-1a to E1-7b as defined in claim 25, 26 or 27.

29. The polymer according to claim 1, characterized in that it is selected from the following subformulae 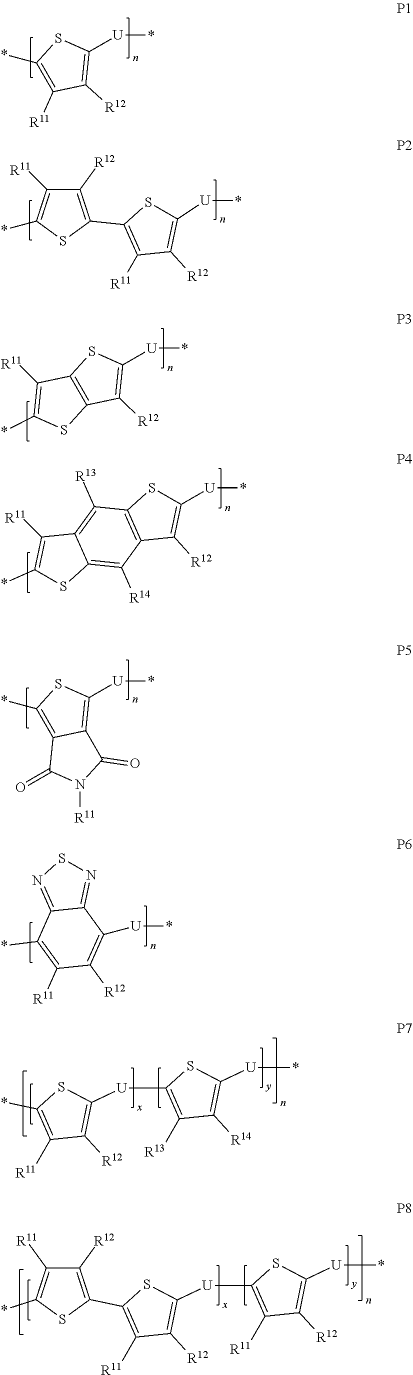
wherein R¹¹⁻¹⁴ independently of each other have the meanings given in claim 21, n is an integer >1, x is >0 and ≤1, y is ≥0 and <1, and U is a unit selected from formula I as defined in any of claims 1 to 14.

30. The polymer according to claim 1, characterized in that it is selected from the following subformulae 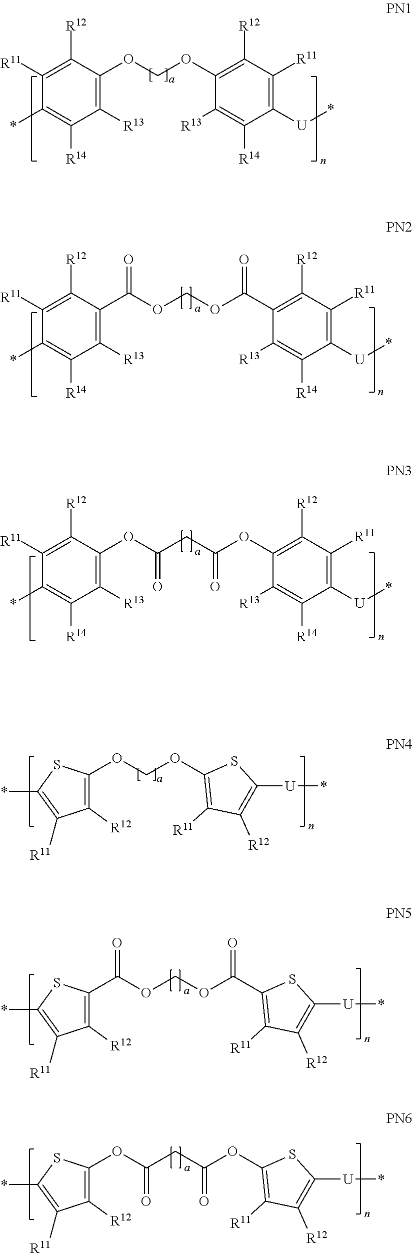
wherein R¹¹⁻¹⁴, U, a and n have the meanings given in claims 24 and 28.

31. The polymer according to claim 1, characterized in that it is selected of formula IV
R^{E1}-chain-R^{E2} IV
wherein “chain” denotes a polymer chain selected from formulae Pi-Px, P1-P8 or PN1-PN6 as defined in any of claims 19-30, and R^{E1} and R^{E2} have independently of each other one of the meanings of L as defined in claim 1, or denote, independently of each other, H, F, Br, Cl, I, —CH₂Cl, —CHO, —CR′═CR″₂, —SiR′R″R′″, —SiR′X′X″, —SiR′R″X′, —SnR′R″R′″, —BR′R″, —B(OR′)(OR″), —B(OH)₂, —O—SO₂—R′, —C≡CH, —C≡C—SiR′₃, —ZnX′ or an endcap group, X′ and X″ denote halogen, R′, R″ and R′″ have independently of each other one of the meanings of R⁰ given in claim 1, and two of R′, R″ and R′″ may also form a cyclosilyl, cyclostannyl, cycloborane or cycloboronate group with 2 to 20 C atoms together with the respective hetero atom to which they are attached.

32. A composition comprising one or more polymers according to claim 1, and further comprising one or more compounds having one or more of a semiconducting, hole or electron transporting, hole or electron blocking, electrically conducting, photoconducting, photoactive or light emitting property, and/or a binder.

33. The composition of claim 32, comprising an n-type semiconductor which is a polymer according to claim 1, and further comprising one or more p-type semiconductors, preferably selected from conjugated polymers.

34. The composition according to claim 32, comprising a second n-type semiconductors which is a fullerene or fullerene derivative, a non-fullerene acceptor small molecule, or an n-type conjugated polymer.

35. A bulk heterojunction (BHJ) formed from a composition according to claim 32.

36. Use of a polymer according to claim 1, or of a composition according to claim 32, in an electronic or optoelectronic device, or in a component of such a device or in an assembly comprising such a device.

37. A formulation comprising one or more polymers according to claim 1, or a composition according to claim 32, and further comprising one or more solvents selected from organic solvents.

38. An electronic or optoelectronic device, or a component thereof, or an assembly comprising it, which comprises a polymer according to claim 1, or a composition according to claim 32.

39. The electronic or optoelectronic device according to claim 38, which is selected from organic field effect transistors (OFET), organic thin film transistors (OTFT), organic light emitting diodes (OLED), organic light emitting transistors (OLET), organic light emitting electrochemical cells (OLEC), organic photovoltaic devices (OPV), organic photodetectors (OPD), organic solar cells, dye-sensitized solar cells (DSSC), perovskite-based solar cells (PSC), organic photoelectrochemical cells (OPEC), laser diodes, Schottky diodes, photoconductors, photodetectors, thermoelectric devices and LC windows.

40. The component according to claim 38, which is selected from charge injection layers, charge transport layers, interlayers, planarizing layers, antistatic films, polymer electrolyte membranes (PEM), conducting substrates and conducting patterns.

41. The assembly according to claim 38, which is selected from integrated circuits (IC), radio frequency identification (RFID) tags, security markings, security devices, flat panel displays, backlights of flat panel displays, electrophotographic devices, electrophotographic recording devices, organic memory devices, sensor devices, biosensors and biochips.

42. A monomer of formula V1 or V2
R^{R1}—(C¹)ₐ—U—(C²)_{b}—(C³)_{c}—(C⁴)_{d}—R^{R2} V1
R^{R1}—(C¹)ₐ—(C²)_{b}—U—(C³)_{c}—(C⁴)_{d}—R^{R2} V2
wherein U, C¹⁻⁴, a, b, c and d have the meanings of claim 17, and R^{R1} and R^{R2} are independently of each other selected from the group consisting of H, an activated C—H bond, Cl, Br, I, O-tosylate, O-triflate, O-mesylate, O-nonaflate, —SiMe₂F, —SiMeF₂, —O—SO₂Z¹, —B(OZ²)₂, —CZ³═C(Z³)₂, —C≡CH, —C≡CSi(Z¹)₃, —ZnX⁰ and —Sn(Z⁴)₃, wherein X⁰ is halogen, Z¹⁻⁴ are selected from the group consisting of alkyl and aryl, each being optionally substituted, and two groups Z² may also form a cycloboronate group having 2 to 20 C atoms together with the B- and O-atoms, and wherein at least one of R^{R1} and R^{R2} is different from H.

43. The monomer of claim 42, which is selected from the following subformulae
R^{R1}—U—R^{R2} V1a
R^{R1}—C¹—U—C²—R^{R2} V1b
R^{R1}—C¹—U—R^{R2} V1c
R^{R1}—U—C²—R^{R2} V1d
wherein U, C¹, C², R^{R1} and R^{R2} are as defined in claim 42.

44. A process of preparing a polymer according claim 1, by copolymerising one or more monomers of formula V1, V2 or V1a-d as defined in claims 42 and 43 with each other or with one or monomers of the following formulae in an aryl-aryl coupling reaction
R^{R1}—C¹—R^{R2} MI
R^{R1}—C²—R^{R2} MII
R^{R1}—C³—R^{R2} MIII
R^{R1}—C⁴—R^{R2} MIV
wherein C¹⁻⁴, R^{R1} and R^{R2} have the meanings given in claim 42.
